(19) United States
(12) Patent Application Publication
(10) Pub. No.: US 2024/0307374 A1
(43) Pub. Date: Sep. 19, 2024

(54) FORMULATION OF FURIN INHIBITOR FOR INHALATION

(71) Applicant: BP ASSET V, INC., Suite 400, MA (US)

(72) Inventors: Sheng Cui, Lexington, MA (US); Dinesh Shyamdeo Mishra, Carmel, IN (US)

(73) Assignee: BP ASSET V. INC., Cambridge, MA (US)

(21) Appl. No.: 18/275,531

(22) PCT Filed: Feb. 3, 2022
(86) PCT No.: PCT/US2022/015098

§ 371 Date: Aug. 2, 2023

Related U.S. Application Data
(60) Provisional application No. 63/145,363, filed on Feb. 3, 2021.

(51) Int. Cl. A61K 31/496; A61K 9/00; A61K 9/19; A61K 47/12; A61K 47/26

(52) U.S. Cl. CPC A61K 31/496; A61K 9/0075; A61K 9/0078; A61K 9/19; A61K 47/12; A61K 47/26

(57) ABSTRACT

Provided herein are pharmaceutical compositions comprising Compound (I), wherein Compound (I) is of the formula: or a pharmaceutically acceptable salt, polymorph, solvate, tautomer, stereoisomer, or isotopically labeled derivative thereof. Also disclosed are polymorphs of Compound (I) and methods of using the pharmaceutical compositions and polymorphs of Compound (I) as described herein to treat a disease (e.g., cystic fibrosis, and fibrotic diseases, such as pulmonary fibrosis) comprising administering to a subject in need thereof a therapeutically effective amount of a polymorph of Compound (I) or a pharmaceutical composition comprising Compound (I) as described herein. In some aspects, the compositions are formulated for inhalation (e.g., oral or nasal inhalation).

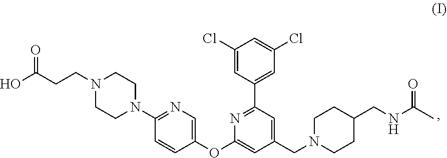 [SMILES: CC(=O)NCC1CCN(Cc2cc(Oc3ccc(N4CCN(CCC(=O)O)CC4)nc3)nc(-c3cc(Cl)cc(Cl)c3)c2)CC1 ; ","]

### RELATED APPLICATIONS

The present application claims priority under 35 U.S.C. § 119(e) to U.S. provisional application, U.S. Ser. No. 63/145,363, filed Feb. 3, 2021 which is incorporated herein by reference.

### BACKGROUND

Inactive precursor proteins of many enzymes, receptors and secreted proteins require processing and maturation to exert their biological functions (Thomas G. Nat. Rev. Mol. Cell. Biol. 2002, 3(10), 753-766). The proteolytic cleavage of pro-peptide sequences is dependent on the proprotein convertase (PC) family of calcium-dependent endoproteases. This PC family consists of the following serine proteases: proprotein convertase subtilisin kexin 1 (PCSK1), PCSK2, furin/PCSK3, PCSK4, PCSK5, PCSK6/paired basic amino acid cleaving enzyme 4 (PACE4), PCSK7, PCSK8/subtilisin kexin isoenzyme 1 (SK-1)/membrane bound transcription factor peptidase site 1 (MBTPS1) and PCSK9 (Thomas G. Nat. Rev. Mol. Cell. Biol. 2002, 3(10), 753-766; Nakayama K. Biochem. J. 1997, 327(3), 625-635; Klein-Szanto A J, Bassi D E. Biochem. Pharmacol. 2017, 140, 8-15; Turpeinen H, Ortutay Z, Pesu M. Curr. Genomics 2013, 14(7), 453-467). Among these PCSKs, furin (PCSK3) is well characterized and the most widely studied family member with diverse biological functions.

Furin is a 794 amino-acid type 1 transmembrane protein that is ubiquitously expressed in many cell types (Thomas G. Nat. Rev. Mol. Cell. Biol. 2002, 3(10), 753-766). It consists of the highly-conserved domain structure commonly found in PCSKs, including an N-terminal signal peptide, an inhibitory prodomain, a catalytic peptidase S8/S53 domain, a P domain, a cysteine-rich region and a cytoplasmic domain (Thomas G. Nat. Rev. Mol. Cell. Biol. 2002, 3(10), 753-766; Turpeinen H, Ortutay Z, Pesu M. Curr. Genomics 2013, 14(7), 453-467). The prodomain is essential for the proper folding, activation, and transport of furin, whereas the P domain regulates enzyme activity of the catalytic domain by modulating pH/calcium-dependent autoproteolytic cleavage process (Thomas G. Nat. Rev. Mol. Cell. Biol. 2002, 3(10), 753-766; Turpeinen H, Ortutay Z, Pesu M. Curr. Genomics 2013, 14(7), 453-467). Lastly, the cytoplasmic domain of furin allows for both efficient internalization from the plasma membrane and fast retrieval from the plasma membrane to the trans-Golgi network (TGN) (Thomas G. Nat. Rev. Mol. Cell. Biol. 2002, 3(10), 753-766).

Furin is predominantly localized in the trans-Golgi network (TGN) and the endosomal system, where it processes most of its diverse substrates in vivo. Furin's endoprotease activity is unmasked by release of its prodomain fragment, enabling furin to functionally process substrates in trans (Thomas G. Nat. Rev. Mol. Cell. Biol. 2002, 3(10), 753-766). Positioned after the carboxy-terminal arginine (Arg) residue, the cleavage site that furin cleaves is the sequence: -Arg-X-Lys/Arg-Arg↓- (Lys is lysine, X is any amino acid and ↓ identifies the cleavage site). Based on this substrate peptide amino acid motif, furin has >400 predicted target protein substrates, including hormones, growth factors, enzymes, receptors, neuropeptides, and infective agents (Turpeinen H, Ortutay Z, Pesu M. Curr. Genomics 2013, 14(7), 453-467; Shiryaev S A, Chernov A V, Golubkov V S, Thomsen E R, Chudin E, Chee M S, et al. PLoS One 2013, 8(1), e54290) (www.ebi.ac.uk/merops). The importance of the biological role of furin-dependent proteolytic processing can be further exemplified by the phenotypes of various studies with knock-out mice.

Germ-line furin knock-out mice studies demonstrate an important role for furin in embryonic development with embryonic lethality occurring between day 10.5 and 11.5. Failure of ventral closure and axial rotation as well as the absence of chorioallantoic fusion was observed. The impact of furin knock-down in endothelial cells resulted in cardiovascular defects, which included septal and valvular defects that may be attributed to impaired processing of TGFβ (Turpeinen H, Ortutay Z, Pesu M. Curr. Genomics 2013, 14(7), 453-467; Roebroek A J, Umans L, Pauli I G, Robertson E J, van Leuven F, Van de Ven W J, et al. Development 1998, 125(24), 4863-4876; Seidah N G, Prat A. Nat. Rev. Drug Discov. 2012, 11(5), 367-383; Constam D B, Robertson E J. Development 2000, 127(2), 245-254; Susan-Resiga D, Essalmani R, Hamelin J, Asselin M C, Benjannet S, Chamberland A, et al. J. Biol. Chem. 2011, 286(26), 22785-22794). However, knock-out of furin in the liver of adult mice (inducible Mx1-Cre transgene) is not lethal, and typical substrates of furin were cleaved although less efficiently pointing to possible redundancy among the PCSKs (Klein-Szanto A J, Bassi D E. Biochem. Pharmacol. 2017, 140, 8-15; Roebroek A J, Taylor N A, Louagie E, Pauli I, Smeijers L, Snellinx A, et al. J. Biol. Chem. 2004, 279(51), 53442-53450). In addition, targeted deletion of furin in T cells caused functional impairment of regulatory and effector T cells as a result of defective TGFβ 1 signaling (Pesu M, Watford W T, Wei L, Xu L, Fuss I, Strober W, et al. Nature 2008, 455(7210), 246-250). These observations implicate the role of furin in TGFβ biology and the potential therapeutic use of furin inhibitors for TGFβ-dependent diseases.

TGFβ family members play a key role in fibrosis (Dubois C M, Blanchette F, Laprise M H, Leduc R, Grondin F, Seidah N G. Am. J. Pathol. 2001, 158(1), 305-316), and TGFβ1 is elevated in fibrotic organs, such as heart, lung, and kidney (Pohlers D, Brenmoehl J, Löffler I, Müller CK, Leipner C, Schultze-Mosgau S, et al. Biochimica et Biophysica Acta (BBA)—Molecular Basis of Disease 2009, 1792(8), 746-756; Thomas B J, Kan O K, Loveland K L, Elias J A, Bardin P G. Am. J. Respir. Cell. Mol. Biol. 2016, 55(6), 759-766). Organ fibrosis is the result of aberrant wound healing response, resulting in excessive collagen deposition. Connective tissue scarring leads to progressive loss of tissue function and eventual organ failure (Nanthakumar C B, Hatley R J, Lemma S, Gauldie J, Marshall R P, Macdonald S J. Nat. Rev. Drug Discov. 2015, 14(10), 693-720). Pre-pro-TGFβ1 is synthesized by most cells as a single 390 amino acid peptide. The furin-dependent processing event is predicted to occur following an Arg-His-Arg-Arg sequence immediately preceding the NH₂-terminal Ala 279 residue of the growth factor (Constam D B. Seminars in Cell & Developmental Biology 2014, 32, 85-97). Mature TGFβ forms a 25 kDa dimer, which is complexed with specific binding proteins, such as the TGFβ latency-associated peptide (LAP) (NH₂-terminal part of the precursor sequence) and the large latent binding protein (LTBP) before secretion into the extracellular matrix (Constam D B. Seminars in Cell & Developmental Biology 2014, 32, 85-97; Robertson I B, Horiguchi M, Zilberberg L, Dabovic B, Hadjiolova K, Rifkin D B. Matrix biology, Journal of the International Society for Matrix Biology 2015, 47, 44-53). Active mature TGFβ1 must be liberated from the latent complex before it can exert its biological effects. The biological effects of TGFβ are mediated through the canonical SMAD-dependent signaling and the noncanonical pathways involving PI3K/ATK, Erk, and p38 upon receptor activation (Zhang Y E. Cell Research 2009, 19(1), 128-139). TGFβ1 drives the profibrotic responses by promoting the transformation of normal epithelial cell to active fibroblasts and the subsequent synthesis and deposition of collagen (Biernacka A, Dobaczewski M, Frangogiannis N G. Growth Factors (Chur, Switzerland) 2011, 29(5), 196-202). Thus, therapeutic intervention using a furin inhibitor would prevent the proper processing of Pre-pro-TGFβ1 and therefore provide benefit by depleting the bioactive TGFβ in fibrotic disease.

Given the diversity in its substrates, therapeutic intervention of furin could also be beneficial for diseases, such as hypertension, cancer, and infectious, respiratory, and neurodegenerative diseases (Thomas G. Nat. Rev. Mol. Cell. Biol. 2002, 3(10), 753-766; Nakayama K. Biochem. J. 1997, 327(3), 625-635; Shiryaev S A, Chernov A V, Golubkov V S, Thomsen E R, Chudin E, Chee M S, et al. PLoS One 2013, 8(1), e54290; Bennett B D, Denis P, Haniu M, Teplow D B, Kahn S, Louis J C, et al. J. Biol. Chem. 2000, 275(48), 37712-37717; Takahashi R H, Nagao T, Gouras G K. Pathology International 2017, 67(4), 185-193). Hypertension is a condition in which blood exerts increased force against the walls of the arteries. The renin-angiotensin system and molecules that regulate sodium-electrolyte balance can impact blood pressure and are associated with furin activity (Turpeinen H, Ortutay Z, Pesu M. Curr. Genomics 2013, 14(7), 453-467; Cousin C, Bracquart D, Contrepas A, Corvol P, Muller L, Nguyen G. Hypertension 2009, 53(6), 1077-1082). Two recent large-scale genetic association studies (GWAS) demonstrated a role for furin genetics as a risk factor for hypertension. One study utilized a GWAS approach to study over 200,000 subjects of European descent, thereby identifying a single nucleotide polymorphism (SNP; rs2521501) in the furin-FES loci associated with elevations in systolic and diastolic blood pressure (Ehret G B, Munroe P B, Rice K M, Bochud M, Johnson A D, et al. Nature 2011, 478(7367), 103-109). Two additional furin polymorphisms, rs2071410 and rs6227, which are associated with systolic and diastolic blood pressure respectively were identified in a second multi-center study that genotyped 50,000 SNPs amongst 2,100 candidate genes (Turpeinen H, Ortutay Z, Pesu M. Curr. Genomics 2013, 14(7), 453-467; Ganesh S K, Tragante V, Guo W, Guo Y, Lanktree M B, Smith E N, et al. Hum. Mol. Genet. 2013, 22(8), 1663-1678). Given such strong human genetic evidence, modulation of furin activity could be a therapeutic approach for hypertension.

Cancer is a set of diseases involving abnormal, uncontrolled growth of cells which may spread to other parts of the body (metastasis). There are furin substrates associated with various processes involved in cancer progression, such as proliferation, anti-apoptosis, migration/invasion, metastasis, and angiogenesis. The substrates that furin targets in these processes are growth factors and their receptors, matrix metalloproteases, cell adhesion molecules, and angiogenic/lymphangiogenic factors (Shiryaev S A, Chernov A V, Golubkov V S, Thomsen E R, Chudin E, Chee M S, et al. PLoS One 2013, 8(1), e54290; Jaaks P, Bernasconi M. Int. J. Cancer 2017, 141(4), 654-663; Bassi D E, Mahloogi H, Al-Saleem L, Lopez De Cicco R, Ridge J A, Klein-Szanto A J. Mol. Carcinog. 2001, 31(4), 224-232). Many growth factors and their receptors are important for the balance between apoptotic and prosurvival mechanisms. Therefore, dysregulation of growth factors plays a role in the development of cancer. In addition to uncontrolled growth, extracellular matrix (ECM) degradation is necessary for cancer cells to escape their primary site. Similarly, ECM remodeling is required for the development of the metastatic niche that enables disseminated cancer cells to survive, colonize, and proliferate at the metastatic site (Bonnans C, Chou J, Werb Z. Nat. Rev. Mol. Cell. Biol. 2014, 15(12), 786-801). Many of such enzymes like MMPs and ADAM proteases that mediate ECM degradation require proteolytic activation by furin (Maquoi E, Noel A, Frankenne F, Angliker H, Murphy G, Foidart J M. FEBS Lett. 1998, 424(3), 262-266; Yana I, Weiss S J. Mol. Biol. Cell 2000, 11(7), 2387-2401; Kang T, Nagase H, Pei D. Cancer Res. 2002, 62(3), 675-681; Wang X, Pei D. J. Biol. Chem. 2001, 276(38), 35953-35960; Loechel F, Gilpin B J, Engvall E, Albrechtsen R, Wewer U M. J. Biol. Chem. 1998, 273(27), 16993-16997; Schlondorff J, Becherer J D, Blobel C P. Biochem. J. 2000, 347(1), 131-138). Finally, angiogenesis, a process of blood vessels formation supports the growth of tumors. Vascular endothelial growth factors VEGF-C and VEGF-D are processed by furin, rendering them capable of promoting VEGF signaling, thereby stimulating angiogenesis and lymphangiogenesis (Joukov V, Sorsa T, Kumar V, Jeltsch M, Claesson-Welsh L, Cao Y, et al. EMBO J. 1997,16(13), 3898-3911; McColl B K, Paavonen K, Karnezis T, Harris N C, Davydova N, Rothacker J, et al. FASEB J. 2007, 21(4), 1088-1098). Therefore, therapeutic intervention of furin activities would limit the growth of cancer cells by blocking multiple key biological processes that promote the growth and spread of cancer cells.

Infectious diseases may be spread from one person to another and are caused by pathogenic microorganisms, such as bacteria, viruses, parasites or fungi. Pathogenicity is the ability of a microbial agent to cause disease, and virulence is the degree to which an organism is pathogenic. In order for viruses to enter host cells and replicate, the envelope glycoproteins must be proteolytically activated (Nakayama K. Biochem. J. 1997, 327(3), 625-635). The processing of envelope glycoproteins may in some cases impact viral pathogenicity (Nakayama K. Biochem. J. 1997, 327(3), 625-635). The glycoprotein precursors of many virulent viruses, such as human immunodeficiency virus (HIV), avian influenza virus, measles virus, respiratory syncytial virus (RSV), Ebola virus, anthrax, and Zika virus (ZIKV) are cleaved at a site marked by a consensus sequence consistent with furin recognition (Thomas G. Nat. Rev. Mol. Cell. Biol. 2002, 3(10), 753-766; 2, 36-38). The cleavage of HIV glycoprotein160 and infectious virus production are blocked when the furin inhibitor α1-PDX is expressed in cells (Nakayama K. Biochem. J. 1997, 327(3), 625-635). Thus, furin inhibitor may be useful in a pandemic or biological warfare.

Cystic fibrosis (CF) is a common life-limiting autosomal-recessive genetic disease in Europe and North America (Hoffman L R, Ramsey B W. CHEST 2013, 143(1), 207-213). A thin film of fluid lines the conducting airways of the lung facilitating mucociliary clearance, which contributes to innate immune defense by removing inhaled pathogens. The volume of this fluid is regulated by chloride and sodium transport across the airway epithelium. This regulation is lost in cystic fibrosis due to the absence of the cystic fibrosis transmembrane conduction regulator (CFTR), which mediates chloride secretion and subsequent sodium reabsorption and fluid balance across the epithelium. Epithelial sodium channel (ENaC) hyperabsorption is a contributing factor in the depletion of the fluid layer beginning the CF pathophysiology. Channel activating proteases (CAPs), such as furin, catalyze endoproteolysis of ENaC, and increase sodium channel conductance which would otherwise remain low (Reihill J A, Walker B, Hamilton R A, Ferguson T E, Elborn J S, Stutts M J, et al. Am. J. Respir. Crit. Care Med. 2016, 194(6), 701-710; Myerburg M M, Harvey P R, Heidrich E M, Pilewski J M, Butterworth M B. Am. J. Respir. Cell. Mol. Biol. 2010, 43(6), 712-719). A furin inhibitor is effective in blocking sodium reabsorption (Reihill J A, Walker B, Hamilton R A, Ferguson T E, Elborn J S, Stutts M J, et al. Am. J. Respir. Crit. Care Med. 2016, 194(6), 701-710) and thus providing proof of concept evidence for the potential use of a furin inhibitor in the treatment of CF.

Alzheimer's disease (AD) is a progressive, multifactorial, and heterogeneous neurodegenerative disease leading to progressive cognitive decline. Amyloid-β (Aβ)-containing plaques and neurofibrillary tangles composed of hyperphosphorylated tau in the brain are the neuropathological hallmarks of AD (Takahashi R H, Nagao T, Gouras G K. Pathology International 2017, 67(4), 185-193; Rangachari V, Dean D N, Rana P, Vaidya A, Ghosh P. Biochimica et Biophysica Acta (BBA)—Biomembranes 2018, doi.org/10.1016/j.bbamem.2018.03.004; Crews L, Masliah E. Human Molecular Genetics 2010, 19(R1), R12-R20). The amyloid precursor protein (APP) is an integral membrane protein containing a single transmembrane domain (Takahashi R H, Nagao T, Gouras G K. Pathology International 2017, 67(4), 185-193; Rangachari V, Dean D N, Rana P, Vaidya A, Ghosh P. Biochimica et Biophysica Acta (BBA)—Biomembranes 2018, doi.org/10.1016/j.bbamem.2018.03.004). Amyloid peptides can form by sequential cleavage of APP by the aspartyl proteases, β-(BACE) and γ-secretases (Takahashi R H, Nagao T, Gouras G K. Pathology International 2017, 67(4), 185-193; Rangachari V, Dean D N, Rana P, Vaidya A, Ghosh P. Biochimica et Biophysica Acta (BBA)—Biomembranes 2018, doi.org/10.1016/j.bbamem.2018.03.004; Fiala J C. Acta Neuropathologica 2007, 114(6), 551-571). Proteolytic cleavage of APP results in the generation of the Aβ1-42 monomer, which under pathological conditions can assemble into potentially toxic oligomers and form plaques (Takahashi R H, Nagao T, Gouras G K. Pathology International 2017, 67(4), 185-193; Rangachari V, Dean D N, Rana P, Vaidya A, Ghosh P. Biochimica et Biophysica Acta (BBA)—Biomembranes 2018, doi.org/10.1016/j.bbamem.2018.03.004; Fiala J C. Acta Neuropathologica 2007, 114(6), 551-571). It is suggested that amyloid deposition is initiated by glia that secrete Aβ. The protein spontaneously aggregates into amyloid filaments that activate microglia. Activated microglia then secrete oxidative species and inflammatory cytokines that cause axonal dystrophy and cell death (Rangachari V, Dean D N, Rana P, Vaidya A, Ghosh P. Biochimica et Biophysica Acta (BBA)—Biomembranes 2018, doi.org/10.1016/j.bbamem.2018.03.004; Crews L, Masliah E. Human Molecular Genetics 2010, 19(R1), R12-R20; Fiala J C. Acta Neuropathologica 2007, 114(6), 551-571). Mutations of APP and presenilins, components of the 7-secretases complex, lead to alteration of the APP processing by secretases and increase in production of pro-plaque forming Aβ peptides (Dai M H, Zheng H, Zeng L D, Zhang Y. Oncotarget 2018, 9(19), 15132-15143), suggesting the importance of secretases in disease progression. Therefore, pharmacological modulation of APP processing has been a prominent strategy for the treatment of AD, with both BACE and 7-secretases inhibitors being evaluated in recent clinical trials (Panza F, Seripa D, Solfrizzi V, Imbimbo B P, Lozupone M, Leo A, et al. Expert Opinion on Emerging Drugs 2016, 21(4), 377-391). BACE pro-peptide shares a consensus sequence for furin, and processing of BACE pro-peptide is shown to be dependent on active furin (Bennett B D, Denis P, Haniu M, Teplow D B, Kahn S, Louis J C, et al. J. Biol. Chem. 2000, 275(48), 37712-37717). Thus, selective furin inhibitors can potentially be used for the treatment of AD and neurodegenerative diseases associated with dysregulated furin processing.

### SUMMARY

Typically, potent furin inhibitors are peptide derivatives or peptidomimetics containing polybasic residues in order to achieve high inhibitory potency. As a consequence of the highly basic nature of the inhibitors, reactivity, and peptide structure, their chemical and pharmacokinetics properties limit their use as clinical therapeutic agents. However, many small molecule inhibitors of furin were reported in PCT publication No.: WO 2019/215341.

In one aspect, provided herein are pharmaceutical compositions comprising Compound (I), of the formula:

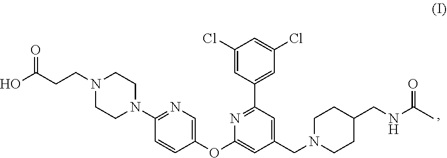 [SMILES: CC(=O)NCC1CCN(Cc2cc(Oc3ccc(N4CCN(CCC(=O)O)CC4)nc3)nc(-c3cc(Cl)cc(Cl)c3)c2)CC1 ; ","]

or pharmaceutically acceptable salts, solvates, tautomers, stereoisomers, or isotopically labeled derivatives thereof, or a polymorph thereof; and an organic acid. Preferably, the organic acid is selected from non-aromatic polycarboxylic acids and non-aromatic hydroxylated polycarboxylic acids, more preferably from non-aromatic hydroxylated di- and tricarboxylic acids, such as citric acid. In certain embodiments, the composition further comprises a pharmaceutically acceptable excipient (e.g., a tonicity agent such as a sugar (e.g., dextrose, lactose, trehalose, sucrose), sugar alcohol (e.g., mannitol), salt (e.g., sodium chloride, potassium chloride), or polyol (e.g., propylene glycol, glycerin)). In certain embodiments, the tonicity agent is a sugar. In certain embodiments, the tonicity agent is lactose. In certain embodiments, the composition is a solution. In certain embodiments, the composition is a powder. In certain embodiments, the composition is a powder obtained by lyophilization of an aqueous solution comprising Compound (I), or pharmaceutically acceptable salt, solvate, tautomer, stereoisomer, or isotopically labeled derivative thereof, or a polymorph thereof.

In another aspect, provided herein are compositions comprising Compound (I) for use in the treatment of a disease (e.g., cystic fibrosis, fibrotic diseases (e.g., pulmonary fibrosis)). In some embodiments, the compositions comprising Compound (I) as described herein are formulated for inhalation (e.g., oral and/or nasal inhalation). In other embodiments, the compositions comprising Compound (I) are formulated for administration via a nebulizer. In other embodiments, the compositions comprising Compound (I) are formulated for administration via an inhaler (e.g., a dry powder inhaler). In some aspects, provided herein are methods of treating a fibrotic disease or condition comprising administering to a subject in need thereof a therapeutically effective amount of the pharmaceutical composition comprising Compound (I).

Also provided herein are polymorphs of Compound (I), wherein Compound (I) is of the formula:

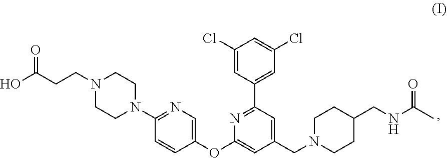 [SMILES: CC(=O)NCC1CCN(Cc2cc(Oc3ccc(N4CCN(CCC(=O)O)CC4)nc3)nc(-c3cc(Cl)cc(Cl)c3)c2)CC1 ; ","]

or a pharmaceutically acceptable salt, solvate, tautomer, stereoisomer, or isotopically labeled derivative. Polymorphic forms of Compound (I) include Freeform Type A, Freeform Type B, Freeform Type C, and Freeform Type D as described herein. Further provided herein are compositions comprising Compound (I) and polymorphs thereof for use in the treatment of a disease (e.g., cystic fibrosis, fibrotic diseases (e.g., pulmonary fibrosis)). In some embodiments, the compositions comprising Compound (I) and polymorphs thereof as described herein are formulated for inhalation (e.g., oral and/or nasal inhalation). In other embodiments, the compositions comprising Compound (I) and polymorphs thereof are formulated for administration via a nebulizer. In other embodiments, the compositions comprising Compound (I) and polymorphs thereof are formulated for administration via an inhaler (e.g., a dry powder inhaler). In some aspects, provided herein are methods of treating a fibrotic disease or condition comprising administering to a subject in need thereof a therapeutically effective amount of the polymorph of Compound (I), or a pharmaceutical composition comprising Compound (I). In another aspect, provided herein are methods of treating cystic fibrosis comprising administering to a subject in need thereof a therapeutically effective amount of the polymorph of Compound (I), or a pharmaceutical composition comprising Compound (I).

In certain embodiments, Freeform Type D is characterized by at least one of:
a. an X-ray powder diffraction pattern obtained by irradiation with Cu-Kα having three or more peaks, expressed in degrees 2-theta±0.2°, selected from 4.07, 10.03, 12.01, 12.53, 14.68, 17.01, 17.27, 18.29, 18.91, 19.89, 20.33, 21.4, 21.62, 22.27, 22.85, 23.25, 24.41, 25.14, 25.65, 26.08, 26.63, 27.18, 28.53, 29.04, 30.45, 32.37, and 35.01; and/or
b. a DSC thermogram showing an endotherm at about 106.7° C.

In certain embodiments, Freeform Type D is characterized by an X-ray powder diffraction pattern substantially identical to the X-ray powder diffraction (XRPD) pattern shown in FIG. 186.

In another aspect, provided herein are pharmaceutical compositions comprising a polymorph of Compound (I) (e.g., Freeform Type D). Further provided herein are pharmaceutical compositions comprising a polymorph of Compound (I) (e.g., Freeform Type D), and a pharmaceutically acceptable excipient (e.g., citric acid) and, optionally, a second pharmaceutically acceptable excipient (e.g., tonicity agent(e.g., lactose)). Also provided herein are pharmaceutical compositions comprising a polymorph of Compound (I), or a solvate or pharmaceutically acceptable salt thereof (e.g., Freeform Type D); a first pharmaceutically acceptable excipient (e.g., citric acid); and a second pharmaceutically acceptable excipient (e.g., tonicity agent (e.g., lactose)).

Also disclosed are methods of using a polymorph of Compound (I), or composition, as described herein to treat a disease (e.g., cystic fibrosis, or fibrotic diseases (e.g., pulmonary fibrosis)) comprising administering to a subject in need thereof a therapeutically effective amount of a polymorph of Compound (I), or a pharmaceutical composition, as described herein. In another aspect, this disclosure provides a polymorph of Compound (I), or a solvate or pharmaceutically acceptable salt thereof, or a composition comprising a polymorph of Compound (I), for use in the manufacture of a medicament for the treatment of a disorder mediated by or associated with furin, such as fibrotic diseases.

The details of certain embodiments of the disclosure are set forth in the Detailed Description of Certain Embodiments, as described below. Other features, objects, and advantages of the disclosure will be apparent from the Brief Description of the Drawings, Definitions, Examples, and Claims.

### BRIEF DESCRIPTION OF THE DRAWINGS

FIG. 1. XRPD overlay of Compound (I) freeform polymorphs.

FIG. 2. XRPD pattern of freeform Type A.

FIG. 3. Thermogravimetric analysis (TGA) and differential scanning calorimetry (DSC) curves of freeform Type A.

FIG. 4. ¹1H NMR spectrum of freeform Type A.

FIG. 5. Dynamic vapor sorption (DVS) plot of freeform Type A.

FIG. 6. XRPD overlay of freeform Type A before and after DVS test.

FIG. 7. XRPD overlay of freeform Type B before and after drying.

FIG. 8. TGA/DSC curves of freeform Type B.

FIG. 9. ¹1H NMR spectrum of freeform Type B.

FIG. 10. XRPD pattern of re-prepared freeform Type B.

FIG. 11. TGA/DSC curves of re-prepared freeform Type B.

FIG. 12. ¹H NMR spectrum of re-prepared freeform Type B.

FIG. 13. Variable temperature-XRPD (VT-XRPD) results of re-prepared freeform Type B.

FIG. 14. TGA/DSC curves of freeform Type B after VT-XRPD test.

FIG. 15. DVS plot of re-prepared freeform Type B.

FIG. 16. XRPD overlay of re-prepared freeform Type B before and after DVS test.

FIG. 17. XRPD overlay of freeform Type C before and after drying.

FIG. 18. XRPD pattern of freeform Type D.

FIG. 19. TGA/DSC curves of freeform Type D.

FIG. 20. ¹H NMR spectrum of freeform Type D.

FIG. 21. DVS plot of freeform Type D.

FIG. 22. XRPD overlay of freeform Type D before and after drying.

FIG. 23. XRPD overlay of humidity induced experiments for freeform Type D.

FIG. 24. XRPD overlay of slurry competition of freeform Type A and Type B.

FIG. 25. XRPD overlay of re-performed slurry competition of freeform Type A and Type B.

FIG. 26. XRPD overlay of slurry competition of freeform Type B and Type D.

FIG. 27. XRPD overslay of slurry competition of freeform Type A and Type D (a_{w} from 0.4 to 1.0).

FIG. 28. XRPD overslay of slurry competition of freeform Type A and Type D (a_{w} from 0.3 to 0.5).

FIG. 29. Speciation diagram of freeform.

FIG. 30. Speciation diagram of freeform.

FIG. 31. Speciation structure of freeform.

FIG. 32. Solubility curves of freeform Type D in CD solutions.

FIG. 33. XRPD results of solid obtained after equilibrium in HPβCD solutions.

FIG. 34. XRPD results of solid obtained after equilibrium in SBECD solutions.

FIG. 35. XRPD pattern of HCl salt Type F.

FIG. 36. TGA/DSC curves of HCl salt Type F.

FIG. 37. ¹H NMR spectrum of HCl salt Type F.

FIG. 38. DVS plot of HCl salt Type F.

FIG. 39. XRPD overlay of HCl salt Type F before and after DVS.

FIG. 40. XRPD pattern of sulfate Type B.

FIG. 41. TGA/DSC curves of sulfate Type B.

FIG. 42. ¹H NMR spectrum of sulfate Type B.

FIG. 43. DVS plot of sulfate Type B.

FIG. 44. XRPD overlay of sulfate Type B before and after DVS.

FIG. 45. XRPD pattern of fumarate Type A.

FIG. 46. TGA/DSC curves of fumarate Type A.

FIG. 47. ¹H NMR spectrum of fumarate Type A.

FIG. 48. DVS plot of fumarate Type A.

FIG. 49. XRPD overlay of fumarate Type A before and after DVS.

FIG. 50. XRPD overlay of freeform Type A before and after grinding.

FIG. 51. XRPD overlay of sulfate Type B before and after grinding.

FIG. 52. XRPD overlay of fumarate Type A before and after grinding.

FIG. 53. XRPD overlay of HCl salt Type F before and after grinding.

FIG. 54. XRPD results of freeform Type A after solubility evaluation.

FIG. 55. XRPD results of sulfate Type B after solubility evaluation (I/III).

FIG. 56. XRPD results of sulfate Type B after solubility evaluation (II/III).

FIG. 57. XRPD results of sulfate Type B after solubility evaluation (III/III).

FIG. 58. XRPD results of fumarate Type A after solubility evaluation (I/II).

FIG. 59. XRPD results of fumarate Type A after solubility evaluation (II/II).

FIG. 60. XRPD results of HCl salt Type F after solubility evaluation (I/II).

FIG. 61. XRPD results of HCl salt Type F after solubility evaluation (II/II).

FIG. 62. XRPD results of freeform Type A after solubility evaluation.

FIG. 63. XRPD results of sulfate Type B after solubility evaluation.

FIG. 64. XRPD results of fumarate Type A after solubility evaluation.

FIG. 65. XRPD results of HCl salt Type F after solubility evaluation.

FIG. 66. Chromatograms overlay of in situ salt formation experiments.

FIG. 67. XRPD results of slurry experiments of amorphous sample.

FIG. 68. ¹H NMR spectrum of freeform Type D.

FIG. 69. ¹H NMR spectrum of amorphous sample.

FIG. 70. ¹H NMR spectrum of mixture of freeform Type D and citric acid.

FIG. 71. ¹H NMR overlay of freeform Type D and amorphous sample.

FIG. 72. ¹H NMR overlay of freeform Type D+citric acid and amorphous sample.

FIG. 73. XPS overlay of freeform Type D and amorphous sample.

FIG. 74. Solubility curve of freeform Type D in citrate buffers.

FIG. 75. XRPD results of solids obtained in 10 mM citrate buffers.

FIG. 76. XRPD results of solids obtained in 20 mM citrate buffers.

FIG. 77. XRPD results of solids obtained in 50 mM citrate buffers.

FIG. 78. XRPD results of solids obtained in 100 mM citrate buffers.

FIG. 79. Visual observation of stability samples in citrate buffers.

FIG. 80. Chromatograms overlay of freeform Type D after stability evaluation in 10 mM pH=4.3 citrate buffer (1 mg/mL).

FIG. 81. Chromatograms overlay of freeform Type D after stability evaluation in 100 mM pH=4.1 citrate buffer (40 mg/mL).

FIG. 82. Impurity (RRT around 1.23) increase plot of formulation 1.

FIG. 83. Impurity (RRT around 1.23) increase plot of formulation 2.

FIG. 84. Impurity (RRT around 1.23) increase plot of formulation 3.

FIG. 85. Impurity (RRT around 1.23) increase plot of formulation 4.

FIG. 86. Impurity (RRT around 1.23) increase plot of formulation 5.

FIG. 87. Chromatograms overlay of solution stability samples in formulation 1 at 25° C.

FIG. 88. Chromatograms overlay of solution stability samples in formulation 1 at 40° C.

FIG. 89. Chromatograms overlay of solution stability samples in formulation 1 at 60° C.

FIG. 90. Chromatograms overlay of solution stability samples in formulation 2 at 25° C.

FIG. 91. Chromatograms overlay of solution stability samples in formulation 2 at 40° C.

FIG. 92. Chromatograms overlay of solution stability samples in formulation 2 at 60° C.

FIG. 93. Chromatograms overlay of solution stability samples in formulation 3 at 25° C.

FIG. 94. Chromatograms overlay of solution stability samples in formulation 3 at 40° C.

FIG. 95. Chromatograms overlay of solution stability samples in formulation 3 at 60° C.

FIG. 96. Chromatograms overlay of solution stability samples in formulation 4 at 25° C.

FIG. 97. Chromatograms overlay of solution stability samples in formulation 4 at 40° C.

FIG. 98. Chromatograms overlay of solution stability samples in formulation 4 at 60° C.

FIG. 99. Chromatograms overlay of solution stability samples in formulation 5 at 25° C.

FIG. 100. Chromatograms overlay of solution stability samples in formulation 5 at 40° C.

FIG. 101. Chromatograms overlay of solution stability samples in formulation 5 at 60° C.

FIG. 102. XRPD results of solid obtained in formulation 1/2/3/4/5 after stability evaluation at 5° C.

FIG. 103. Chromatograms overlay of solution stability samples in formulation 1 at 5° C.

FIG. 104. Chromatograms overlay of solution stability samples in formulation 2 at 5° C.

FIG. 105. Chromatograms overlay of solution stability samples in formulation 3 at 5° C.

FIG. 106. Chromatograms overlay of solution stability samples in formulation 4 at 5° C.

FIG. 107. Chromatograms overlay of solution stability samples in formulation 5 at 5° C.

FIG. 108. ¹H NMR spectrum of solid obtained in formulation 1 at 5° C.

FIG. 109. ¹H NMR spectrum of solid obtained in formulation 2 at 5° C.

FIG. 110. ¹H NMR spectrum of solid obtained in formulation 3 at 5° C.

FIG. 111. ¹H NMR spectrum of solid obtained in formulation 4 at 5° C.

FIG. 112. ¹H NMR spectrum of solid obtained in formulation 5 at 5° C.

FIG. 113. Chromatograms overlay of 40 mg/mL freeform+citric acid+lactose formulation stability experiments.

FIG. 114. XRPD results of freeform Type D after stability evaluation at 25° C./60% RH.

FIG. 115. XRPD results of freeform Type D after stability evaluation at 40° C./75% RH.

FIG. 116. XRPD results of freeform Type D after stability evaluation at 60° C.

FIG. 117. Chromatograms overlay of freeform Type D after stability evaluation at 25° C./60% RH.

FIG. 118. Chromatograms overlay of freeform Type D after stability evaluation at 40° C./75% RH.

FIG. 119. Chromatograms overlay of freeform Type D after stability evaluation at 60° C.

FIG. 120. XRPD pattern of Compound (I) starting material.

FIG. 121. TGA/DSC curves of Compound (I) starting material.

FIG. 122. LC-MS result of Compound (I) starting material.

FIG. 123. PLM image of Compound (I) starting material.

FIG. 124. ¹H NMR spectrum of Compound (I) starting material (MeOH-d3).

FIG. 125. XRPD overlay of solid obtained during freeform isolation procedure optimization.

FIG. 126. XRPD overlay solid obtained in step 3 during freeform isolation on 7 g scale.

FIG. 127. XRPD overlay of Compound (I) starting material and freeform Type D.

FIG. 128. TGA/DSC curves of Compound (I) starting material.

FIG. 129. ¹H NMR spectrum of Compound (I) starting material.

FIG. 130. PLM image of Compound (I) starting material.

FIG. 131. DVS plot of Compound (I) starting material.

FIG. 132. XRPD overlay of Compound (I) starting material before and after DVS test.

FIG. 133. TGA overlay of Compound (I) starting material before and after storage under ambient condition.

FIG. 134. XRPD overlay of HCl salt forms.

FIG. 135. TGA/DSC curves of HCl salt Type A.

FIG. 136. ¹H NMR spectrum of HCl salt Type A.

FIG. 137. TGA/DSC curves of HCl salt Type D.

FIG. 138. ¹H NMR spectrum of HCl salt Type D.

FIG. 139. TGA/DSC curves of HCl salt Type E.

FIG. 140. ¹H NMR spectrum of HCl salt Type E.

FIG. 141. XRPD overlay of sulfate forms.

FIG. 142. ¹H NMR spectrum of sulfate Type A.

FIG. 143. TGA/DSC curves of sulfate Type B.

FIG. 144. ¹H NMR spectrum of sulfate Type B.

FIG. 145. XRPD overlay of maleate forms.

FIG. 146. TGA/DSC curves of maleate Type A.

FIG. 147. ¹H NMR spectrum of maleate Type A.

FIG. 148. TGA/DSC curves of maleate Type B.

FIG. 149. ¹H NMR spectrum of maleate Type B.

FIG. 150. XRPD pattern of tartrate Type A.

FIG. 151. TGA/DSC curves of tartrate Type A.

FIG. 152. ¹H NMR spectrum of tartrate Type A.

FIG. 153. XRPD overlay of fumarate forms.

FIG. 154. TGA/DSC curves of fumarate Type A.

FIG. 155. ¹H NMR spectrum of fumarate Type A.

FIG. 156. TGA/DSC curves of fumarate Type B.

FIG. 157. ¹H NMR spectrum of fumarate Type B.

FIG. 158. TGA/DSC curves of fumarate Type C.

FIG. 159. ¹H NMR spectrum of fumarate Type C.

FIG. 160. XRPD overlay of succinate forms.

FIG. 161. ¹H NMR spectrum of succinate Type A.

FIG. 162. TGA/DSC curves of succinate Type B.

FIG. 163. ¹H NMR spectrum of succinate Type B.

FIG. 164. TGA/DSC curves of succinate Type C.

FIG. 165. ¹H NMR spectrum of succinate Type C.

FIG. 166. XRPD pattern of triphenylacetate Type A.

FIG. 167. TGA/DSC curves of triphenylacetate Type A.

FIG. 168. ¹H NMR spectrum of triphenylacetate Type A.

FIG. 169. XRPD pattern of xinafoic salt Type A.

FIG. 170. TGA/DSC curves of xinafoic salt Type A.

FIG. 171. ¹H NMR spectrum of xinafoic salt Type A.

FIG. 172. XRPD pattern of Ca²⁺ salt Type A.

FIG. 173. TGA/DSC curves of Ca²⁺ salt Type A.

FIG. 174. ¹H NMR spectrum of Ca²⁺ salt Type A.

FIG. 175. XRPD pattern of tromethamine salt forms.

FIG. 176. TGA/DSC curves of tromethamine salt Type A.

FIG. 177. ¹H NMR spectrum of tromethamine salt Type A.

FIG. 178. TGA/DSC curves of tromethamine salt Type B.

FIG. 179. ¹H NMR spectrum of tromethamine salt Type B.

FIG. 180. XRPD result of solid obtained in pH=4.0 citrate/phosphate buffer.

FIG. 181. XRPD result of solid obtained in pH=5.0 citrate/phosphate buffer.

FIG. 182. XRPD pattern of form X.

FIG. 183. XRPD pattern of form Y.

FIG. 184. XRPD pattern of freeform Type B.

FIG. 185. XRPD pattern of freeform Type C.

FIG. 186. XRPD pattern of freeform Type D.

FIG. 187 Single crystal structure of Compound (I) trihydrate.

### DEFINITIONS

Terms are used within their ordinary and accepted meanings. The following definitions are meant to clarify, but not limit, the terms defined herein.

The term “about,” as used herein, is used to describe a range (e.g., of temperatures, of molarities, of mass, of weight) and is given its ordinary meaning in the art, typically referring to the error associated with an instrument to collect a measurement or reading. The term “about” may also refer to a 10 to 20% variation around a given value. In general, the term “about” when referring to temperature provides a deviation of ±0-2° C.

As used herein, the term “salt” refers to an acid or base salt of Compound (I). Pharmaceutically acceptable salts can be derived, for example, from mineral acids (hydrochloric acid, hydrobromic acid, phosphoric acid, and the like), organic acids (acetic acid, propionic acid, glutamic acid, citric acid and the like), and quaternary ammonium ions. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The neutral form of a compound may be regenerated by contacting the salt with a base or acid and isolating the parent compound in the conventional manner. The parent form of the compound differs from the various salt forms in certain physical properties, such as solubility in polar solvents.

The term “room temperature” or “RT” refers to a temperature within the range of 19-26° C.

The term “solvate” refers to forms of the compound that are associated with a solvent, usually by a solvolysis reaction. This physical association may include hydrogen bonding. Conventional solvents include water, methanol, ethanol, acetic acid, dimethyl sulfoxide (DMSO), tetrahydrofuran (THF), diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates. In certain aspects, a solvate is a distinct polymorph. In some aspects, a solvate is not a distinct polymorph, i.e., a defined polymorph with a distinct crystal structure may contain residual solvent molecules.

The term “amorphous” or “amorphous form” refers to a form of a solid (“solid form”), the form substantially lacking three-dimensional order. In certain embodiments, an amorphous form of a solid is a solid form that is substantially not crystalline. In certain embodiments, the X-ray powder diffraction (XRPD) pattern of an amorphous form includes a wide scattering band with a peak at 20 of, e.g., between 20 and 70°, inclusive, using CuKα radiation. In certain embodiments, the XRPD pattern of an amorphous form further includes one or more peaks attributed to crystalline structures. In certain embodiments, the maximum intensity of any one of the one or more peaks attributed to crystalline structures observed at a 2θ of between 20 and 70°, inclusive, is not more than 300-fold, not more than 100-fold, not more than 30-fold, not more than 10-fold, or not more than 3-fold of the maximum intensity of the wide scattering band. In certain embodiments, the XRPD pattern of an amorphous form includes no peaks attributed to crystalline structures.

The term “polymorph” or “polymorphic form”, as used herein, refers to a crystalline form of a compound (or a salt, hydrate, or solvate thereof) in a particular crystal packing arrangement. All polymorphs have the same elemental composition. Different crystalline forms usually have different X-ray diffraction patterns, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

A “freeform” of Compound (I) (e.g., Freeform Type D) is a polymorphic form of the free base of Compound (I) or a solvate, tautomer, stereoisomer, or isotopically labeled derivative thereof. Freeforms of Compound (I) include Freeform Type A, Freeform Type B, Freeform Type C, and Freeform Type D.

The term “crystalline” refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (melting point). The term “crystalline” or “crystalline form” refers to a solid form substantially exhibiting three-dimensional order. In certain embodiments, a crystalline form of a solid is a solid form that is substantially not amorphous. In certain embodiments, the X-ray powder diffraction (XRPD) pattern of a crystalline form includes one or more sharply defined peaks.

When a polymorphic form is described, it is meant to refer to the identified polymorph as described herein, which is substantially free of any other polymorph. “Substantially free of” another polymorph indicates at least a 70/30 molar ratio of the two polymorphs, more preferably 80/20, 90/10, 95/5, 99/1, or more. In some embodiments, one of the polymorphs will be present in an amount of at least 99%.

The polymorphs of Compound (I) may also contain unnatural proportions of atomic isotopes at one or more of the atoms that constitute such compounds. Unnatural proportions of an isotope may be defined as ranging from the amount found in nature to an amount consisting of 100% of the atom in question. For example, the compounds may incorporate radioactive isotopes, such as tritium (³H) or carbon-14 (¹⁴C), or non-radioactive isotopes, such as deuterium (²H) or carbon-13 (¹³C). Such isotopic variations can provide additional utilities to those described elsewhere within this application. For instance, isotopic variants of the Compound (I) may find additional utility, including, but not limited to, as diagnostic and/or imaging reagents, or as cytotoxic/radiotoxic therapeutic agents. Additionally, isotopic variants of Compound (I) can have altered pharmacokinetic and pharmacodynamic characteristics, which can contribute to enhanced safety, tolerability, or efficacy during treatment. All isotopic variations of Compound (I), whether radioactive or not, are intended to be encompassed within the scope of the present disclosure. When specifically referred to, such as, C₁-C₄ deuteroalkyl—the term refers to an alkyl group with the indicated number of carbon atoms and having hydrogen atoms replaced by deuterium in a number of from one to a per-deutero form, wherein the deuterium replacement is greater than the natural abundance of deuterium—typically 50%, 60%, 70%, 80%, 90%, 95% or more deuterium replacement. Examples of C₁-C₄ deuteroalkyl are —CD₃, —CH₂CD₃, —CD₂CD₃, —CH₂CH₂CH₂D, and the like.

As used herein, the term “pharmaceutically acceptable” refers to a substance that is compatible with Compound (I), as well as with any other ingredients with which the compound is formulated. Furthermore, a pharmaceutically acceptable substance is not deleterious to the recipient of the substance. The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference.

Pharmaceutically acceptable salts of Compound (I) include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid, or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, the term “pharmaceutical composition” refers to a product comprising Compound (I), optionally, an excipient and/or a second pharmaceutically acceptable excipient (e.g., a tonicity agent, organic acid), and other optional ingredients in specified amounts, as well as any product which results directly or indirectly from combination of the specified ingredients in the specified amounts. In certain embodiments, the pharmaceutical composition comprises Compound (I), or a pharmaceutically acceptable salt, solvate, tautomer, stereoisomer, or isotopically labeled derivative thereof. In certain embodiments, the pharmaceutical composition comprises a polymorph of Compound (I). In certain embodiments, the pharmaceutical composition comprises an amorphous form of Compound (I).

As used herein, the term “excipient” refers to a substance that aids the administration of an active agent to a subject. Pharmaceutically acceptable excipients useful in the present disclosure include, but are not limited to, those described herein. One of skill in the art will recognize that other excipients can be useful in the present disclosure.

As used herein, the term “tonicity agent” refers to an agent which functions to render a solution similar in osmotic characteristics to physiologic fluids. Tonicity agents include, but are not limited to, sugars (e.g., dextrose, lactose, trehalose, sucrose), sugar alcohols (e.g., mannitol), salts (e.g., sodium chloride, potassium chloride), and polyols (e.g., propylene glycol, glycerin).

The term “sugar” refers to monosaccharides, disaccharides, or polysaccharides. Monosaccharides are the simplest carbohydrates in that they cannot be hydrolyzed to smaller carbohydrates. Most monosaccharides can be represented by the general formula C_{y}H_{2y}O_{y} (e.g., C₆H₁₂O₆ (a hexose such as glucose)), wherein y is an integer equal to or greater than 3. Certain polyhydric alcohols not represented by the general formula described above may also be considered monosaccharides. For example, deoxyribose is of the formula C₅H₁₀O₄ and is a monosaccharide. Monosaccharides usually consist of five or six carbon atoms and are referred to as pentoses and hexoses, receptively. If the monosaccharide contains an aldehyde it is referred to as an aldose; and if it contains a ketone, it is referred to as a ketose. Monosaccharides may also consist of three, four, or seven carbon atoms in an aldose or ketose form and are referred to as trioses, tetroses, and heptoses, respectively. Glyceraldehyde and dihydroxyacetone are considered to be aldotriose and ketotriose sugars, respectively. Examples of aldotetrose sugars include erythrose and threose; and ketotetrose sugars include erythrulose. Aldopentose sugars include ribose, arabinose, xylose, and lyxose; and ketopentose sugars include ribulose, arabulose, xylulose, and lyxulose. Examples of aldohexose sugars include glucose (for example, dextrose), mannose, galactose, allose, altrose, talose, gulose, and idose; and ketohexose sugars include fructose, psicose, sorbose, and tagatose. Ketoheptose sugars include sedoheptulose. Each carbon atom of a monosaccharide bearing a hydroxyl group (—OH), with the exception of the first and last carbons, is asymmetric, making the carbon atom a stereocenter with two possible configurations (R or S). Because of this asymmetry, a number of isomers may exist for any given monosaccharide formula. The aldohexose D-glucose, for example, has the formula C₆H₁₂O₆, of which all but two of its six carbons atoms are stereogenic, making D-glucose one of the 16 (i.e., 2⁴) possible stereoisomers. The assignment of D or L is made according to the orientation of the asymmetric carbon furthest from the carbonyl group: in a standard Fischer projection if the hydroxyl group is on the right the molecule is a D sugar, otherwise it is an L sugar. The aldehyde or ketone group of a straight-chain monosaccharide will react reversibly with a hydroxyl group on a different carbon atom to form a hemiacetal or hemiketal, forming a heterocyclic ring with an oxygen bridge between two carbon atoms. Rings with five and six atoms are called furanose and pyranose forms, respectively, and exist in equilibrium with the straight-chain form. During the conversion from the straight-chain form to the cyclic form, the carbon atom containing the carbonyl oxygen, called the anomeric carbon, becomes a stereogenic center with two possible configurations: the oxygen atom may take a position either above or below the plane of the ring. The resulting possible pair of stereoisomers is called anomers. In an a anomer, the —OH substituent on the anomeric carbon rests on the opposite side (trans) of the ring from the —CH₂OH side branch. The alternative form, in which the —CH₂OH substituent and the anomeric hydroxyl are on the same side (cis) of the plane of the ring, is called a R anomer. A carbohydrate including two or more joined monosaccharide units is called a disaccharide or polysaccharide (e.g., a trisaccharide), respectively. The two or more monosaccharide units bound together by a covalent bond known as a glycosidic linkage formed via a dehydration reaction, resulting in the loss of a hydrogen atom from one monosaccharide and a hydroxyl group from another. Exemplary disaccharides include sucrose, lactulose, lactose, maltose, isomaltose, trehalose, cellobiose, xylobiose, laminaribiose, gentiobiose, mannobiose, melibiose, nigerose, or rutinose. Exemplary trisaccharides include, but are not limited to, isomaltotriose, nigerotriose, maltotriose, melezitose, maltotriulose, raffinose, and kestose. The term carbohydrate also includes other natural or synthetic stereoisomers of the carbohydrates described herein.

The term “carbohydrate” or “saccharide” refers to an aldehydic or ketonic derivative of polyhydric alcohols. Carbohydrates include compounds with relatively small molecules (e.g., sugars) as well as macromolecular or polymeric substances (e.g., starch, glycogen, and cellulose polysaccharides).

In some embodiments, treatment may be administered after one or more signs or symptoms of the disease have developed or have been observed. As used herein, the terms “treat,” “treating,” and “treatment” refer to any indicia of success in the treatment or amelioration of a pathology, injury, condition, or symptom related to pulmonary disorders, including any objective or subjective parameter, such as abatement; remission; diminishing of symptoms; making the pathology, injury, condition, or symptom more tolerable to the patient; decreasing the frequency or duration of the pathology, injury, condition, or symptom; or, in some situations, preventing the onset of the pathology, injury, condition, or symptom. Treatment or amelioration can be based on any objective or subjective parameter; including, e.g., the result of a physical examination.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. A “patient” refers to a human subject in need of treatment of a disease.

The terms “administer,” “administering,” or “administration” refers to implanting, absorbing, ingesting, injecting, inhaling, or otherwise introducing a polymorphic form of Compound (I) described herein, or a composition thereof, in or on a subject.

The terms “condition,” “disease,” and “disorder” are used interchangeably.

An “effective amount” of a polymorphic form described herein refers to an amount sufficient to elicit the desired biological response, i.e., treating the condition. As will be appreciated by those of ordinary skill in this art, the effective amount of a polymorphic form of Compound (I) described herein may vary depending on such factors as the desired biological endpoint, the pharmacokinetics of the polymorphic form, the condition being treated, the mode of administration, and the age and health of the subject. In certain embodiments, an effective amount is a therapeutically effective amount. In certain embodiments, an effective amount is the amount of a polymorphic form of Compound (I) described herein in a single dose. In certain embodiments, an effective amount is the combined amounts of a polymorphic form of Compound (I) described herein in multiple doses.

A “therapeutically effective amount” of a polymorphic form of Compound (I) described herein is an amount sufficient to provide a therapeutic benefit in the treatment of a condition or to delay or minimize one or more symptoms associated with the condition. A therapeutically effective amount of a polymorphic form means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

The term “prevent,” “preventing,” or “prevention” refers to a prophylactic treatment of a subject who is not and/or was not suffering from a disease, but is at risk of developing the disease, or a subject who is at risk of progression of the disease. In certain embodiments, the subject is at a higher risk of developing the disease or at a higher risk of progression of the disease than an average healthy member of a population.

The term “inhibition,” “inhibiting,” “inhibit,” or “inhibitor” refers to the ability of a compound to reduce, slow, halt, or prevent activity of a particular biological process (e.g., furin activity, viral infectivity, viral replication, toxin activation, and/or activity) in a subject relative to vehicle.

### DETAILED DESCRIPTION

Provided herein are pharmaceutical compositions comprising Compound (I), of the formula:

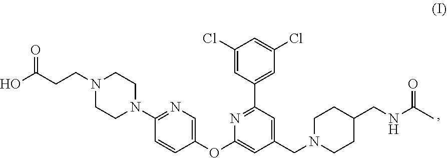 [SMILES: CC(=O)NCC1CCN(Cc2cc(Oc3ccc(N4CCN(CCC(=O)O)CC4)nc3)nc(-c3cc(Cl)cc(Cl)c3)c2)CC1 ; ","]

or pharmaceutically acceptable salts, solvates, tautomers, stereoisomers, or isotopically labeled derivatives thereof, or a polymorph thereof. In certain embodiments, the composition comprises Compound (I), or a pharmaceutically acceptable salt thereof. In certain embodiments, the composition comprises Compound (I), wherein at least a portion of Compound (I) is in the form of a fumarate salt. In certain embodiments, the composition further comprises a pharmaceutically acceptable excipient (e.g., a buffering agent (e.g., an organic acid (e.g., citric acid))). In certain embodiments, the composition further comprises a second pharmaceutically acceptable excipient (e.g., a tonicity agent (e.g., sugars (e.g., dextrose, lactose, trehalose, sucrose), sugar alcohols (e.g., mannitol), salts (e.g., sodium chloride, potassium chloride), and polyols (e.g., propylene glycol, glycerin))).

In another aspect, provided herein are compositions comprising Compound (I) or pharmaceutically acceptable salts, solvates, tautomers, stereoisomers, or isotopically labeled derivatives thereof, or a polymorph thereof for use in the treatment of a disease (e.g., cystic fibrosis, fibrotic diseases (e.g., pulmonary fibrosis)). In some embodiments, the compositions comprising Compound (I) as described herein are formulated for inhalation (e.g., oral and/or nasal inhalation). In other embodiments, the compositions comprising Compound (I) or pharmaceutically acceptable salts, solvates, tautomers, stereoisomers, or isotopically labeled derivatives thereof, or a polymorph thereof, are formulated for administration via a nebulizer. In other embodiments, the compositions comprising Compound (I) or pharmaceutically acceptable salts, solvates, tautomers, stereoisomers, or isotopically labeled derivatives thereof, or a polymorph thereof, are formulated for administration via an inhaler (e.g., a dry powder inhaler). In some aspects, provided herein are methods of treating a fibrotic disease or condition comprising administering to a subject in need thereof a therapeutically effective amount of the pharmaceutical composition comprising Compound (I). In another aspect, provided herein are methods of treating cystic fibrosis comprising administering to a subject in need thereof a therapeutically effective amount of the pharmaceutical composition comprising Compound (I).

Also, provided herein are polymorphs of Compound (I), wherein Compound (I) is of the formula:

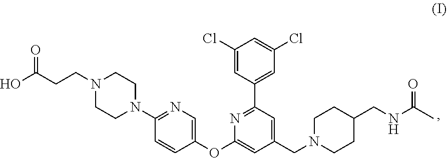 [SMILES: CC(=O)NCC1CCN(Cc2cc(Oc3ccc(N4CCN(CCC(=O)O)CC4)nc3)nc(-c3cc(Cl)cc(Cl)c3)c2)CC1 ; ","]

or a pharmaceutically acceptable salt, solvate, tautomer, stereoisomer, or isotopically labeled derivative thereof. In certain embodiments, the compositions comprising Compound (I) described herein comprise a polymorph of Compound (I). Polymorphic forms of Compound (I) include Freeform Type A, Freeform Type B, Freeform Type C, Freeform Type D, HCl salt Type A, HCl salt Type B, HCl salt Type C, HCl salt Type D, HCl salt Type E, HCl salt Type F, sulfate salt Type A, sulfate salt Type B, maleate salt Type A, maleate salt Type B, tartrate salt Type A, fumarate salt Type A, fumarate salt Type B, fumarate salt Type C, fumarate salt Type D, succinate salt Type A, succinate salt Type B, succinate salt Type C, triphenylacetate salt Type A, xinafoic salt Type A, Ca salt Type A, tromethamine salt Type A, and tromethamine salt Type B as detailed herein.

The compositions and polymorphs of Compound (I) can be prepared by methods as described in the Examples. One skilled in the art will appreciate that the compositions, compounds, and polymorphs thereof of the disclosure can be prepared using other synthetic methods as substitutes for transformations provided in the Examples.

### Freeform Type A

The present disclosure provides a polymorph of Compound (I) characterized as Freeform Type A. In certain embodiments, Freeform Type A is characterized by at least one of:
a. an X-ray powder diffraction pattern obtained by irradiation with Cu-Kα having three or more peaks, expressed in degrees 2-theta±0.2°, selected from 3.96, 7.9, 11.85, 15.83, 16.26, 17.78, 19.82, 20.66, 22.76, 23.83, 24.86, 25.71, 26.83, 27.87, 28.65, 29.36, 30.06, 31.95, 33.92, and 36.07; and/or
b. a DSC thermogram showing an endotherm at about 110.3° C.

In certain embodiments, Freeform Type A is characterized by an X-ray powder diffraction pattern obtained by irradiation with Cu-Kα having each of the peaks expressed in degrees 2-theta±0.2° selected from 3.96, 7.9, 11.85, 15.83, 16.26, 17.78, 19.82, 20.66, 22.76, 23.83, 24.86, 25.71, 26.83, 27.87, 28.65, 29.36, 30.06, 31.95, 33.92, and 36.07. In certain embodiments, Freeform Type A is characterized by an X-ray powder diffraction pattern obtained by irradiation with Cu-Kα having each of the peaks expressed in degrees 2-theta±0.2° selected from 3.96, 7.9, 11.85, 15.83, 16.26, 17.78, 19.82, 20.66, 22.76, 23.83, 24.86, 25.71, 26.83, 27.87, 30.06, 31.95, 33.92, and 36.07. In certain embodiments, Freeform Type A is characterized by an X-ray powder diffraction pattern obtained by irradiation with Cu-Kα having each of the peaks expressed in degrees 2-theta±0.2° selected from 3.96, 7.9, 11.85, 15.83, 16.26, 19.82, 23.83, 26.83, 31.95, and 36.07. In certain embodiments, Freeform Type A is characterized by an X-ray powder diffraction pattern obtained by irradiation with Cu-Kα having each of the peaks expressed in degrees 2-theta±0.2° selected from 3.96, 7.9, 11.85, 15.83, 16.26, 19.82, 23.83, and 31.95. In certain embodiments, Freeform Type A is characterized by an X-ray powder diffraction pattern obtained by irradiation with Cu-Kα having each of the peaks expressed in degrees 2-theta±0.2° selected from 3.96, 7.9, 11.85, 15.83, and 19.82.

In certain embodiments, Freeform Type A has an X-ray powder diffraction pattern obtained by irradiation with Cu-Kα having at least three peaks expressed in degrees 2-theta±0.2° selected from 3.96, 7.9, 11.85, 15.83, 16.26, 17.78, 19.82, 20.66, 22.76, 23.83, 24.86, 25.71, 26.83, 27.87, 28.65, 29.36, 30.06, 31.95, 33.92, and 36.07. In certain embodiments, Freeform Type A has an X-ray powder diffraction pattern obtained by irradiation with Cu-Kα having at least three peaks expressed in degrees 2-theta±0.2° selected from 3.96, 7.9, 11.85, 15.83, 16.26, 17.78, 19.82, 20.66, 22.76, 23.83, 24.86, 25.71, 26.83, 27.87, 30.06, 31.95, 33.92, and 36.07. In certain embodiments, Freeform Type A has an X-ray powder diffraction pattern obtained by irradiation with Cu-Kα having at least three peaks expressed in degrees 2-theta±0.2° selected from 3.96, 7.9, 11.85, 15.83, 16.26, 19.82, 23.83, 26.83, 31.95, and 36.07. In certain embodiments, Freeform Type A has an X-ray powder diffraction pattern obtained by irradiation with Cu-Kα having at least three peaks expressed in degrees 2-theta±0.2° selected from 3.96, 7.9, 11.85, 15.83, 16.26, 19.82, 23.83, and 31.95. In certain embodiments, Freeform Type A has an X-ray powder diffraction pattern obtained by irradiation with Cu-Kα having at least three peaks expressed in degrees 2-theta±0.2° selected from 3.96, 7.9, 11.85, 15.83, and 19.82. In certain embodiments, Freeform Type A is characterized by 4 or more peaks, 5 or more peaks, 6 or more peaks, 7 or more peaks, 8 or more peaks, 16 or more peaks, or 20 or more peaks expressed in degrees 2-theta±0.2° and selected from 3.96, 7.9, 11.85, 15.83, 16.26, 17.78, 19.82, 20.66, 22.76, 23.83, 24.86, 25.71, 26.83, 27.87, 28.65, 29.36, 30.06, 31.95, 33.92, and 36.07.

In certain embodiments, Freeform Type A is characterized by an X-ray powder diffraction pattern obtained by irradiation with Cu-Kα lacking peaks expressed in degrees 2-theta±0.050 at each of 12.0 to 15.0, 18.0 to 19.5, and 34.0 to 36.0. In certain embodiments, Freeform Type A is characterized by an X-ray powder diffraction pattern obtained by irradiation with Cu-Kα lacking peaks expressed in degrees 2-theta±0.050 at each of 12.0 to 15.0, and 18.0 to 19.5. In certain embodiments, Freeform Type A is characterized by an X-ray powder diffraction pattern obtained by irradiation with Cu-Kα lacking peaks expressed in degrees 2-theta±0.050 at 12.0 to 15.0. In certain embodiments, Freeform Type A is characterized by an X-ray powder diffraction pattern obtained by irradiation with Cu-Kα lacking peaks expressed in degrees 2-theta±0.050 at 8.0 to 19.5. In certain embodiments, Freeform Type A is characterized by an X-ray powder diffraction pattern obtained by irradiation with Cu-Kα lacking peaks expressed in degrees 2-theta±0.050 at 34.0 to 36.0.

In certain embodiments, Freeform Type A is characterized by an X-ray powder diffraction pattern substantially identical to the XRPD pattern shown in FIG. 2.

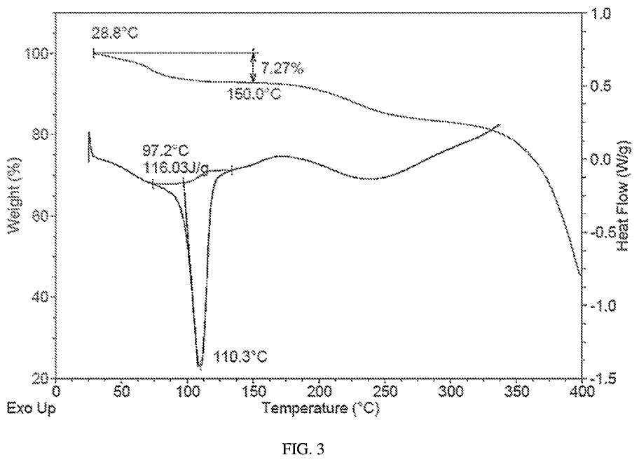

In some aspects, Freeform Type A is characterized by a DSC thermogram essentially the same as shown in FIG. 3. In some aspects, Freeform Type A is characterized by a DSC thermogram showing an endotherm at about 105° C. to about 115° C. In some aspects, Freeform Type A is characterized by a DSC thermogram showing an endotherm at about 110.3° C.

In certain aspects of the disclosure, Freeform Type A is substantially free of other forms of Compound (I) (e.g., other polymorphs, amorphous forms). In certain embodiments, Freeform Type A is substantially free of Freeform Type B. In certain embodiments, Freeform Type A is substantially free of Freeform Type C. In certain embodiments, Freeform Type A is substantially free of Freeform Type D. In certain embodiments, Freeform Type A is substantially free of Freeform Type B and Freeform Type C. In certain embodiments, Freeform Type A is substantially free of Freeform Type B and Freeform Type D. In certain embodiments, Freeform Type A is substantially free of Freeform Type C and Freeform Type D. In certain embodiments, Freeform Type A is substantially free of Freeform Type B, Freeform Type C, and Freeform Type D. In certain embodiments, Freeform Type A is substantially free of amorphous forms of Compound (I).

### Freeform Type B

The present disclosure provides a polymorph of Compound (I) characterized as Freeform Type B. In certain embodiments, Freeform Type B is characterized by at least one of:
a. an X-ray powder diffraction pattern obtained by irradiation with Cu-Kα having three or more peaks, expressed in degrees 2-theta±0.2°, selected from 5.21, 5.77, 8.26, 9.37, 11.6, 12.96, 15.65, 16.61, 17.23, 18.51, 19.65, 20.8, 22.03, 23.2, 24.24, 24.63, 25.15, 26.26, 28.37, 29.74, and 34.85; and/or
b. a DSC thermogram showing an endotherm at about 190.6° C.

In certain embodiments, Freeform Type B is characterized by an X-ray powder diffraction pattern obtained by irradiation with Cu-Kα having each of the peaks expressed in degrees 2-theta±0.2° selected from 5.21, 5.77, 8.26, 9.37, 11.6, 12.96, 15.65, 16.61, 17.23, 18.51, 19.65, 20.8, 22.03, 23.2, 24.24, 24.63, 25.15, 26.26, 28.37, 29.74, and 34.85. In certain embodiments, Freeform Type B is characterized by an X-ray powder diffraction pattern obtained by irradiation with Cu-Kα having each of the peaks expressed in degrees 2-theta±0.2° selected from 5.21, 5.77, 8.26, 9.37, 11.6, 12.96, 15.65, 16.61, 17.23, 18.51, 19.65, 20.8, 22.03, 23.2, 24.24, 24.63, and 26.26. In certain embodiments, Freeform Type B is characterized by an X-ray powder diffraction pattern obtained by irradiation with Cu-Kα having each of the peaks expressed in degrees 2-theta±0.2° selected from 5.21, 8.26, 11.6, 12.96, 16.61, 17.23, 19.65, 20.8, and 22.03. In certain embodiments, Freeform Type B is characterized by an X-ray powder diffraction pattern obtained by irradiation with Cu-Kα having each of the peaks expressed in degrees 2-theta±0.2° selected from 8.26, 16.61, 17.23, and 22.03. In certain embodiments, Freeform Type B is characterized by an X-ray powder diffraction pattern obtained by irradiation with Cu-Kα having each of the peaks expressed in degrees 2-theta±0.2° selected from 17.23, and 22.03.

In certain embodiments, Freeform Type B has an X-ray powder diffraction pattern obtained by irradiation with Cu-Kα having at least three peaks expressed in degrees 2-theta±0.2° selected from 5.21, 5.77, 8.26, 9.37, 11.6, 12.96, 15.65, 16.61, 17.23, 18.51, 19.65, 20.8, 22.03, 23.2, 24.24, 24.63, 25.15, 26.26, 28.37, 29.74, and 34.85. In certain embodiments, Freeform Type B has an X-ray powder diffraction pattern obtained by irradiation with Cu-Kα having at least three peaks expressed in degrees 2-theta±0.2° selected from 5.21, 5.77, 8.26, 9.37, 11.6, 12.96, 15.65, 16.61, 17.23, 18.51, 19.65, 20.8, 22.03, 23.2, 24.24, 24.63, and 26.26. In certain embodiments, Freeform Type B has an X-ray powder diffraction pattern obtained by irradiation with Cu-Kα having at least three peaks expressed in degrees 2-theta±0.2° selected from 5.21, 8.26, 11.6, 12.96, 16.61, 17.23, 19.65, 20.8, and 22.03. In certain embodiments, Freeform Type B has an X-ray powder diffraction pattern obtained by irradiation with Cu-Kα having at least three peaks expressed in degrees 2-theta±0.2° selected from 8.26, 16.61, 17.23, and 22.03. In certain embodiments, Freeform Type D is characterized by 4 or more peaks, 5 or more peaks, 6 or more peaks, 7 or more peaks, 8 or more peaks, 16 or more peaks, or 20 or more peaks expressed in degrees 2-theta±0.2° and selected from 5.21, 5.77, 8.26, 9.37, 11.6, 12.96, 15.65, 16.61, 17.23, 18.51, 19.65, 20.8, 22.03, 23.2, 24.24, 24.63, 25.15, 26.26, 28.37, 29.74, and 34.85.

In certain embodiments, Freeform Type B is characterized by an X-ray powder diffraction pattern obtained by irradiation with Cu-Kα lacking peaks expressed in degrees 2-theta±0.050 at each of 0.0 to 5.0, 20.0 to 20.5, and 30.0 to 34.0. In certain embodiments, Freeform Type B is characterized by an X-ray powder diffraction pattern obtained by irradiation with Cu-Kα lacking peaks expressed in degrees 2-theta±0.050 at each of 0.0 to 5.0, and 30.0 to 34.0. In certain embodiments, Freeform Type B is characterized by an X-ray powder diffraction pattern obtained by irradiation with Cu-Kα lacking peaks expressed in degrees 2-theta±0.050 at 0.0 to 5.0. In certain embodiments, Freeform Type B is characterized by an X-ray powder diffraction pattern obtained by irradiation with Cu-Kα lacking peaks expressed in degrees 2-theta±0.050 at 20.0 to 20.5. In certain embodiments, Freeform Type B is characterized by an X-ray powder diffraction pattern obtained by irradiation with Cu-Kα lacking peaks expressed in degrees 2-theta±0.050 at 30.0 to 34.0.

In certain embodiments, Freeform Type B is characterized by an X-ray powder diffraction pattern substantially identical to the XRPD pattern shown in FIG. 184.

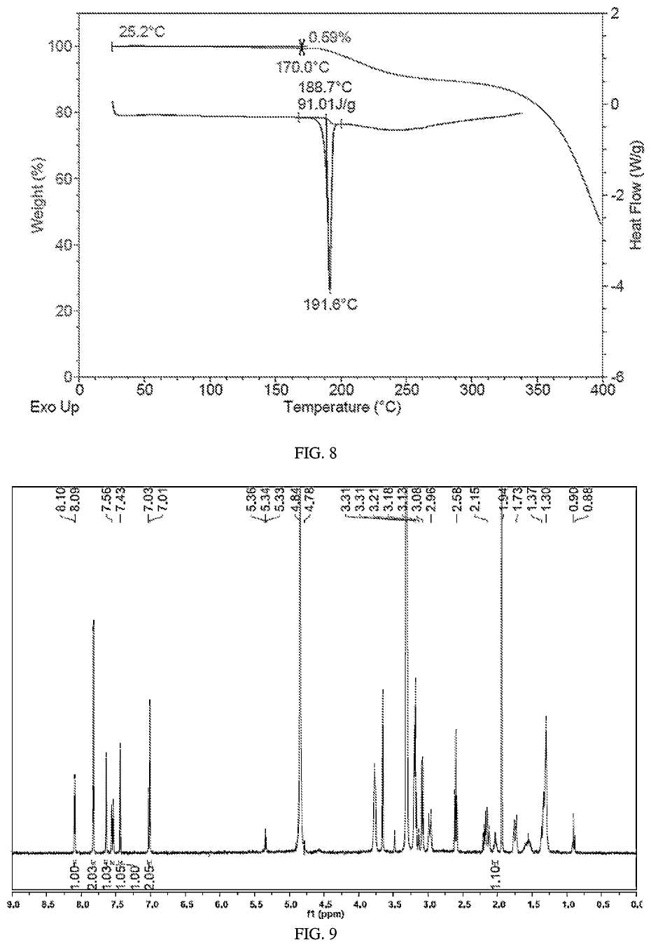

In some aspects, Freeform Type B is characterized by a DSC thermogram essentially the same as shown in FIG. 8. In some aspects, Freeform Type B is characterized by a DSC thermogram essentially the same as shown in FIG. 11. In some aspects, Freeform Type B is characterized by a DSC thermogram showing an endotherm at about 185° C. to about 195° C. In some aspects, Freeform Type B is characterized by a DSC thermogram showing an endotherm at about 190.6° C.

In certain aspects of the disclosure, Freeform Type B is substantially free of other forms of Compound (I) (e.g., other polymorphs, amorphous forms). In certain embodiments, Freeform Type B is substantially free of Freeform Type A. In certain embodiments, Freeform Type B is substantially free of Freeform Type C. In certain embodiments, Freeform Type B is substantially free of Freeform Type D. In certain embodiments, Freeform Type B is substantially free of Freeform Type A and Freeform Type C. In certain embodiments, Freeform Type B is substantially free of Freeform Type A and Freeform Type D. In certain embodiments, Freeform Type B is substantially free of Freeform Type C and Freeform Type D. In certain embodiments, Freeform Type D is substantially free of Freeform Type A, Freeform Type C, and Freeform Type D. In certain embodiments, Freeform Type B is substantially free of amorphous forms of Compound (I).

### Freeform Type C

The present disclosure provides a polymorph of Compound (I) characterized as Freeform Type C. In certain embodiments, Freeform Type C is characterized by an X-ray powder diffraction pattern obtained by irradiation with Cu-Kα having three or more peaks, expressed in degrees 2-theta±0.2°, selected from 3.9, 12.18, 13.27, 16.16, 17.35, 18.76, 19.37, 19.84, 20.41, 20.74, 21.91, 24.12, 26.07, 27.12, 28.67, 30.45, 31.9, 33.86, and 35.05.

In certain embodiments, Freeform Type C is characterized by an X-ray powder diffraction pattern obtained by irradiation with Cu-Kα having each of the peaks expressed in degrees 2-theta±0.2° selected from 3.9, 12.18, 13.27, 16.16, 17.35, 18.76, 19.37, 19.84, 20.41, 20.74, 21.91, 24.12, 26.07, 27.12, 28.67, 30.45, 31.9, 33.86, and 35.05. In certain embodiments, Freeform Type C is characterized by an X-ray powder diffraction pattern obtained by irradiation with Cu-Kα having each of the peaks expressed in degrees 2-theta±0.2° selected from 13.27, 16.16, 17.35, 28.67, 30.45, 31.9, 33.86, and 35.05. In certain embodiments, Freeform Type C is characterized by an X-ray powder diffraction pattern obtained by irradiation with Cu-Kα having each of the peaks expressed in degrees 2-theta±0.2° selected from 12.18, 18.76, 19.37, 19.84, 21.91, 24.12, 26.07, and 27.12. In certain embodiments, Freeform Type C is characterized by an X-ray powder diffraction pattern obtained by irradiation with Cu-Kα having each of the peaks expressed in degrees 2-theta±0.2° selected from 3.9, 20.41, and 20.74.

In certain embodiments, Freeform Type C has an X-ray powder diffraction pattern obtained by irradiation with Cu-Kα having at least three peaks expressed in degrees 2-theta±0.2° selected from 3.9, 12.18, 13.27, 16.16, 17.35, 18.76, 19.37, 19.84, 20.41, 20.74, 21.91, 24.12, 26.07, 27.12, 28.67, 30.45, 31.9, 33.86, and 35.05. In certain embodiments, Freeform Type C has an X-ray powder diffraction pattern obtained by irradiation with Cu-Kα having at least three peaks expressed in degrees 2-theta±0.2° selected from 13.27, 16.16, 17.35, 28.67, 30.45, 31.9, 33.86, and 35.05. In certain embodiments, Freeform Type C has an X-ray powder diffraction pattern obtained by irradiation with Cu-Kα having at least three peaks expressed in degrees 2-theta±0.2° selected from 12.18, 18.76, 19.37, 19.84, 21.91, 24.12, 26.07, and 27.12. In certain embodiments, Freeform Type C is characterized by 4 or more peaks, 8 or more peaks, 16 or more peaks, or 20 or more peaks expressed in degrees 2-theta±0.2° and selected from 3.9, 12.18, 13.27, 16.16, 17.35, 18.76, 19.37, 19.84, 20.41, 20.74, 21.91, 24.12, 26.07, 27.12, 28.67, 30.45, 31.9, 33.86, and 35.05.

In certain embodiments, Freeform Type C is characterized by an X-ray powder diffraction pattern obtained by irradiation with Cu-Kα lacking peaks expressed in degrees 2-theta±0.050 at each of 4.0 to 11.0, 22.0 to 24.0, and 30.0 to 34.0. In certain embodiments, Freeform Type C is characterized by an X-ray powder diffraction pattern obtained by irradiation with Cu-Kα lacking peaks expressed in degrees 2-theta±0.050 at each of 4.0 to 11.0, and 30.0 to 34.0. In certain embodiments, Freeform Type C is characterized by an X-ray powder diffraction pattern obtained by irradiation with Cu-Kα lacking peaks expressed in degrees 2-theta±0.050 at 4.0 to 11.0. In certain embodiments, Freeform Type C is characterized by an X-ray powder diffraction pattern obtained by irradiation with Cu-Kα lacking peaks expressed in degrees 2-theta±0.050 at 22.0 to 24.0. In certain embodiments, Freeform Type C is characterized by an X-ray powder diffraction pattern obtained by irradiation with Cu-Kα lacking peaks expressed in degrees 2-theta±0.050 at 30.0 to 34.0.

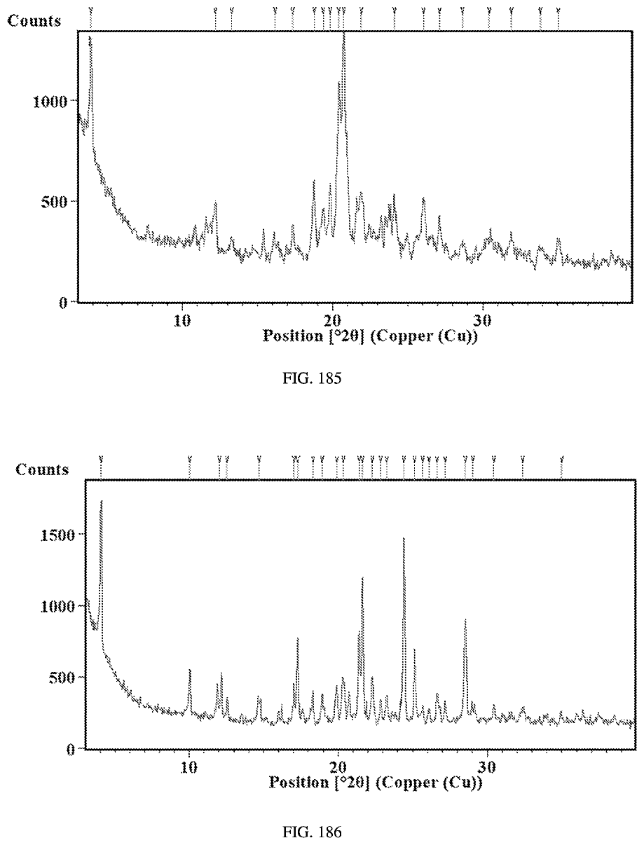

In certain embodiments, Freeform Type C is characterized by an X-ray powder diffraction pattern substantially identical to the XRPD pattern shown in FIG. 185.

In certain aspects of the disclosure, Freeform Type C is substantially free of other forms of Compound (I) (e.g., other polymorphs, amorphous forms). In certain embodiments, Freeform Type C is substantially free of Freeform Type A. In certain embodiments, Freeform Type C is substantially free of Freeform Type B. In certain embodiments, Freeform Type C is substantially free of Freeform Type D. In certain embodiments, Freeform Type C is substantially free of Freeform Type A and Freeform Type B. In certain embodiments, Freeform Type C is substantially free of Freeform Type A and Freeform Type D. In certain embodiments, Freeform Type C is substantially free of Freeform Type B and Freeform Type D. In certain embodiments, Freeform Type C is substantially free of Freeform Type A, Freeform Type B, and Freeform Type D. In certain embodiments, Freeform Type C is substantially free of amorphous forms of Compound (I).

### Freeform Type D

The present disclosure provides a polymorph of Compound (I) characterized as Freeform Type D. In certain embodiments, Freeform Type D is characterized by at least one of:
a. an X-ray powder diffraction pattern obtained by irradiation with Cu-Kα having three or more peaks, expressed in degrees 2-theta±0.2°, selected from 4.07, 10.03, 12.01, 12.53, 14.68, 17.01, 17.27, 18.29, 18.91, 19.89, 20.33, 21.4, 21.62, 22.27, 22.85, 23.25, 24.41, 25.14, 25.65, 26.08, 26.63, 27.18, 28.53, 29.04, 30.45, 32.37, and 35.01; and/or
b. a DSC thermogram showing an endotherm at about 106.7° C.

In certain embodiments, Freeform Type D is characterized by an X-ray powder diffraction pattern obtained by irradiation with Cu-Kα having each of the peaks expressed in degrees 2-theta±0.2° selected from 4.07, 10.03, 12.01, 12.53, 14.68, 17.01, 17.27, 18.29, 18.91, 19.89, 20.33, 21.4, 21.62, 22.27, 22.85, 23.25, 24.41, 25.14, 25.65, 26.08, 26.63, 27.18, 28.53, 29.04, 30.45, 32.37, and 35.01. In certain embodiments, Freeform Type D is characterized by an X-ray powder diffraction pattern obtained by irradiation with Cu-Kα having each of the peaks expressed in degrees 2-theta±0.2° selected from 4.07, 10.03, 12.01, 12.53, 14.68, 17.01, 17.27, 18.29, 18.91, 19.89, 20.33, 21.4, 21.62, 22.27, 22.85, 23.25, 24.41, 25.14, 26.63, 27.18, 28.53, and 30.45. In certain embodiments, Freeform Type D is characterized by an X-ray powder diffraction pattern obtained by irradiation with Cu-Kα having each of the peaks expressed in degrees 2-theta±0.2° selected from 4.07, 17.27, 21.4, 21.62, 24.41, 25.14, and 28.53. In certain embodiments, Freeform Type D is characterized by an X-ray powder diffraction pattern obtained by irradiation with Cu-Kα having each of the peaks expressed in degrees 2-theta±0.2° selected from 4.07, 21.62, and 24.41.

In certain embodiments, Freeform Type D has an X-ray powder diffraction pattern obtained by irradiation with Cu-Kα having at least three peaks expressed in degrees 2-theta±0.2° selected from 4.07, 10.03, 12.01, 12.53, 14.68, 17.01, 17.27, 18.29, 18.91, 19.89, 20.33, 21.4, 21.62, 22.27, 22.85, 23.25, 24.41, 25.14, 25.65, 26.08, 26.63, 27.18, 28.53, 29.04, 30.45, 32.37, 35.01. In certain embodiments, Freeform Type D has an X-ray powder diffraction pattern obtained by irradiation with Cu-Kα having at least three peaks expressed in degrees 2-theta±0.2° selected from 4.07, 10.03, 12.01, 12.53, 14.68, 17.01, 17.27, 18.29, 18.91, 19.89, 20.33, 21.4, 21.62, 22.27, 22.85, 23.25, 24.41, 25.14, 26.63, 27.18, 28.53, and 30.45. In certain embodiments, Freeform Type D has an X-ray powder diffraction pattern obtained by irradiation with Cu-Kα having at least three peaks expressed in degrees 2-theta±0.2° selected from 4.07, 17.27, 21.4, 21.62, 24.41, 25.14, and 28.53. In certain embodiments, Freeform Type D has an X-ray powder diffraction pattern obtained by irradiation with Cu-Kα having at least three peaks expressed in degrees 2-theta±0.2° selected from 4.07, 17.27, 21.4, 21.62, 24.41, 25.14, and 28.53. In certain embodiments, Freeform Type D is characterized by 4 or more peaks, 8 or more peaks, 16 or more peaks, or 20 or more peaks expressed in degrees 2-theta±0.2° and selected from 4.07, 10.03, 12.01, 12.53, 14.68, 17.01, 17.27, 18.29, 18.91, 19.89, 20.33, 21.4, 21.62, 22.27, 22.85, 23.25, 24.41, 25.14, 25.65, 26.08, 26.63, 27.18, 28.53, 29.04, 30.45, 32.37, 35.01.

In certain embodiments, Freeform Type D is characterized by an X-ray powder diffraction pattern obtained by irradiation with Cu-Kα lacking peaks expressed in degrees 2-theta±0.050 at each of 4.50 to 9.50, 12.1 to 12.3, and 20.40 to 20.60. In certain embodiments, Freeform Type D is characterized by an X-ray powder diffraction pattern obtained by irradiation with Cu-Kα lacking peaks expressed in degrees 2-theta±0.05° at each of 12.1 to 12.3, and 20.40 to 20.60. In certain embodiments, Freeform Type D is characterized by an X-ray powder diffraction pattern obtained by irradiation with Cu-Kα lacking peaks expressed in degrees 2-theta±0.050 at 4.50 to 9.50. In certain embodiments, Freeform Type D is characterized by an X-ray powder diffraction pattern obtained by irradiation with Cu-Kα lacking peaks expressed in degrees 2-theta±0.050 at 12.1 to 12.3. In certain embodiments, Freeform Type D is characterized by an X-ray powder diffraction pattern obtained by irradiation with Cu-Kα lacking peaks expressed in degrees 2-theta±0.050 at 20.40 to 20.60.

In certain embodiments, Freeform Type D is characterized by an X-ray powder diffraction pattern substantially identical to the XRPD pattern shown in FIG. 186.

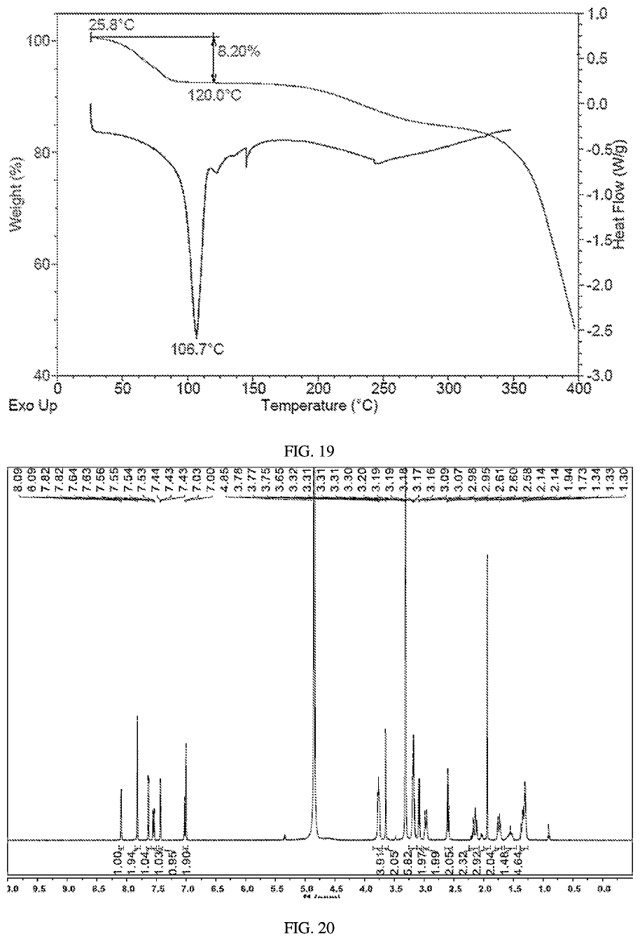

In some aspects, Freeform Type D is characterized by a DSC thermogram essentially the same as shown in FIG. 19. In some aspects, Freeform Type D is characterized by a DSC thermogram showing an endotherm at about 95° C. to about 115° C. In some aspects, Freeform Type D is characterized by a DSC thermogram showing an endotherm at about 100° C. to about 110° C. In some aspects, Freeform Type D is characterized by a DSC thermogram showing an endotherm at about 106.7° C. In some aspects, Freeform Type D is characterized by a melting point of 106.7° C.±2° C.

In certain embodiments, Freeform Type D is characterized by at least one of:
a. an X-ray powder diffraction pattern obtained by irradiation with Cu-Kα having three or more peaks, expressed in degrees 2-theta±0.2°, selected from 4.07, 17.27, 21.4, 21.62, 24.41, 25.14, and 28.53; and/or
b. a DSC thermogram showing an endotherm at about 106.7° C.

In certain embodiments, Freeform Type D is characterized by at least one of:
a. an X-ray powder diffraction pattern obtained by irradiation with Cu-Kα having three or more peaks, expressed in degrees 2-theta±0.2°, selected from 4.07, 21.62, and 24.41; and/or
b. a DSC thermogram showing an endotherm at about 106.7° C.

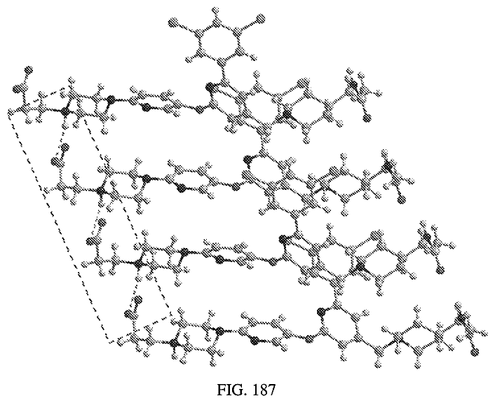

In certain aspects, Freeform Type D is characterized by the single crystal structure shown in FIG. 187. In some aspects, Freeform Type D has a monoclinic crystal system and a space group of P2₁/c. In certain aspects, Freeform Type D has unit cell dimensions of a=21.65166(12) Å, b=14.93710(10) Å, c=10.35008(6) Å, a=90°, =90.5133(5°), 7=90°, and V=3347.22(3) Å3.

In certain aspects of the disclosure, Freeform Type D is substantially free of other forms of Compound (I). In certain embodiments, Freeform Type D is substantially free of Freeform Type A. In certain embodiments, Freeform Type D is substantially free of Freeform Type B. In certain embodiments, Freeform Type D is substantially free of Freeform Type C. In certain embodiments, Freeform Type D is substantially free of Freeform Type A and Freeform Type B. In certain embodiments, Freeform Type D is substantially free of Freeform Type A and Freeform Type C. In certain embodiments, Freeform Type D is substantially free of Freeform Type B and Freeform Type C. In certain embodiments, Freeform Type D is substantially free of Freeform Type A, Freeform Type B, and Freeform Type C.

### Fumarate Type A

The present disclosure provides a polymorph of Compound (I) characterized as Fumarate Type A. In certain embodiments, Fumarate Type A is characterized by at least one of:
a. an X-ray powder diffraction pattern obtained by irradiation with Cu-Kα having three or more peaks, expressed in degrees 2-theta±0.2°, selected from 11.67, 17.67, 19.18, 22.45, 23.26, and 27.14; and/or
b. a DSC thermogram showing an endotherm at about 158.9° C.

In certain embodiments, Fumarate Type A is characterized by an X-ray powder diffraction pattern obtained by irradiation with Cu-Kα having each of the peaks expressed in degrees 2-theta±0.2° selected from 11.67, 17.67, 19.18, 22.45, 23.26, and 27.14. In certain embodiments, Fumarate Type A is characterized by an X-ray powder diffraction pattern obtained by irradiation with Cu-Kα having each of the peaks expressed in degrees 2-theta±0.2° selected from 17.67, 19.18, 22.45, and 23.26.

In certain embodiments, Fumarate Type A has an X-ray powder diffraction pattern obtained by irradiation with Cu-Kα having at least three peaks expressed in degrees 2-theta±0.2° selected from 11.67, 17.67, 19.18, 22.45, 23.26, and 27.14. In certain embodiments, Fumarate Type A has an X-ray powder diffraction pattern obtained by irradiation with Cu-Kα having at least three peaks expressed in degrees 2-theta±0.2° selected from 17.67, 19.18, 22.45, and 23.26.

In certain embodiments, Fumarate Type A is characterized by an X-ray powder diffraction pattern obtained by irradiation with Cu-Kα lacking peaks expressed in degrees 2-theta±0.05° at each of 0 to 11.50, 18.0 to 19.0, and 28.0 to 35.0. In certain embodiments, Fumarate Type A is characterized by an X-ray powder diffraction pattern obtained by irradiation with Cu-Kα lacking peaks expressed in degrees 2-theta±0.050 at each of 0 to 11.50, and 18.0 to 19.0. In certain embodiments, Fumarate Type A is characterized by an X-ray powder diffraction pattern obtained by irradiation with Cu-Kα lacking peaks expressed in degrees 2-theta±0.050 at 0 to 11.50. In certain embodiments, Fumarate Type A is characterized by an X-ray powder diffraction pattern obtained by irradiation with Cu-Kα lacking peaks expressed in degrees 2-theta±0.050 at 18.0 to 19.0. In certain embodiments, Fumarate Type A is characterized by an X-ray powder diffraction pattern obtained by irradiation with Cu-Kα lacking peaks expressed in degrees 2-theta±0.050 at 0 to 28.0 to 35.0.

In certain embodiments, Fumarate Type A is characterized by an X-ray powder diffraction pattern substantially identical to the XRPD pattern shown in FIG. 45.

In some aspects, Freeform Type D is characterized by a DSC thermogram essentially the same as shown in FIG. 154. In some aspects, Freeform Type D is characterized by a DSC thermogram showing an endotherm at about 150° C. to about 170° C.

In some aspects, Freeform Type D is characterized by a DSC thermogram showing an endotherm at about 155° C. to about 165° C. In some aspects, Freeform Type D is characterized by a DSC thermogram showing an endotherm at about 158.9° C.

In certain aspects of the disclosure, Fumarate Type A is substantially free of other forms of Compound (I). In certain embodiments, Fumarate Type A is substantially free of Fumarate Type B. In certain embodiments, Fumarate Type A is substantially free of Fumarate Type C. In certain embodiments, Fumarate Type A is substantially free of Fumarate Type D. In certain embodiments, Fumarate Type A is substantially free of Fumarate Type B, Fumarate Type C, and Fumarate Type D.

### Pharmaceutical Compositions, Kits, Uses, and Administration

Provided herein is a pharmaceutical composition (also referred to as a pharmaceutical formulation) comprising Compound (I), or a pharmaceutically acceptable salt, solvate, tautomer, stereoisomer, or isotopically labeled derivative thereof, or a polymorph thereof.

In some aspects, a pharmaceutical composition as described herein comprises Compound (I), or a pharmaceutically acceptable salt, solvate, tautomer, stereoisomer, or isotopically labeled derivative thereof, or a polymorph thereof, and a pharmaceutically acceptable excipient. In some aspects, a pharmaceutical composition as described herein comprises a polymorph of Compound (I), or a pharmaceutically acceptable salt, polymorph, solvate, tautomer, stereoisomer, or isotopically labeled derivative thereof, and one or more pharmaceutically acceptable excipients. In certain embodiments, the pharmaceutical composition comprises Compound (I), or a pharmaceutically acceptable salt, polymorph, solvate, tautomer, stereoisomer, or isotopically labeled derivative thereof, a first excipient, and a second excipient.

In certain embodiments, the excipient is a buffering agent (e.g., an organic acid (e.g., citric acid)). In certain embodiments, the excipient is an organic acid (e.g., citric acid). In certain embodiments, In some aspects, a pharmaceutical composition as described herein comprises Compound (I), or a pharmaceutically acceptable salt, solvate, tautomer, stereoisomer, or isotopically labeled derivative thereof, or a polymorph thereof, and an organic acid. In certain embodiments, the excipient is an organic acid selected from vitamin C, citric acid, fumaric acid, acetic acid, dehydroacetic acid, ascorbic acid, sorbic acid, and phytic acid. In certain embodiments, the excipient is citric acid.

In certain embodiments, In some aspects, a pharmaceutical composition as described herein comprises Compound (I), or a pharmaceutically acceptable salt, solvate, tautomer, stereoisomer, or isotopically labeled derivative thereof, or a polymorph thereof, an organic acid, and a pharmaceutically acceptable excipient.

In certain embodiments, the excipient is a tonicity agent. In certain embodiments, the tonicity agent is selected from sugars (e.g., dextrose, lactose, trehalose, sucrose), sugar alcohols (e.g., mannitol), salts (e.g., sodium chloride, potassium chloride), and polyols (e.g., propylene glycol, glycerin). In certain embodiments, the tonicity agent is a sugar. In certain embodiments, the tonicity agent is dextrose. In certain embodiments, the tonicity agent is lactose. In certain embodiments, the tonicity agent is trehalose. In certain embodiments, the tonicity agent is sucrose. In certain embodiments, the tonicity agent is a sugar alcohol. In certain embodiments, the tonicity agent is mannitol. In certain embodiments, the tonicity agent is a salt. In certain embodiments, the tonicity agent is sodium chloride. In certain embodiments, the tonicity agent is potassium chloride. In certain embodiments, the tonicity agent is a polyol. In certain embodiments, the tonicity agent is propylene glycol. In certain embodiments, the tonicity agent is glycerin.

In certain embodiments, the pharmaceutical composition is formulated as an aqueous solution. In certain embodiments, the pharmaceutical composition is formulated as a powder. In certain embodiments, an aqueous pharmaceutical composition as described herein may be lyophilized to provide a dry composition comprising Compound (I). In certain embodiments, the pharmaceutical composition is formulated for inhalation (e.g., oral or nasal inhalation).

In some aspects, a pharmaceutical composition as described herein comprises Compound (I), or a pharmaceutically acceptable salt, polymorph, solvate, tautomer, stereoisomer, or isotopically labeled derivative thereof; and a pharmaceutically acceptable excipient (e.g., a buffering agent, or a tonicity agent). In certain embodiments, the pharmaceutical composition as described herein comprises Compound (I), or a pharmaceutically acceptable salt, polymorph, solvate, tautomer, stereoisomer, or isotopically labeled derivative thereof; and a buffering agent (e.g., an organic acid). In certain embodiments, the pharmaceutical composition as described herein comprises Compound (I), or a pharmaceutically acceptable salt, polymorph, solvate, tautomer, stereoisomer, or isotopically labeled derivative thereof; and a tonicity agent (e.g., a sugar (e.g., lactose)). In some aspects, a pharmaceutical composition as described herein comprises Compound (I), or a pharmaceutically acceptable salt, polymorph, solvate, tautomer, stereoisomer, or isotopically labeled derivative thereof; a first pharmaceutically acceptable excipient (e.g., a buffering agent); and a second pharmaceutically acceptable excipient (e.g., a tonicity agent).

In certain embodiments, the pharmaceutical composition comprises Compound (I), or a pharmaceutically acceptable salt, polymorph, solvate, tautomer, stereoisomer, or isotopically labeled derivative thereof, a buffering agent (e.g., citric acid), and a second pharmaceutically acceptable excipient. In certain embodiments, the pharmaceutical composition comprises Compound (I), or a pharmaceutically acceptable salt, polymorph, solvate, tautomer, stereoisomer, or isotopically labeled derivative thereof, a pharmaceutically acceptable excipient (e.g., a buffering agent), and a tonicity agent (e.g., lactose). In certain embodiments, the pharmaceutical composition comprises Compound (I), or a pharmaceutically acceptable salt, polymorph, solvate, tautomer, stereoisomer, or isotopically labeled derivative thereof; citric acid; and a second pharmaceutically acceptable excipient. In certain embodiments, the pharmaceutical composition comprises Compound (I), or a pharmaceutically acceptable salt, polymorph, solvate, tautomer, stereoisomer, or isotopically labeled derivative thereof; lactose; and a second pharmaceutically acceptable excipient. In certain embodiments, the pharmaceutical composition comprises Compound (I), or a pharmaceutically acceptable salt, polymorph, solvate, tautomer, stereoisomer, or isotopically labeled derivative thereof; citric acid; and lactose.

In certain embodiments, the composition comprises an amorphous form of Compound (I). In certain embodiments, the pharmaceutical composition comprises a polymorph of Compound (I). In certain embodiments, the pharmaceutical composition comprises a polymorph of Compound (I), or a pharmaceutically acceptable salt, polymorph, solvate, tautomer, stereoisomer, or isotopically labeled derivative thereof, and one or more excipients. In certain embodiments, the pharmaceutical composition comprises a polymorph of Compound (I), or a pharmaceutically acceptable salt, polymorph, solvate, tautomer, stereoisomer, or isotopically labeled derivative thereof; a first excipient; and a second excipient.

In certain embodiments, the polymorph of Compound (I) is Freeform Type A, Freeform Type B, Freeform Type C, or Freeform Type D. In certain embodiments, the polymorph of Compound (I) is Freeform Type A. In certain embodiments, the polymorph of Compound (I) is Freeform Type B. In certain embodiments, the polymorph of Compound (I) is Freeform Type C. In certain embodiments, the polymorph of Compound (I) is Freeform Type D. In certain embodiments, the polymorph of Compound (I) is HCl salt Type A, HCl salt Type B, HCl salt Type C, HCl salt Type D, HCl salt Type E, or HCl salt Type F. In certain embodiments, the polymorph of Compound (I) is HCl salt Type A. In certain embodiments, the polymorph of Compound (I) is HCl salt Type B. In certain embodiments, the polymorph of Compound (I) is HCl salt Type C. In certain embodiments, the polymorph of Compound (I) is HCl salt Type D. In certain embodiments, the polymorph of Compound (I) is HCl salt Type E. In certain embodiments, the polymorph of Compound (I) is HCl salt Type F. In certain embodiments, the polymorph of Compound (I) is sulfate salt Type A, or sulfate salt Type B. In certain embodiments, the polymorph of Compound (I) is sulfate salt Type A. In certain embodiments, the polymorph of Compound (I) is sulfate salt Type B. In certain embodiments, the polymorph of Compound (I) is maleate salt Type A, or maleate salt Type B. In certain embodiments, the polymorph of Compound (I) is maleate salt Type A. In certain embodiments, the polymorph of Compound (I) is maleate salt Type B. In certain embodiments, the polymorph of Compound (I) is tartrate salt Type A. In certain embodiments, the polymorph of Compound (I) is fumarate salt Type A, fumarate salt Type B, fumarate salt Type C, or fumarate salt Type D. In certain embodiments, the polymorph of Compound (I) is fumarate salt Type A. In certain embodiments, the polymorph of Compound (I) is fumarate salt Type B. In certain embodiments, the polymorph of Compound (I) is fumarate salt Type C. In certain embodiments, the polymorph of Compound (I) is fumarate salt Type D. In certain embodiments, the polymorph of Compound (I) is succinate salt Type A, succinate salt Type B, or succinate salt Type C. In certain embodiments, the polymorph of Compound (I) is succinate salt Type A. In certain embodiments, the polymorph of Compound (I) is succinate salt Type B. In certain embodiments, the polymorph of Compound (I) is succinate salt Type C. In certain embodiments, the polymorph of Compound (I) is triphenylacetate salt Type A. In certain embodiments, the polymorph of Compound (I) is xinafoic salt Type A. In certain embodiments, the polymorph of Compound (I) is Ca salt Type A. In certain embodiments, the polymorph of Compound (I) is tromethamine salt Type A, or tromethamine salt Type B. In certain embodiments, the polymorph of Compound (I) is tromethamine salt Type A. In certain embodiments, the polymorph of Compound (I) is tromethamine salt Type B.

In certain embodiments, the pharmaceutical composition comprises Freeform Type D in essentially pure form. In certain embodiments, the pharmaceutical composition comprises Freeform Type D essentially free of other polymorphs. In certain embodiments, the pharmaceutical composition comprises greater than or equal to 90% Freeform Type D by weight as compared to the total of other polymorphs of Compound (I) in the composition. In certain embodiments, the pharmaceutical composition comprises greater than or equal to 95% Freeform Type D by weight as compared to the total of other polymorphs of Compound (I) in the composition. In certain embodiments, the pharmaceutical composition comprises greater than or equal to 96% Freeform Type D by weight as compared to the total of other polymorphs of Compound (I) in the composition. In certain embodiments, the pharmaceutical composition comprises greater than or equal to 97% Freeform Type D by weight as compared to the total of other polymorphs of Compound (I) in the composition. In certain embodiments, the pharmaceutical composition comprises greater than or equal to 98% Freeform Type D by weight as compared to the total of other polymorphs of Compound (I) in the composition. In certain embodiments, the pharmaceutical composition comprises greater than or equal to 99% Freeform Type D by weight as compared to the total of other polymorphs of Compound (I) in the composition. In certain embodiments, the pharmaceutical composition comprises greater than or equal to 99.5% Freeform Type D by weight as compared to the total of other polymorphs of Compound (I) in the composition. In certain embodiments, the pharmaceutical composition comprises greater than or equal to 90% Freeform Type D by weight as compared to the total of other forms of Compound (I) in the composition. In certain embodiments, the pharmaceutical composition comprises greater than or equal to 95% Freeform Type D by weight as compared to the total of other forms of Compound (I) in the composition. In certain embodiments, the pharmaceutical composition comprises greater than or equal to 96% Freeform Type D by weight as compared to the total of other forms of Compound (I) in the composition. In certain embodiments, the pharmaceutical composition comprises greater than or equal to 97% Freeform Type D by weight as compared to the total of other forms of Compound (I) in the composition. In certain embodiments, the pharmaceutical composition comprises greater than or equal to 98% Freeform Type D by weight as compared to the total of other forms of Compound (I) in the composition. In certain embodiments, the pharmaceutical composition comprises greater than or equal to 99% Freeform Type D by weight as compared to the total of other forms of Compound (I) in the composition. In certain embodiments, the pharmaceutical composition comprises greater than or equal to 99.5% Freeform Type D by weight as compared to the total of other forms of Compound (I) in the composition.

In certain embodiments, the pharmaceutical composition comprises a polymorph of Compound (I), wherein the molar ratio of the amount of Freeform Type D to the sum of the amounts of other forms of Compound (I) is equal to or greater than about 80:20. In certain embodiments, the pharmaceutical composition comprises a polymorph of Compound (I), wherein the molar ratio of the amount of Freeform Type D to the sum of the amounts of other forms of Compound (I) is equal to or greater than about 90:10. In certain embodiments, the pharmaceutical composition comprises a polymorph of Compound (I), wherein the molar ratio of the amount of Freeform Type D to the sum of the amounts of other forms of Compound (I) is equal to or greater than about 95:5. In certain embodiments, the pharmaceutical composition comprises a polymorph of Compound (I), wherein the molar ratio of the amount of Freeform Type D to the sum of the amounts of other forms of Compound (I) is equal to or greater than about 99:1.

In certain embodiments, the polymorph is a polymorph of a fumarate salt of Compound (I). In certain embodiments, the polymorph of Compound (I) is Fumarate Type A, Fumarate Type B, Fumarate Type C, or Fumarate Type D. In certain embodiments, the polymorph of Compound (I) is Fumarate Type A. In certain embodiments, the polymorph of Compound (I) is Fumarate Type B. In certain embodiments, the polymorph of Compound (I) is Fumarate Type C. In certain embodiments, the polymorph of Compound (I) is Fumarate Type D.

In certain embodiments, the pharmaceutical composition comprises Fumarate Type A in essentially pure form. In certain embodiments, the pharmaceutical composition comprises Fumarate Type A essentially free of other polymorphs. In certain embodiments, the pharmaceutical composition comprises greater than or equal to 90% Fumarate Type A by weight as compared to the total of other polymorphs of Compound (I) in the composition. In certain embodiments, the pharmaceutical composition comprises greater than or equal to 95% Fumarate Type A by weight as compared to the total of other polymorphs of Compound (I) in the composition. In certain embodiments, the pharmaceutical composition comprises greater than or equal to 96% Fumarate Type A by weight as compared to the total of other polymorphs of Compound (I) in the composition. In certain embodiments, the pharmaceutical composition comprises greater than or equal to 97% Fumarate Type A by weight as compared to the total of other polymorphs of Compound (I) in the composition. In certain embodiments, the pharmaceutical composition comprises greater than or equal to 98% Fumarate Type A by weight as compared to the total of other polymorphs of Compound (I) in the composition. In certain embodiments, the pharmaceutical composition comprises greater than or equal to 99% Fumarate Type A by weight as compared to the total of other polymorphs of Compound (I) in the composition. In certain embodiments, the pharmaceutical composition comprises greater than or equal to 99.5% Fumarate Type A by weight as compared to the total of other polymorphs of Compound (I) in the composition. In certain embodiments, the pharmaceutical composition comprises greater than or equal to 90% Fumarate Type A by weight as compared to the total of other forms of Compound (I) in the composition. In certain embodiments, the pharmaceutical composition comprises greater than or equal to 95% Fumarate Type A by weight as compared to the total of other forms of Compound (I) in the composition. In certain embodiments, the pharmaceutical composition comprises greater than or equal to 96% Fumarate Type A by weight as compared to the total of other forms of Compound (I) in the composition. In certain embodiments, the pharmaceutical composition comprises greater than or equal to 97% Fumarate Type A by weight as compared to the total of other forms of Compound (I) in the composition. In certain embodiments, the pharmaceutical composition comprises greater than or equal to 98% Fumarate Type A by weight as compared to the total of other forms of Compound (I) in the composition. In certain embodiments, the pharmaceutical composition comprises greater than or equal to 99% Fumarate Type A by weight as compared to the total of other forms of Compound (I) in the composition. In certain embodiments, the pharmaceutical composition comprises greater than or equal to 99.5% Fumarate Type A by weight as compared to the total of other forms of Compound (I) in the composition.

In certain embodiments, the pharmaceutical composition comprises a polymorph of Compound (I), wherein the molar ratio of the amount of Fumarate Type A to the sum of the amounts of other forms of Compound (I) is equal to or greater than about 80:20. In certain embodiments, the pharmaceutical composition comprises a polymorph of Compound (I), wherein the molar ratio of the amount of Fumarate Type A to the sum of the amounts of other forms of Compound (I) is equal to or greater than about 90:10. In certain embodiments, the pharmaceutical composition comprises a polymorph of Compound (I), wherein the molar ratio of the amount of Fumarate Type A to the sum of the amounts of other forms of Compound (I) is equal to or greater than about 95:5. In certain embodiments, the pharmaceutical composition comprises a polymorph of Compound (I), wherein the molar ratio of the amount of Fumarate Type A to the sum of the amounts of other forms of Compound (I) is equal to or greater than about 99:1.

Typically, but not absolutely, the salts of the present disclosure are pharmaceutically acceptable salts. Salts of Compound (I) may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid, or the like.

Examples of pharmaceutically acceptable salts include sulfates, pyrosulfates, bisulfates, sulfites, bisulfites, phosphates, chlorides, bromides, iodides, acetates, propionates, decanoates, caprylates, acrylates, formates, isobutyrates, caproates, heptanoates, propiolates, oxalates, malonates succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, phenylacetates, phenylpropionates, phenylbutrates, citrates, lactates, γ-hydroxybutyrates, glycolates, tartrates mandelates, and sulfonates, such as xylenesulfonates, methanesulfonates, propanesulfonates, naphthalene-1-sulfonates, and naphthalene-2-sulfonates.

Salts of Compound (I) may also be prepared by reacting Compound (I) with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases, such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acid such as lysine and arginine.

In certain embodiments, the pharmaceutically acceptable salt is a hydrochloride, sulfate, phosphoric acid, maleic acid, tartartic acid, fumaric acid, citric acid, succinic acid, acetic acid, methanesulfonic acid, isethionic acid, triphenyl acetic acid, or xinafoic acid salt. In certain embodiments, the pharmaceutically acceptable salt is a hydrochloride, sulfate, maleic acid, tartartic acid, fumaric acid, succinic acid, triphenyl acetic acid, or xinafoic acid salt. In certain embodiments, the pharmaceutically acceptable salt is a hydrochloride, sulfate, or fumaric acid salt.

In certain embodiments, the pharmaceutically acceptable salt is a calcium hydroxide, sodium hydroxide, or tromethamine salt. In certain embodiments, the pharmaceutically acceptable salt is a calcium hydroxide salt.This disclosure further provides a pharmaceutical composition (also referred to as pharmaceutical formulation) comprising Compound (I), or a pharmaceutically acceptable salt, solvate, tautomer, stereoisomer, or isotopically labeled derivative thereof, and one or more excipients. The excipients are acceptable in the sense of being compatible with the other ingredients of the formulation and not deleterious to the recipient thereof (i.e., the patient). In certain embodiments, the pharmaceutical composition comprises a polymorph of Compound (I), or a pharmaceutically acceptable salt, solvate, tautomer, stereoisomer, or isotopically labeled derivative thereof, and one or more excipients. In certain embodiments, the pharmaceutical composition comprises Compound (I), or a pharmaceutically acceptable salt, solvate, tautomer, stereoisomer, or isotopically labeled derivative thereof, a first excipient, and a second excipient. In certain embodiments, the pharmaceutical composition comprises a polymorph of Compound (I), or a pharmaceutically acceptable salt, solvate, tautomer, stereoisomer, or isotopically labeled derivative thereof, a first excipient, and a second excipient.

The excipients described herein are acceptable in the sense of being compatible with the other ingredients of the formulation and not deleterious to the recipient thereof (i.e., the patient). Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting of the compound or compounds of this disclosure once administered to the patient from one organ, or portion of the body, to another organ, or portion of the body. Certain pharmaceutically acceptable excipients may be chosen for their ability to enhance patient compliance. Certain pharmaceutically acceptable excipients may be chosen for their ability to facilitate the production of stable dosage forms for inhalation. Certain pharmaceutically acceptable excipients may be chosen for their ability to facilitate the production of stable dosage forms for oral inhalation. Certain pharmaceutically acceptable excipients may be chosen for their ability to facilitate the production of stable dosage forms for nasal inhalation. Certain pharmaceutically acceptable excipients may be chosen for their ability to facilitate the production of stable dosage forms for administration with a nebulizer. Other pharmaceutically acceptable excipients may be chosen for their ability to facilitate the production of stable dosage forms for administration with an inhaler (e.g., a dry powder inhaler).

Suitable pharmaceutically acceptable excipients include the following types of excipients: tonicity agents, carriers, diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other ingredients are present in the formulation.

In some aspects, a pharmaceutical composition as described herein comprises a polymorph of Compound (I), or a pharmaceutically acceptable salt or solvate thereof; a pharmaceutically acceptable excipient (e.g., and organic acid); and a second pharmaceutically acceptable excipient (e.g., a tonicity agent pharmaceutically acceptable carrier). In certain embodiments, the pharmaceutical composition comprises Freeform Type D of Compound (I), a first pharmaceutically acceptable excipient, and a second pharmaceutically acceptable excipient. In certain embodiments, the pharmaceutical composition comprises Freeform Type D of Compound (I), citric acid, and a second pharmaceutically acceptable excipient. In certain embodiments, the pharmaceutical composition comprises Freeform Type D of Compound (I), a pharmaceutically acceptable excipient, and lactose. In certain embodiments, the pharmaceutical composition comprises a polymorph of Compound (I), or a solvate, or pharmaceutically acceptable salt thereof, citric acid, and a second pharmaceutically acceptable excipient. In certain embodiments, the pharmaceutical composition comprises a polymorph of Compound (I), or a solvate, or pharmaceutically acceptable salt thereof, a pharmaceutically acceptable excipient, and lactose. In certain embodiments, the pharmaceutical composition comprises a polymorph of Compound (I), or a solvate, or pharmaceutically acceptable salt thereof, citric acid, and lactose. In certain embodiments, the pharmaceutical composition comprises Freeform Type D of Compound (I), citric acid, and lactose.

In certain embodiments, the composition comprises 1 molar equivalent of Compound (I), or a pharmaceutically acceptable salt, solvate, tautomer, stereoisomer, or isotopically labeled derivative thereof, or a polymorph thereof and about 1 to about 2 molar equivalents of an organic acid (e.g., citric acid). In certain embodiments, the composition comprises 1 molar equivalent of Compound (I), or a pharmaceutically acceptable salt, solvate, tautomer, stereoisomer, or isotopically labeled derivative thereof, and about 1 to about 1.5 equivalents of an organic acid (e.g., citric acid). In certain embodiments, the composition comprises 1 molar equivalent of Compound (I), or a pharmaceutically acceptable salt, solvate, tautomer, stereoisomer, or isotopically labeled derivative thereof, and between about 0.8 to about 1.2 molar equivalents of an organic acid (e.g., citric acid), preferably between about 0.9 to about 1.1 molar equivalents. In certain embodiments, the composition comprises 1 molar equivalent of Compound (I), or a pharmaceutically acceptable salt, solvate, tautomer, stereoisomer, or isotopically labeled derivative thereof, and about 1 to about 1.2 equivalents of an organic acid (e.g., citric acid). In certain embodiments, the composition comprises 1 molar equivalent of Compound (I), or a pharmaceutically acceptable salt, solvate, tautomer, stereoisomer, or isotopically labeled derivative thereof, and about 0.9 to about 1.1 equivalents of an organic acid (e.g., citric acid). In certain embodiments, the composition comprises 1 molar equivalent of Compound (I), or a pharmaceutically acceptable salt, solvate, tautomer, stereoisomer, or isotopically labeled derivative thereof, and about 1 to about 1.1 equivalents of an organic acid (e.g., citric acid). In certain embodiments, the composition comprises 1 molar equivalent of Compound (I), or a pharmaceutically acceptable salt, solvate, tautomer, stereoisomer, or isotopically labeled derivative thereof, and about 1.05 equivalents of an organic acid.

In certain embodiments, the composition comprises 1 molar equivalent of Compound (I), or a pharmaceutically acceptable salt, solvate, tautomer, stereoisomer, or isotopically labeled derivative thereof, and about 1 to about 5 molar equivalents of a pharmaceutically acceptable excipient (e.g., a buffering agent or a tonicity agent (e.g., lactose)). In certain embodiments, the composition comprises 1 molar equivalent of Compound (I), or a pharmaceutically acceptable salt, solvate, tautomer, stereoisomer, or isotopically labeled derivative thereof, and about 1 to about 5 equivalents of a pharmaceutically acceptable excipient (e.g., a buffering agent or a tonicity agent (e.g., lactose)). In certain embodiments, the composition comprises 1 molar equivalent of Compound (I), or a pharmaceutically acceptable salt, solvate, tautomer, stereoisomer, or isotopically labeled derivative thereof, and about 2 to about 4 equivalents of a pharmaceutically acceptable excipient. In certain embodiments, the composition comprises 1 molar equivalent of Compound (I), or a pharmaceutically acceptable salt, solvate, tautomer, stereoisomer, or isotopically labeled derivative thereof, and about 2 to about 3 equivalents of a pharmaceutically acceptable excipient. In certain embodiments, the composition comprises 1 molar equivalent of Compound (I), or a pharmaceutically acceptable salt, solvate, tautomer, stereoisomer, or isotopically labeled derivative thereof, and about 2.5 to about 3.0 equivalents of a pharmaceutically acceptable excipient.

In certain embodiments, the composition comprises 1 molar equivalent of Compound (I), or a pharmaceutically acceptable salt, solvate, tautomer, stereoisomer, or isotopically labeled derivative thereof, about 1 to about 2 molar equivalents of an organic acid (e.g., citric acid), and 1 to 5 molar equivalents of a tonicity agent (e.g., a sugar (e.g., lactose)). In certain embodiments, the composition comprises 1 molar equivalent of Compound (I), or a pharmaceutically acceptable salt, solvate, tautomer, stereoisomer, or isotopically labeled derivative thereof, 1 to 2 molar equivalents of an organic acid (e.g., citric acid), and about 2 to about 3 molar equivalents of a tonicity agent (e.g., lactose). In certain embodiments, the composition comprises 1 molar equivalent of Compound (I), or a pharmaceutically acceptable salt, solvate, tautomer, stereoisomer, or isotopically labeled derivative thereof, about 0.8 to about 1.2 molar equivalents of an organic acid (e.g., citric acid), and about 2.5 to about 3.0 molar equivalents of a tonicity agent (e.g., lactose).

In certain embodiments, the composition comprises 1 molar equivalent of a polymorph of Compound (I), or a pharmaceutically acceptable salt, solvate, tautomer, stereoisomer, or isotopically labeled derivative thereof, and about 1 to about 2 molar equivalents of a pharmaceutically acceptable excipient (e.g., citric acid). In certain embodiments, the composition comprises 1 molar equivalent of a polymorph of Compound (I), or a pharmaceutically acceptable salt, solvate, tautomer, stereoisomer, or isotopically labeled derivative thereof, and about 1 to about 1.5 equivalents of a pharmaceutically acceptable excipient. In certain embodiments, the composition comprises 1 molar equivalent of a polymorph of Compound (I), or a pharmaceutically acceptable salt, solvate, tautomer, stereoisomer, or isotopically labeled derivative thereof, and about 1 to about 1.2 equivalents of a pharmaceutically acceptable excipient. In certain embodiments, the composition comprises 1 molar equivalent of a polymorph of Compound (I), or a pharmaceutically acceptable salt, solvate, tautomer, stereoisomer, or isotopically labeled derivative thereof, and about 1 to about 1.1 equivalents of a pharmaceutically acceptable excipient. In certain embodiments, the composition comprises 1 molar equivalent of a polymorph of Compound (I), or a pharmaceutically acceptable salt, solvate, tautomer, stereoisomer, or isotopically labeled derivative thereof, and about 1.05 equivalents of a pharmaceutically acceptable excipient.

In certain embodiments, the composition comprises 1 molar equivalent of a polymorph of Compound (I), or a pharmaceutically acceptable salt, solvate, tautomer, stereoisomer, or isotopically labeled derivative thereof, and about 1 to about 5 molar equivalents of a pharmaceutically acceptable excipient (e.g., a buffering agent or a tonicity agent (e.g., lactose)). In certain embodiments, the composition comprises 1 molar equivalent of a polymorph of Compound (I), or a pharmaceutically acceptable salt, solvate, tautomer, stereoisomer, or isotopically labeled derivative thereof, and about 1 to about 5 equivalents of a pharmaceutically acceptable excipient. In certain embodiments, the composition comprises 1 molar equivalent of a polymorph of Compound (I), or a pharmaceutically acceptable salt, solvate, tautomer, stereoisomer, or isotopically labeled derivative thereof, and about 2 to about 4 equivalents of a pharmaceutically acceptable excipient. In certain embodiments, the composition comprises 1 molar equivalent of a polymorph of Compound (I), or a pharmaceutically acceptable salt, solvate, tautomer, stereoisomer, or isotopically labeled derivative thereof, and about 2 to about 3 equivalents of a pharmaceutically acceptable excipient. In certain embodiments, the composition comprises 1 molar equivalent of a polymorph of Compound (I), or a pharmaceutically acceptable salt, solvate, tautomer, stereoisomer, or isotopically labeled derivative thereof, and about 2.5 to about 3.0 equivalents of a pharmaceutically acceptable excipient.

In certain embodiments, the composition comprises 1 molar equivalent of a polymorph of Compound (I), or a pharmaceutically acceptable salt, solvate, tautomer, stereoisomer, or isotopically labeled derivative thereof, about 1 to about 2 molar equivalents of a first pharmaceutically acceptable excipient (e.g., citric acid), and about 1 to about 5 molar equivalents of a second pharmaceutically acceptable excipient (e.g., lactose)). In certain embodiments, the composition comprises 1 molar equivalent of a polymorph of Compound (I), or a pharmaceutically acceptable salt, solvate, tautomer, stereoisomer, or isotopically labeled derivative thereof, about 1 to about 2 molar equivalents of a first pharmaceutically acceptable excipient (e.g., citric acid), and about 1 to about 5 molar equivalents of a tonicity agent (e.g., lactose).

In certain embodiments, the composition comprises 1 molar equivalent of Freeform Type D of Compound (I), about 1 to about 2 molar equivalents of citric acid, and about 1 to about 5 molar equivalents of lactose. In certain embodiments, the composition comprises 1 molar equivalent of Freeform Type D, about 1.05 molar equivalents of citric acid, and about 2.5 to about 3 molar equivalents of lactose.

In some aspects, a composition described herein is provided as a solution comprising a polymorph of Compound (I), or a pharmaceutically acceptable salt or solvate thereof, a first pharmaceutically acceptable excipient (e.g., citric acid), and a second pharmaceutically acceptable excipient (e.g., lactose). In some aspects, a composition described herein is provided as a solution comprising a polymorph of Compound (I), or a pharmaceutically acceptable salt, solvate, tautomer, stereoisomer, or isotopically labeled derivative thereof, a first pharmaceutically acceptable excipient (e.g., citric acid), and a second pharmaceutically acceptable excipient (e.g., lactose). In certain embodiments, the pharmaceutical composition is an aqueous solution. In certain embodiments, the solution comprises about 40 mg/mL of a polymorph of Compound (I), or a pharmaceutically acceptable salt, solvate, tautomer, stereoisomer, or isotopically labeled derivative thereof, about 10 to about 20 mg/mL of a first pharmaceutically acceptable excipient (e.g., citric acid), and about 50 to about 80 mg/mL of a second pharmaceutically acceptable excipient (e.g., lactose). In certain embodiments, the solution comprises about 40 mg/mL of a polymorph of Compound (I), or a pharmaceutically acceptable salt, solvate, tautomer, stereoisomer, or isotopically labeled derivative thereof, about 12 to about 13 mg/mL of a first pharmaceutically acceptable excipient (e.g., citric acid), and about 55 to about 65 mg/mL of a second pharmaceutically acceptable excipient (e.g., lactose). In certain embodiments, the solution comprises about 40 mg/mL of a polymorph of Compound (I), or a pharmaceutically acceptable salt, solvate, tautomer, stereoisomer, or isotopically labeled derivative thereof, about 12 to about 13 mg/mL of citric acid, and about 50 to about 80 mg/mL of lactose.

In certain embodiments, the composition comprises 1 molar equivalent of Compound (I), or a pharmaceutically acceptable salt, solvate, tautomer, stereoisomer, or isotopically labeled derivative thereof, and about 1 to about 2 molar equivalents of a pharmaceutically acceptable excipient (e.g., citric acid). In certain embodiments, the composition comprises 1 molar equivalent of Compound (I), or a pharmaceutically acceptable salt, solvate, tautomer, stereoisomer, or isotopically labeled derivative thereof, and about 1 to about 1.5 equivalents of a pharmaceutically acceptable excipient. In certain embodiments, the composition comprises 1 molar equivalent of Compound (I), or a pharmaceutically acceptable salt, solvate, tautomer, stereoisomer, or isotopically labeled derivative thereof, and about 1 to about 1.2 equivalents of a pharmaceutically acceptable excipient. In certain embodiments, the composition comprises 1 molar equivalent of Compound (I), or a pharmaceutically acceptable salt, solvate, tautomer, stereoisomer, or isotopically labeled derivative thereof, and about 1 to about 1.1 equivalents of a pharmaceutically acceptable excipient. In certain embodiments, the composition comprises 1 molar equivalent of Compound (I), or a pharmaceutically acceptable salt, solvate, tautomer, stereoisomer, or isotopically labeled derivative thereof, and about 1.05 equivalents of a pharmaceutically acceptable excipient.

In certain embodiments, the composition comprises 1 molar equivalent of Compound (I), or a pharmaceutically acceptable salt, solvate, tautomer, stereoisomer, or isotopically labeled derivative thereof, and about 1 to about 5 molar equivalents of a pharmaceutically acceptable excipient (e.g., a buffering agent or a tonicity agent(e.g., lactose)). In certain embodiments, the composition comprises 1 molar equivalent of Compound (I), or a pharmaceutically acceptable salt, solvate, tautomer, stereoisomer, or isotopically labeled derivative thereof, and about 1 to about 5 equivalents of a pharmaceutically acceptable excipient. In certain embodiments, the composition comprises 1 molar equivalent of Compound (I), or a pharmaceutically acceptable salt, solvate, tautomer, stereoisomer, or isotopically labeled derivative thereof, and about 2 to about 4 equivalents of a pharmaceutically acceptable excipient. In certain embodiments, the composition comprises 1 molar equivalent of Compound (I), or a pharmaceutically acceptable salt, solvate, tautomer, stereoisomer, or isotopically labeled derivative thereof, and about 2 to about 3 equivalents of a pharmaceutically acceptable excipient. In certain embodiments, the composition comprises 1 molar equivalent of Compound (I), or a pharmaceutically acceptable salt, solvate, tautomer, stereoisomer, or isotopically labeled derivative thereof, and about 2.5 to about 3.0 equivalents of a pharmaceutically acceptable excipient.

In certain embodiments, the composition comprises 1 molar equivalent of Compound (I), or a pharmaceutically acceptable salt, solvate, tautomer, stereoisomer, or isotopically labeled derivative thereof, about 1 to about 2 molar equivalents of a first pharmaceutically acceptable excipient (e.g., citric acid), and about 1 to about 5 molar equivalents of a second pharmaceutically acceptable excipient (e.g., lactose)). In certain embodiments, the composition comprises 1 molar equivalent of Compound (I), or a pharmaceutically acceptable salt, solvate, tautomer, stereoisomer, or isotopically labeled derivative thereof, about 1 to about 2 molar equivalents of a first pharmaceutically acceptable excipient (e.g., citric acid), and about 1 to about 5 molar equivalents of a tonicity agent (e.g., lactose).

In certain embodiments, the composition comprises 1 molar equivalent of Freeform Type D of Compound (I), about 1 to about 2 molar equivalents of citric acid, and about 1 to about 5 molar equivalents of lactose. In certain embodiments, the composition comprises 1 molar equivalent of Freeform Type D, about 1.05 molar equivalents of citric acid, and about 2.5 to about 3 molar equivalents of lactose.

In some aspects, a composition described herein is provided as a solution comprising Compound (I), or a pharmaceutically acceptable salt or solvate thereof, a first pharmaceutically acceptable excipient (e.g., citric acid), and a second pharmaceutically acceptable excipient (e.g., lactose). In some aspects, a composition described herein is provided as a solution comprising Compound (I), or a pharmaceutically acceptable salt, solvate, tautomer, stereoisomer, or isotopically labeled derivative thereof, a first pharmaceutically acceptable excipient (e.g., citric acid), and a second pharmaceutically acceptable excipient (e.g., lactose). In certain embodiments, the pharmaceutical composition is an aqueous solution. In certain embodiments, the pH of the aqueous solution is between about pH 2 and about pH 8. In certain embodiments, the pH of the aqueous solution is between about pH 3.5 and about pH 6. In certain embodiments, the pH of the aqueous solution is between about pH 4.5 and about pH 5.5.

In certain embodiments, the aqueous solution comprises about 40 mg/mL of Compound (I), or a pharmaceutically acceptable salt, solvate, tautomer, stereoisomer, or isotopically labeled derivative thereof, about 10 to about 20 mg/mL of a first pharmaceutically acceptable excipient (e.g., citric acid), and about 50 to about 80 mg/mL of a second pharmaceutically acceptable excipient (e.g., lactose). In certain embodiments, the aqueous solution comprises about 40 mg/mL of Compound (I), or a pharmaceutically acceptable salt, solvate, tautomer, stereoisomer, or isotopically labeled derivative thereof, about 12 to about 13 mg/mL of a first pharmaceutically acceptable excipient (e.g., citric acid), and about 55 to about 65 mg/mL of a second pharmaceutically acceptable excipient (e.g., lactose). In certain embodiments, the aqueous solution comprises about 40 mg/mL of Compound (I), or a pharmaceutically acceptable salt, solvate, tautomer, stereoisomer, or isotopically labeled derivative thereof, about 12 to about 13 mg/mL of citric acid, and about 50 to about 80 mg/mL of lactose.

In certain embodiments, the aqueous solution comprises Compound (I) in a concentration between about 10 and about 50 mM. In certain embodiments, the aqueous solution comprises Compound (I) in a concentration between about 35 and about 45 mM. In certain embodiments, the aqueous solution comprises Compound (I) in a concentration between 10 and 50 mM, and the concentration of citric acid is about 40 mM. In certain embodiments, the aqueous solution comprises Compound (I) in a concentration between about 10 and about 50 mM, and the concentration of lactose is about 173 mM. In certain embodiments, the aqueous solution comprises Compound (I) in a concentration between about 10 and about 50 mM, and the concentration of citric acid is about 40 mM, and the concentration of lactose is about 173 mM. In certain embodiments, the concentration of Compound (I) is about 40 mM, the concentration of citric acid is about 40 mM, and the concentration of lactose is about 173 mM.

In certain embodiments, the aqueous solution is isotonic with human bodily fluid (e.g., blood). In certain embodiments, the aqueous solution is isotonic with human blood. In certain embodiments, the aqueous solution is isotonic with a human tissue (e.g., human lung or nasal tissue). In certain embodiments, the aqueous solution is isotonic with human lung tissue. In certain embodiments, the aqueous solution is isotonic with human nasal tissue.

Pharmaceutical compositions may be adapted for administration by any appropriate route, for example, by oral (including buccal or sublingual), rectal, nasal, topical (including buccal, sublingual, or transdermal), vaginal, or parenteral (including subcutaneous, intramuscular, intravenous, or intradermal) routes. Such compositions may be prepared by any method known in the art of pharmacy, for example, by bringing into association the active ingredient with the excipient(s). In certain embodiments, the composition is an aqueous solution. In certain embodiments, the pharmaceutical composition is formulated for oral inhalation. In certain embodiments, the pharmaceutical composition is formulated nasal inhalation. In certain embodiments, the pharmaceutical composition is formulated for administration by a nebulizer. In certain embodiments, the pharmaceutical composition is formulated for administration by an inhaler (e.g., a dry powder inhaler).

When adapted for oral administration, pharmaceutical compositions may be in discrete units, such as tablets or capsules; powders or granules; solutions or suspensions in aqueous or non-aqueous liquids; edible foams or whips; oil-in-water liquid emulsions or water-in-oil liquid emulsions. The compound or salt thereof of the disclosure or the pharmaceutical composition of the disclosure may also be incorporated into a candy, a wafer, and/or tongue tape formulation for administration as a “quick-dissolve” medicine.

For instance, for oral administration in the form of a tablet or capsule, the active drug component can be combined with an oral, non-toxic pharmaceutically acceptable inert carrier such as ethanol, glycerol, water, and the like. Powders or granules are prepared by comminuting the compound to a suitable fine size and mixing with a similarly comminuted pharmaceutically acceptable carrier such as an edible carbohydrate, as, for example, starch or mannitol. Flavoring, preservative, dispersing, and coloring agents can also be present.

Capsules are made by preparing a powder mixture, as described above, and filling formed gelatin or non-gelatinous sheaths. Glidants and lubricants, such as colloidal silica, talc, magnesium stearate, calcium stearate, solid polyethylene glycol, can be added to the powder mixture before the filling operation. A disintegrating or solubilizing agent, such as agar-agar, calcium carbonate, or sodium carbonate, can also be added to improve the availability of the medicine when the capsule is ingested.

Moreover, when desired or necessary, suitable binders, lubricants, disintegrating agents, and coloring agents can also be incorporated into the mixture. Suitable binders include starch, gelatin, natural sugars, such as glucose or beta-lactose, corn sweeteners, natural and synthetic gums such as acacia, tragacanth, sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methylcellulose, agar, bentonite, xanthan gum, and the like.

Tablets are formulated, for example, by preparing a powder mixture, granulating or slugging, adding a lubricant and disintegrant, and pressing into tablets. A powder mixture is prepared by mixing the compound, suitably comminuted, with a diluent or base as described above, and optionally, with a binder such as carboxymethylcellulose, and aliginate, gelatin, or polyvinyl pyrrolidone, a solution retardant such as paraffin, a resorption accelerator such as a quaternary salt, and/or an absorption agent such as bentonite, kaolin, or dicalcium phosphate. The powder mixture can be granulated by wetting a binder such as syrup, starch paste, acadia mucilage, or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc, or mineral oil. The lubricated mixture is then compressed into tablets. The compound or salt of the present disclosure can also be combined with a free-flowing inert carrier and compressed into tablets directly without going through the granulating or slugging steps. A clear opaque protective coating consisting of a sealing coat of shellac, a coating of sugar, or polymeric material, and a polish coating of wax can be provided. Dyestuffs can be added to these coatings to distinguish different dosages.

Oral fluids such as solutions, syrups, and elixirs can be prepared in dosage unit form so that a given quantity contains a predetermined amount of active ingredient. Syrups can be prepared by dissolving a polymorph of Compound (I) in a suitably flavored aqueous solution, while elixirs are prepared through the use of a non-toxic alcoholic vehicle. Suspensions can be formulated by dispersing the polymorph of Compound (I) in a non-toxic vehicle. Solubilizers and emulsifiers, such as ethoxylated isostearyl alcohols and polyoxyethylene sorbitol ethers, preservatives, flavor additives such as peppermint oil, natural sweeteners, saccharin, or other artificial sweeteners, and the like, can also be added.

Where appropriate, dosage unit formulations for oral administration can be microencapsulated. The formulation can also be prepared to prolong or sustain the release as, for example, by coating or embedding particulate material in polymers, wax, or the like.

In another aspect, the polymorphs and compositions as described herein can be adapted for administration to a patient by inhalation. Inhalation refers to administration into the patient's lungs whether inhaled through the mouth or through the nasal passages. For example, a polymorph of Compound (I) may be inhaled into the lungs as a dry powder, an aerosol, a suspension, or a solution.

Dry powder compositions for delivery to the lung by inhalation typically comprise Compound (I) as a finely divided powder together with one or more pharmaceutically acceptable excipients as finely divided powders. Pharmaceutically acceptable excipients particularly suited for use in dry powders are known to those skilled in the art and include lactose, starch, mannitol, and mono-, di-, and polysaccharides.

The dry powder may be administered to the patient via a reservoir dry powder inhaler (RDPI) having a reservoir suitable for storing multiple (un-metered doses) of medicament in dry powder form. RDPIs typically include a means for metering each medicament dose from the reservoir to a delivery position. For example, the metering means may comprise a metering cup, which is movable from a first position where the cup may be filled with medicament from the reservoir to a second position where the metered medicament dose is made available to the patient for inhalation.

Alternatively, the dry powder may be presented in capsules (e.g. gelatin or plastic), cartridges, or blister packs for use in a multi-dose dry powder inhaler (MDPI). MDPIs are inhalers wherein the medicament is comprised within a multi-dose pack containing (or otherwise carrying) multiple defined doses (or parts thereof) of medicament. When the dry powder is presented as a blister pack, it comprises multiple blisters for containment of the medicament in dry powder form. The blisters are typically arranged in regular fashion for ease of release of the medicament therefrom. For example, the blisters may be arranged in a generally circular fashion on a disc-form blister pack, or the blisters may be elongate in form, for example comprising a strip or a tape. Each capsule, cartridge, or blister may, for example, contain between 20 μg-10 mg of Compound (I).

Aerosols may be formed by suspending or dissolving a polymorph of Compound (I) in a liquefied propellant. Suitable propellants include halocarbons, hydrocarbons, and other liquefied gases. Representative propellants include: trichlorofluoromethane (propellant 11), dichlorofluoromethane (propellant 12), dichlorotetrafluoroethane (propellant 114), tetrafluoroethane (HFA-134a), 1,1-difluoroethane (HFA-152a), difluoromethane (HFA-32), pentafluoroethane (HFA-12), heptafluoropropane (HFA-227a), perfluoropropane, perfluorobutane, perfluoropentane, butane, isobutane, and pentane. Aerosols comprising a polymorph of Compound (I) as described herein will typically be administered to a patient via a metered dose inhaler (MDI). Such devices are known to those skilled in the art.

The aerosol may contain additional pharmaceutically acceptable excipients typically used with multiple dose inhalers such as tonicity agents, carriers, surfactants, lubricants, cosolvents and other excipients to improve the physical stability of the formulation, to improve valve performance, to improve solubility, or to improve taste.

Suspensions and solutions comprising a polymorph or composition as described herein may also be administered to a patient via a nebulizer. The solvent or suspension agent utilized for nebulization may be any pharmaceutically acceptable liquid such as water, aqueous saline, alcohols or glycols, e.g. ethanol, isopropyl alcohol, glycerol, propylene glycol, polyethylene glycol, etc. or mixtures thereof. Saline solutions utilize salts which display little or no pharmacological activity after administration. Both organic or inorganic salts may be used for this purpose.

Suspensions and solutions comprising a polymorph or composition as described herein may also be administered to a patient via an inhaler (e.g., a dry powder inhaler).

Other pharmaceutically acceptable excipients may be added to the suspension or solution. The polymorphs of Compound (I) as described herein may be stabilized by the addition of an inorganic acid, e.g. hydrochloric acid, nitric acid, sulfuric acid and/or phosphoric acid; an organic acid, e.g. ascorbic acid, citric acid, acetic acid, and tartaric acid, etc., a complexing agent, such as EDTA or citric acid, and salts thereof; or an antioxidant, such as vitamin E or ascorbic acid. These may be used alone or together to stabilize the polymorphs of Compound (I) as described herein. Preservatives may be added such as benzalkonium chloride or benzoic acid and salts thereof.

Also disclosed are methods of using the polymorphs or compositions described herein to treat a disease or condition comprising administering to a subject in need thereof a therapeutically effective amount of a polymorph of Compound (I) or a pharmaceutical composition as described herein.

In another aspect, this disclosure provides a polymorph of Compound (I) or a pharmaceutically acceptable salt or solvate thereof, or a composition comprising a polymorph of Compound (I), for use in the manufacture of a medicament for use in the treatment of a disorder mediated by furin, such as fibrotic diseases (e.g., pulmonary fibrosis).

In another aspect, this disclosure provides a polymorph of Compound (I) or a pharmaceutically acceptable salt or solvate thereof, or a composition comprising a polymorph of Compound (I), for use in the manufacture of a medicament for use in the treatment of a disorder mediated by furin, such as cystic fibrosis.

In another aspect, this disclosure provides a polymorph of Compound (I) or a pharmaceutically acceptable salt or solvate thereof, or a composition comprising a polymorph of Compound (I), for use in the treatment of diseases mediated by furin. In another aspect, this disclosure provides a polymorph of Compound (I) or a pharmaceutically acceptable salt or solvate thereof, or a composition comprising a polymorph of Compound (I), for as an active therapeutic substance for use in the treatment of a disease mediated by or associated with furin.

In another aspect, this disclosure provides a polymorph of Compound (I), or a pharmaceutically acceptable salt or solvate thereof, or a composition comprising a polymorph of Compound (I) for use in therapy.

In another aspect, this disclosure provides a polymorph of Compound (I) or a solvate, or pharmaceutically acceptable salt thereof, or a composition comprising a polymorph of Compound (I), for use in the treatment of fibrotic diseases.

In another aspect, this disclosure provides a polymorph of Compound (I) or a pharmaceutically acceptable salt or solvate thereof, or a composition comprising a polymorph of Compound (I) for use in the treatment of pulmonary fibrosis.

In another aspect, this disclosure provides a polymorph of Compound (I) or a solvate, or pharmaceutically acceptable salt thereof, or a composition comprising a polymorph of Compound (I), for use in the treatment of cystic fibrosis.

In another aspect, this disclosure provides methods of co-administering a polymorph of Compound (I) or a pharmaceutically acceptable salt or solvate thereof, or a composition comprising a polymorph of Compound (I), with other active ingredients.

Disease states which can be treated by the methods and compositions provided herein include, but are not limited to, fibrotic diseases. Fibrotic diseases involve the formation of excess fibrous connective tissue in an organ or tissue in a reparative or reactive process. Diseases may include, but are not limited to, pulmonary fibrosis, e.g., idiopathic pulmonary fibrosis, non-specific interstitial pneumonia (NSIP), usual interstitial pneumonia (UIP), Hermansky-Pudlak syndrome, progressive massive fibrosis (a complication of coal workers' pneumoconiosis), connective tissue disease-related pulmonary fibrosis, airway fibrosis in asthma and COPD, acute respiratory distress syndrome (ARDS) associated fibrosis, acute lung injury (e.g., radiation-induced acute lung injury, chemical lung injury); systemic sclerosis associated interstitial lung disease; radiation-induced fibrosis; familial pulmonary fibrosis; pulmonary hypertension); renal fibrosis (diabetic nephropathy, IgA nephropathy, lupus nephritis; focal segmental glomerulosclerosis (FSGS), transplant nephropathy, autoimmune nephropathy, drug-induced nephropathy, hypertension-related nephropathy, nephrogenic systemic fibrosis); liver fibrosis (viral-induced fibrosis (e.g. hepatitis C or B), autoimmune hepatitis, primary biliary cirrhosis, alcoholic liver disease, non-alcoholic fatty liver disease including non-alcoholic steatohepatitis (NASH), congenital hepatic fibrosis, primary sclerosing cholangitis, drug-induced hepatitis, hepatic cirrhosis); skin fibrosis (hypertrophic scars, scleroderma, keloids, dermatomyositis, eosinophilic fasciitis, Dupytrens contracture, Ehlers-Danlos syndrome, Peyronie's disease epidermolysis bullosa dystrophica, oral submucous fibrosis); non-cystic fibrosis bronchiectasis (NCFBC); ocular fibrosis (AMD, diabetic macular oedema, dry eye, glaucoma); cardiac fibrosis (congestive heart failure, endomyocardial fibrosis, hypertrophic cardiomyopathy (HCM), dilated cardiomyopathy (DCM), arrhythmogenic right ventricular cardiomyopathy (ARVC), hypertensive heart disease, cardiac sarcoidosis and other forms of heart failure), and other miscellaneous fibrotic conditions (mediastinal fibrosis, myelofibrosis, retroperitoneal fibrosis, Crohn's disease, neurofibromatosis, uterine leiomyomas (fibroids), chronic organ transplant rejection).

In certain embodiments, the disease is cystic fibrosis. In certain embodiments, the disease is chronic obstructive pulmonary disease (COPD). In certain embodiments, the disease is non-cystic fibrosis bronchiectasis (NCFBC). In certain embodiments, the disease is asthma. In certain embodiments, the disease is pulmonary fibrosis, e.g., idiopathic pulmonary fibrosis. In certain embodiments, the disease is idiopathic pulmonary fibrosis. In certain embodiments, the disease is radiation induced acute lung injury. In certain embodiments, the disease is chemical acute lung injury. In certain embodiments, the disease is systemic sclerosis associated interstitial lung disease.

Additional disease states which can be treated by the methods and compositions provided herein include, but are not limited to, hypertension, cancer, infectious diseases (such as human immunodeficiency virus (HIV), nipah virus, avian influenza virus, measles virus, respiratory syncytial virus (RSV), Ebola virus, anthrax, and Zika virus (ZIKV)), respiratory diseases (such as cystic fibrosis (CF)), and neurodegenerative diseases (such as Alzheimer's disease (AD)). In certain embodiments, the disease is hypertension. In certain embodiments, the disease is cancer. In certain embodiments, the disease is infectious diseases (such as human immunodeficiency virus (HIV), nipah virus, avian influenza virus, measles virus, respiratory syncytial virus (RSV), Ebola virus, coronaviruses, anthrax, and Zika virus (ZIKV)). In certain embodiments, the disease is respiratory diseases (such as cystic fibrosis (CF)), and neurodegenerative diseases (such as Alzheimer's disease (AD)).

The polymorphs and compositions described herein can be combined with or co-administered with other therapeutic agents, particularly agents that may enhance the activity of the polymorphs. Combination therapies comprise the administration of at least one polymorph of Compound (I) as described herein and the use of at least one other treatment method, including administration of one or more other therapeutic agents. Other therapeutic agents which may be used in combination with the polymorphs or compositions comprising Compound (I) as described herein include, but are not limited to, antigen immunotherapy, anti-histamines, corticosteroids (e.g., fluticasone propionate, fluticasone furoate, beclomethasone dipropionate, budesonide, ciclesonide, mometasone furoate, triamcinolone, flunisolide), NSAIDs, leukotriene modulators (e.g. montelukast, zafirlukast, pranlukast) iNOS inhibitors, tryptase inhibitors, IKK2 inhibitors, p38 inhibitors, Syk inhibitors, elastase inhibitors, beta-2 integrin antagonists, adenosine a2a agonists, chemokine antagonists such as CCR3 antagonists or CCR4 antagonists, mediator release inhibitors such as sodium chromoglycate, 5-lipoxygenase inhibitors (zyflo), DP1 antagonists, DP2 antagonists, pI3K delta inhibitors, ITK inhibitors, LP (lysophosphatidic) inhibitors or FLAP (5-lipoxygenase activating protein) inhibitors (e.g., sodium 3-(3-(tert-butylthio)-1-(4-(6-ethoxypyridin-3-yl)benzyl)-5-((5-methylpyridin-2-yl)methoxy)-1H-indol-2-yl)-2,2-dimethylpropanoate), methotrexate, and similar agents; monoclonal antibody therapy such as anti-IgE, anti-TNF, anti-IL-5, anti-IL-6, anti-IL-12, anti-IL-1 and similar agents; receptor therapies e.g., etanercept and similar agents; antigen non-specific immunotherapies (e.g., interferon or other cytokines/chemokines, cytokine/chemokine receptor modulators, cytokine agonists or antagonists, TLR agonists and similar agents)), inhibitors of TGFβ synthesis, for example pirfenidone, tyrosine kinase inhibitors targeting the vascular endothelial growth factor (VEGF), platelet-derived growth factor (PDGF) and fibroblast growth factor (FGF) receptor kinases, for example intedanib (BIBF-1120) and imatinib mesylate (Gleevec), endothelin receptor antagonists, for example, ambrisentan or macitentan, antioxidants, such as N-acetylcysteine (NAC or fluimucil), antibiotics, such as tetracyclines, for example minocycline hydrochloride, phosphodiesterase 5 (PDE5) inhibitors for example sildenafil, or αᵥβ₆ integrin antagonists, e.g. monoclonal antibodies such as those described in WO 2003/100033 A2.

By the term “co-administration” and derivatives thereof as used herein refers to either simultaneous administration or any manner of separate sequential administration of a furin inhibiting compound, as described herein, and a further active ingredient or ingredients. The term further active ingredient or ingredients, as used herein, includes any compound or therapeutic agent known to or that demonstrates advantageous properties when administered to a patient in need of treatment. Preferably, if the administration is not simultaneous, the compounds are administered in a close time proximity to each other. Furthermore, it does not matter if the compounds are administered in the same dosage form, e.g. one compound may be administered orally and another compound may be administered intravenously.

The exact amount of a polymorph of composition comprising Compound (I) required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, the duration between the first dose and last dose of the multiple doses is three months, six months, or one year. In certain embodiments, the duration between the first dose and last dose of the multiple doses is the lifetime of the subject. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A therapeutically effective amount of a compound of the present disclosure will depend upon a number of factors including, for example, the age and weight of the intended recipient, the precise condition requiring treatment and its severity, the nature of the formulation, and the route of administration, and will ultimately be at the discretion of the attendant prescribing the medication.

Also encompassed by the present disclosure are kits (e.g., pharmaceutical packs). In certain embodiments, the kit comprises a polymorph of Compound (I) or a pharmaceutical composition as described herein, and instructions for using the polymorph or pharmaceutical composition. In certain embodiments, the kit comprises a first container, wherein the first container includes the polymorph of Compound (I) or pharmaceutical composition comprising Compound (I). In some embodiments, the kit further comprises a second container. In certain embodiments, the second container includes an excipient (e.g., an excipient for dilution or suspension of the compound or pharmaceutical composition). In certain embodiments, each of the first or second containers are independently a vial, ampule, bottle, syringe, dispenser package, tube, nebulizer, or inhaler (e.g., a dry powder inhaler). In certain embodiments, the kit comprises a polymorph of Compound (I) or a pharmaceutical composition comprising Compound (I) as described herein, a first pharmaceutically acceptable excipient (e.g., citric acid), and a second pharmaceutically acceptable excipient (e.g., lactose).

In certain embodiments, a kit described herein includes a first container comprising a polymorph of Compound (I) or a pharmaceutical composition as described herein. In certain embodiments, a kit described herein is useful in treating and/or preventing pulmonary fibrosis.

In certain embodiments, the kit comprises a polymorph of Compound (I), or a pharmaceutical composition thereof; and instructions for using the polymorph or pharmaceutical composition as described herein.

In certain embodiments, the kit comprises an amorphous form of Compound (I), or a pharmaceutical composition thereof; and instructions for using the amorphous form or pharmaceutical composition as described herein.

In certain embodiments, a kit described herein further includes instructions for using the polymorph of Compound (I) or pharmaceutical composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating pulmonary fibrosis.

In certain embodiments, the instructions are for administering the polymorph of Compound (I) or pharmaceutical composition to a subject (e.g., a subject in need of treatment or prevention of a disease described herein). In certain embodiments, the instructions comprise information required by a regulatory agency, such as the U.S. Food and Drug Administration (FDA) or the European Agency for the Evaluation of Medicinal Products (EMA). In certain embodiments, the instructions comprise prescribing information.

### EXAMPLES

TABLE 1-1
Summary of Compound (I) salt forms and freeforms

| | Weight | Endo- | |
|---|---|---|---|
| | loss in | therm | |
| | TGA, (wt %, | in DSC, | |
| Form | temp.) | (peak) | Moisture Uptake (25° C.) |
| Freeform | 7.3 (150° C.) | 110.3° C. | 6.8% (from 0% RH to 80% RH) |
| Type A | | | 2.3 (from 10% RH to 80% RH) |
| Freeform | 8.2 (120° C.) | 106.7° C. | 6.3% (from 0% RH to 80% RH) |
| Type D | | | 0.3% (from 10% RH to 80% RH) |
| HCl salt | 3.4 (190° C.) | 249.5° C., | 2.3% (from 0% RH to 80% RH) |
| Type F | | 288.1° C. | 1.4 (from 20% RH to 80% RH) |
| Sulfate | 4.8 (200° C.) | 86.4° C., | 7.2% (from 0% RH to 80% RH) |
| Type B | | 255.3° C., | |
| | | 279.7° C. | |
| Fumarate | 6.6 (170° C.) | 158.9° C. | 6.1% (from 0% RH to 80% RH) |
| Type A | | | 2.5 (from 30% RH to 80% RH) |

TABLE 1-2
Summary of HPLC purity by area % results in
stability evaluation of formulations 1-5

| | 5° C. for | 25° C. for | 40° C. for | 60° C. for |
|---|---|---|---|---|
| Formulation Number | 37 days | 28 days | 28 days | 16 days |
| ~30 mM API + 30 mM citric acid | 99.06 | 99.01 | 97.74 | 91.87 |
| (~20 mg/mL, formulation 1) | | | | |
| ~30 mM API + 30 mM citric | 99.06 | 98.91 | 97.78 | 91.81 |
| acid + 240 mM lactose | | | | |
| (~20 mg/mL, formulation 2) | | | | |
| ~30 mM API + 30 mM citric | 99.07 | 99.00 | 97.74 | 92.06 |
| acid + 240 mM trehalose | | | | |
| (~20 mg/mL, formulation 3) | | | | |
| ~30 mM API + 30 mM citric | 99.07 | 99.02 | 97.71 | 91.94 |
| acid + 240 mM sucrose | | | | |
| (~20 mg/mL, formulation 4) | | | | |
| ~30 mM API pH = 4.0 | 98.98 | 98.84 | 97.05 | 92.11 |
| citrate/phosphate buffer | | | | |
| (~20 mg/mL, formulation 5) | | | | |

| | |
|---|---|
| ~40 mg/mL Compound (I) | 99.01 (stored under RT for 1 week) |
| freeform (weight adjusted) + 1.05 | |
| equiv. citric acid + 173 mM lactose | |

### Example 1. Polymorph Formation and Characterization

Compound (I) Freeform Type A and freeform Type D were obtained from freeform isolation (Example 7). Using Compound (I) freeform Type A as starting material, 100 polymorph formation experiments were performed via slurry at RT/50° C., slow evaporation, solid/liquid vapor diffusion, temperature cycling, polymer induced crystallization and anti-solvent addition. Experimental details are provided in Example 7.

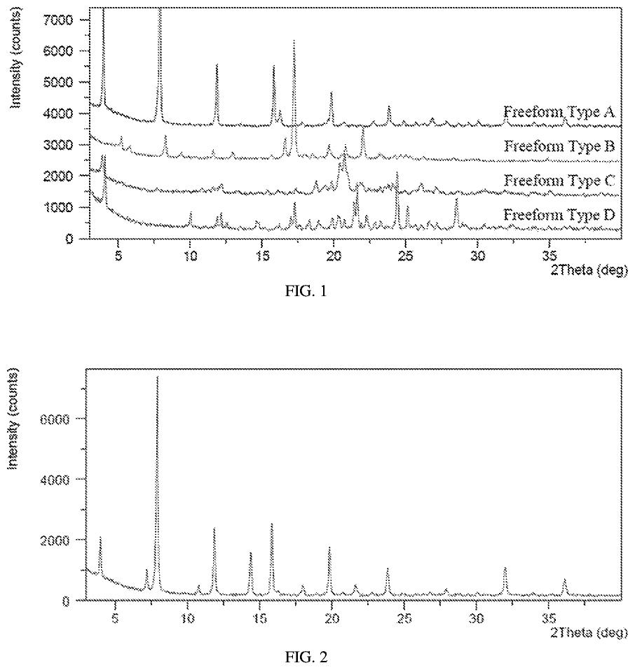

Two new crystalline forms obtained were named freeform Type B and Type C. The XRPD overlay of the four forms are shown in FIG. 1. TGA/DSC/¹H NMR characterization of the four forms were performed, and the detailed characterization results are summarized in Table 2-1 and below.. The data show that freeform Type A and freeform Type D are hydrates, freeform Type B is an anhydrate, and freeform Type C is a meta-stable form.

Thermodynamic relationships study was performed on freeform Type A/B/D. Freeform Type B was obtained when a_{W} below 0.2, freeform Type D was obtained when a_{W}above 0.34. Freeform Type D is the thermodynamic form at the ambient conditions.

TABLE 2-1
Summary of Compound (I) freeform polymorphs
characterization results

| | | Endotherm | |
|---|---|---|---|
| | Weight loss in TGA, | in DSC, | Form |
| Form | (wt %, temp.) | (peak) | identification |
| Freeform Type A | 7.3 (150° C.) | 110.3° C. | Hydrate |
| Freeform Type B | 0.6 (170° C.) | 191.6° C. | Anhydrate |
| Freeform Type C | Transferred to freeform Type A after drying at RT | | |
| Freeform Type D | 8.2 (120° C.) | 106.7° C. | Trihydrate |

### Freeform Polymorphs Characterization

### Freeform Type A

Freeform Type A sample was obtained in a freeform isolation experiment using CHCl₃, and the detailed preparation procedure is shown in Table 9-3.

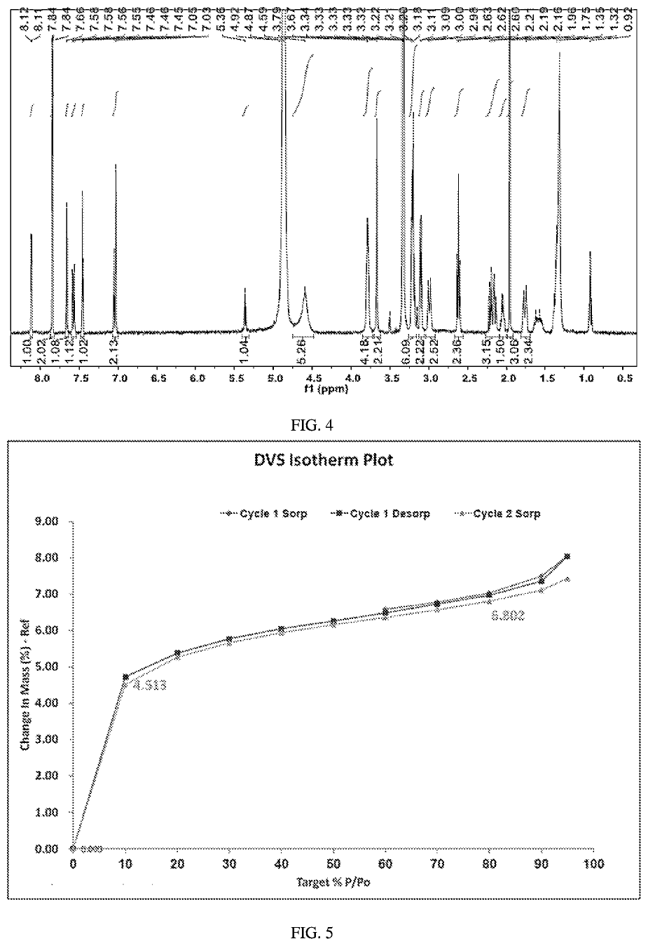

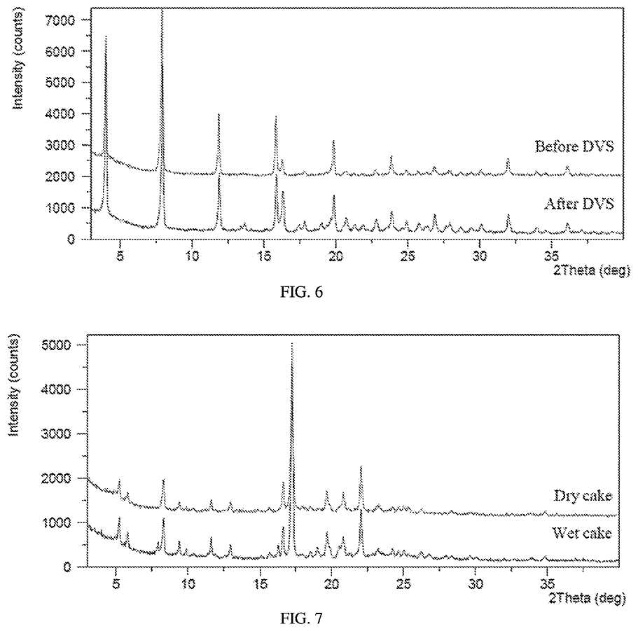

XRPD pattern of freeform Type A is displayed in FIG. 2 with the peaks listed below. TGA/DSC results (FIG. 3) shows a weight loss of 7.3% up to 150° C. and one endotherm at 110.3° C. (peak). ¹H NMR spectrum (FIG. 4) showed no signals of CHCl₃. DVS (dynamic vapor sorption) plot of freeform Type A showed a moisture uptake of 2.3% from 10% RH to 80% RH at 25° C. (FIG. 5). Sample weight decreased rapidly when humidity decreased from 10% to 0%, which might be caused by loss of crystalline water. No form change was observed after DVS test (FIG. 6, 60% RH-95% RH-0% RH-95% RH). These data show that Freeform Type A is a hydrate.

TABLE 2-1a
XRPD Peak List for Freeform Type A:

| | | FWHM | | |
|---|---|---|---|---|
| Pos. [°2θ] | Height [cts] | Left [°2θ] | d-spacing [Å] | Rel. Int. [%] |
| 3.96 | 2935.54 | 0.1535 | 22.34 | 42.57 |
| 7.90 | 6895.92 | 0.1023 | 11.19 | 100.00 |
| 11.85 | 1874.92 | 0.1023 | 7.47 | 27.19 |
| 15.83 | 1893.64 | 0.1023 | 5.60 | 27.46 |
| 16.26 | 504.92 | 0.1279 | 5.45 | 7.32 |
| 17.78 | 117.25 | 0.1535 | 4.99 | 1.70 |
| 19.82 | 1074.78 | 0.1279 | 4.48 | 15.59 |
| 20.66 | 105.40 | 0.2558 | 4.30 | 1.53 |
| 22.76 | 159.98 | 0.1535 | 3.91 | 2.32 |
| 23.83 | 640.23 | 0.1023 | 3.73 | 9.28 |
| 24.86 | 164.01 | 0.1535 | 3.58 | 2.38 |
| 25.71 | 118.43 | 0.1535 | 3.46 | 1.72 |
| 26.83 | 245.41 | 0.1535 | 3.32 | 3.56 |
| 27.87 | 113.09 | 0.2047 | 3.20 | 1.64 |
| 28.65 | 95.92 | 0.2047 | 3.12 | 1.39 |
| 29.36 | 69.10 | 0.3070 | 3.04 | 1.00 |
| 30.06 | 156.52 | 0.2047 | 2.97 | 2.27 |
| 31.95 | 531.50 | 0.1279 | 2.80 | 7.71 |
| 33.92 | 106.25 | 0.1535 | 2.64 | 1.54 |
| 36.07 | 264.96 | 0.1279 | 2.49 | 3.84 |

### Freeform Type B

The freeform Type B sample obtained by slurry of freeform Type A in DMAc/ACN (1:9, v/v) for 7 days was selected for characterization. No form change was observed before and after drying at RT overnight (FIG. 7). TGA/DSC results (FIG. 8) showed a weight loss of 0.6% up to 170° C. and one endotherm at 191.6° C. (peak). ¹H NMR spectrum (FIG. 9) showed no signals of ACN and DMAc.

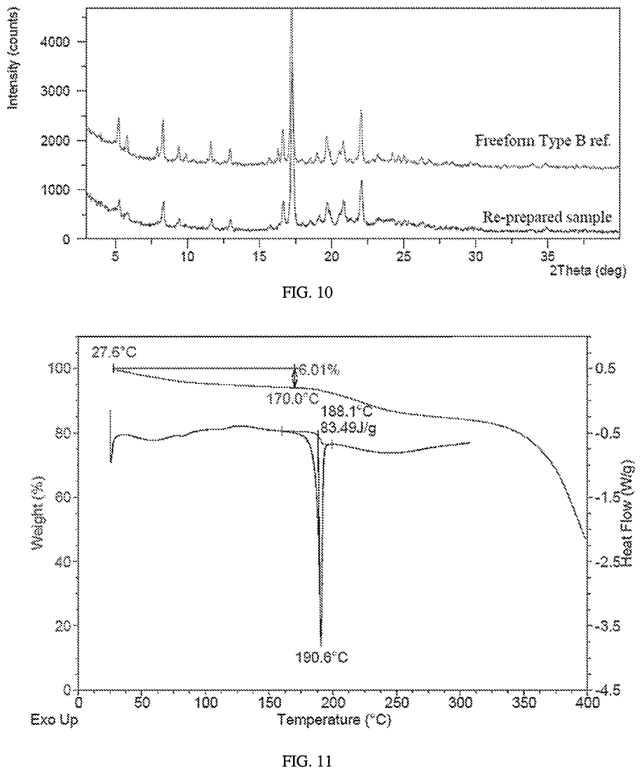

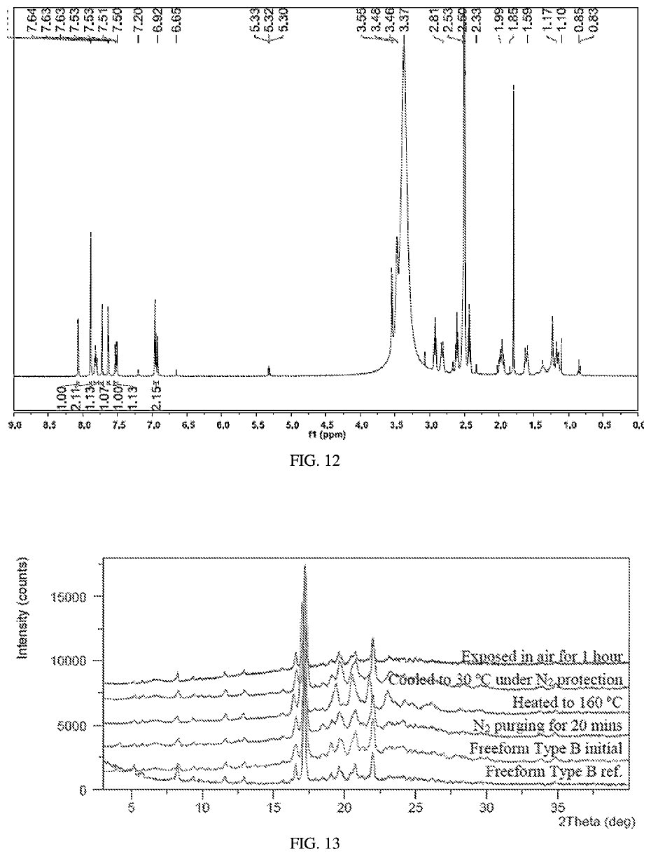

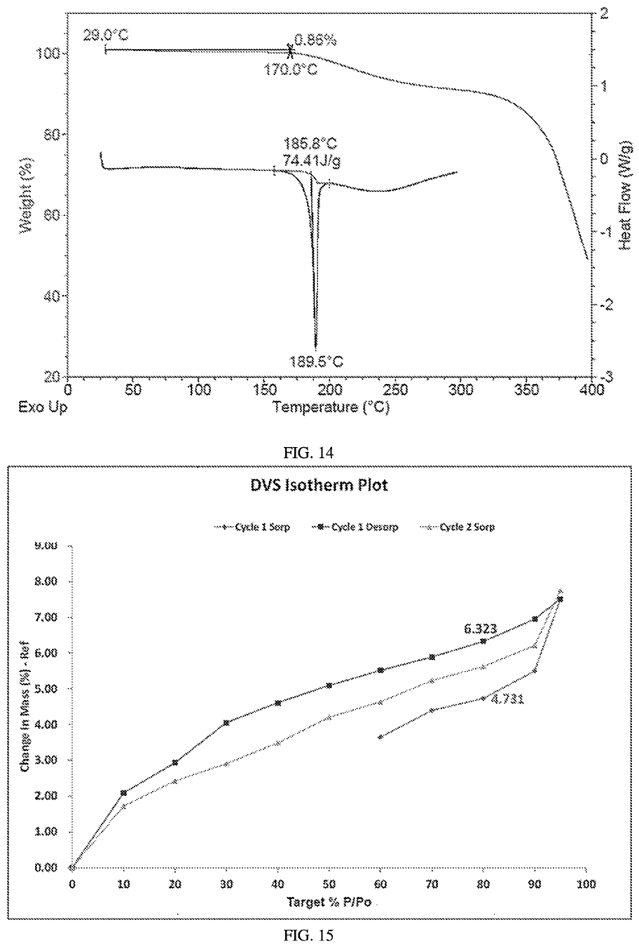

Freeform Type B was re-prepared on 500 mg scale for further characterization and thermodynamic relationships study. About 595.7 mg freeform Type A was dissolved in 1 mL MeOH and 4 mL MTBE was added into the solution slowly under magnetic stirring (1000 rpm). The suspension obtained was stirred at RT for 6 days, and the solid was separated by vacuum filtration. The wet cake obtained was vacuum dried under RT for 8 hours, and about 340 mg freeform Type B was obtained (FIG. 10). The re-prepared freeform Type B was characterized by TGA/DSC/¹H NMR/DVS. TGA/DSC results (FIG. 11) showed a weight loss of 6.0% up to 170° C. and one endotherm at 190.6° C. (peak). ¹H NMR spectrum (FIG. 12) showed no signals of MeOH and MTBE. No form change of freeform Type B was observed after heating to 160° C. by VT-XRPD (FIG. 13). The freeform Type B sample after VT-XRPD test was characterized by TGA/DSC, and the results are shown in FIG. 14.

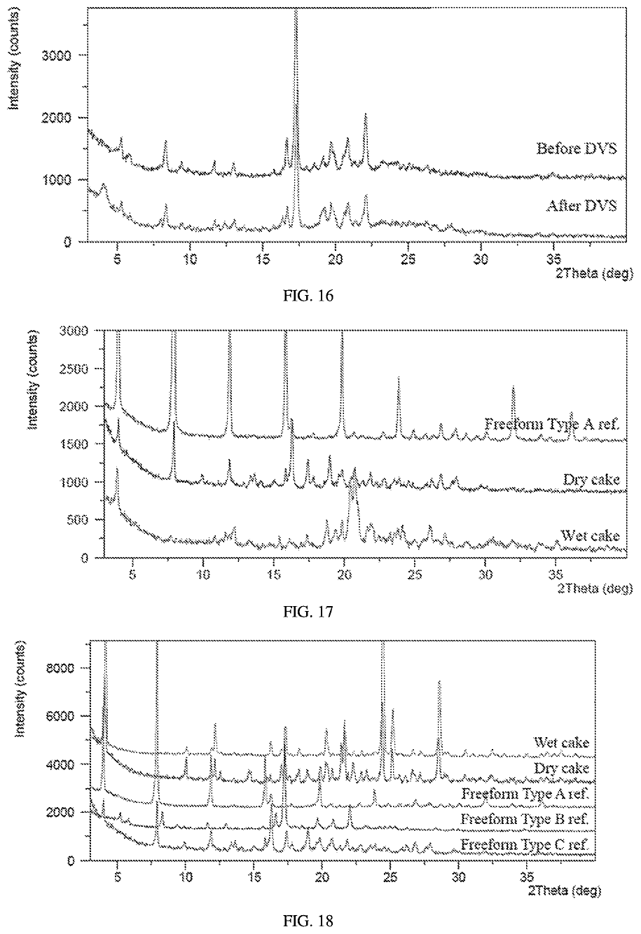

TGA/DSC results show a weight loss of 0.9% up to 170° C. and one endotherm at 189.5° C. (peak). DVS plot of freeform Type B showed a moisture uptake of 6.3% from 0% RH to 80% RH at 25° C. (FIG. 15), indicating that freeform Type B was hygroscopic (European pharmacopoeia 5.0, Example 4). No form change was observed after DVS test (FIG. 16, 60% RH˜95% RH˜0% RH˜95% RH). These data show that Freeform Type B is an anhydrate.

TABLE 2-1b
XRPD Peak List for Freeform Type B (dried):

| | | FWHM | | |
|---|---|---|---|---|
| Pos. [°2θ] | Height [cts] | Left [°2θ] | d-spacing [Å] | Rel. Int. [%] |
| 5.21 | 378.46 | 0.1023 | 16.96 | 10.01 |
| 5.77 | 151.90 | 0.1535 | 15.31 | 4.02 |
| 8.26 | 601.95 | 0.1535 | 10.70 | 15.92 |
| 9.37 | 144.31 | 0.1535 | 9.44 | 3.82 |
| 11.60 | 247.31 | 0.1023 | 7.63 | 6.54 |
| 12.96 | 206.07 | 0.1023 | 6.83 | 5.45 |
| 15.65 | 106.35 | 0.1535 | 5.66 | 2.81 |
| 16.61 | 650.49 | 0.1023 | 5.34 | 17.21 |
| 17.23 | 3780.64 | 0.1279 | 5.15 | 100.00 |
| 18.51 | 145.12 | 0.1535 | 4.79 | 3.84 |
| 19.65 | 480.82 | 0.1023 | 4.52 | 12.72 |
| 20.80 | 458.22 | 0.1023 | 4.27 | 12.12 |
| 22.03 | 1033.62 | 0.1279 | 4.03 | 27.34 |
| 23.20 | 171.18 | 0.2047 | 3.83 | 4.53 |
| 24.24 | 141.36 | 0.1535 | 3.67 | 3.74 |
| 24.63 | 161.11 | 0.1535 | 3.61 | 4.26 |
| 25.15 | 93.24 | 0.4093 | 3.54 | 2.47 |
| 26.26 | 113.59 | 0.1535 | 3.39 | 3.00 |
| 28.37 | 67.48 | 0.3070 | 3.15 | 1.78 |
| 29.74 | 35.81 | 0.6140 | 3.00 | 0.95 |
| 34.85 | 54.97 | 0.6140 | 2.57 | 1.45 |

### Freeform Type C

Freeform Type C was obtained via solid vapor diffusion of freeform Type A in EtOH. Approximately 20 mg of freeform Type A was added to a 3-mL vial. The solid was then placed into a 20-mL vial with 4 mL of EtOH. The 20-mL vial was sealed with a cap and kept at RT allowing organic vapor to interact with the solid. Freeform Type C was obtained after solid vapor diffusion at RT for 17 days.

XRPD pattern of freeform Type C is displayed in FIG. 17 with the peaks listed below. The wet cake of freeform Type C transformed to freeform Type A after vacuum drying at RT for about 3 hours. These data show that freeform Type C is a metastable form.

TABLE 2-1c
XRPD Peak List for Freeform Type C:

| | | FWHM | | |
|---|---|---|---|---|
| Pos. [°2θ] | Height [cts] | Left [°2θ] | d-spacing [Å] | Rel. Int. [%] |
| 3.90 | 830.93 | 0.1023 | 22.67 | 73.45 |
| 12.18 | 221.19 | 0.1535 | 7.26 | 19.55 |
| 13.27 | 64.53 | 0.3070 | 6.67 | 5.70 |
| 16.16 | 86.04 | 0.3070 | 5.48 | 7.61 |
| 17.35 | 155.98 | 0.1535 | 5.11 | 13.79 |
| 18.76 | 388.59 | 0.1023 | 4.73 | 34.35 |
| 19.37 | 241.37 | 0.2047 | 4.58 | 21.34 |
| 19.84 | 371.56 | 0.1023 | 4.48 | 32.84 |
| 20.41 | 879.87 | 0.1279 | 4.35 | 77.78 |
| 20.74 | 1131.28 | 0.1279 | 4.28 | 100.00 |
| 21.91 | 335.94 | 0.2047 | 4.06 | 29.70 |
| 24.12 | 282.84 | 0.1535 | 3.69 | 25.00 |
| 26.07 | 278.00 | 0.2047 | 3.42 | 24.57 |
| 27.12 | 218.57 | 0.1535 | 3.29 | 19.32 |
| 28.67 | 86.76 | 0.3070 | 3.11 | 7.67 |
| 30.45 | 114.79 | 0.6140 | 2.94 | 10.15 |
| 31.90 | 119.73 | 0.3070 | 2.81 | 10.58 |
| 33.86 | 63.71 | 0.5117 | 2.65 | 5.63 |
| 35.05 | 106.38 | 0.2558 | 2.56 | 9.40 |

### Freeform Type D

Freeform Type D sample was obtained in an isolation experiment using CHCl₃, and the detailed preparation procedure is shown in Table 9-3.

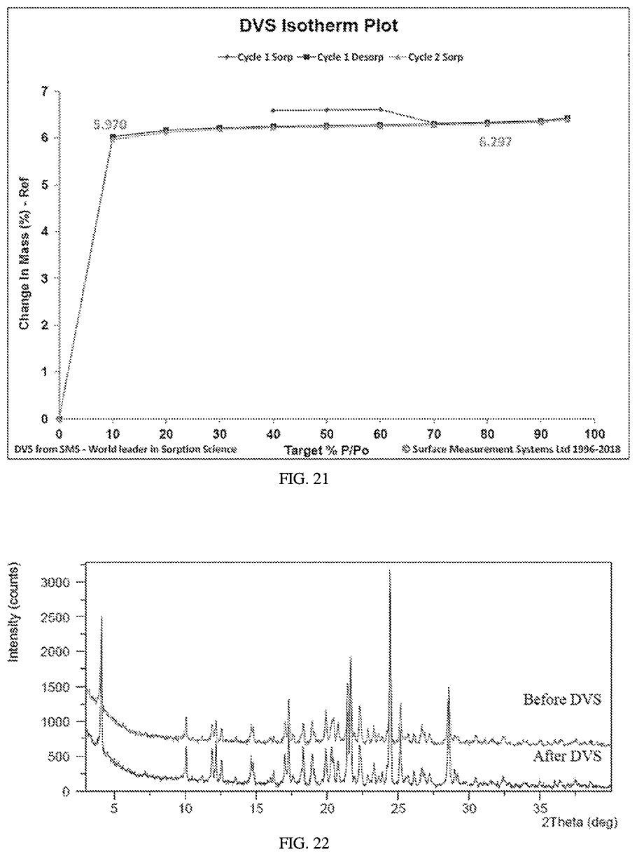

XRPD pattern of freeform Type D is displayed in FIG. 18 and FIG. 186 with the peaks for the dried cake listed below. TGA/DSC results (FIG. 19) showed a weight loss of 8.2% up to 120° C. and one endotherm at 106.7° C. (peak). ¹1H NMR spectrum (FIG. 20) showed no signals of CHCl₃. DVS plot of freeform Type D showed a moisture uptake of 0.3% from 10% RH to 80% RH at 25° C. (FIG. 21). Sample weight decreased rapidly when humidity decreased from 10% to 0%, which might be caused by loss of crystalline water. No form change was observed after DVS test (FIG. 22, 40% RH˜95% RH˜O % RH˜95% RH). Single crystal determination of freeform Type D was performed, demonstrating that freeform Type D was a trihydrate.

To check the solid-state stability of freeform Type D under different humidities, humidity induced experiments under ˜7% RH (desiccator with silica gel), ˜22% RH, ˜43% RH, ˜58% RH and ˜84% RH of freeform Type D were performed. Approximately 20 mg of freeform Type D was added in a 3-mL vial. The 3-mL vial was then placed into a 20-mL vial with 4 mL of the corresponding saturated salt solution. The 20-mL vial was sealed with a cap and kept at RT for the water vapor to interact with the solid. The XRPD results are shown in FIG. 23, and no form change of freeform Type D was observed after exposure to the different humidity levels for 6 weeks.

TABLE 2-1d
XRPD Peak List for Freeform Type D (dry):

| | | FWHM | | |
|---|---|---|---|---|
| Pos. [°2θ] | Height [cts] | Left [°2θ] | d-spacing [Å] | Rel. Int. [%] |
| 4.07 | 1436.92 | 0.1023 | 21.69 | 100.00 |
| 10.03 | 322.72 | 0.1023 | 8.82 | 22.46 |
| 12.01 | 119.22 | 0.4093 | 7.37 | 8.30 |
| 12.53 | 136.95 | 0.1535 | 7.06 | 9.53 |
| 14.68 | 151.64 | 0.2558 | 6.03 | 10.55 |
| 17.01 | 279.53 | 0.1023 | 5.21 | 19.45 |
| 17.27 | 599.35 | 0.1023 | 5.13 | 41.71 |
| 18.29 | 217.84 | 0.1023 | 4.85 | 15.16 |
| 18.91 | 201.19 | 0.1023 | 4.69 | 14.00 |
| 19.89 | 252.91 | 0.1535 | 4.46 | 17.60 |
| 20.33 | 293.75 | 0.2047 | 4.37 | 20.44 |
| 21.40 | 631.59 | 0.1023 | 4.15 | 43.95 |
| 21.62 | 1018.08 | 0.1023 | 4.11 | 70.85 |
| 22.27 | 319.41 | 0.1791 | 3.99 | 22.23 |
| 22.85 | 155.31 | 0.1023 | 3.89 | 10.81 |
| 23.25 | 194.07 | 0.1023 | 3.83 | 13.51 |
| 24.41 | 1300.21 | 0.1023 | 3.65 | 90.49 |
| 25.14 | 516.17 | 0.1023 | 3.54 | 35.92 |
| 25.65 | 97.14 | 0.1535 | 3.47 | 6.76 |
| 26.08 | 89.47 | 0.1535 | 3.42 | 6.23 |
| 26.63 | 202.98 | 0.1279 | 3.35 | 14.13 |
| 27.18 | 128.48 | 0.1535 | 3.28 | 8.94 |
| 28.53 | 728.24 | 0.1279 | 3.13 | 50.68 |
| 29.04 | 89.75 | 0.3070 | 3.07 | 6.25 |
| 30.45 | 114.68 | 0.1535 | 2.94 | 7.98 |
| 32.37 | 93.77 | 0.3070 | 2.77 | 6.53 |
| 35.01 | 45.82 | 0.3070 | 2.56 | 3.19 |

### Thermodynamic Relationships Study

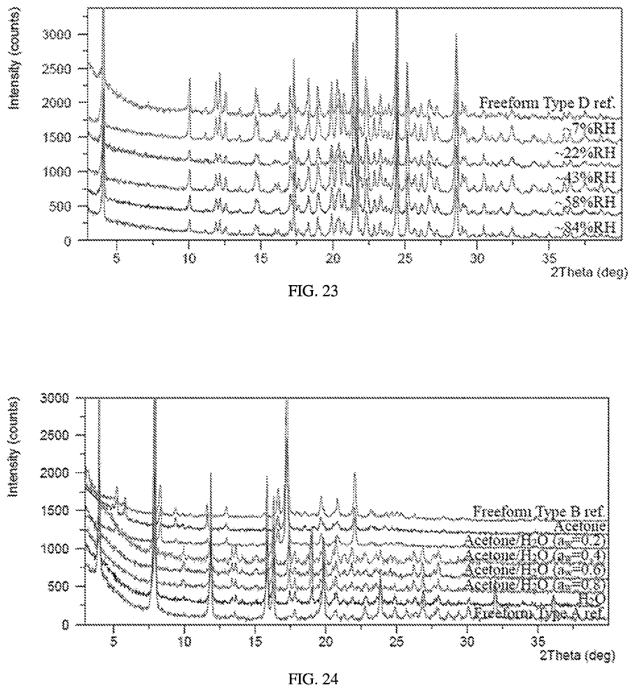

Slurry competition was conducted in acetone/H₂O systems with various water activities (0.0-1.0). A mixture of hydrate freeform Type A and anhydrate freeform Type B with equal mass ratio was suspended in a saturated solution (prepared with freeform Type A) and then stirred at RT for 2 days. Solids were separated and tested by XRPD. As summarized in Table 2-2 and shown in FIG. 24, the results show freeform Type B was obtained in acetone and acetone/H₂O (a_{w}=0.2), and freeform Type A was obtained in H₂O and acetone/H₂O (a_{w}=0.4/0.6/0.8).

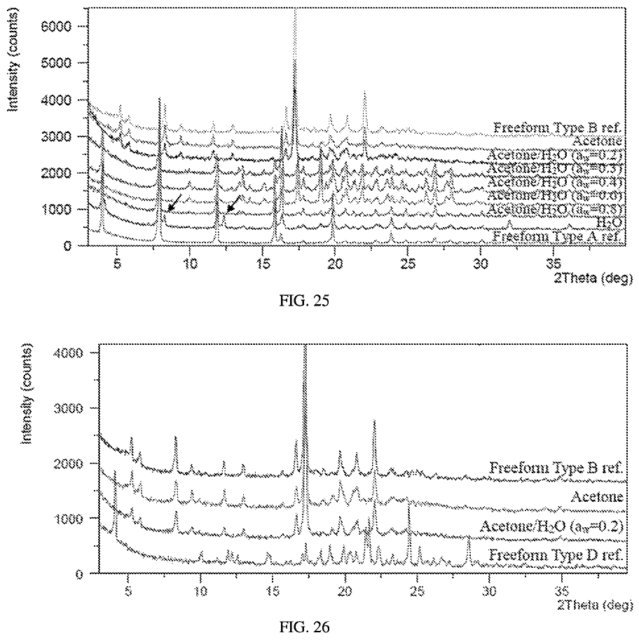

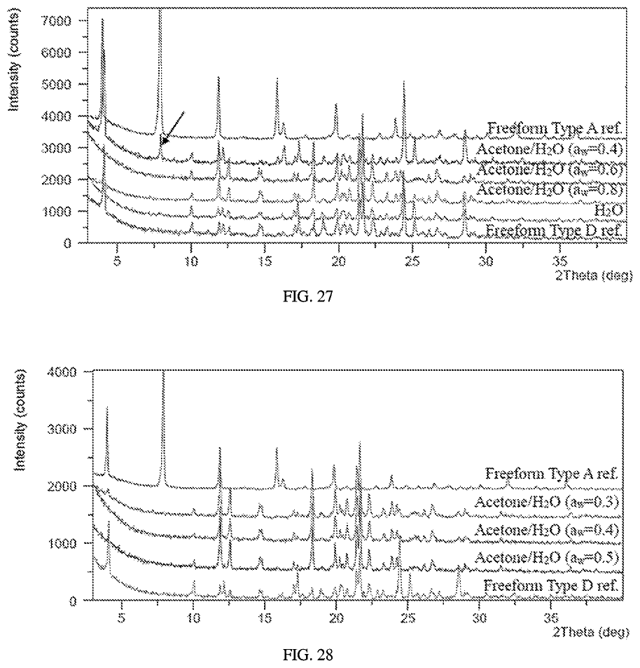

To measure the solubility of freeform in acetone/H₂O systems with various water activities, the above experiments were re-performed with addition of a_{w}=0.3 acetone/H₂O system. The suspension sample was centrifuged to obtain precipitate and supernatants. Solubility was tested for the supernatants after filtration, and isolated precipitate was tested by XRPD. As summarized in Table 2-3 and shown in FIG. 25, freeform Type B was obtained in acetone and acetone/H₂O (a_{w}=0.2), freeform Type A was obtained in acetone/H₂O (a_{w}=0.3/0.4/0.6/0.8), and freeform Type A with additional peaks was observed for experiment in H₂O. Freeform Type A showed highest solubility (10.3 mg/mL) in acetone/H₂O (a_{w}=0.8). About 5 mg of freeform Type D was added into the slurry competition experiments with freeform Type A or Type B, and the samples were re-stirred at RT for 10 days. As summarized in Table 2-4 and shown in FIG. 26 and FIG. 27, freeform Type B was obtained in acetone and acetone/H₂O (a_{w}=0.2), freeform Type D was obtained in H₂O and acetone/H₂O (a_{w}=0.6/0.8), and a mixture of freeform Type A and Type D was obtained in acetone/H₂O (a_{w}=0.4).

To determine the thermodynamic relationship of hydrate freeform Type A and Type D within the a_{w} range from 0.3 to 0.5, slurry competition was conducted in acetone/H₂O (a_{w}=0.3/0.4/0.5). A mixture of hydrate freeform Type A and freeform Type D with equal mass ratio was suspended in a saturated solution and then stirred at RT for 4 days. Solids were separated and tested by XRPD. As summarized in Table 2-5 and shown in FIG. 28, freeform Type D was obtained in acetone/H₂O (a_{w}=0.3/0.4/0.5). The supernatant of acetone/H₂O (a_{w}=0.3) system was checked by KF after slurry competition experiments, and KF results showed the final water activity was about 0.34.

According to the slurry competition experiments of freeform Type A and Type B, freeform Type B was obtained when a_{W} below 0.2, and freeform Type A was obtained when a_{W} above 0.3. According to the slurry competition experiments of freeform Type A and Type D, freeform Type D was obtained when a_{W} above 0.3. Freeform type D is a more stable hydrate form compared to freeform Type A.

TABLE 2-2
Summary of thermodynamic relationships
study of freeform Type A and Type B

| | Solvent system | Water | |
|---|---|---|---|
| Starting Form | (v/v) | activity (a_{w}) | Results |
| Freeform Type | Acetone | 0.0 | Freeform Type B |
| A + B | Acetone/H₂O | 0.2 | Freeform Type B |
| | (986:14) | | |
| | Acetone/H₂O | 0.4 | Freeform Type A |
| | (950:50) | | |
| | Acetone/H₂O | 0.6 | Freeform Type A |
| | (858:142) | | |
| | Acetone/H₂O | 0.8 | Freeform Type A |
| | (605:395) | | |
| | H₂O | 1.0 | Freeform Type A |

TABLE 2-3
Summary of thermodynamic relationships study of freeform
Type A and Type B (including solubility test)

| | Solvent system | Water | Solubility |
|---|---|---|---|
| Starting Form | (v/v) | activity (a_{w}) | (mg/mL) |
| Freeform Type | Acetone | 0 | 0.810 (Type B) |
| A + B | Acetone/H₂O | 0.2 | 3.962 (Type B) |
| | (986:14) | | |
| | Acetone/H₂O | 0.3 | 1.439 (Type A) |
| | (973:27) | | |
| | Acetone/H₂O | 0.4 | 1.155 (Type A) |
| | (950:50) | | |
| | Acetone/H₂O | 0.6 | 3.286 (Type A) |
| | (858:142) | | |
| | Acetone/H₂O | 0.8 | 10.265 (Type A) |
| | (605:395) | | |
| | H₂O | 1 | 0.010 (Type A) |

TABLE 2-4
Summary of thermodynamic relationships
study of freeform Types A, B, and D

| | Solvent system | Water | |
|---|---|---|---|
| Starting Form | (v/v) | activity (a_{w}) | Results |
| Freeform Type | Acetone | 0 | Freeform Type B |
| B + D | Acetone/H₂O | 0.2 | Freeform Type B |
| | (986:14) | | |
| Freeform Type | Acetone/H₂O | 0.4 | Freeform Type |
| A + D | (950:50) | | A + D |
| | Acetone/H₂O | 0.6 | Freeform Type D |
| | (858:142) | | |
| | Acetone/H₂O | 0.8 | Freeform Type D |
| | (605:395) | | |
| | H₂O | 1 | Freeform Type D |

TABLE 2-5
Summary of thermodynamic relationships study of
freeform Type A and Type D (a_{w} from 0.3 to 0.5)

| | Solvent system | Water | |
|---|---|---|---|
| Starting Form | (v/v) | activity (a_{w}) | Results |
| Freeform Type | Acetone/H₂O | 0.3 | Freeform Type D |
| A + D | (972:28) | | |
| | Acetone/H₂O | 0.4 | Freeform Type D |
| | (950:50) | | |
| | Acetone/H₂O | 0.5 | Freeform Type D |
| | (914:86) | | |

### Example 2. Characterization of Compound (I) Freeform

### pKa Test

The spectrophotometric method (UV-metric pKa) and potentiometric method (pH-metric pKa) were used for pKa test of Compound (I) freeform.

1. For UV-metric pKa, the pKa values were determined by monitoring the change in UV absorbance with pH as the compound undergoes ionization.

2. For pH-metric pKa, the pKa values were determined from an examination of the shape of the resultant titration curves and fitting a suitable theoretical model for the compound's ionization behavior onto the titration data.

MeOH as cosolvent was used for pKa testing, psKa represents apparent pKa values of compounds measured in water/co-solvent mixtures, and psKa values were tested with MeOH concentration of ˜30%, ˜40% and ˜50%. The psKa values were extrapolated to 0% organic content using the Yasuda-Shedlovsky extrapolation procedure for pKa values.

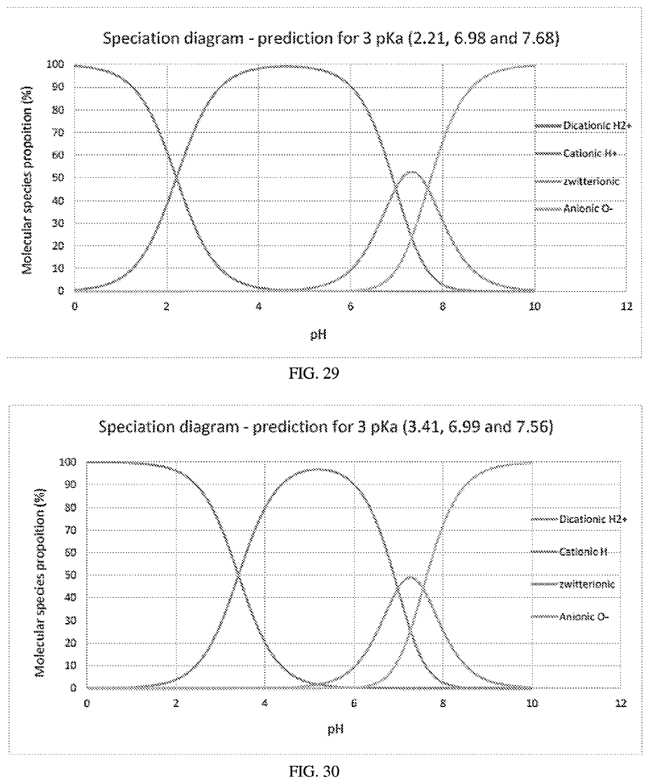

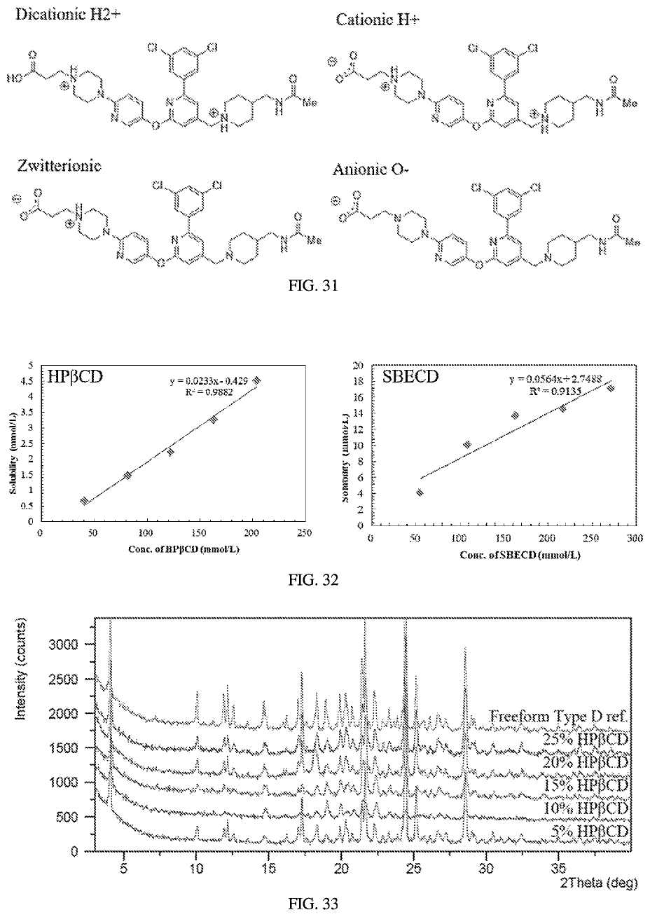

The tested pKa results and calculated pKa results are summarized in Table 3-1. One pKa result (2.21) tested by UV-metric was out of the effective pH range, and the pKa results tested by pH-metric were recommended. The speciation diagram of freeform is shown in FIG. 29 (UV-metric) and FIG. 30 (pH-metric). The speciation structures of the freeform is shown in FIG. 31.

TABLE 3-1
Summary of pKa test Results

| | | Results | | |
|---|---|---|---|---|
| Sample | pKa | UV-metric | pH-metric | Calculated results² |
| Compound (I) | pKa 1 | 2.21¹ | 3.41 | 3.00 |
| freeform | pKa 2 | 6.98 | 6.99 | 4.24 |
| | pKa 3 | 7.68 | 7.56 | 7.78 |
| | pKa 4 | No value | No value | 8.44 |

¹pKa1 obtained by UV-metric should be taken as reference because the psKa value used for Yasuda-Shedlovsky extrapolation procedure was out of the effective pH range 2-12 during titration. (The psKa values were 1.84, 1.56 and 1.27 at MeOH concentration of 36.4%, 48.2%, and 63.9%, respectively)
²Calculated with MarvinBeans 5.6.0.2.

### Log D_{7.4} Test

To determine the distribution coefficient of Compound (I) freeform, Log D_{7.4} of freeform Type D was determined in n-octanol/aqueous (pH=7.4) systems at room temperature by partitioning shake flask method. The detailed procedure is summarized below.
1. Pre-equilibrate n-octanol and aqueous buffer by adding 10 mL n-octanol and 10 mL aqueous buffer into a glass vial and keep it rolling for 24 h. The mutually saturated n-octanol and aqueous buffer are obtained after phase separation.
2. Weigh approximately 1.0 mg sample into a 3-mL glass vial with 1.0 mL of saturated n-octanol inside (obtained in step 1 above) and accelerate dissolution by ultrasonication.
3. Add 1.0 mL of complementary saturated aqueous buffer into the vial.
4. Prepare samples in triplicate. Seal the glass vial and mix on a rotary mixer at 25° C. for 24 h.
5. The phases are separated after rolling.
6. The concentrations of the compound in each phase are determined by HPLC. Samples in n-octanol phase are diluted 10 times by adding 100 μL of sample solution and 900 L of ACN/H₂O (v/v, 3:1) diluent and mixed well.
7. Distribution coefficient, referred to as D_{ow}, is calculated as the concentration of the test compound (both ionized and un-ionized) in the n-octanol phase divided by the corresponding concentration in the aqueous phase. Log D_{7.4} is calculated as an average of the log 10 of D_{ow} based on three runs.
Detailed results are shown in FIG. 30, and the results showed the Log D_{7.4} of freeform is 2.23.

TABLE 3-2
Summary of LogD_{7.4} test results

| | | Concentration | | | | |
|---|---|---|---|---|---|---|
| | | (mg/mL) | | Final pH in | | Average of |
| Sample | # | Oil | Aqueous | aqueous phase | LogD_{7.4} | LogD_{7.4} |
| Compound (I) | 1 | 1.14 | 0.007 | 7.3 | 2.24 | 2.23 |
| freeform | 2 | 1.10 | 0.006 | 7.3 | 2.23 | (RSD = 0.26%) |
| | 3 | 1.07 | 0.006 | 7.3 | 2.23 | |

LOQ: 0.02 μg/mL

### Complexation Stability Constant Test

To determine the complexation stability constant (K^{1:1}) of Compound (I) freeform, solubility of freeform Type D in different concentration of HPβCD solutions and SBECD solutions was tested at RT. ˜5 mg of solid was suspended into 1 mL of each medium with dosing conc. at ˜5 mg/mL. Additional solid was added into the sample to generate suspension if clear solution was obtained. The suspension was equilibrated via stirring (1000 rpm) for 24 hours. The suspension was centrifuged to obtain precipitate and supernatants. Solubility and pH was determined for the supernatants after filtration, and XRPD was obtained for the isolated precipitate.

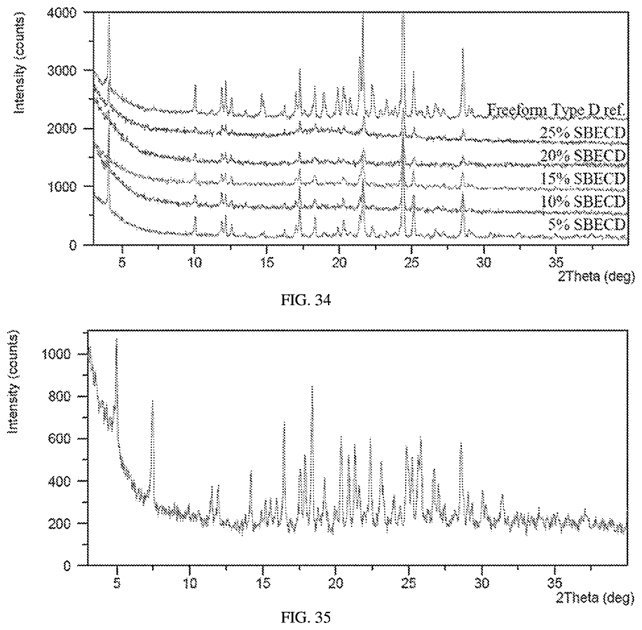

The detailed solubility results are summarized in Table 3-3. Solubility curves of freeform Type D in different concentration HPβCD solutions and SBECD solutions are shown in FIG. 32. The complexation stability constant (K¹¹) was calculated as 1529 M⁻¹ and 3831 M⁻¹ for HPβCD and SBECD, respectively (calculated by Higuchi-Connors phase-solubility method), and the date suggests that Type A_{L} complex is formed with HPβCD and SBECD. No form change was observed during the solubility evaluation in HPβCD solutions and SBECD solutions, and the XRPD patterns are shown in FIG. 33 and FIG. 34.

TABLE 3-3
Summary of complexation stability constant test results

| | Conc. | Solubility | Initial | Final pH | |
|---|---|---|---|---|---|
| Medium | (w/w) | (mg/mL) | pH | (24 hours) | Form |
| HPβCD | 25% | 2.89 | 7.0 | 7.0 | Freeform |
| solutions | 20% | 2.09 | 6.9 | 7.0 | Type D |
| | 15% | 1.42 | 7.0 | 7.1 | |
| | 10% | 0.94 | 7.0 | 7.2 | |
| | 5% | 0.42 | 7.1 | 7.3 | |
| SBECD | 25% | 10.97 | 8.0 | 7.9 | |
| solutions | 20% | 9.35 | 8.1 | 7.9 | |
| | 15% | 8.81 | 8.0 | 7.9 | |
| | 10% | 6.47 | 8.0 | 7.9 | |
| | 5% | 2.61 | 8.1 | 7.8 | |

TABLE 3-4
Summary of complexation stability constant calculation

| | Buffer | Buffer | API | API | |
|---|---|---|---|---|---|
| | Conc.* | Conc. | Solubility | Solubility | |
| Medium | (w/w, g/g) | (mmol/L) | (mg/L) | (mmol/L) | K^{1:1} (M⁻¹) |
| HPβCD | 25% | 203.89 | 2.89 | 4.51 | 1529 |
| solutions | 20% | 163.11 | 2.09 | 3.26 | |
| | 15% | 122.34 | 1.42 | 2.22 | |
| | 10% | 81.56 | 0.94 | 1.47 | |
| | 5% | 40.78 | 0.42 | 0.66 | |
| SBECD | 25% | 271.16 | 10.97 | 17.11 | 3831 |
| solutions | 20% | 216.93 | 9.35 | 14.59 | |
| | 15% | 162.70 | 8.81 | 13.74 | |
| | 10% | 108.46 | 6.47 | 10.09 | |
| | 5% | 54.23 | 2.61 | 4.07 | |

*The 25% HPβCD (or SBECD) solutions (w/w) was prepared by adding 2.5 g HPβCD (or SBECD) into 7.5 mL water. The 20%/15%/10%/5% solutions were obtained by dilution of 25% solutions. The molecular weight used for calculation was 1618.5 and 1217 for HPβCD and SBECD, respectively.

### Example 3. Salt Formation and Characterization

According to the approximate solubility of freeform Type A (Table 9-5) and simulated pKa of Compound (I) (3.00/carboxylic acid, 4.24/pyridine, 7.78/piperazine and 8.44/piperidine, simulated with MarvinBeans 5.6.0.2), salt screening was designed and performed under 96 conditions using 16 counterions in 6 solvent systems. Freeform Type A and corresponding counterions were mixed in a molar ratio of 1:1 in six solvents (MeOH, THF, EtOAc, acetone, IPA and ACN/H₂O (19:1, v/v)), and then stirred at RT for 3 days. After centrifugation, resulting solids were dried under vacuum at RT overnight, and then analyzed by XRPD (Table 4-1). Slurry at 5° C. and evaporation were applied to induce solid precipitation for clear solution obtained in salt screening. Based on XRPD results, 10 crystalline salts (17 forms) were obtained and characterized, and the characterization results are summarized in Table 4-2. Detailed characterization results are shown in Example 7.

Six additional HCl salt formation experiments were performed with different charge ratio. The experiments and results are summarized in Table 4-3. HCl salt Type B/C/D/E forms were observed and characterized, with the characterization results summarized in Table 4-2. Detailed characterization results are shown in Example 7.

TABLE 4-1
Summary of salt formation experiments

| | | Solvent | | | | | |
|---|---|---|---|---|---|---|---|
| | | | | | | | ACN/H₂O |
| Counterion source | | MeOH | THF | EtOAc | Acetone | IPA | (19:1, v/v) |
| 0 | BLK | Freeform | Low | Freeform | Freeform | Freeform | Freeform |
| | | Type A* | crystallinity | Type B | Type A + B | Type A + B | Type A |
| 1 | HCl | HCl salt | HCl salt | Freeform | Freeform | Freeform | Freeform |
| | | Type A | Type A | Type A | Type A + B | Type B | Type A |
| 2 | Sulfuric acid | Sulfate | Low | Freeform | Sulfate | Freeform | Low |
| | | Type A | crystallinity | Type A | Type B | Type B | crystallinity |
| 3 | Phosphoric acid | Freeform | Low | Freeform | Freeform | Freeform | Freeform |
| | | Type B | crystallinity | Type A | Type B | Type B | Type A |
| 4 | Maleic acid | Gel* | Maleate | Freeform | Amorphous | Amorphous | Maleate |
| | | | Type A | Type B | | | Type B |
| 5 | Tartaric acid | Gel* | Amorphous | Freeform | Freeform | Freeform | Tartrate |
| | | | | Type B | Type A | Type A | Type A |
| 6 | Fumaric acid | Fumarate | Fumarate | Fumarate | Fumarate | One peak | Fumarate |
| | | Type A | Type B | Type B | Type B | | Type C |
| 7 | Citric acid | Amorphous | Amorphous | Freeform | Freeform | Freeform | Amorphous |
| | | | | Type A | Type A | Type A | |
| 8 | Succinic acid | Succinate | Succinate | Freeform | Freeform | Succinate | Succinate |
| | | Type A* | Type A | Type A | Type B | Type B | Type C |
| 9 | Acetic acid | Freeform | Low | Freeform | Freeform | Freeform | Freeform |
| | | Type B | crystallinity | Type B | Type B | Type B | Type A |
| 10 | Methanesulfonic | Freeform | Low | Freeform | Freeform | Freeform | Freeform |
| | acid | Type A* | crystallinity | Type A | Type B | Type B | Type A |
| 11 | Isethionic acid | Freeform | Low | Freeform | Freeform | Freeform | Freeform |
| | | Type A^{#} | crystallinity | Type A + B | Type B | Type B | Type A |
| 12 | Triphenyl acetic | Amorphous* | Triphenyl | Low | Freeform | Freeform | Freeform |
| | | | acetate salt | crystallinity | Type B | Type B | Type A |
| | | | Type A | | | | |
| 13 | Xinafoic acid | Gel* | Xinafoic salt | Freeform | Freeform | Low | Freeform |
| | | | Type A | Type B + | Type B | crystallinity | Type A |
| | | | | additional | | | |
| | | | | peaks | | | |
| 14 | Calcium | Calcium | Calcium | Freeform | Freeform | Freeform | Ca²⁺ salt |
| | hydroxide | hydroxide | hydroxide | Type B | Type B + | Type A | Type A |
| | | | | | two peaks | | |
| 15 | Sodium | Low | Amorphous | Freeform | Low | Low | Low |
| | hydroxide | crystallinity* | | Type A | crystallinity | crystallinity | crystallinity |
| 16 | Tromethamine | Tromethamine | Tromethamine | Freeform | Freeform | Tromethamine | Tromethamine |
| | | salt Type B* | | Type B | Type B | salt Type A | salt Type B |

*Solid was obtained after evaporation at RT.
^{#}Solid was obtained after evaporation at 5° C.

TABLE 4-2
Characterization of salt forms of Compound (I)

| | Crystallinity | Weight loss | Endotherm | Molar ratio |
|---|---|---|---|---|
| Salt forms | by XRPD | (%, temp) | (° C., peak) | (counter ion:API) |
| HCl salt Type A | Medium | 3.6 (140° C.) | 70.9, 98.5, 221.2* | 0.3 |
| HCl salt Type D | High | 4.4 (200° C.) | 231.6, 263.0, | 1.8 |
| | | | 286.5* | |
| HCl salt Type E | High | 3.2 (200° C.) | 272.4, 281.2 | 2.4 |
| Sulfate Type A | Medium | The sample amount was not enough for characterization | | |
| Sulfate Type B | High | 2.6 (200° C.) | 66.7, 259.5, 280.0 | 1.0 |
| Maleate Type A | High | 7.3 (130° C.) | 66.1, 116.4, 140.9 | 1.1 |
| Maleate Type B | Medium | 10.3 (140° C.) | 56.0*, 193.6 | 1.0 |
| Tartrate Type A | Weak | 16.1 (160° C.) | 89.5, 124.0, | 1.2 |
| | | | 141.9, 209.8 | |
| Fumarate Type A | Medium | 6.2 (175° C.) | 72.6, 165.1, 214.4 | 1.1 |
| Fumarate Type B | High | 7.3 (160° C.) | 107.2*, 134.1*, | 0.9 |
| | | | 218.5 | |
| Fumarate Type C | Medium | 9.9 (170° C.) | 70.8, 167.0 | 1.2 |
| Succinate Type A | Weak | The sample amount was not enough | | 1.0 |
| | | for characterization | | |
| Succinate Type B | Weak | 5.4 (130° C.) | 76.0, 117.6, | 0.3 |
| | | | 179.0, 147.8^{#} | |
| Succinate Type C | Weak | 6.4 (150° C.) | 72.9, 102.4, | 0.8 |
| | | | 119.3, 130.7, | |
| | | | 205.3 | |
| Triphenyl acetate Type A | Weak | 6.1 (140° C.) | 58.4, 148.8, 222.5 | 1.0 |
| Xinafoic salt Type A | Medium | 3.8 (130° C.) | 67.9, 180.4, | 1.1 |
| | | | 213.6, 243.6 | |
| Ca²⁺salt Type A | Medium | 5.6 (150° C.) | 76.2, 171.4 | 0.4 |
| Tromethamine salt Type A | Weak | 3.6 (140° C.) | 65.3, 77.8, 86.2, | 1.8 |
| | | | 110.5, 124.7, | |
| | | | 203.7 | |
| Tromethamine salt Type B | High | 9.2 (150° C.) | 90.2, 104.6, 127.9 | 1.3 |

*Onset temperature.
^{#}exothermic peak.

TABLE 4-3
Summary of additional HCl salt formation experiments

| | Solvent | | |
|---|---|---|---|
| Stoichiometry | MeOH/H₂O | | |
| (HCl/API) | (19:1, v/v) | EtOAc | ACN |
| 1:1 | Low | HCl salt Type B | HCl salt Type C |
| | crystallinity | (wet cake) | (wet cake) |
| | | HCl salt Type E | HCl salt Type E |
| | | (dry cake) | (dry cake) |
| 2:1 | Low | Freeform Type A | HCl salt Type D |
| | crystallinity | | |

### Salt Preparation and Characterization

HCl salt Type D, fumarate Type B and sulfate Type B showed better solid-state properties when compared with other salt forms, and the three salts were selected for re-preparation on 500 mg scale. HCl salt Type F (new form), fumarate Type A and sulfate Type B were obtained during the re-preparation experiments, and the three forms were selected for solubility evaluation. The preparation procedures of the three salts are summarized in Table 4-4.

TABLE 4-4
Preparation procedures of three salt forms

| Salt Form | Procedures |
|---|---|
| HCl salt | 1. Weigh ~500 mg of freeform Type A into a 5-mL |
| | glass vial. |
| Type F | 2. Add ~69 μL of hydrochloric acid and 4.0 mL |
| | of ACN to the vial to form a suspension. |
| | 3. Stir the suspension magnetically (~1000 rpm) |
| | at RT for 2 days. |
| | 4. Centrifuge to isolate the solid and vacuum drying |
| | at RT for ~4 hrs. |
| Sulfate | 1. Weigh ~500 mg of freeform Type A into a 5.0-mL |
| | glass vial. |
| Type B | 2. Add ~194 μL of sulfuric acid and 2.0 mL of |
| | acetone to the vial to form a suspension. |
| | 3. Stir the suspension magnetically (~1000 rpm) at |
| | RT for ~3 days. |
| | 4. Centrifuge to isolate the solid and vacuum drying |
| | at RT for ~1 day. |
| Fumarate | 1. Weigh ~500 mg of freeform Type A into a 5.0-mL |
| | glass vial. |
| Type A | 2. Add ~91 mg of fumaric acid and 2.0 mL of EtOAc |
| | to the vial to form a suspension. |
| | 3. Stir the suspension magnetically (~1000 rpm) at |
| | RT for ~3 days. |
| | 4. Centrifuge to isolate the solid and vacuum drying |
| | at RT for 1 day. And mixture was obtained. |
| | 5. Add 4.0 mL acetone to the solid and stir the suspension |
| | magnetically (~1000 rpm) at RT for 1 day. |
| | 6. Centrifuge to isolate the solid and vacuum drying at |
| | RT overnight. |

### HCl Salt Type F

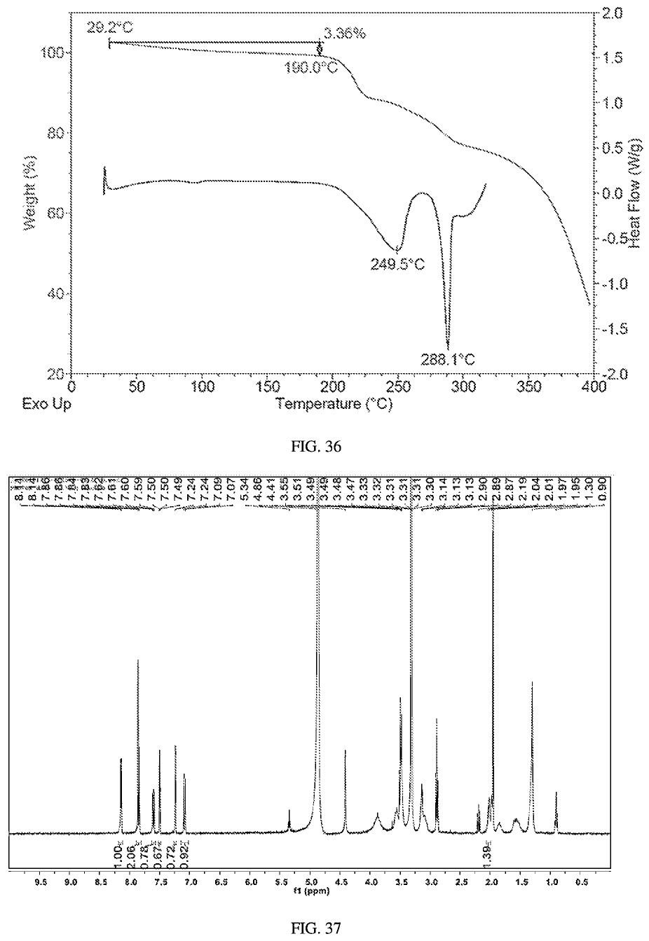

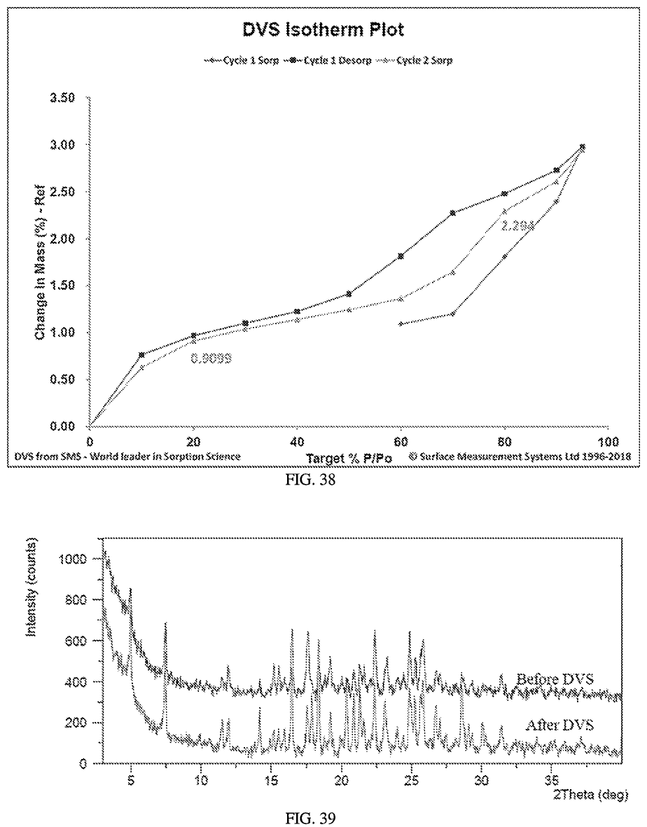

HCl salt Type F was obtained during re-preparation of HCl salt Type D, with the detailed preparation procedure summarized in Table 4-4. The XRPD pattern of HCl salt Type F is displayed in FIG. 35. TGA/DSC results (FIG. 36) of the sample showed a weight loss of 3.4% up to 190° C. and two endotherms at 249.5° C. (peak) and 288.1° C. (peak). ¹H NMR spectrum (FIG. 37) showed no signals of ACN. HPLC/IC results showed the molar ratio of Cl⁻ to freeform was 2.2:1.0. DVS plot of HCl salt Type F showed a moisture uptake of 2.3% from 0% RH to 80% RH at 25° C. (FIG. 38). No form change was observed after the DVS test (FIG. 39).

### Sulfate Type B

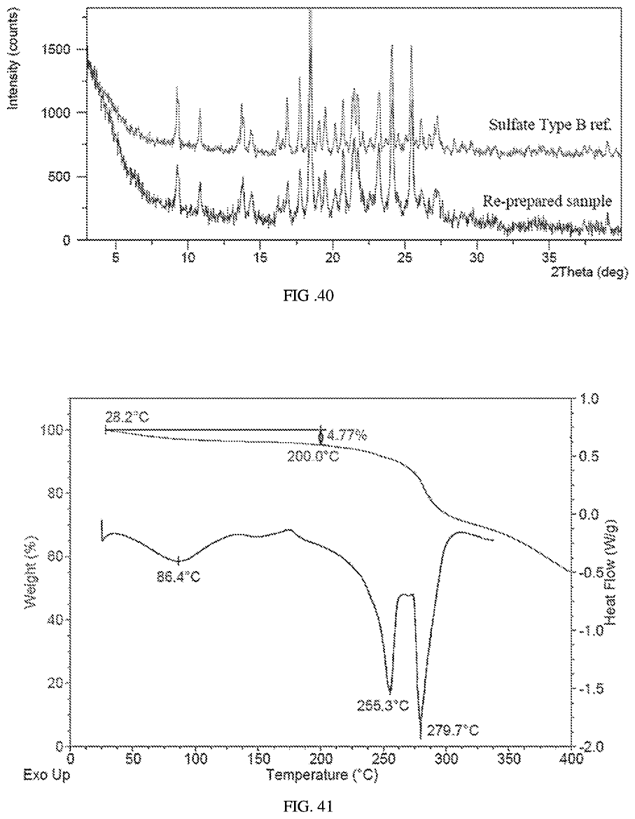

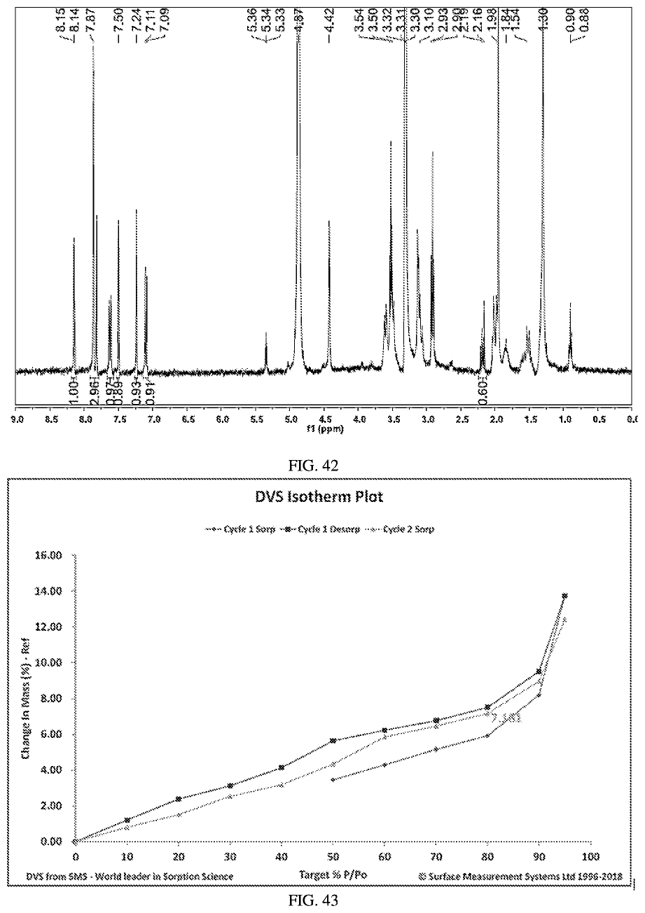

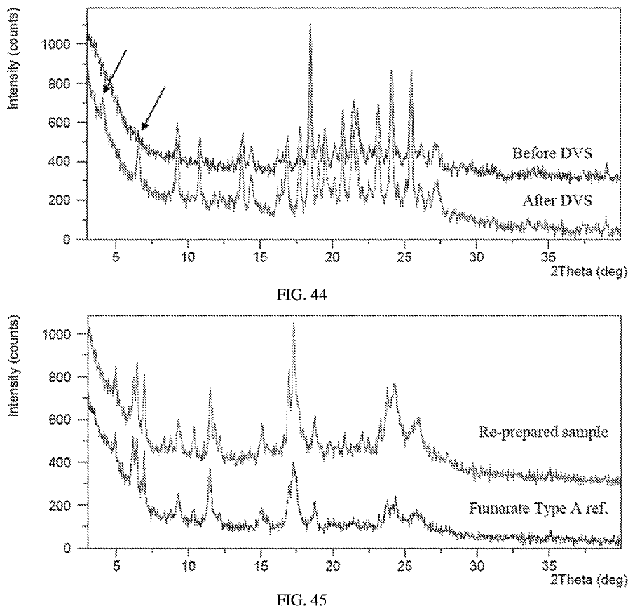

Sulfate Type B was obtained during salt formation experiments in acetone, and the form was selected for re-preparation on 500 mg scale. The detailed preparation procedure of sulfate Type B is summarized in Table 4-4. The XRPD pattern of re-prepared sulfate Type B is displayed in FIG. 40. TGA/DSC results (FIG. 41) of the sample showed a weight loss of 4.8% up to 200° C. and three endotherms at 86.4° C. (peak), 255.3° C. (peak) and 279.7° C. (peak). ¹H NMR spectrum (FIG. 42) showed no signals of acetone. HPLC/IC results showed the molar ratio of SO₄²⁻ to freeform was 1.0:1.0. DVS plot of sulfate Type B showed a moisture uptake of 7.2% from 0% RH to 80% RH at 25° C. (FIG. 43). Two additional peaks (marked with arrow) were observed after DVS test (FIG. 44).

### Fumarate Type A

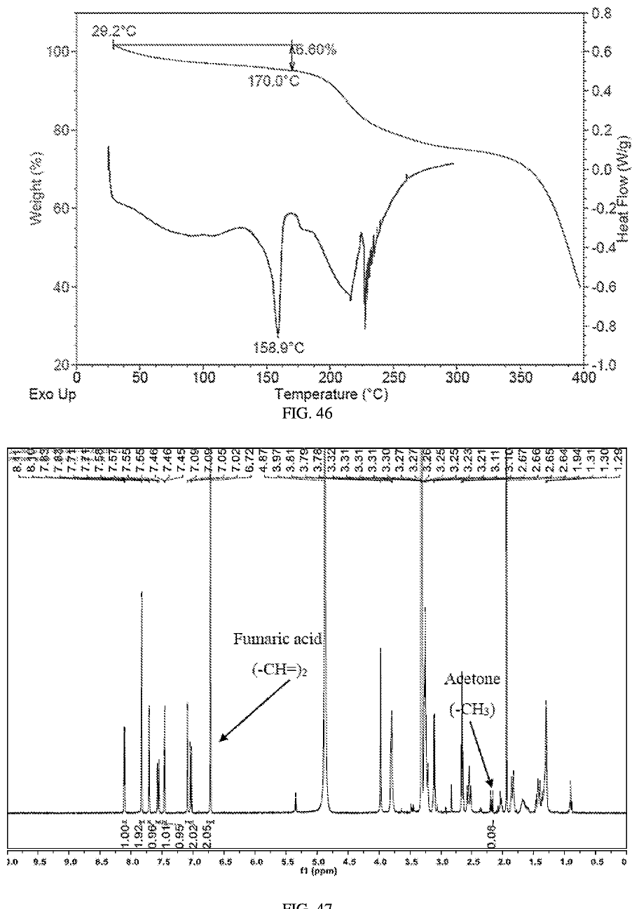

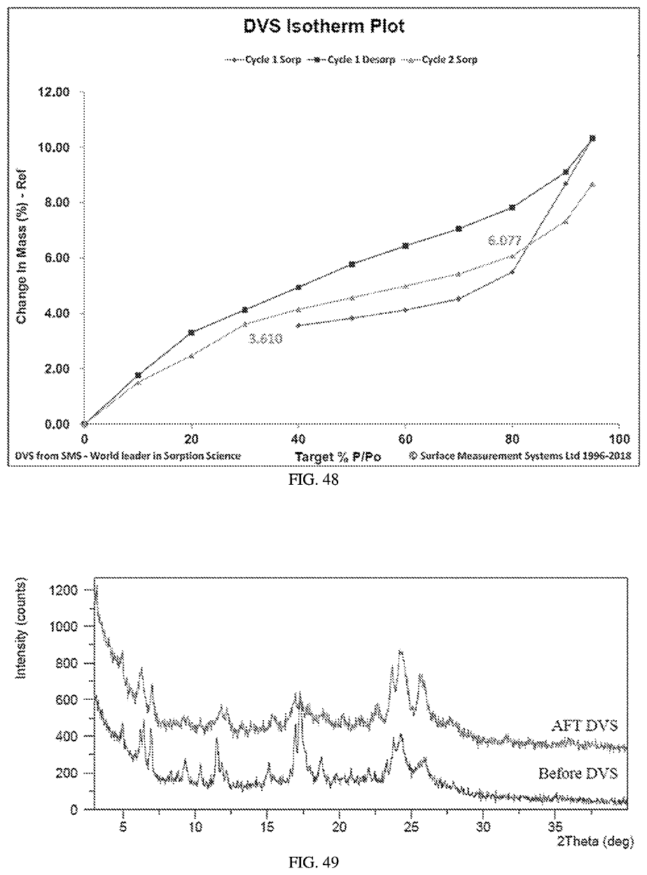

Fumarate salt Type B samples were obtained during salt formation experiments in THF/EtOAc/acetone. The form was selected for re-preparation on 500 mg scale. The detailed preparation procedure of fumarate Type B is summarized in Table 4-4, although fumarate Type A was obtained. The XRPD pattern of fumarate Type A is displayed in FIG. 45. TGA/DSC results (FIG. 46) of the sample showed a weight loss of 6.6% up to 170° C. and one endotherm at 158.9° C. (peak). ¹H NMR spectrum (FIG. 47) showed the molar ratio of acetone to freeform in fumarate Type A was about 0.01:1.0 (˜0.08%), and the molar ratio of fumaric acid to freeform was 1.0:1.0. DVS plot of fumarate Type A showed a moisture uptake of 6.1% from 0% RH to 80% RH at 25° C. (FIG. 48). The crystallinity of fumarate Type A decreased after DVS test (FIG. 49).

### Example 4. Evaluation of Salt Forms and Freeform

HCl salt Type F, fumarate Type A and sulfate Type B were selected as salt forms to compare the grinding stability and solubility of different salts and freeform Type A.

### Grinding Stability

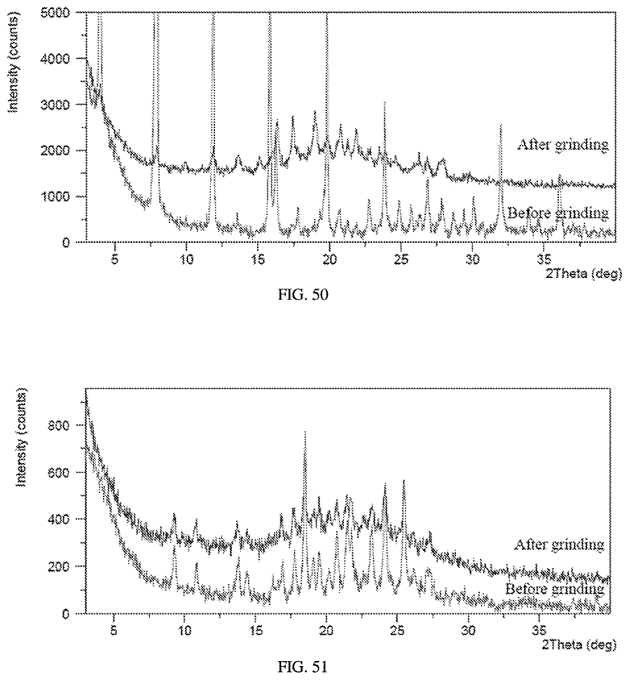

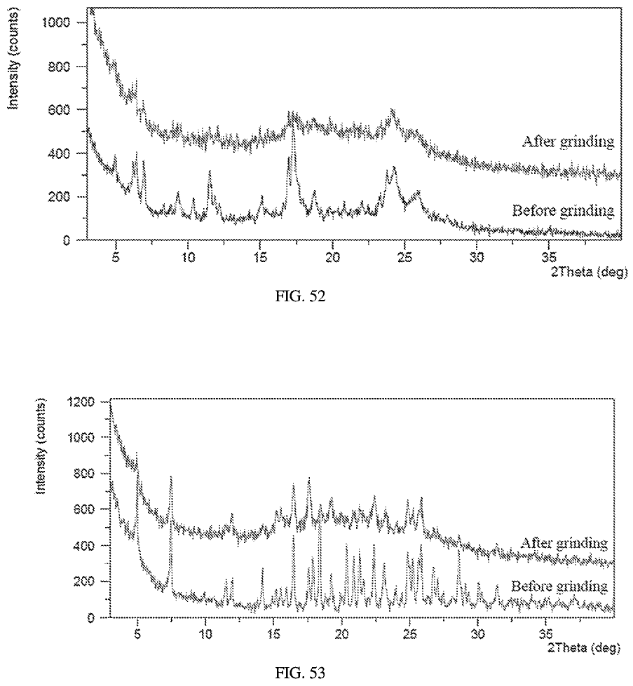

To compare the grinding stability of freeform Type A, sulfate Type B, fumarate Type A and HCl salt Type F, approximately 30 mg of each solid sample was added into a mortar, and then ground for about 5 minutes manually. The crystallinity of freeform Type A, sulfate Type B, fumarate Type A and HCl salt Type F decreased after grinding, and the results are shown in FIGS. 50-53.

### Equilibrium Solubility in pH Buffers (3.0-8.0), 20% Captisol and Water

Equilibrium solubility of freeform Type A, sulfate Type B, fumarate Type A, and HCl salt Type F in 50 mM pH buffers (pH=3.0, 4.0, 5.0, 6.0, 7.0, 8.0), 20% captisol (pH=5.0, w/v) and water was performed with sampling time at 24 hrs at RT (about 21° C.). ˜5 mg of solids were suspended into 1 mL of each medium with dosing conc. at ˜5 mg/mL. Additional solids were added into the sample to generate suspension if clear solution obtained. The suspension was equilibrated via stirring (1000 rpm). The suspension was centrifuged to obtain precipitate and supernatants.

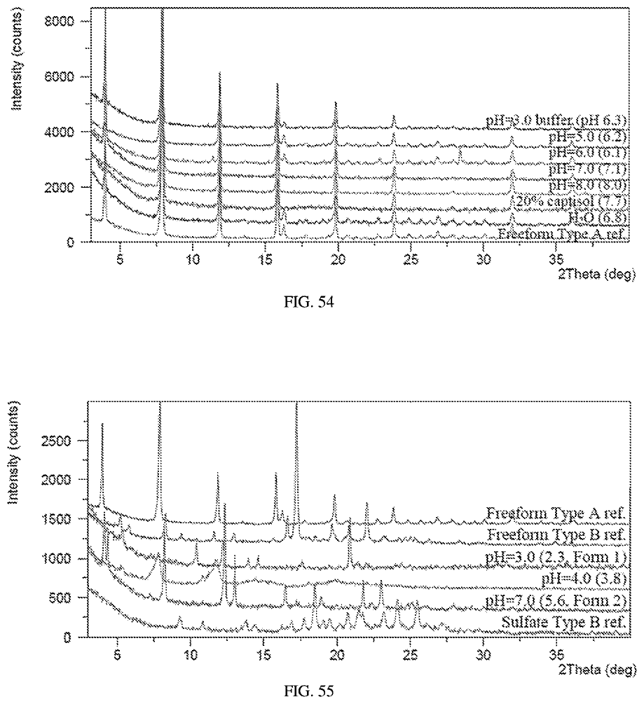

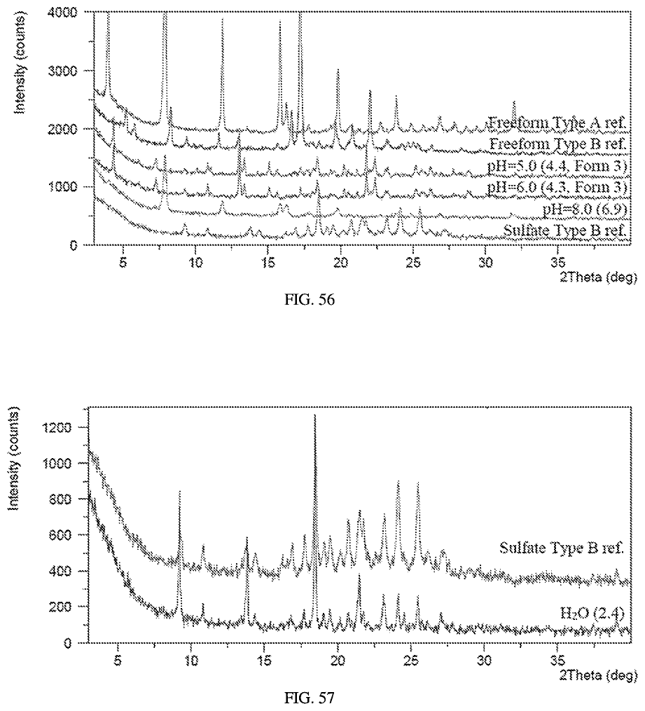

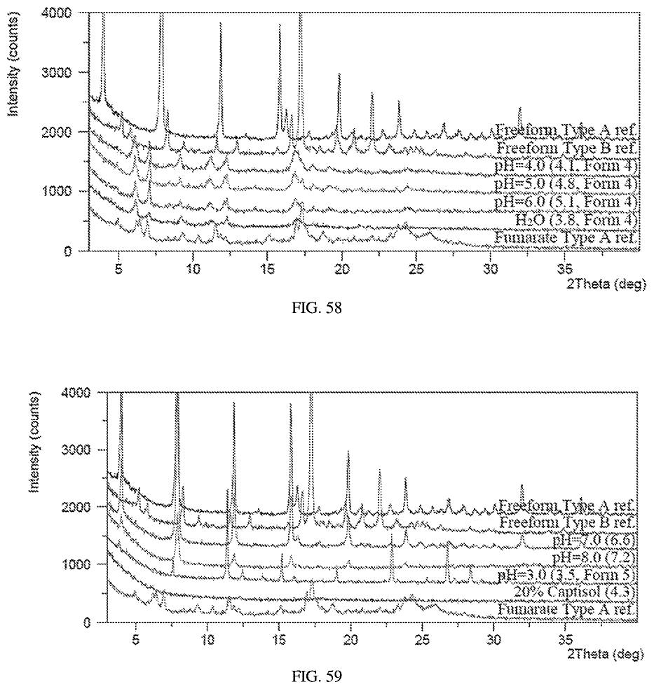

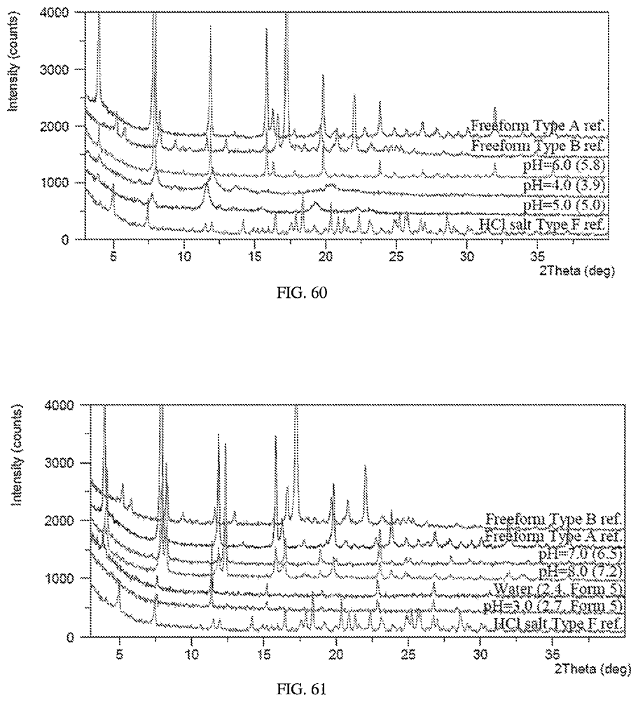

Solubility and pH were tested for the supernatants after filtration, and isolated precipitate was tested by XRPD. The equilibrium solubility results are summarized in Table 5-1. The XRPD results are shown in FIGS. 54-61.

1. No form change was observed for freeform Type A after equilibrium in all media (clear solution was obtained in pH=4.0 buffer) for 24 hours. Form change was observed for the three salt forms after equilibrium in all media for 24 hours (except for sulfate Type B in water).

2. Freeform Type A showed highest solubility in pH=4.0 buffer (>17.9 mg/mL, 50 mM citrate buffer, final pH=4.8). Fumarate Type A showed highest solubility in pH=6.0 buffer (20.6 mg/mL, 50 mM citrate buffer, final pH=5.1). HCl salt Type F and sulfate Type B showed low solubility in pH buffers (<8 mg/mL).

TABLE 5-1
Summary of equilibrium solubility results at RT

| | Freeform Type A | | | Sulfate Type B | | | Fumarate Type A | | | HCl salt Type F | | |
|---|---|---|---|---|---|---|---|---|---|---|---|---|
| Buffer | S | pH | FC | S | pH | FC | S | pH | FC | S | pH | FC |
| pH = 3.0 buffer | 0.05 | 6.3 | N | 8.00 | 2.3 | 1 | 0.65 | 3.5 | 5 | 2.77 | 2.7 | 5 |
| (50 mM HCl + KCl) | | | | | | | | | | | | |
| pH = 4.0 buffer | >17.9^{#} | 4.8 | — | 2.33 | 3.8 | Low | 3.45* | 4.1 | 4 | 1.12 | 3.9 | Low |
| (50 mM citrate) | | | | | | | | | | | | |
| pH = 5.0 buffer | 3.31 | 5.2 | N | 1.66* | 4.4 | 3 | 7.79 | 4.8 | 4 | 2.64 | 5.0 | Low |
| (50 mM citrate) | | | | | | | | | | | | |
| pH = 6.0 buffer | 0.18 | 6.1 | N | 0.93* | 4.3 | 3 | 20.6 | 5.1 | 4 | 0.62 | 5.8 | A |
| (50 mM citrate) | | | | | | | | | | | | |
| pH = 7.0 buffer | 0.02 | 7.1 | N | 0.61* | 5.6 | 2 | 0.05 | 6.6 | A | 0.06 | 6.5 | A⁺ |
| (50 mM phosphate) | | | | | | | | | | | | |
| pH = 8.0 buffer | 0.02 | 8.0 | N | 0.04* | 6.9 | A | 0.02 | 7.2 | A | 0.02 | 7.2 | A⁺ |
| (50 mM phosphate) | | | | | | | | | | | | |
| 20% Captisol | 12.1 | 7.7 | N | >44.5¹ | 2.6 | — | 29.6 | 4.3 | L | >48.0¹ | 3.5 | — |
| (pH = 5.0, w/v) | | | | | | | | | | | | |
| H₂O | 0.02 | 6.8 | N | 1.97 | 2.4 | N | 1.85 | 3.8 | 4 | 4.24 | 2.4 | 5 |

S: solubility (mg/mL);
FC: form change;
N: solid form not changed;
—: sample was not enough for test;
1/2/3/4/5: new Form 1/2/3/4/5;
A: freeform Type A;
A⁺: freeform Type A + additional peaks;
Low: low crystallinity;
¹Clear solution was obtained after adding about ~50 mg sample.
^{#}Suspension was obtained after adding 20 mg freeform Type A to 1 mL buffer and equilibrate for about 20 minutes. Then the suspension transferred to clear solution after 24 hours equilibration.
*After adding 5 mg sample, clear solution was obtained. More sample was added to generate suspension. The solubility was lower than 5 mg/mL, solid might precipitate out during the solubility test.

Equilibrium Solubility in pH Buffers (pH 4.0-6.0) with pH Adjustment

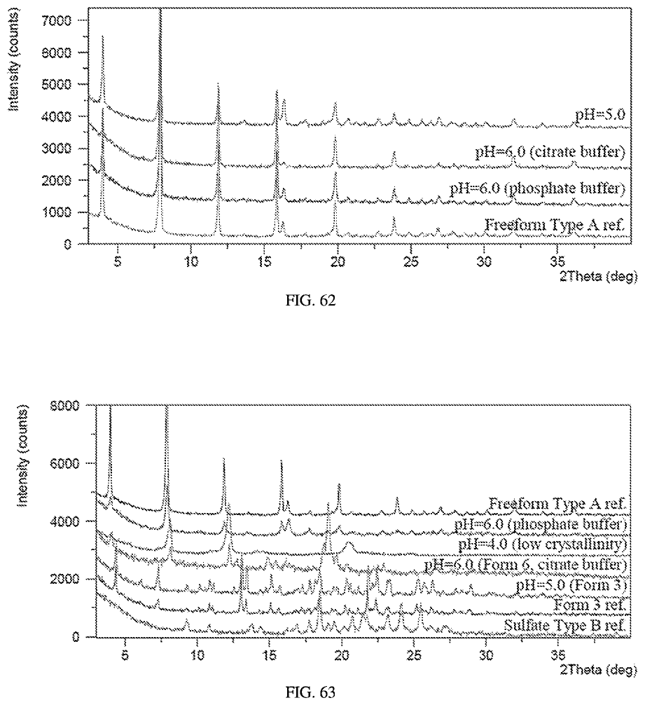

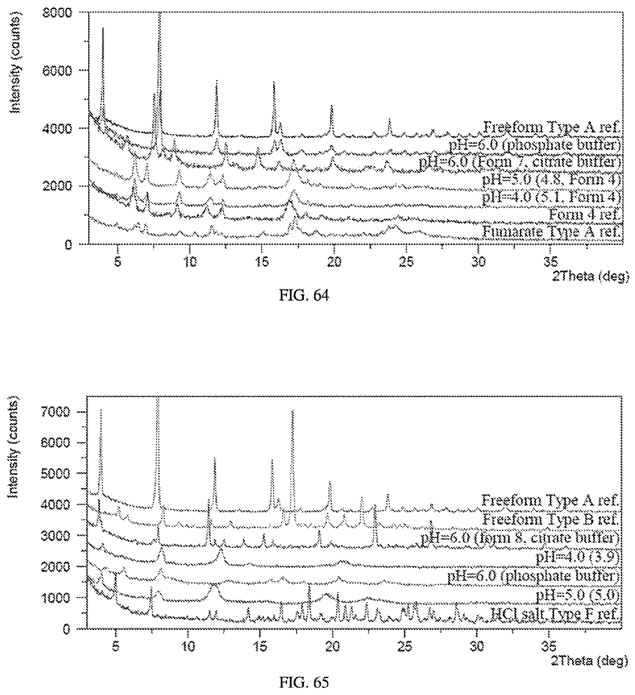

Freeform Type A and fumarate Type A showed higher solubility in 50 mM pH=4.0 and pH=6.0 buffer. To differentiate solubility under different pH in the range pH 4.0 to 6.0, solubility of all four materials were tested in 50 mM pH=4.0, 5.0 and 6.0 citrate buffers and pH=6.0 phosphate buffer with pH adjustment. In details, ˜5 mg of solids were suspended into 1 mL of each medium with dosing conc. at ˜5 mg/mL. Additional solids were added into the sample to generate suspension if clear solution was obtained. The suspension was equilibrated via stirring (1000 rpm) for 24 hours. The pH of the suspension was adjusted when the final pH shifted above 0.3, and the samples were stirred for another 1.5 hours after pH adjustment. The suspension was centrifuged to obtain precipitate and supernatants. Solubility, purity and pH were tested for the supernatants after filtration, and isolated precipitate was tested by XRPD. The equilibrium solubility results are summarized in Table 5-2. The XRPD results are shown in FIGS. 62-65.

1. No form change was observed for freeform Type A after equilibrium in all media (Clear solution was obtained in pH=4.0 buffer) for 24 hours. Form change was observed for the three salt forms after equilibrium in all media for 24 hours (sulfate Type B showed no form change in water).

2. Freeform Type A showed highest solubility in pH=4.0 buffer (>36.9 mg/mL, 50 mM citrate buffer, final pH=4.2). Fumarate Type A showed highest solubility in pH=5.0 buffer (19.3 mg/mL, 50 mM citrate buffer, final pH=4.9). HCl salt Type F and sulfate Type B showed low solubility in pH buffers (<6.8 mg/mL).

TABLE 5-2
Summary of equilibrium solubility results at RT in pH = 4.0/5.0/6.0 buffers

| | Freeform Type A | | | | Sulfate Type B | | | |
|---|---|---|---|---|---|---|---|---|
| Buffer | S | pH | FC | Area % | S | pH | FC | Area % |
| pH = 4.0 buffer | >36.9^{#} | (5.0) | — | 99.9 | 2.29 | 3.9 | Low | 99.2 |
| (50 mM citrate) | | 4.2* | | | | | | |
| pH = 5.0 buffer | 3.31 | 5.2 | N | 99.6 | 6.82^{#} | (4.5) | 3 | 99.4 |
| (50 mM citrate) | | | | | | 5.0* | | |
| pH = 6.0 buffer | 0.15 | 6.0 | N | 99.6 | 0.07 | (4.4) | 6 | 92.6 |
| (50 mM citrate) | | | | | | 6.1* | | |
| pH = 6.0 buffer | 0.11 | 6.0 | N | 99.3 | 0.06 | (3.0) | A | 88.2 |
| (50 mM phosphate) | | | | | | 6.2* | | |

| | Fumarate Type A | | | | HCl salt Type F | | | |
|---|---|---|---|---|---|---|---|---|
| Buffer | S | pH | FC | Area % | S | pH | FC | Area % |
| pH = 4.0 buffer | 3.04 | 4.0 | 4 | 99.3 | 1.52 | 4.0 | Low | 99.0 |
| (50 mM citrate) | | | | | | | | |
| pH = 5.0 buffer | 19.3^{#} | (4.1) | 4 | 99.4 | 2.32 | 4.9 | Low | 99.6 |
| (50 mM citrate) | | 4.9* | | | | | | |
| pH = 6.0 buffer | 0.24 | (5.0) | 7 | 98.6 | 1.15 | 5.7 | 8 | 99.0 |
| (50 mM citrate) | | 6.1* | | | | | | |
| pH = 6.0 buffer | 0.09 | (4.6) | A | 95.6 | 0.43 | (3.9) | Low | 98.9 |
| (50 mM phosphate) | | 6.2* | | | | 5.9* | | |

S: solubility (mg/mL), FC: form change, N: solid form not changed, —: sample was not enough for test. 3/4/6/7/8: new Form 3/4/6/7/8. A: freeform Type A. Low; low crystallinity.
*pH shift (>0.3) was observed and the pH was adjusted to target pH with corresponding solid (citric acid or trisodium citrate or Na₂HPO₄ or NaH₂PO₄).
^{#}Clear solution was obtained after pH adjustment and additional solid was added to generated suspension. The sample was stirred for another ~1.5 hour.

### Example 5. Pre-formulation Development

### In Situ Salt Solubility Test

Freeform showed high solubility in pH=4.0 buffer (>36.9 mg/mL, 50 mM citrate buffer, final pH=4.2), and fumarate Type A showed high solubility in pH=5.0 buffer (19.3 mg/mL, 50 mM citrate buffer, final pH=4.9). Citric acid and fumaric acid were selected for in situ salt solubility tests with freeform Type D.

For citric acid in situ salt formation, ˜50 mg freeform Type D and 1.1 equivalent of citric acid was added into a 5 mL vial and 1.1 mL water was added to dissolve the sample. The sample was almost clear (little solid was observed) and filtered for a solubility test. The tested concentration of freeform Type D with 1.1 equiv. of citric acid in water was 39.3 mg/mL (final pH=3.4).

For fumaric acid in situ salt formation, ˜5 mg freeform Type D and 1.1 equivalent of fumaric acid was added into a 5 mL vial and 4.2 mL water was added to dissolve the sample. The sample was almost clear (little solid was observed) and filtered for a solubility test. The tested concentration of freeform Type D with 1.1 equivalent of fumaric acid in water was 1.1 mg/mL (final pH=4.0).

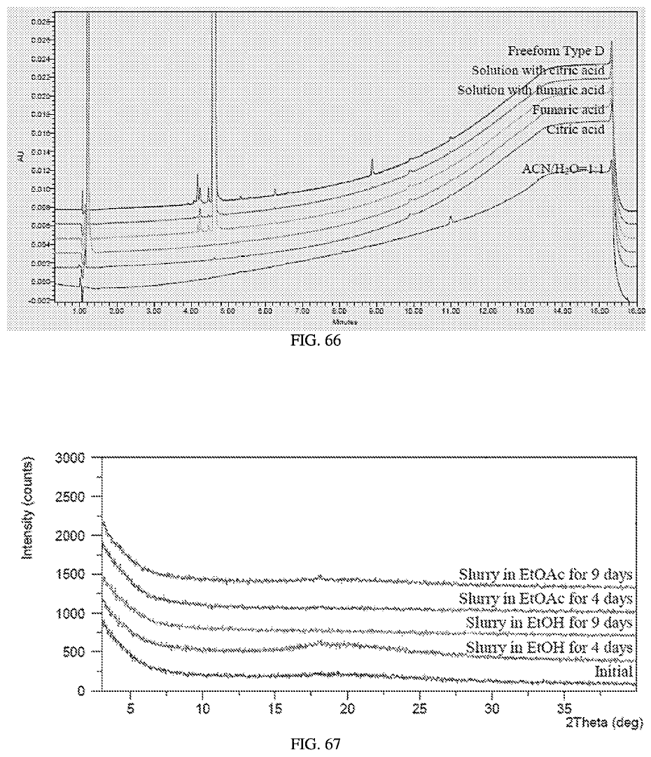

The little solid observed during sample preparation may be insoluble impurity according to data from the in situ salt formation samples (FIG. 66). The results show that one impurity with retention time around 9 mins in freeform Type D disappeared in the solution with citric acid and the solution with fumaric acid, thus the impurity may be filtered before the purity and concentration tests.

The in situ salt formation and solubility evaluation results are summarized in Table 6-1. In situ salt formation with citric acid showed higher solubility, and citric acid was selected for further in situ salt formation and further pre-formulation study.

TABLE 6-1
Summary of in situ salt solubility test results

| | | | | HPLC purity |
|---|---|---|---|---|
| | | Concentration | | of filtrates |
| Sample | Counter ion | (mg/mL) | pH | (Area %) |
| Freeform Type D* | Citric Acid | 39.3 | 3.4 | 99.9% |
| | Fumaric Acid | 1.1 | 4.0 | 99.8% |

*The initial purity of freeform Type D was 99.6%.

### Preparation of Solid Citrate Salt Sample

About 35 mg freeform Type D and 9.6 mg citric acid (˜1 equiv.) were dissolved in 1 mL water (˜50 mM), and the sample was slightly cloudy and filtered to obtain supernatant. The supernatant obtained was vacuum dried at RT, and amorphous sample was obtained. The amorphous sample was stirred in EtOH and EtOAc for 9 days, respectively. XRPD results showed the sample after stirring in solvent was still amorphous (FIG. 67).

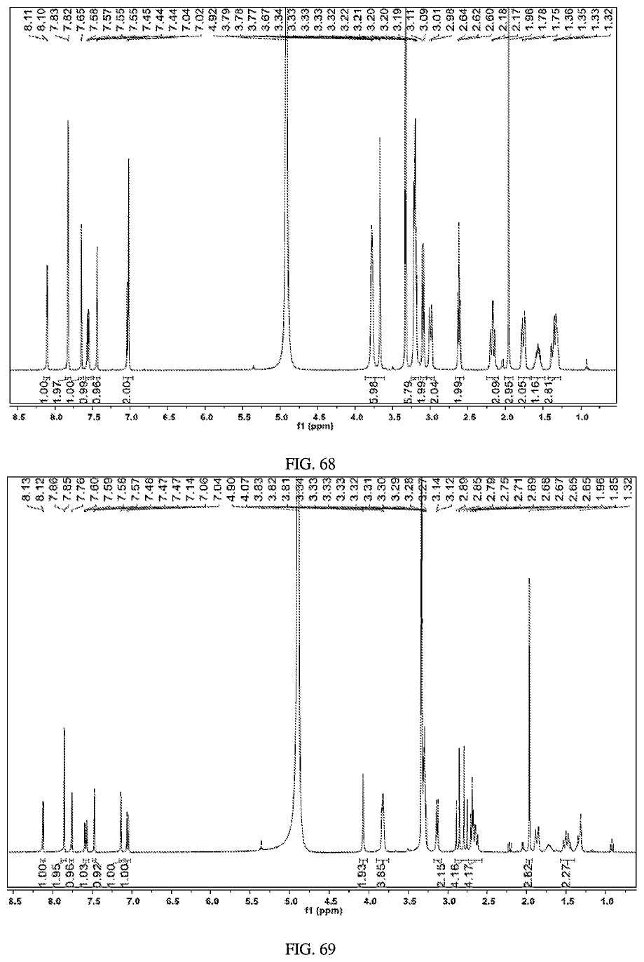

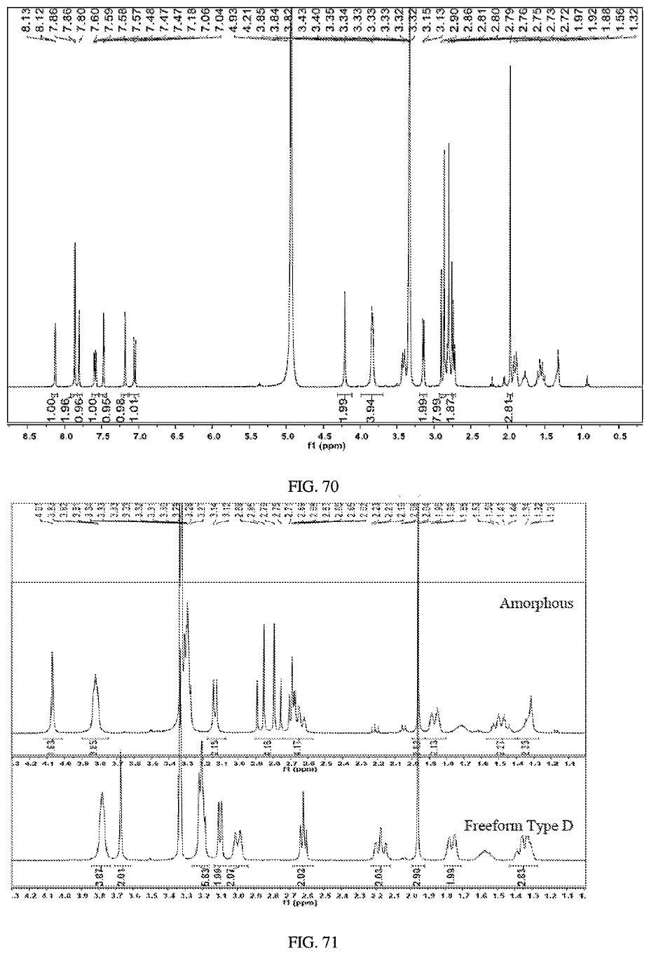

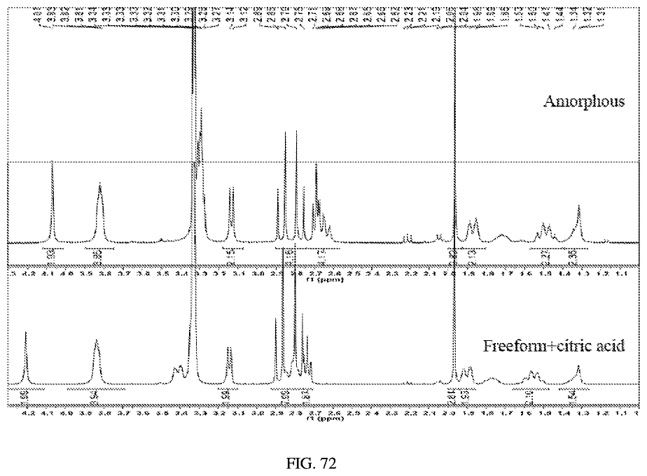

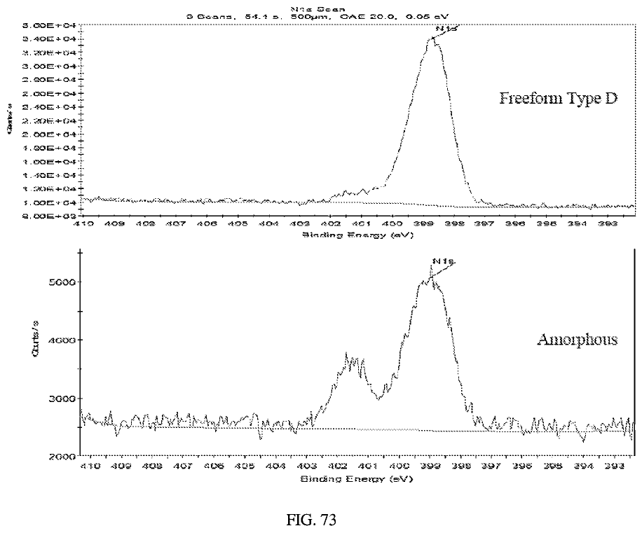

¹H NMR of freeform Type D, amorphous sample, and mixture of freeform Type D+1 equiv. citric acid were obtained. The results were shown in FIGS. 68-72. Shifted ¹H NMR signals were observed for the samples. XPS of freeform Type D and amorphous sample (FIG. 73) show that the nitrogen peak of the two samples was shifted, indicating potential salt formation.

### Solubility Profile of Freeform Type D in Citrate Buffers

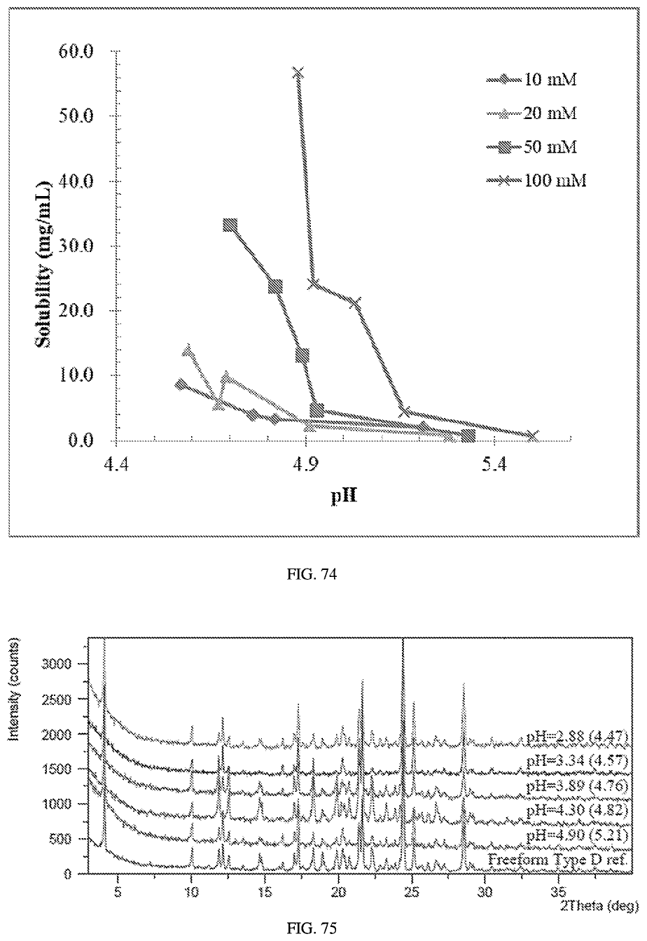

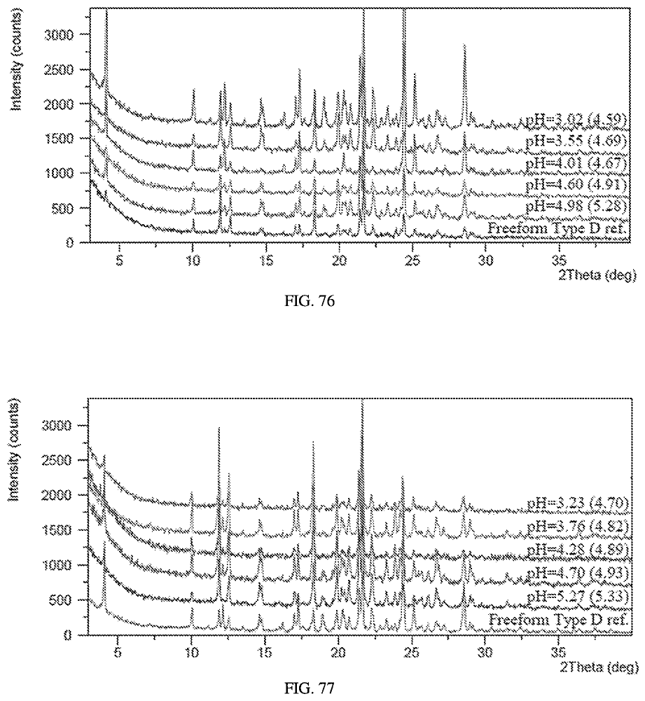

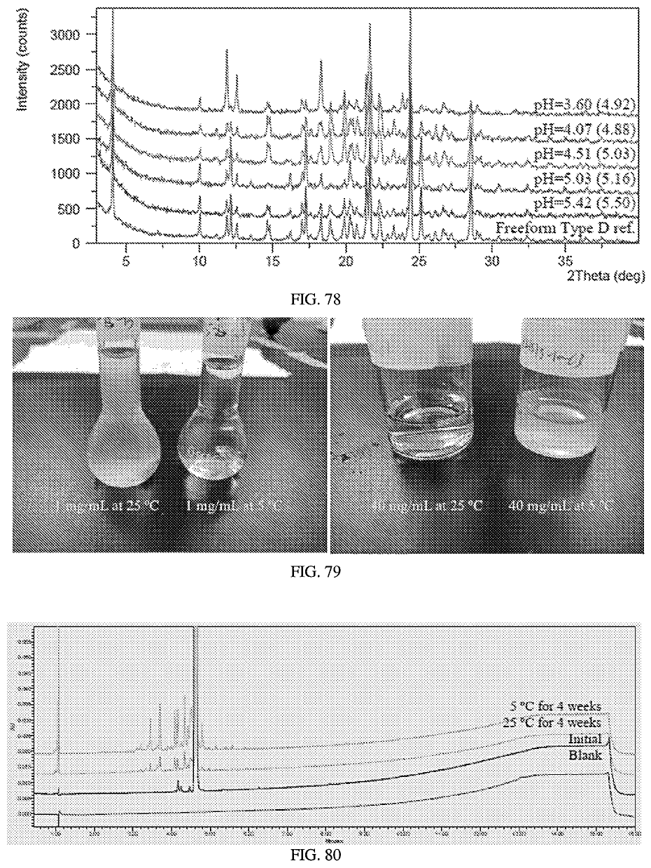

To understand the solubility profile of freeform Type D in citrate buffers with different concentration and pH, equilibrium solubility of freeform Type D in citrate buffer with different buffer concentration (10/20/50/100 mM) and different pH (2.8-5.5) were tested at RT. ˜5 mg of solids was suspended into 1 mL of each medium with dosing conc. at ˜5 mg/mL. Additional solids were added into the sample to generate suspension if clear solution was obtained. The suspension was equilibrated via stirring (1000 rpm) for 24 hours. The suspension was centrifuged to obtain precipitate and supernatants. Solubility and pH were tested for the supernatants after filtration, and the isolated precipitate was tested by XRPD. The equilibrium solubility results are summarized in Table 6-2, and the solubility curve of freeform Type D in citrate buffers is shown in FIG. 74. The XRPD results are shown in FIG. 75 to FIG. 78. No form change of freeform Type D was observed after equilibrium in all buffers for 24 hours. The solubility of freeform Type D increased when pH decreased or buffer concentration increased. The highest solubility tested was 56.8 mg/mL (100 mM citrate buffer, final pH=4.9).

TABLE 6-2
Summary of equilibrium solubility results in citrate buffers at RT

| Buffer | Buffer | Solubility | Initial | End pH | |
|---|---|---|---|---|---|
| Concentration | pH | (mg/mL) | pH | (24 hours) | Form |
| 10 mM | 2.88 | 8.6 | 4.56 | 4.47 | Freeform |
| (citrate | 3.34 | 4.0 | 4.52 | 4.57 | Type D |
| buffer) | 3.89 | 3.4 | 4.69 | 4.76 | |
| | 4.30 | 2.1 | 4.75 | 4.82 | |
| | 4.90 | 0.7 | 5.17 | 5.21 | |
| 20 mM | 3.02 | 14.0 | 4.60 | 4.59 | |
| (citrate | 3.55 | 9.8 | 4.73 | 4.69 | |
| buffer) | 4.01 | 5.5 | 4.64 | 4.67 | |
| | 4.60 | 2.3 | 4.80 | 4.91 | |
| | 4.98 | 0.8 | 5.20 | 5.28 | |
| 50 mM | 3.23 | 33.2 | 4.64 | 4.70 | |
| (citrate | 3.76 | 23.8 | 4.82 | 4.82 | |
| buffer) | 4.28 | 13.2 | 4.88 | 4.89 | |
| | 4.70 | 4.7 | 4.93 | 4.93 | |
| | 5.27 | 0.8 | 5.33 | 5.33 | |
| 100 mM | 3.60 | 24.3 | 4.92 | 4.92 | |
| (citrate | 4.07 | 56.8 | 4.86 | 4.88 | |
| buffer) | 4.51 | 21.2 | 5.00 | 5.03 | |
| | 5.03 | 4.4 | 5.26 | 5.16 | |
| | 5.42 | 0.6 | 5.45 | 5.50 | |

### Example 6. Stability Evaluation of Freeform

### Solution Stability in Citrate Buffers

Solution stability of the freeform Type D in 10 mM citrate buffer (pH 4.3, 1 mg/mL) and 100 mM citrate buffer (pH 4.1, 40 mg/mL) was determined. The stock solution of 40 mg/mL freeform Type D in 100 mM pH=4.1 citrate buffer was slightly cloudy, and the stability experiment was set up after the suspension was filtrated via a 0.45 m PTFE filter. The stock solution of 1 mg/mL freeform Type D in 10 mM pH=4.3 citrate buffer was clear and not filtered before the stability experiment set up. The stability samples were stored under 5° C. and 25° C. for 28 days, respectively. After 28 days storage, the stability samples were taken out for HPLC test and pH test.

The 1 mg/mL freeform Type D in 10 mM citrate buffer (pH 4.3) became cloudy and yellow at 25° C. for 28 days, and became a yellow solution at 5° C. for 28 days. The 40 mg/mL freeform Type D in 100 mM citrate buffer (pH 4.1) became cloudy at 5° C. for 28 days, and remained a clear solution at 25° C. for 28 days. The visual observation is shown in FIG. 79.

Stability results are summarized in Table 7-1. The assay of 1 mg/mL freeform Type D in citrate buffer was decreased to 68.4% and 11.6% after being stored at 5° C. and 25° C. for 28 days. No significant degradation was observed for 40 mg/mL freeform Type D in 100 mM citrate buffer at 5° C. and 25° C. for 28 days. The assay of the stability samples decreased (95.0% and 98.9% for 5° C. and 25° C. stability sample). Chromatogram overlays of stability samples are shown in FIG. 80 and FIG. 81.

TABLE 7-1
Summary of solution stability experiments in citrate buffer

| | | | Test Items | | | |
|---|---|---|---|---|---|---|
| | | | Purity | Assay | | |
| Sample | Conditions | | (Area %) | (%) | Appearance | pH |
| ~1 mg/mL | 5° | C. | 89.0 | 68.4 | Yellow | 4.7 |
| Freeform Type D | | | | | | |
| in 10 mM citrate | 25° | C. | 78.9 | 11.6 | Cloudy, | 4.6 |
| buffer (pH 4.3) | | | | | yellow | |
| ~40 mg/mL | 5° | C. | 99.7 | 95.0 | Cloudy | 4.8 |
| Freeform Type D | | | | | | |
| in 100 mM citrate | 25° | C. | 99.5 | 98.9 | Clear | 4.9 |
| buffer (pH 4.1) | | | | | | |

The initial purity of freeform Type D was 99.6%.

### Formulation Solution Stability

Solution stability of freeform Type D in five formulations under 5° C., 25° C., 40° C. and 60° C. was tested. The five formulations are shown in Table 7-2, and the characterization results are summarized in Example 7. The procedure is summarized below.
1. Weigh about 544 mg freeform Type D into a 25 mL volumetric flask and prepare 5 copies (named as formulation 1/2/3/4/5).
2. Add about 149 mg citric acid into the volumetric flask of formulation 1/2/3/4.
3. Add about 2037 mg of the corresponding sugar into the volumetric flask of formulation 2/3/4. (The solubility of freeform Type D in the presence of sugars were shown in Example 7)
4. Dilute to volume with water for formulation 1/2/3/4.
5. Dilute to volume with pH=4.0 citrate/phosphate buffer for formulation 5. (The solubility of freeform Type D in citrate/phosphate buffers were shown in Example 7.)
6. Sonicate the samples for about 2 mins, and filter with 0.45 m filter to obtain a clear solution.
7. Fill and seal about 0.7 mL solutions into 32 separate HPLC vials per each formulation and store under corresponding conditions.

The stability samples were stored at 5° C., 25° C., 40° C. and 60° C. for 28 days. After 1 day, 3 days, 7 days, 14 days and 28 days storage, the stability samples at 25° C., 40° C. and 60° C. were taken out for HPLC and pH tests. After 28 days storage, the stability samples at 5° C. were taken out for HPLC and pH tests.

TABLE 7-2
Summary of the five formulations used for stability test

| Formulation | | | |
|---|---|---|---|
| Number | Freeform | Citric Acid | Excipients |
| Formulation 1 | 30 mM | 30 mM | Not added |
| Formulation 2 | 30 mM | 30 mM | 240 mM lactose |
| Formulation 3 | 30 mM | 30 mM | 240 mM trehalose |
| Formulation 4 | 30 mM | 30 mM | 240 mM sucrose |

| | | |
|---|---|---|
| Formulation 5 | 30 mM | pH = 4.0 citrate/phosphate buffer |
| | | (61 mM citric acid + 77 mM phosphate) |

Stability Experiments under 25° C., 40° C., and 60° C.

All the stability samples at 25° C., 40° C. and 60° C. were still clear solutions. Stability results of freeform in formulation 1 to formulation 5 under 25° C., 40° C. and 60° C. are summarized in Tables 7-3 to 7-7.
1. No significant degradation was observed after 28 days storage at 25° C. in all 5 formulations.
2. 1.3%-2% degradation was observed after 28 days storage at 40° C. in all 5 formulations.
3. ˜7% degradation was observed after 16 days storage at 60° C. in all 5 formulations.

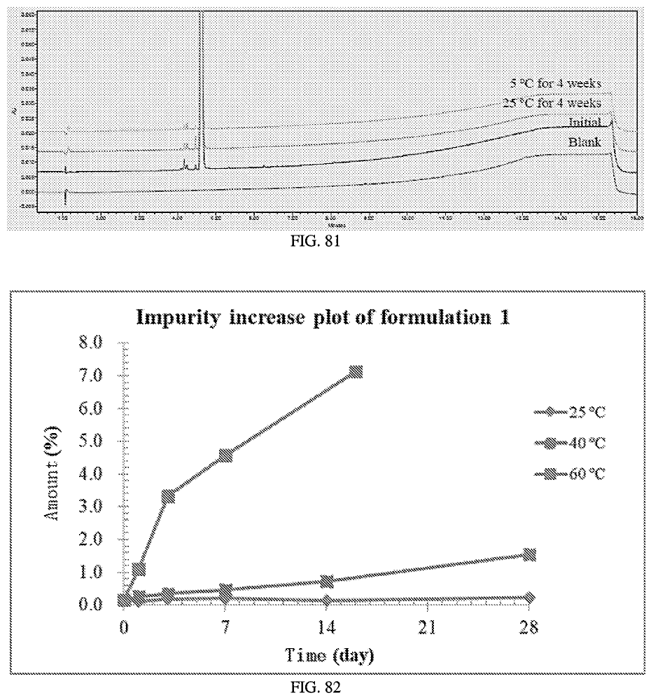

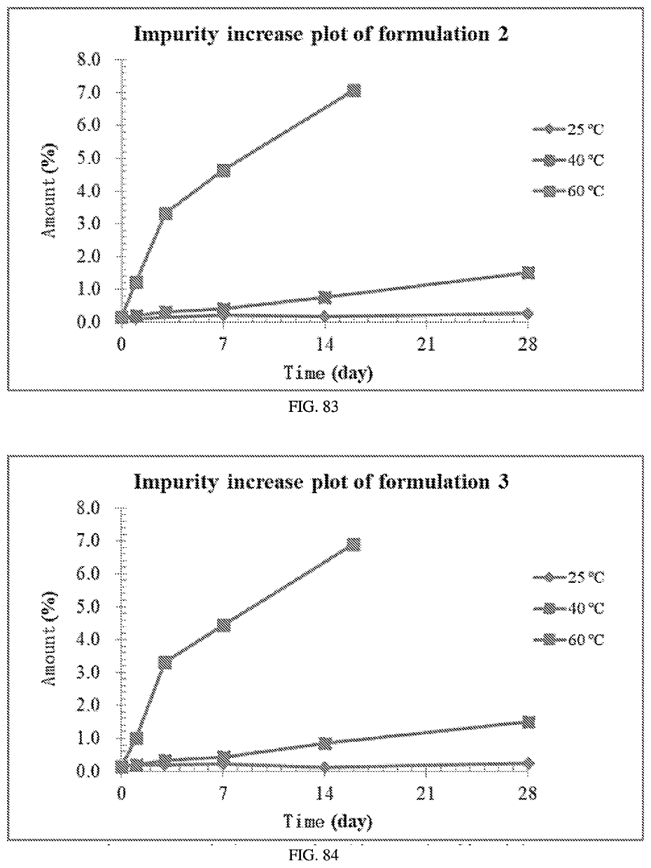

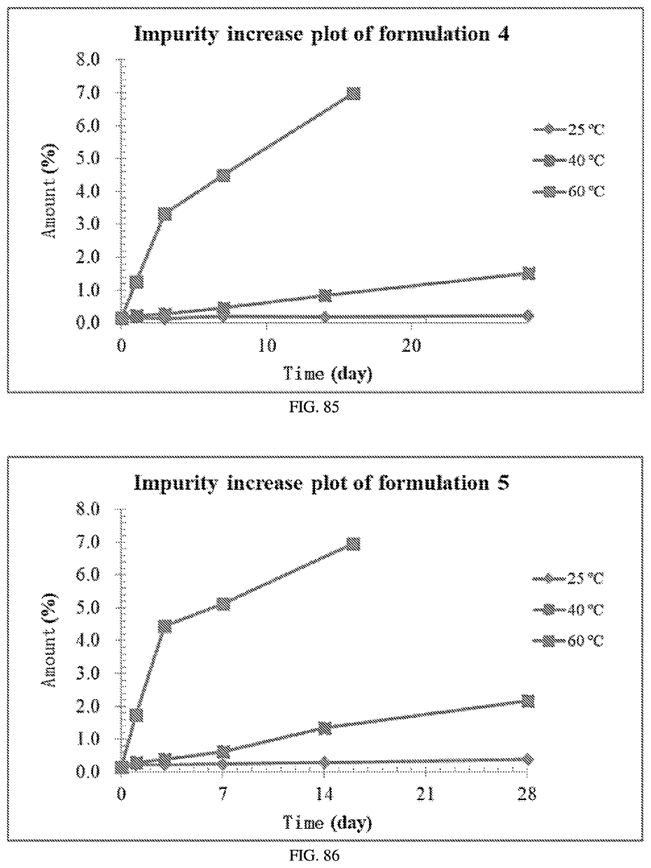

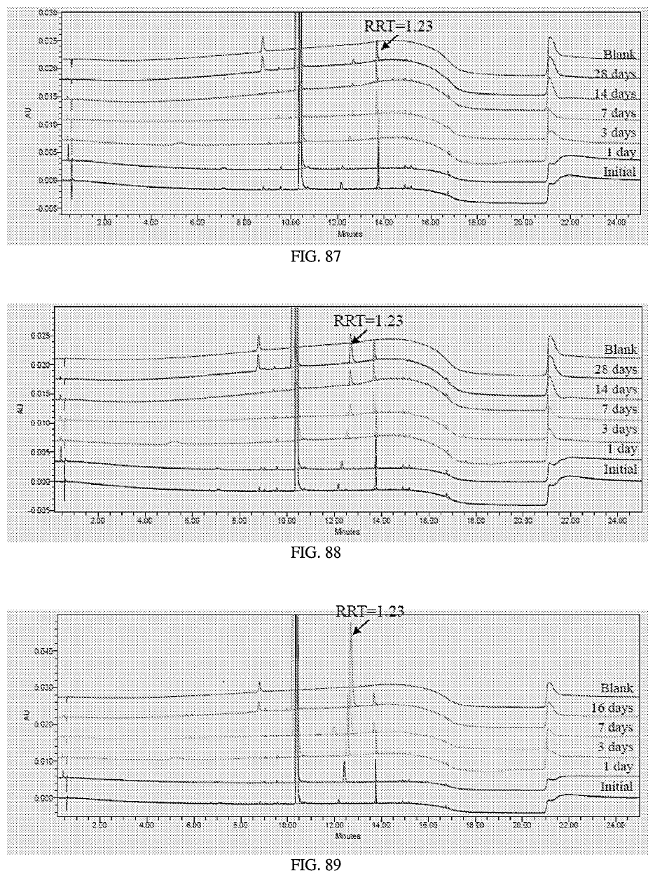

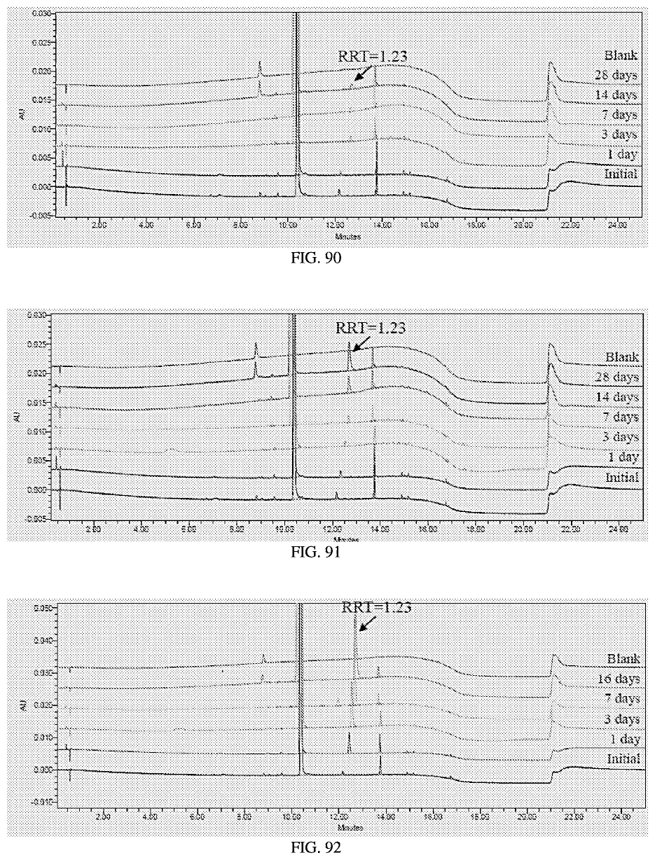

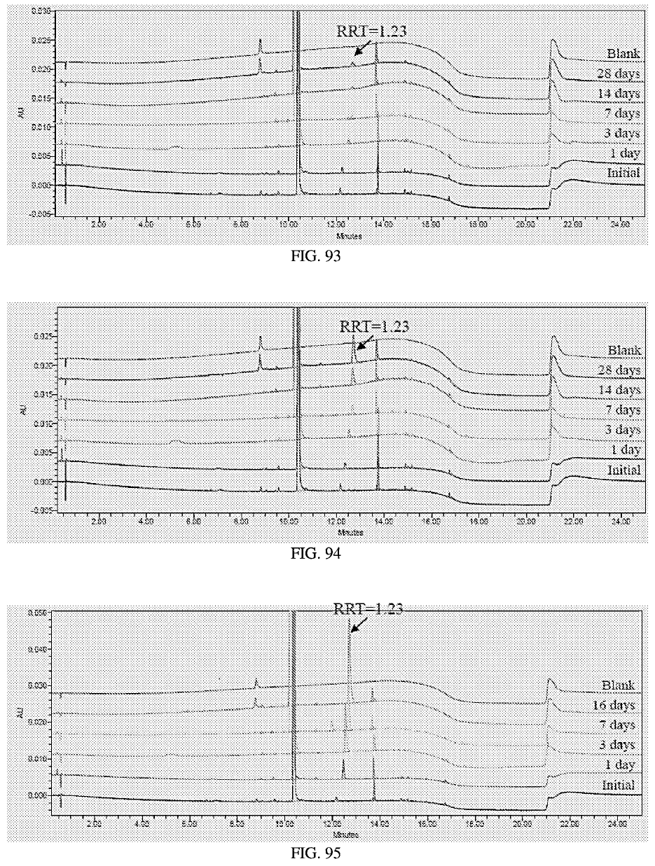

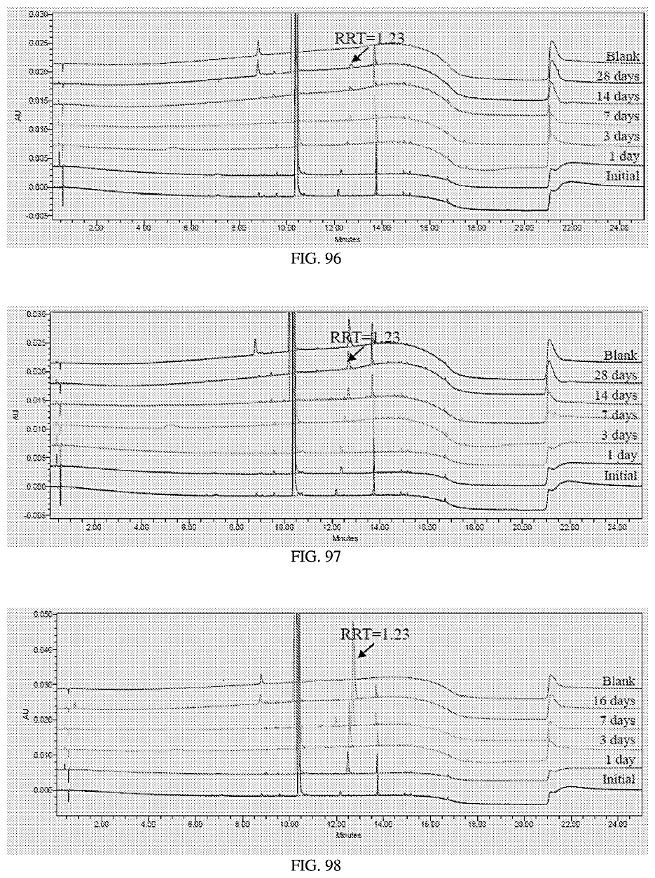

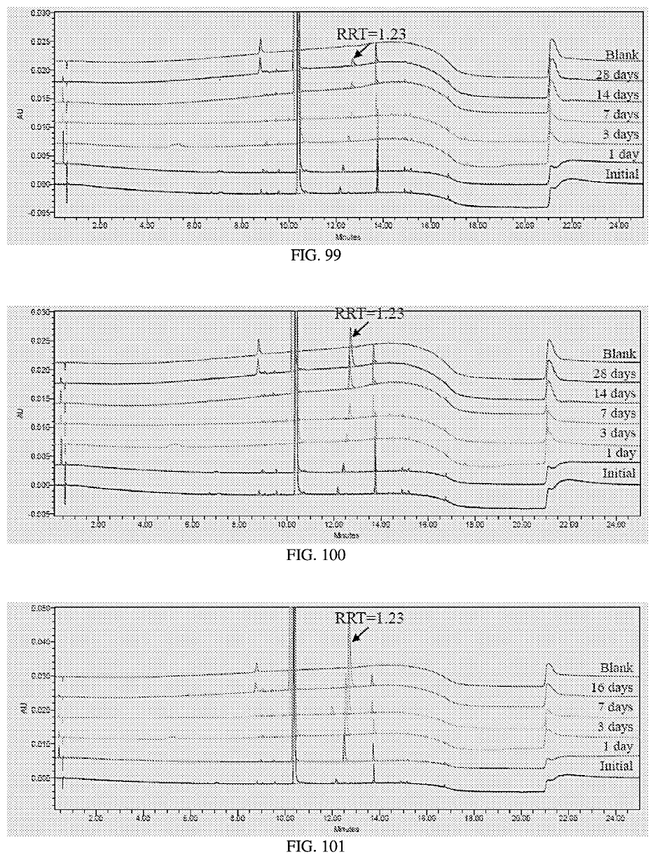

The impurity at RRT around 1.23 was the main growing impurity. The impurity increased as temperature and time increased, and the impurity increase plots in formulation 1 to 5 are shown in FIG. 82 to FIG. 86. Chromatogram overlays of stability samples are shown in FIG. 87 to FIG. 101. The results show that the presence of sugars in the formulations do not affect stability.

TABLE 7-3
Summary of solution stability evaluation experiments for formulation 1

| | | | 1 day | | | 3 days | | | 7 days | | |
|---|---|---|---|---|---|---|---|---|---|---|---|
| | | | Purity | Assay | | Purity | Assay* | | Purity | Assay* | |
| Sample | Temp. | | (area %) | (%) | pH | (area %) | (%) | pH | (area %) | (%) | pH |
| | 25° | C. | 99.13 | 102.7 | 4.1 | 98.89 | 90.0 | 4.1 | 98.90 | 100.6 | 4.0 |
| | 40° | C. | 98.98 | 103.7 | 4.1 | 98.80 | 102.3 | 4.1 | 98.58 | 93.7 | 4.2 |
| | 60° | C. | 98.16 | 94.2 | 4.0 | 95.80 | 96.7 | 4.1 | 93.54 | 98.6 | 4.1 |
| | | | 14 days | | | 28 days | | | | | |
| | | | Purity | Assay | | Purity | Assay | | | | |
| | Temp. | | (Area %) | (%) | pH | (Area %) | (%) | pH | | | |
| ~30 mM | 25° | C. | 99.06 | 98.5 | 4.0 | 99.01 | 96.4 | 4.0 | NA | | |
| API + 30 mM | 40° | C. | 98.51 | 100.2 | 4.1 | 97.74 | 100.2 | 4.0 | | | |

| | | | | | | |
|---|---|---|---|---|---|---|
| citric acid | 60° | C.^{#} | 91.87 | 94.7 | 4.0 | Not tested |
| (~20 mg/mL, | | | | | | |
| formulation 1) | | | | | | |

The initial purity was 99.01%.
*Assay of 1/3/7 days stability samples were not calculated due to the calibration curve error. The stability samples were stored under 5° C. after stability experiments and tested with 14 days stability samples.
^{#}The stability samples were stored under 60° C. for 16 days.

TABLE 7-4
Summary of solution stability evaluation experiments for formulation 2

| | | | 1 day | | | 3 days | | | 7 days | | |
|---|---|---|---|---|---|---|---|---|---|---|---|
| | | | Purity | Assay | | Purity | Assay* | | Purity | Assay* | |
| Sample | Temp. | | (area %) | (%) | pH | (area %) | (%) | pH | (area %) | (%) | pH |
| | 25° | C. | 99.11 | 100.8 | 4.1 | — | 100.2 | 4.1 | 98.92 | 101.2 | 4.1 |
| | 40° | C. | 99.01 | 100.3 | 4.1 | 98.83 | 97.7 | 4.1 | 98.63 | 101.2 | 4.2 |
| | 60° | C. | 98.00 | 101.8 | 4.1 | 95.92 | 95.0 | 4.1 | 93.46 | 84.4 | 4.1 |
| | | | 14 days | | | 28 days | | | | | |
| | | | Purity | Assay | | Purity | Assay | | | | |
| | Temp. | | (Area %) | (%) | pH | (Area %) | (%) | pH | | | |
| ~30 mM | 25° | C. | 99.03 | 96.8 | 4.1 | 98.91 | 97.6 | 4.0 | NA | | |
| API + 30 mM | 40° | C. | 98.51 | 97.5 | 4.1 | 97.78 | 98.2 | 4.0 | | | |

| | | | | | | |
|---|---|---|---|---|---|---|
| citric acid + | 60° | C.^{#} | 91.81 | 93.3 | 4.0 | Not tested |
| 240 mM lactose | | | | | | |
| (~20 mg/mL, | | | | | | |
| formulation 2) | | | | | | |

The initial purity was 99.01%.
*Assay of 1/3/7 days stability samples were not calculated due to the calibration curve error. The stability samples were stored under 5° C. after stability experiments and tested with 14 days stability samples.
—: Baseline fluctuation was observed in the chromatogram of the sample, and the purity could not be integrated.
^{#}The stability samples were stored under 60° C. for 16 days.

TABLE 7-5
Summary of solution stability evaluation experiments for formulation 3

| | | | 1 day | | | 3 days | | | 7 days | | |
|---|---|---|---|---|---|---|---|---|---|---|---|
| | | | Purity | Assay | | Purity | Assay* | pH | Purity | Assay* | |
| Sample | Temp. | | (area %) | (%) | pH | (area %) | (%) | | (area %) | (%) | pH |
| | 25° | C. | 99.03 | 103.5 | 4.1 | 98.92 | 100.6 | 4.1 | 98.86 | 102.9 | 4.1 |
| | 40° | C. | 99.04 | 101.7 | 4.0 | 98.82 | 98.8 | 4.1 | 98.51 | 100.9 | 4.1 |
| | 60° | C. | 98.17 | 104.7 | 4.1 | 95.75 | 99.9 | 4.1 | 93.55 | 98.3 | 4.1 |
| | | | 14 days | | | 28 days | | | | | |
| | | | Purity | Assay | | Purity | Assay | | | | |
| | Temp. | | (Area %) | (%) | pH | (Area %) | (%) | pH | | | |
| ~30 mM | 25° | C. | 99.10 | 98.1 | 4.1 | 99.00 | 100.1 | 4.0 | NA | | |
| API + 30 mM | 40° | C. | 98.33 | 99.1 | 4.0 | 97.74 | 97.4 | 4.0 | | | |

| | | | | | | |
|---|---|---|---|---|---|---|
| citric acid + | 60° | C.^{#} | 92.06 | 96.4 | 4.0 | Not tested |
| 240 mM trehalose | | | | | | |
| (~20 mg/mL, | | | | | | |
| formulation 3) | | | | | | |

The initial purity was 99.01%.
*Assay of 1/3/7 days stability samples were not calculated due to the calibration curve error. The stability samples were stored under 5° C. after stability experiments and tested with 14 days stability samples.
^{#}The stability samples were stored under 60° C. for 16 days.

TABLE 7-6
Summary of solution stability evaluation experiments for formulation 4

| | | | 1 day | | | 3 days | | | 7 days | | |
|---|---|---|---|---|---|---|---|---|---|---|---|
| | | | Purity | Assay | | Purity | Assay* | | Purity | Assay* | |
| Sample | Temp. | | (area %) | (%) | pH | (area %) | (%) | pH | (area %) | (%) | pH |
| | 25° | C. | 99.07 | 104.5 | 4.1 | 99.05 | — | 4.1 | 98.90 | 102.7 | 4.1 |
| | 40° | C. | 99.01 | 92.9 | 4.1 | 98.84 | 101.0 | 4.0 | 98.48 | 103.5 | 4.1 |
| | 60° | C. | 97.86 | 102.3 | 4.1 | 95.79 | 97.2 | 4.1 | 93.54 | 96.0 | 4.1 |
| | | | 14 days | | | 28 days | | | | | |
| | | | Purity | Assay | | Purity | Assay | | | | |
| | Temp. | | (Area %) | (%) | pH | (Area %) | (%) | pH | | | |
| ~30 mM | 25° | C. | 99.02 | 102.2 | 4.1 | 99.02 | 99.0 | 4.0 | NA | | |
| API + 30 mM | 40° | C. | 98.37 | 101.4 | 4.1 | 97.71 | 99.8 | 4.0 | | | |

| | | | | | | |
|---|---|---|---|---|---|---|
| citric acid + | 60° | C.^{#} | 91.94 | 93.7 | 4.0 | Not tested |
| 240 mM sucrose | | | | | | |
| (~20 mg/mL, | | | | | | |
| formulation 4) | | | | | | |

The initial purity was 99.01%.
*Assay of 1/3/7 days stability samples were not calculated due to the calibration curve error. The stability samples were stored under 5° C. after stability experiments and tested with 14 days stability samples.
—: Injection error and the assay could not be calculated.
^{#}The stability samples were stored under 60° C. for 16 days.

TABLE 7-7
Summary of solution stability evaluation experiments for formulation 5

| | | | 1 day | | | 3 days | | | 7 days | | |
|---|---|---|---|---|---|---|---|---|---|---|---|
| | | | Purity | Assay | | Purity | Assay* | | Purity | Assay* | |
| Sample | Temp. | | (area %) | (%) | pH | (area %) | (%) | pH | (area % | (%) | pH |
| | 25° | C. | 98.93 | 102.7 | 4.8 | 98.83 | 100.1 | 4.8 | 98.75 | 101.3 | 4.9 |
| | 40° | C. | 98.88 | 101.8 | 4.8 | 98.79 | 96.4 | 4.9 | 98.26 | 100.0 | 4.9 |
| | 60° | C. | 97.45 | 101.5 | 4.8 | 94.65 | 93.4 | 4.8 | 92.75 | 93.6 | 4.8 |
| | | | 14 days | | | 28 days | | | | | |
| | | | Purity | Assay | | Purity | Assay | | | | |
| | Temp. | | (Area %) | (%) | pH | (Area %) | (%) | pH | | | |
| ~30 mM API | 25° | C. | 98.92 | 101.6 | 4.8 | 98.84 | 97.2 | 4.7 | NA | | |
| pH = 4.0 citrate/ | 40° | C. | 97.84 | 99.9 | 4.8 | 97.05 | 97.2 | 4.7 | | | |

| | | | | | | |
|---|---|---|---|---|---|---|
| phosphate buffer | 60° | C.^{#} | 92.11 | 93.0 | 4.7 | Not tested |
| (~20 mg/mL, | | | | | | |
| formulation 5) | | | | | | |

The initial purity was 99.01%.
*Assay of 1/3/7 days stability samples were not calculated due to the calibration curve error. The stability
samples were stored under 5° C. after stability experiments and tested with 14 days stability samples.
^{#}The stability samples were stored under 60° C. for 16 days.

### Stability Experiments at 5° C.

Some of the stability samples at 5° C. became a suspension after storage for 37 days. The samples were then stirred (1000 rpm) at 5° C. for two days, resulting in all samples becoming a suspension. Three of the samples under each condition were characterized to determine the identity of precipitate, solubility of citrate salt at 5° C., and stability at 5° C.
1. Sample 1 of formulations 1/2/3/4/5 after stirring was isolated, XPRD/¹H NMR of the solid was obtained, and purity/assay/concentration/pH of the supernatant were tested.
2. Sample 2 of formulations 1/2/3/4/5 after stirring was stored at RT for about 1 hour, and the solid dissolved. Purity/assay/concentration of the solution was tested.
3. Sample 3 of formulations 1/2/3/4/5 after stirring was sonicated at RT for about 2-3 mins, and the solid dissolved.

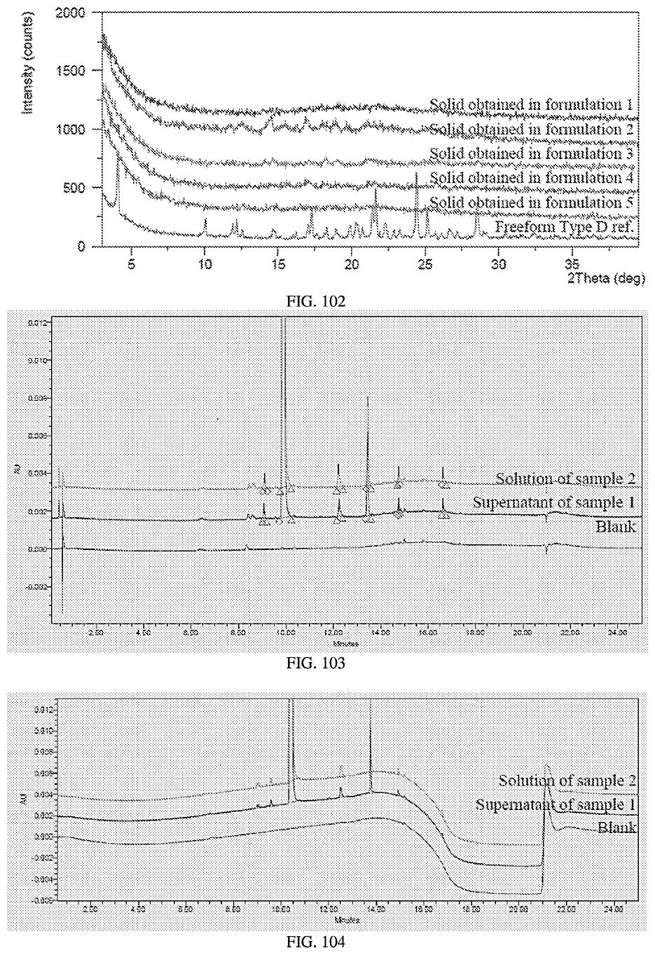

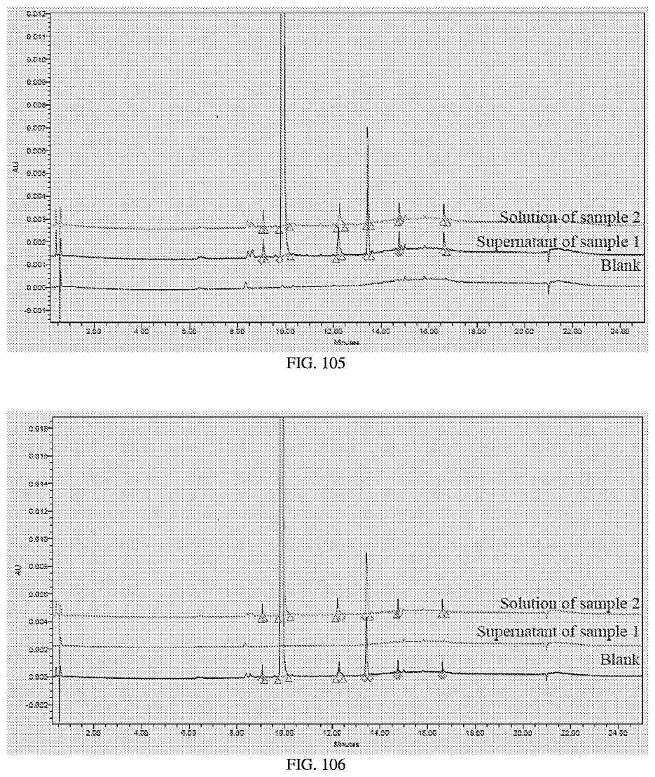

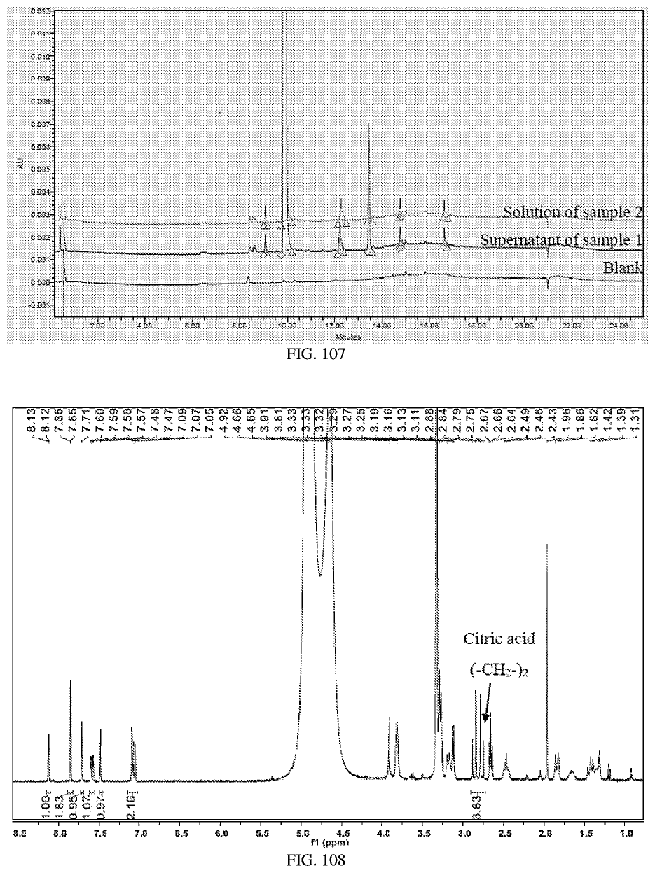

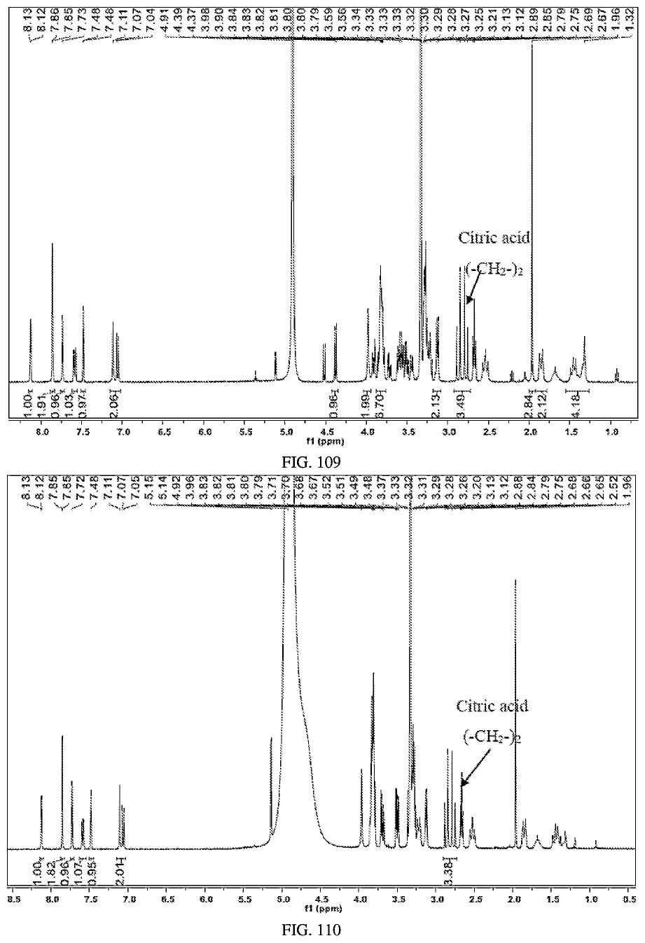

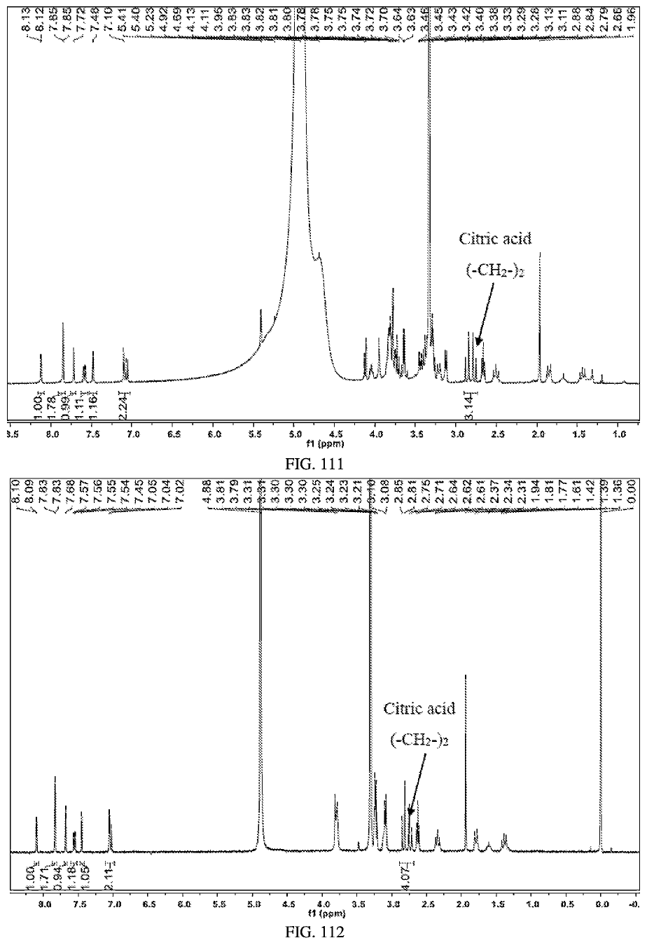

The characterization of stability samples in formulations 1 to 5 at 5° C. are summarized in Tables 7-8 to 7-12. XRPD results show the solid obtained at 5° C. in formulations 1/2/3/4/5 is amorphous (FIG. 102), and ¹H NMR showed the precipitate in all cases may be citrate salt with molar ratio of citric acid and freeform in the solid of about 0.8:1 to 1:1 (FIG. 108 to FIG. 112). The solid amount obtained in formulations 1 and 5 was less than the amount in formulations 2-4 as confirmed by the supernatant concentration results shown in table 7-8 and 7-12, which suggests that the presence of sugars decrease citrate solubility at 5° C. from 16.9 mg/ml to as low as 10.4 mg/mL. No significant degradation was observed for formulations 1-5 after storage at 5° C. for 37 days. All solids dissolved in all formulations once warmed up to room temperature suggest the solubility of citrate salt at rt is higher than 20 mg/mL. Chromatogram overlays of stability samples are shown in FIG. 103 to FIG. 107.

TABLE 7-8
Summary of solution stability evaluation experiments in formulation 1 at 5° C.

| | | | 37 days | | | |
|---|---|---|---|---|---|---|
| | | | Purity | Assay | | Conc. |
| Formulation 1 | Temp. | Samples | (Area %) | (%) | pH | (mg/mL) |
| | 5° C. | Sample 1 | 99.02 | 84.7 | 3.8 | 16.9 |
| | | (supernatant) | | | | |
| 30 mM API + 30 | | Sample 1 | XRPD | | NMR | |
| mM citric acid | | (Solid) | Amorphous | | 1:1 (citric acid/API) | |
| (~20 mg/mL) | | Sample 2 | 99.06 | 96.0 | 3.8 | 19.1 |
| | | (Solution) | | | | |

The initial purity was 99.01%.

TABLE 7-9
Summary of solution stability evaluation experiments in formulation 2 at 5° C.

| | | | 37 days | | | |
|---|---|---|---|---|---|---|
| | | | Purity | Assay | | Conc. |
| Formulation 2 | Temp. | Samples | (Area %) | (%) | pH | (mg/mL) |
| | 5° C. | Sample 1 | 98.29 | 51.7 | 3.8 | 10.6 |
| | | (supernatant) | | | | |
| ~30 mM API + | | Sample 1 | XRPD | | NMR | |
| 30 mM citric acid + | | (Solid) | Amorphous | | 0.9:1 (citric acid/API) | |
| 240 mM lactose | | Sample 2 | 99.06 | 100.1 | 3.9 | 20.6 |
| (~20 mg/mL) | | (Solution) | | | | |

The initial purity was 99.01%.

TABLE 7-10
Summary of solution stability evaluation experiments in formulation 3 at 5° C.

| | | | 37 days | | | |
|---|---|---|---|---|---|---|
| | | | Purity | Assay | | Conc. |
| Formulation 3 | Temp. | Samples | (Area %) | (%) | pH | (mg/mL) |
| | 5° C. | Sample 1 | 98.75 | 65.8 | 3.8 | 13.4 |
| | | (supernatant) | | | | |
| ~30 mM API + 30 | | Sample 1 | XRPD | | NMR | |
| mM citric acid + | | (Solid) | Amorphous | | 0.8:1 (citric acid/API) | |
| 240 mM trehalose | | Sample 2 | 99.07 | 97.3 | 3.8 | 19.7 |
| (~20 mg/mL) | | (Solution) | | | | |

The initial purity was 99.01%.

TABLE 7-11
Summary of solution stability evaluation experiments in formulation 4 at 5 ° C.

| | | | 37 days | | | |
|---|---|---|---|---|---|---|
| | | | Purity | Assay | | Conc. |
| Formulation 4 | Temp. | Samples | (Area %) | (%) | pH | (mg/mL) |
| | 5° C. | Sample 1 | 98.76 | 63.8 | 3.8 | 12.7 |
| | | (supernatant) | | | | |
| | | Sample 1 | XRPD | | NMR | |
| ~30 mM API + 30 | | (Solid) | Amorphous | | 0.9:1 (citric acid/API) | |
| mM citric acid + | | Sample 2 | 99.07 | 90.4 | 3.8 | 18.0 |
| 240 mM sucrose | | (Solution) | | | | |
| (~20 mg/mL) | | | | | | |

The initial purity was 99.01%.

TABLE 7-12
Summary of solution stability evaluation experiments in formulation 5 at 5 ° C.

| | | | 37 days | | | |
|---|---|---|---|---|---|---|
| | | | Purity | Assay | | Conc. |
| Formulation 5 | Temp. | Samples | (Area %) | (%) | pH | (mg/mL) |
| | 5° C. | Sample 1 | 98.92 | 84.6 | 4.5 | 16.9 |
| | | (supernatant) | | | | |
| ~30 mM API in | | Sample 1 | XRPD | | NMR | |
| pH = 4.0 citrate/ | | (Solid) | Amorphous | | 1:1 (citric acid/API) | |
| phosphate buffer | | Sample 2 | 98.98 | 95.6 | 4.6 | 19.1 |
| (~20 mg/mL) | | (Solution) | | | | |

The initial purity was 99.01%.

Solution Stability in Formulation with Lactose (Dosing Conc. 40 mg/mL)

Physical and chemical stability of formulation 2 in Example 6 at 40 mg/mL concentration was tested. The 40 mg/mL freeform+citric acid+lactose formulation was prepared by transferring 217.5 mg freeform sample (equivalent to ˜200 mg API), 62.5 mg citric acid and 297 mg lactose into a 5 mL volumetric flask and diluted to volume with water. The sample obtained was sonicated for about 2 mins and filtrated through 0.22 um filter. The tested concentration was 39.3 mg/mL, and the pH of the solution was 3.6. About 5 mg amorphous wet sample (the solid obtained in formulation 2 at 5° C., Example 6) was added into 1 mL of the 40 mg/mL formulation, and the solid was dissolved after stirring for 1 hour. Another 5 mg amorphous wet sample was added into the 40 mg/mL formulation, and the solid was dissolved after stirring for 1 hour. The tested concentration of solution obtained was 41.6 mg/mL, and the pH of the solution was 3.6. About 5 mg wet cake was added into the 40 mg/mL formulation (˜41.6 mg/mL), the sample was stirred at 5° C. overnight, and a suspension was obtained. The suspension was stirred at RT for 1 hour, and a clear solution was obtained. The pH of the solution obtained was 3.7, and concentration of the solution was 42.8 mg/mL. This result suggests that the formulation at 40 mg/mL concentration is physically stable and not supersaturated. No precipitation of citrate salt is expected when the solution is stored at room temperature.

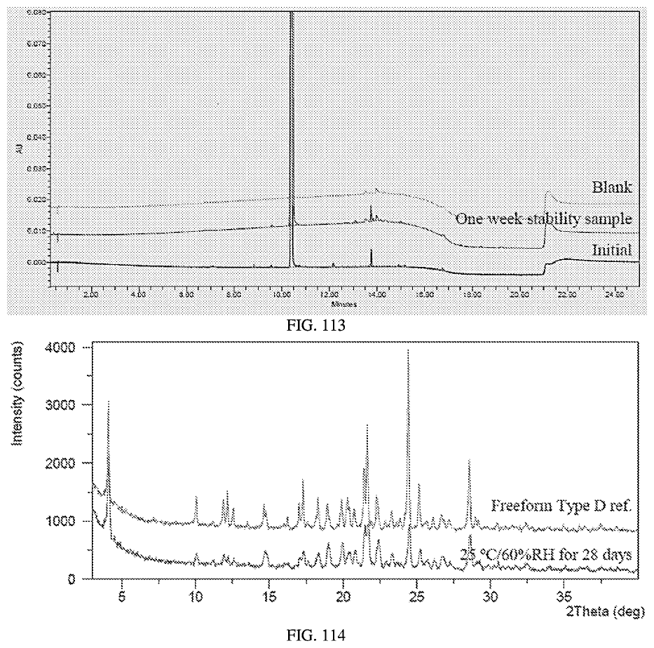

Another 1 mL of the 40 mg/mL freeform+citric acid+lactose formulation was stored at RT (covered with aluminum foil) for 1 week. The purity of the one-week stability sample was 99.01%, and the pH was 3.7. No significant degradation was observed. Chromatogram overlay of stability samples are shown in FIG. 113.

### Solid State Stability

Solid state stability of the freeform Type D was determined. Approximately 30 mg of each solid sample was added to an HPLC vial (sealed by Parafilm© and poked with several pinholes) and then stored at 25° C./60% RH, 40° C./75% RH and 60° C. for 28 days.

After 1/3/7/14/28 days storage, solids were taken out for HPLC and XRPD to evaluate chemical and physical stability, respectively.

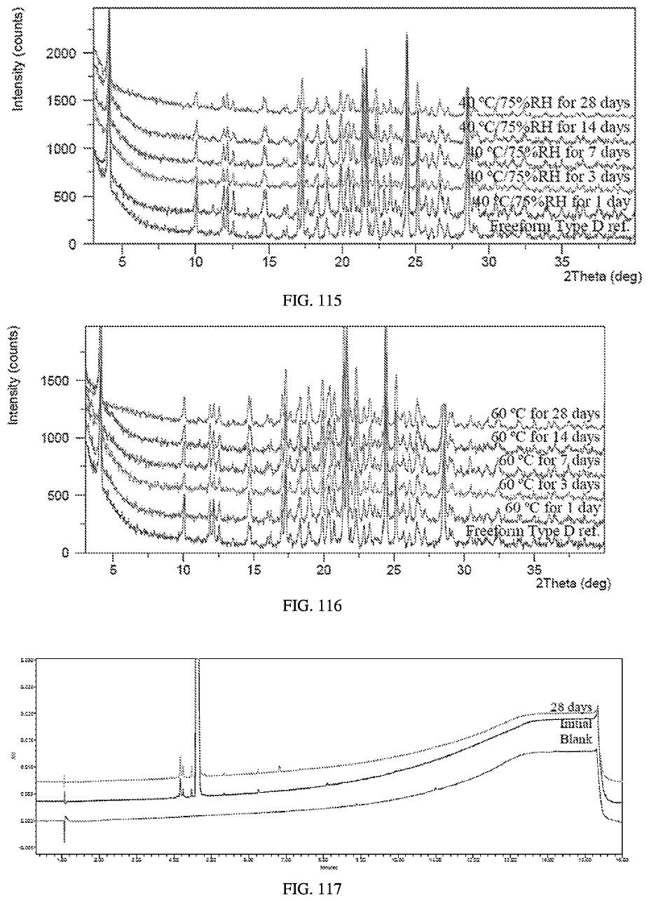

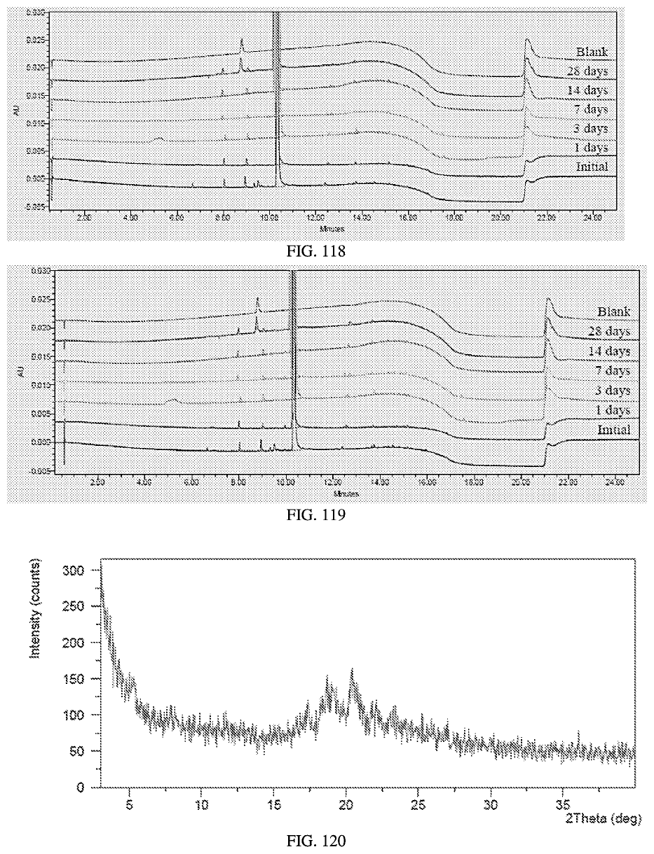

Stability results are summarized in Table 7-13. No significant degradation or form change was observed after storage at 25° C./60% RH, 40° C./75% RH and 60° C. for 28 days. XRPD overlay of stability samples are shown in FIG. 114 to FIG. 116, and chromatogram overlays of stability samples are shown in FIG. 117 to FIG. 119.

TABLE 7-13
Summary of solid-state stability evaluation experiments

| | | 1 day | | 3 days | | 7 days | | 14 days | | 28 days | |
|---|---|---|---|---|---|---|---|---|---|---|---|
| Sample | | Purity | | Purity | | Purity | | Purity | | Purity | |
| (ID) | Conditions | (area %) | FC | (area %) | FC | (area %) | FC | (area %) | FC | (area %) | FC |
| Freeform | 25° C./ | Not tested | | | | | | | | 99.6 | N |
| Type D | 60% RH | | | | | | | | | | |
| | 40° C./ | 99.69 | N | 99.56 | N | 99.59 | N | 99.69 | N | 99.73 | N |
| | 75% RH | | | | | | | | | | |
| | 60° C. | 99.74 | N | 99.57 | N | 99.51 | N | 99.62 | N | 99.66 | N |

* The initial purity of freeform Type D was 99.62%.
FC: Form change. N: The form was not changed.

In summary, freeform isolation and 100 polymorph and salt formation experiments of Compound (I) freeform were performed by different crystallization methods. Four crystalline forms of freeform (named as freeform Type A, B, C and D) and 10 crystalline salts (19 forms) were obtained. pKa, Log D_{7.4} and complexation stability constants in HPβCD and SBECD of freeform Type D were also determined.

Equilibrium solubility evaluation was performed for freeform Type A, HCl salt Type F, fumarate Type A, and sulfate Type B. Freeform Type A showed high solubility in pH=4.0 buffer (>36.9 mg/mL, 50 mM citrate buffer, final pH=4.2). Freeform was selected for further development. Freeform and citric acid was selected for further pre-formulation study according to the solubility results. Solubility, stability (solid and solution) and pre-formulation experiments of freeform Type D with citric acid were performed. Formulation of Compound (I) freeform with 1.05 equiv. citric acid and 173 mM lactose (40 mg/mL) was selected for further development, and the formulation showed no degradation after storage under RT for 7 days.

Trihydrate freeform Type D was selected for further development. Freeform Type D was obtained when a_{W}>0.3 during the thermodynamic relationship study and was kinetically stable for at least 6 weeks under a wide range of RH (7% to 84%). The citrate salt offers significant aqueous solubility enhancement (>50 mg/mL vs freeform 0.02 mg/mL) and good solution stability. In addition, it can be conveniently prepared in situ by simply mixing citric acid and freeform in water. As a result, a solution of 40 mg/mL Compound (I) freeform (weight adjusted), 1.05 equiv. citric acid and 173 mM lactose (QS for isotonic) was identified as a formulation to be employed in toxicology studies.

### Example 7. Characterization Starting Material

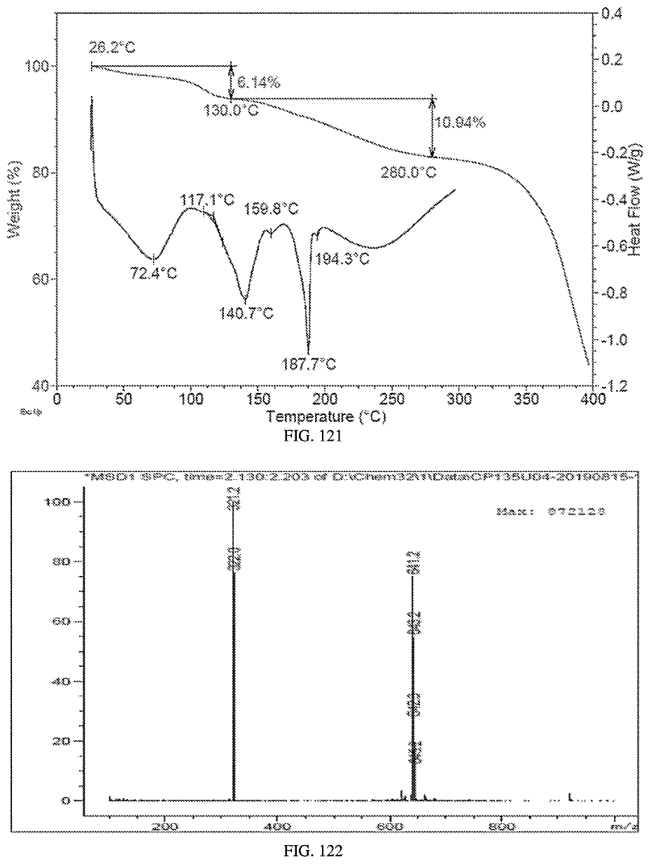

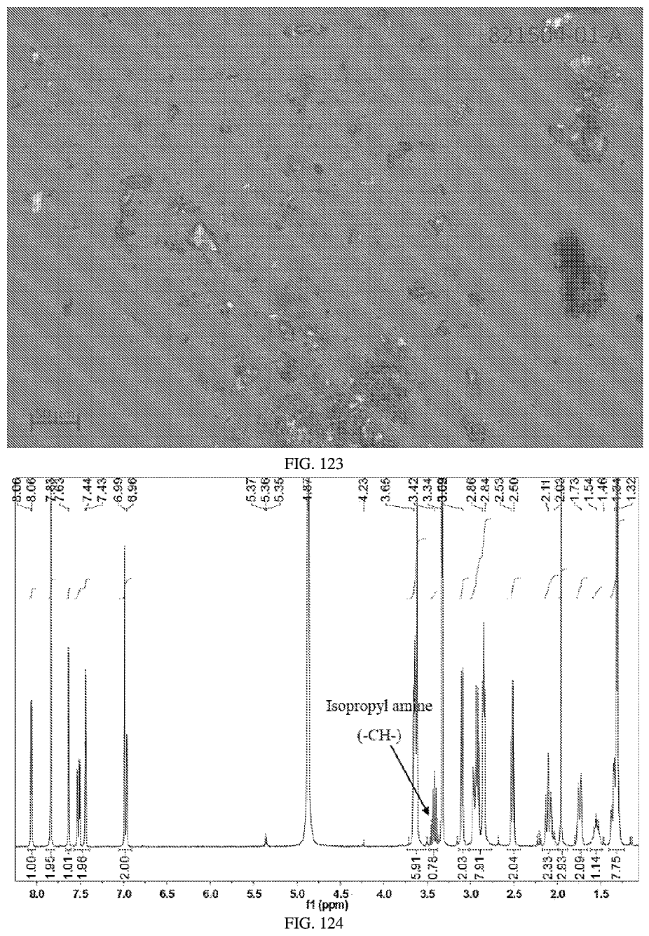

Compound (I) starting material was characterized by XRPD, TGA, DSC, LC-MS, PLM and ¹H NMR. XRPD pattern (FIG. 120) showed the sample was low crystallinity. In FIG. 121, TGA curve showed a weight loss of 6.1% up to 130° C., and then a continued weight loss of 10.9% from 130° C. to 280° C., and DSC curve showed five endotherms at 72.4° C. (peak), 140.7° C. (peak), 159.8° C. (peak), 187.7° C. (peak) and 194.3° C. (peak). LC-MS result (FIG. 122) showed m/z of the sample is 641.2. PLM (FIG. 123) showed the sample was irregular particles with agglomeration. ¹H NMR (FIG. 124) showed the molar ratio of isopropyl amine to freeform in the sample was about 0.8:1.0.

### Freeform Isolation of Starting Material

The freeform isolation procedure are summarized as below:
1. Dissolve or disperse starting material (SM) in corresponding solvent.
2. Add corresponding acid (with ratio in the summary table) to the solution or suspension.
3. Stir the experiments for about 10 minutes to 1 hour at 5° C. and solids were collected with centrifugation or filtration, followed by water rinse. Solids were dried in vacuum and characterized.

Four freeform isolation experiments were performed in different solvent with different acids. Solids were obtained in DCM/HCl and CHCl₃/HCl system. The solids were characterized by XRPD/¹H NMR/IC, and the two samples were crystalline sample (named as freeform Type A) with no isopropyl amine and residual Cl⁻. The experiments and results are summarized in Table 9-1. Approximate solubility of starting material and freeform Type A was tested at RT, and starting material showed higher solubility in CHCl₃ and freeform Type A showed lower solubility in CHCl₃ (Table 9-2).

Considering that residual Cl⁻ was detected in freeform Type A and HCl might react with freeform during the isolation, the ratio between freeform and HCl was adjusted. Another four freeform isolation experiments were performed with different charging ratio of acid and starting material in CHCl₃. The experiments and results are summarized in Table 9-3. The solid obtained in CHCl₃ with charging ratio of HCl to staring material as 0.5:1 showed no residual isopropyl amine and Cl⁻, and XRPD results showed the solid was freeform Type A.

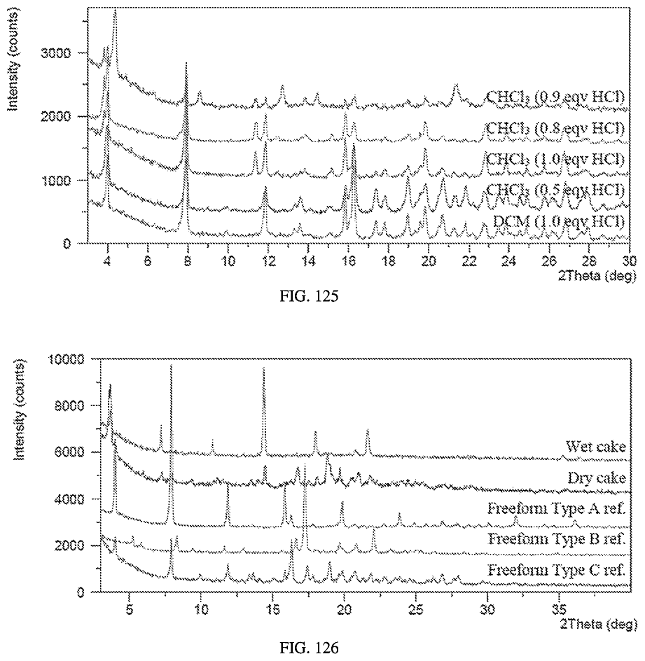

All the solid obtained in the freeform isolation procedure is shown in FIG. 125. The solid obtained in CHCl₃ (0.5 equiv. HCl) and DCM (1 equiv. HCl) were consistent and named as freeform Type A. Additional diffraction peaks were observed in CHCl₃ (0.9 equiv. HCl), CHCl₃ (0.8 equiv. HCl) and CHCl₃ (1 equiv. HCl) which might be attributed to the partial formation of HCl salt.

CHCl₃ was selected as the final freeform isolation solvent, and HCl with a charge ratio of 0.5:1 was selected as the final acid. Freeform isolation was performed on 8 g scale, and the detailed procedure is summarized in Table 9-4. Freeform Type A with no residual isopropyl amine and 0.35% Cl⁻ was obtained (The calculated molar ratio of Cl⁻ to freeform in the solid was about 0.06:1). The characterization results of 8 g freeform Type A is shown in Example 1, and the sample was used for polymorph and salt formation.

The 8 g freeform Type A was used for the formation experiments, and freeform isolation was re-performed, and the detailed procedure are summarized in Table 9-4. The wet cake obtained in step 3 of the procedure was a new crystal form, and the new crystal form transferred to low crystallinity sample after vacuum drying (FIG. 126). IC result showed the weight percentage of Cl⁻ in the sample was about 1.6% (The calculated molar ratio of Cl⁻ to freeform in the solid was about 0.3:1). The solid was stirred in H₂O/acetone (10:1, v/v) for 3 days to remove potential HCl salt and then vacuum dried. Another new crystal form with no residual isopropyl amine and Cl⁻ was obtained finally, and the form was named as freeform Type D. The characterization results of freeform Type D are shown in Example 1, and the sample was used for solubility and stability evaluation.

TABLE 9-1
Freeform isolation experiments and results (I/II)

| | | | | Charging | Characterization Results | | |
|---|---|---|---|---|---|---|---|
| | | | | Ratio of | Isopropyl | Yield | Cl⁻:API |
| Solvent | Scale | | Acid | Acid:SM | Amine | (%) | (Cl⁻ %) |
| DCM | 100 | mg | HCl | 1:1 | Not | 58% | 0.23 |
| | | | | | detected | | (1.3%) |
| EtOAc/H₂O | 100 | mg | HCl | 1:1 | Emulsion was obtained. | | |
| (1:1, v/v) | | | | | | | |
| DCM | 50 | mg | Citric | 1:1 | Clear solution | | |
| | | | acid | | was obtained | | |
| CHCl₃ | 50 | mg | HCl | 1:1 | Not | 58% | 0.42 |
| | | | | | detected | | (2.3%) |

TABLE 9-2
Approximate solubility results of solvent for freeform isolation

| | | Solubility |
|---|---|---|
| Sample | Solvent | (mg/mL) |
| Starting material | CHCl₃ | S > 50.5 |
| | DCM | 20.8 < S < 52.0 |
| Freeform Type A | CHCl₃ | S < 1.2 |
| | DCM | 1.1 < S < 2.4 |

TABLE 9-3
Freeform isolation experiments and results (II/II)

| | | | | Charging | Characterization Results | | |
|---|---|---|---|---|---|---|---|
| | | | | Ratio of | Isopropyl | Yield | Cl⁻:API |
| Solvent | Scale | | Acid | Acid:SM | Amine | (%) | (Cl⁻ %) |
| CHCl₃ | 500 | mg | HCl | 0.9:1 | Not | 90% | 0.41 |
| | | | | | detected | | (2.3%) |
| CHCl₃ | 500 | mg | | 0.8:1 | | 98% | 0.31 |
| | | | | | | | (1.7%) |
| CHCl₃ | 50 | mg | | 0.5:1 | | 68% | 0.02 |
| | | | | | | | (0.13%) |

| | | | | | |
|---|---|---|---|---|---|
| CHCl₃ | 50 | mg | Methanesulfonic | 0.8:1 | Clear solution |
| | | | Acid | | was obtained |

TABLE 9-4
Freeform isolation procedure

| Sample | Procedure |
|---|---|
| Freeform | 1. Dissolve 10 g starting material in 250 mL CHCl₃. |
| Type A | 2. Add 13.1 mL 0.5 M HCl into the solution slowly, and |
| | then stir the sample under 5° C. for 1 hour. |
| | 3. Separate solid by vacuum filtration and then stirred |
| | the wet cake in 35 mL H₂O. |
| | 4. Separate solid by vacuum filtration and then dry the |
| | solid by vacuum drying overnight. |
| Freeform | 1. Dissolve 7.3 g starting material in 180 mL CHCl₃. |
| Type D | 2. Add 4.8 mL 1M HCl into the solution slowly, and then |
| | stir the sample overnight under 5° C. |
| | 3. Separate solid by vacuum filtration and the wet cake |
| | showed a new XRPD pattern. Dry the wet cake by vacuum |
| | drying overnight and the dry cake was low crystallinity |
| | sample. |
| | 4. Stir the low crystallinity solid in 44 mL H₂O/acetone |
| | (10:1, v/v) for 10 days. |
| | 5. Separate solid by vacuum filtration and then dry the |
| | solid by vacuum drying for two hours. |

### Characterization of Starting Material

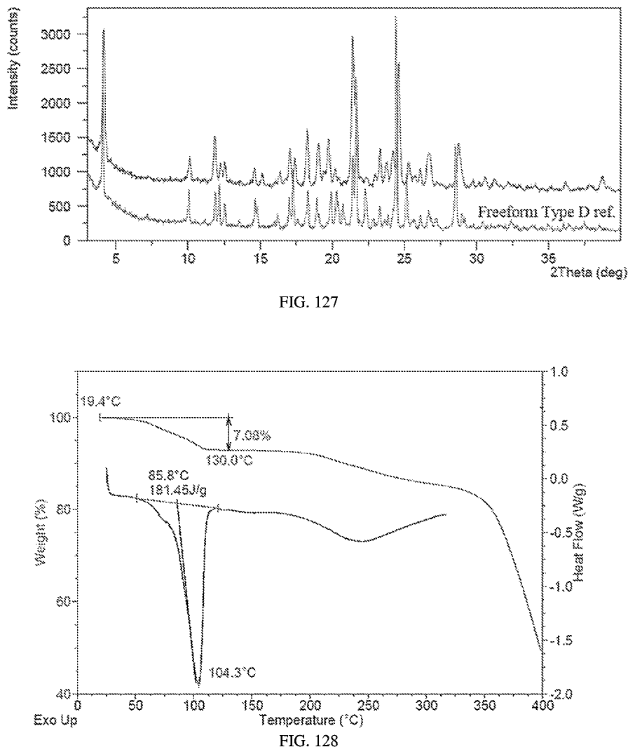

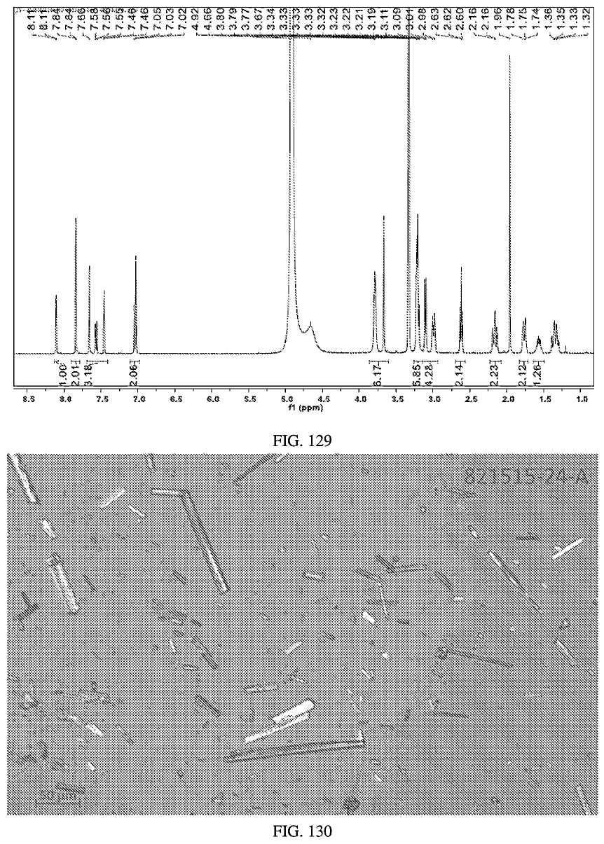

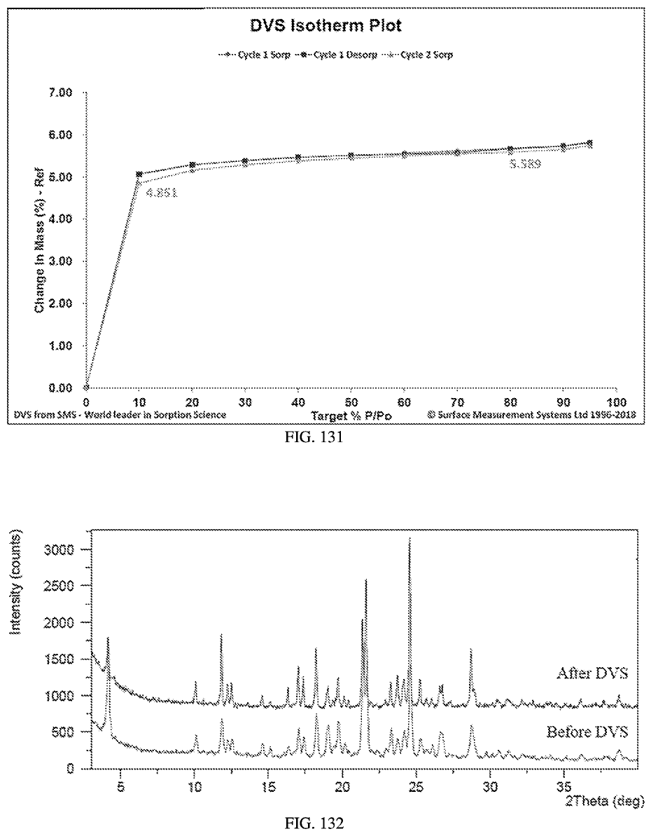

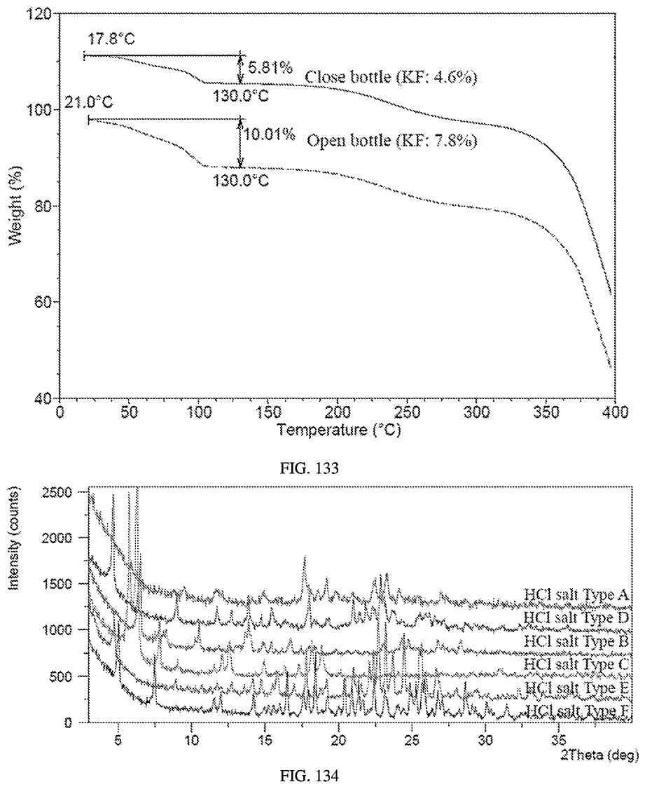

The starting material was characterized by XRPD, TGA, DSC, PLM, 1H NMR, DVS and KF. XRPD pattern (FIG. 127) showed the diffraction peaks of starting material are similar to freeform Type D, but some of the diffraction peaks were shifted. In FIG. 128, TGA curve showed a weight loss of 7.1% up to 130° C., and DSC curve showed one endotherm at 85.8° C. (onset). ¹H NMR result is shown in FIG. 129. PLM (FIG. 130) showed the sample as rod-like particles. DVS result (FIG. 131) showed a moisture uptake of 0.7% when humidity varied from 10% RH to 80% RH, and no form change was observed after DVS test (FIG. 132). KF result showed water content in the sample was 2.7%. Some experiments were performed as the KF result was not consistent with the TGA result. About 100 mg of the starting material was stored under ambient conditions in an open bottle and a closed bottle over a weekend. TGA and KF tests were performed for the two samples (FIG. 133). Water content of the sample correlates with ambient humidity according to the characterization results, and single crystal determination was performed to confirm the water content.

### Approximate Solubility and Solvent Abbreviations

The approximate solubility (Table 9-5) of Compound (I) freeform Type A was tested at RT to guide the polymorph and salt formation experiments. Approximately 2 mg solids were added into a 3-mL glass vial. Solvents in the following table were then added stepwise (50-50-200-700-1000 μL for each step) into the vials and stirred until the solids were dissolved or a total volume of 1 mL was reached. The solubility results were used to guide the solvent selection in polymorph formation.

TABLE 9-5
Approximate solubility of freeform Type A at RT

| | Solubility | | Solubility |
|---|---|---|---|
| Solvent | (mg/mL) | Solvent | (mg/mL) |
| MeOH | S > 40.0 | 1,4-Dioxane | S < 1.9 |
| EtOH | 7.3 < S < 22.0 | ACN | S < 1.9 |
| IPA | 1.5 < S < 1.8 | CHCl₃ | S < 2.1 |
| Acetone | S < 1.9 | DCM | S < 1.8 |
| MIBK | S < 2.1 | n-Heptane | S < 2.2 |
| EtOAc | S < 2.0 | Toluene | S < 2.1 |
| IPAc | S < 1.9 | Anisole | S < 2.0 |
| MTBE | S < 1.9 | DMSO | 17.0 < S < 34.0 |
| THF | 4.0 < S < 8.0 | CPME | S < 2.0 |
| 2-MeTHF | S < 1.7 | H₂O | S < 2.0 |

TABLE 9-6
Solvent abbreviation list

| Abbreviation | Solvent | Abbreviation | Solvent |
|---|---|---|---|
| MeOH | Methanol | THF | Tetrahydrofuran |
| EtOH | Ethanol | 2-MeTHF | 2-Methyltetrahydrofuran |
| IPA | Isopropyl | ACN | Acetonitrile |
| | alcohol | | |
| MEK | Methyl ethyl | CHCl₃ | Chloroform |
| | ketone | | |
| MIBK | 4-Methyl-2- | DMSO | Dimethylsulfoxide |
| | pentanone | | |
| EtOAc | Ethyl acetate | DMAc | Dimethylacetamide |
| IPAc | Isopropyl acetate | NMP | Methylpyrrolidone |
| MTBE | Methyl tert-butyl | H₂O | Water |
| | ether | | |
| DCM | Dichloromethane | CPME | Cycloamyl methyl ether |
| DMF | Dimethyl | n-Butanol | Normal butanol |
| | formamide | | |

### Polymorphs of Compound (I) Freeform

Polymorph formation experiments were performed under 100 conditions using Compound (I) freeform Type A as starting material. The methods utilized and crystal forms identified are summarized in Table 9-7.

TABLE 9-7
Summary of freeform polymorph formation

| Method | Amount | Result |
|---|---|---|
| Slurry at RT | 19 | Type A, Type B |
| Slurry at 50° C. | 18 | Type A, Type B |
| Slow evaporation | 9 | Type A, Type B |
| Liquid Vapor | 10 | Type A, Type B |
| Diffusion | | |
| Temperature | 12 | Type A, Type B |
| cycling | | |
| Polymer induced | 4 | Type A |
| crystallization | | |
| Solid vapor | 12 | Type A, Type B, Type C |
| diffusion | | |
| Anti-solvent | 16 | Type A, Type B |
| addition | | |
| Total | 100 | Type A, Type B, Type C |

### Slurry at RT

Slurry experiments were conducted at RT in 19 different solvent systems. ˜20 mg of freeform Type A was suspended in 0.5 mL of the corresponding solvent in a HPLC vial. After the suspension was stirred magnetically (˜1000 rpm) for about 7 days at RT, the remaining solids were isolated for XRPD analysis. Results summarized in Table 9-8 indicate that freeform Type A and freeform Type B were generated.

TABLE 9-8
Summary of slurry conversion experiments at RT

| Solvent (v/v) | Results |
|---|---|
| IPA | Type A |
| Acetone | Type B |
| EtOAc | Type B |
| THF | Type A* |
| ACN | Type A |
| DCM | Type A |
| m-Xylene | Type A |
| EtOH | Low crystallinity |
| DMSO/MIBK, (1:2) | Type B* |
| DMAc/ACN, (1:9) | Type B |
| DMF/Toluene, (1:49) | Type A |
| NMP/IPAc, (2:23) | Type B |
| EtOH/n-Heptane, (1:1) | Type B |
| DMSO/CHCl₃, (1:1) | Type A* |
| EtOH/H₂O, (49:1) | Type A |
| EtOH/H₂O, (19:1) | Type A |
| EtOH/H₂O, (9:1) | Type A |
| EtOH/H₂O, (4:1) | Type A* |
| H₂O | Type A |

*Solid was obtained after evaporation.

### Slurry at 50° C.

Slurry experiments were conducted at 50° C. in 18 different solvent systems. About 20 mg of freeform Type A was suspended in 0.5 mL of the corresponding solvent in a HPLC vial. After the suspension was magnetically stirred (˜1000 rpm) for about 3 days at 50° C., freeform Type A and freeform Type B were observed. Results are summarized in Table 9-9.

TABLE 9-9
Summary of slurry conversion experiments at 50° C.

| Solvent (v/v) | Results |
|---|---|
| IPA | Type B |
| MIBK | Type B |
| IPAc | Type A + B |
| MTBE | Type A |
| 2-MeTHF | Type B |
| 1,4-Dioxane | amorphous |
| ACN | Type B |
| Toluene | Type A |
| n-Heptane | Type A |
| DMSO/Anisole, (1:49) | Type B |
| DMSO/Acetone, (1:49) | Type B |
| DMSO/EtOAc, (1:69) | Type B |
| MeOH/CHCl₃, (1:49) | Clear solution |
| ACN/H₂O, (99:1) | Type A |
| ACN/H₂O, (98:2) | Type A |
| ACN/H₂O, (96:4) | Type A |
| ACN/H₂O, (92:8) | Type A |
| H₂O | Type A |

*Solid was obtained after evaporation.

### Slow Evaporation

Slow evaporation experiments were performed under 9 conditions. Approximately 20 mg freeform Type A was dissolved in corresponding solvent in a 3-mL glass vial. All samples were filtered using a PTFE membrane (pore size of 0.45 m) and the filtrates were used for the follow-up steps. The vials were sealed by Parafilm® (poked with several pin-holes) and slow evaporation at RT. Freeform Type A was observed, and the results are summarized in Table 9-10.

TABLE 9-10
Summary of slow evaporation experiments

| Solvent (v/v) | Results |
|---|---|
| MeOH/EtOAc, (1:1) | Type A |
| MeOH/ACN, (8:5) | Type A |
| MeOH/H₂O, (97:75) | Type A |
| MeOH/Toluene, (1:1) | Type A |
| MeOH/Acetone, (31:25) | Type A |
| MeOH/2-MeTHE, (1:1) | Type A |
| EtOH | Type A |
| EtOH/DCM, (2:1) | Type A |

### Liquid Vapor Diffusion

Liquid vapor diffusion was performed under 10 conditions. Approximately 20 mg of freeform Type A was dissolved in 0.5-1.2 mL of an appropriate solvent in a 3-mL vial. The solution was filtered to obtain a clear solution. This solution was then placed into a 20-mL vial with 4 mL of the corresponding volatile solvents. The 20-mL vial was sealed with a cap and kept at RT allowing sufficient time for the organic vapor to interact with the solution. Freeform Type A and freeform Type B were observed, and the results are summarized in Table 9-11.

TABLE 9-11
Summary of liquid vapor diffusion experiments

| Solvent (v/v) | Anti-solvent | Results |
|---|---|---|
| DMSO | H₂O | Type A + B |
| | 2-MeTHF | Type A |
| | EtOAc | Type B |
| | IPA | Type B |
| | CHCl₃ | Type B |
| | MIBK | Type B |
| EtOH | MTBE | Type B |
| | DCM | Type A |
| | MEK | Type A |
| | Acetone | Type A |

*Solid was obtained after evaporation.

### Temperature Cycling

Temperature cycling experiments were conducted from 50° C. to 5° C. (0.1° C./min, three cycles) in 12 different solvent systems. About 20 mg of freeform Type A was suspended in 0.5 mL of the corresponding solvent in a HPLC vial. After the suspension was magnetically stirred (˜000 rpm) for about 5 days, freeform Type A and freeform Type B were observed. Results are summarized in Table 9-12.

TABLE 9-12
Summary of temperature cycling experiments

| Solvent (v/v) | Results |
|---|---|
| IPA | Type B |
| MIBK | Type B |
| EtOAc | Type B |
| Anisole | Type A |
| CPME | Type A |
| CHCl₃ | Type A |
| MeOH/m-Xylene, (1:4) | Type A* |
| MeOH/MEK, (1:4) | Type B |
| MeOH/IPAc, (1:4) | Type B |
| DMSO/H₂O, (1:3) | Type A |
| DMSO/ACN, (1:3) | Type B |
| DMSO/Toluene, (1:3) | Type A + B* |

*Solid was obtained after evaporation.

### Polymer Induced Crystallization

Polymer induced crystallization was performed under 4 conditions. Approximately 20 mg freeform Type A was dissolved in corresponding solvent in a 3-mL glass vial. All samples were filtered using a PTFE membrane (pore size of 0.45 m) and the filtrates were used for the follow-up steps. About 2 mg corresponding polymer was added into the filtrates, and then the vials were sealed by Parafilm© (poked with several pin-holes) and slow evaporation at RT. Freeform Type A was observed, and the results are summarized in Table 9-13.

TABLE 9-13
Summary of polymer induced crystallization experiments

| RH % | Salt | Results |
|---|---|---|
| MeOH/CPME, (1:1) | Polymer | Type A |
| EtOH/MIBK, (2:1) | mixture A | Type A |
| THF | Polymer | Type A |
| MeOH/m-Xylene, (1:1) | mixture B | Type A |

Polymer mixture A: polyvinyl pyrrolidone (PVP), polyvinyl alcohol (PVA), polyvinylchloride (PVC), polyvinyl acetate (PVAC), hypromellose (HPMC), methyl cellulose (MC) (mass ratio of 1:1:1:1:1:1) Polymer mixture B: polycaprolactone (PCL), polyethylene glycol (PEG), poly(methyl methacrylate) (PMMA) sodium alginate (SA), and hydroxyethyl cellulose (HEC) (mass ratio of 1:1:1:1:1).

### Solid Vapor Diffusion

Solid vapor diffusion was performed under 12 conditions. Approximately 20 mg of freeform Type A was added in a 3-mL vial. The solid were then placed into a 20-mL vial with 4 mL of the corresponding volatile solvents. The 20-mL vial was sealed with a cap and kept at RT allowing sufficient time for the organic vapor to interact with the solid. After 20 days evaporation, freeform Type A and freeform Type B was observed, and the results are summarized in Table 9-14.

TABLE 9-14
Summary of solid vapor diffusion experiments

| Solvent | Results |
|---|---|
| H₂O | Type A |
| DCM | Type A |
| CHCl₃ | Type A |
| EtOH | Type C |
| MeOH | Type A |
| ACN | Type A |
| THF | Type A |
| Acetone | Type A |
| EtOAc | Type A |
| EtOAc | Type A |
| IPA | Type B |
| DMSO | Type A |

### Anti-Solvent Addition

Anti-solvent addition was performed under 16 conditions. About 20 mg of freeform Type A was dissolved in corresponding solvent. The solution was filtered to obtain a clear solution and the solution was magnetically stirred (˜1000 rpm). This was followed by the slow addition of anti-solvent until either precipitate appeared, or the total volume of anti-solvent reached 5 mL. The obtained precipitate was isolated for XRPD analysis. Results in Table 9-15 showed that freeform Type A and freeform Type B were generated.

TABLE 9-15
Summary of anti-solvent addition experiments

| Solvent (v/v) | Anti-solvent | Results |
|---|---|---|
| DMSO | n-Butanol | Clear solution |
| | Acetone | Type B |
| | EtOAc | Type B |
| | Anisole | Type A |
| | H₂O | Type A |
| | ACN | Type A |
| | DCM | Type A* |
| | Toluene | Type B |
| MeOH | MIBK | Type B |
| | 2-MeTHF | Type A* |
| | H2O | Type A |
| | Methyl acetate | Type A + B |
| | MTBE | Type B |
| | CHCl₃ | Type A |
| | ACN | Type A |
| | m-Xylene | Type A |

*Solid was obtained after evaporation.

### Salt Hits Characterization

### HCl Salt

Six HCl salt forms named as HCl salt Type A, Type B, Type C, Type D, Type E and Type F were obtained during salt formation and re-preparation experiments. XRPD overlay of the forms is displayed in FIG. 134. HCl salt Type A, Type B and Type C samples were obtained by reaction of freeform with 1 equiv. HCl in MeOH or THF, EtOAc and ACN, respectively. HCl salt Type B/C transformed to HCl salt Type E after vacuum drying at RT for 5 hours. HCl salt Type D was obtained by reaction of freeform with 2 equiv. HCl in ACN. HCl salt Type F was obtained during re-preparation of HCl salt Type D. TGA/DSC/¹H NMR/HPLC/IC of HCl salt Type A, Type D, Type E and Type F was performed.

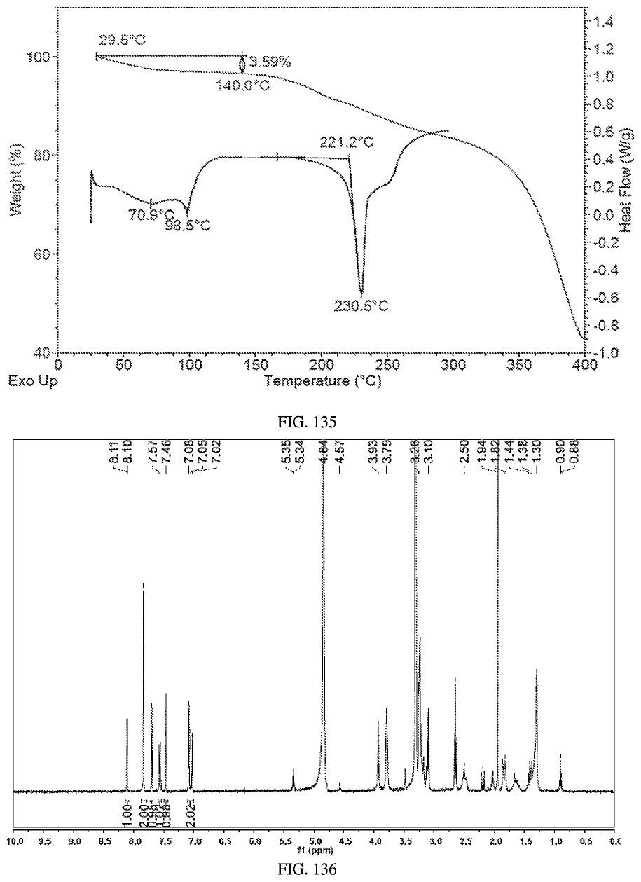

TGA/DSC curves of HCl salt Type A in FIG. 135 showed a weight loss of 3.6% up to 140° C. and three endotherms at 70.9° C. (peak), 98.5° C. (peak) and 221.2° C. (onset). ¹H NMR spectrum (FIG. 136) showed no signals of THF. HPLC/IC results showed the molar ratio of Cl⁻ to freeform was 0.3:1.0.

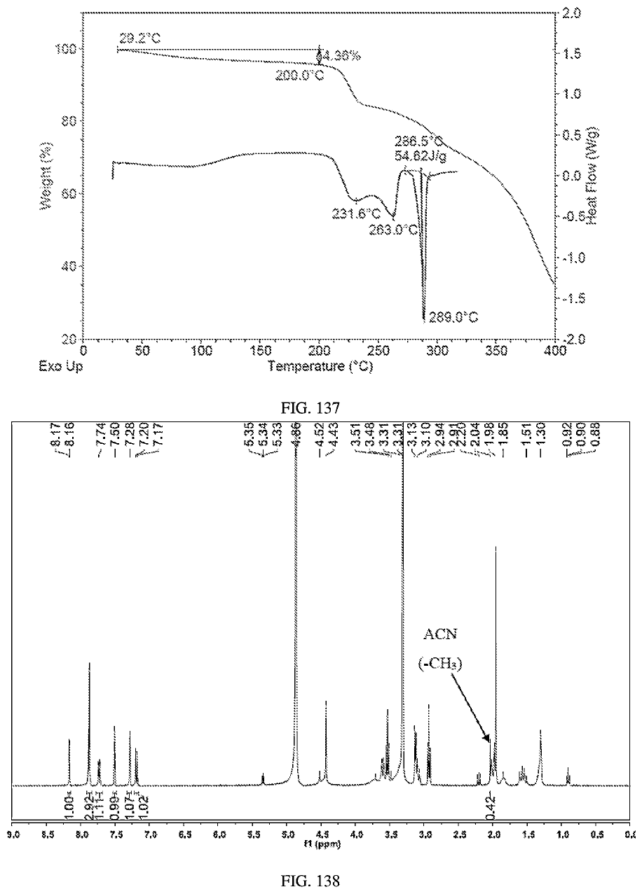

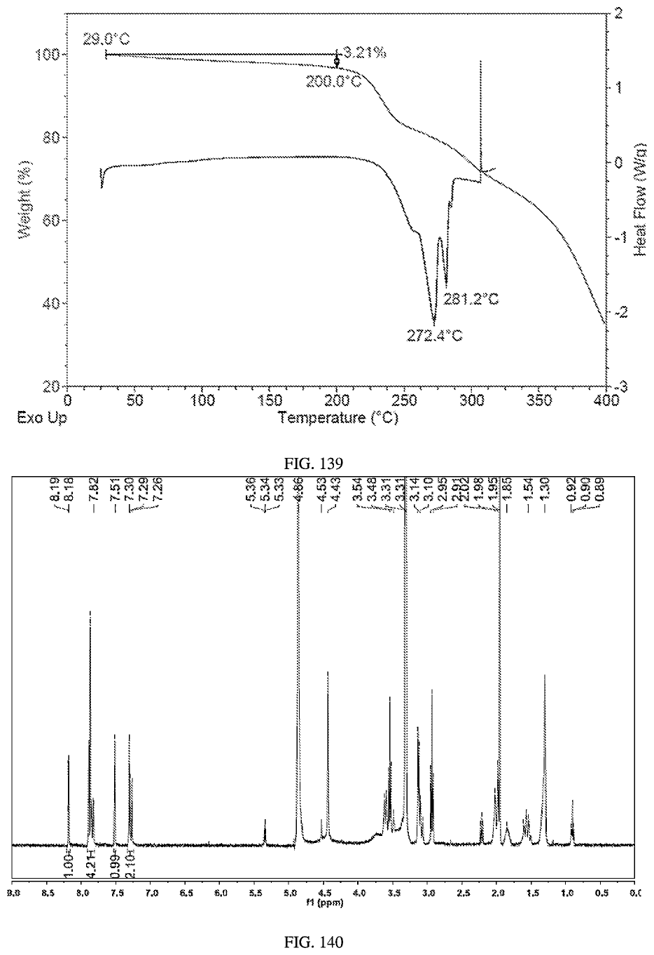

TGA/DSC curves of HCl salt Type D in FIG. 137 showed a weight loss of 4.4% up to 200° C. and three endotherms at 231.6° C. (peak), 263.0° C. (peak) and 286.6° C. (onset). ¹H NMR spectrum (FIG. 138) showed the molar ratio of ACN to freeform in the sample was 0.1:1 (0.8%). HPLC/IC results showed the molar ratio of Cl⁻ to freeform was 1.8:1.0. TGA/DSC curves of HCl salt Type E in FIG. 139 showed a weight loss of 3.2% up to 200° C. and two endotherms at 272.4° C. (peak) and 281.2° C. (peak). ¹H NMR spectrum (FIG. 140) showed no signals of EtOAc. HPLC/IC results showed the molar ratio of Cl⁻ to freeform was 2.4:1.0.

The characterization results of HCl salt Type F are summarized in Example 3.

### Sulfate

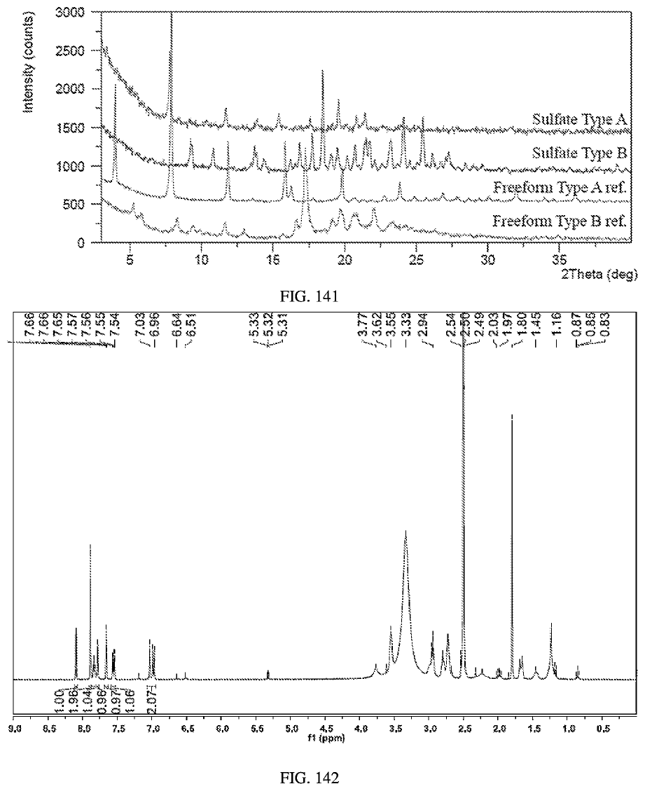

Two sulfate forms named as sulfate salt Type A and sulfate B were obtained during salt formation experiments. XRPD overlay of the forms is displayed in FIG. 141. Sulfate salt Type A and Type B samples were obtained by reaction of freeform with 1 equiv. sulfuric acid in MeOH and EtOAc, respectively. TGA/DSC/¹H NMR/HPLC/IC of sulfate Type A and Type B was performed.

¹H NMR spectrum (FIG. 142) of sulfate Type A showed no signals of MeOH. The sample amount of sulfate Type A was not enough for TGA/DSC/HPLC/IC tests.

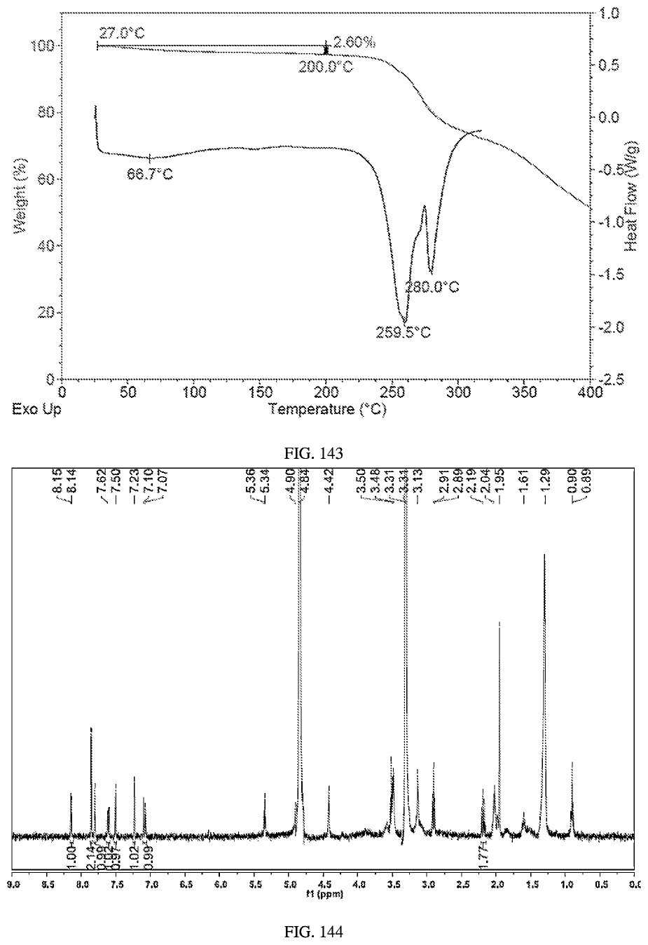

TGA/DSC curves of sulfate Type B in FIG. 143 showed a weight loss of 2.6% up to 200° C. and three endotherms at 66.7° C. (peak), 259.5° C. (peak) and 280.0° C. (peak). ¹H NMR spectrum (FIG. 144) showed no signals of acetone. HPLC/IC results showed the molar ratio of SO₄²⁻ to freeform was 1.0:1.0.

### Maleate

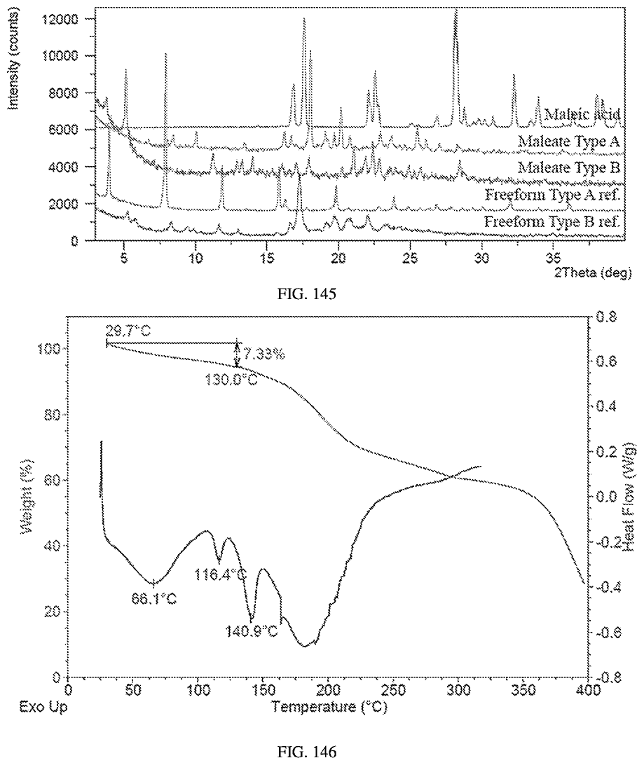

Two maleate forms named as maleate Type A and Type B were obtained during salt formation experiments. XRPD overlay of the forms is displayed in FIG. 145. Maleate Type A and Type B samples were obtained by reaction of freeform with 1 equiv. maleic acid in THF and ACN/H₂O (19:1, v/v), respectively. TGA/DSC/¹H NMR of maleate Type A and Type B was performed.

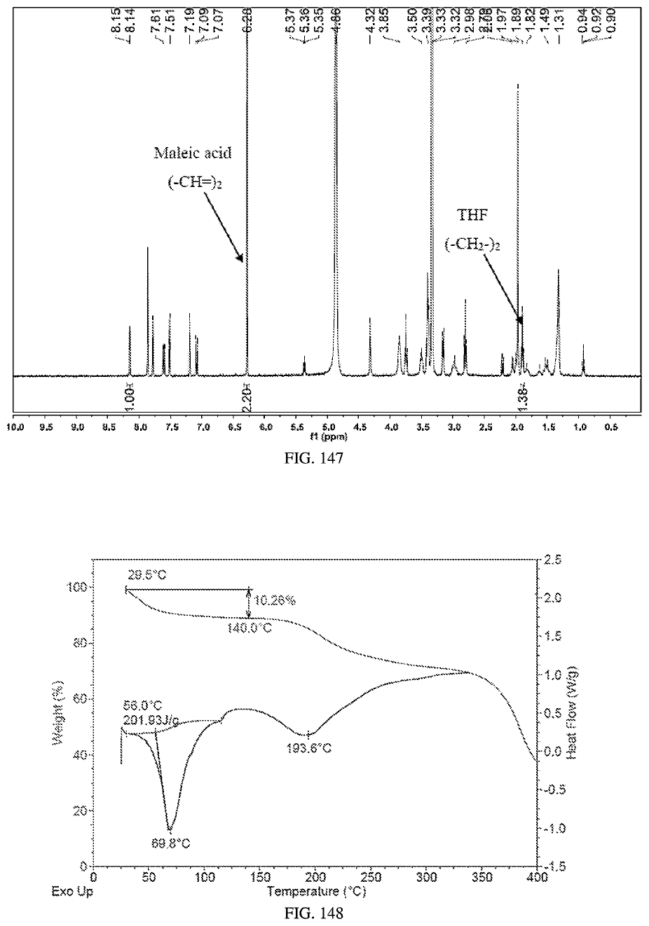

TGA/DSC curves of maleate Type A in FIG. 146 showed a weight loss of 7.3% up to 130° C. and three endotherms at 66.1° C. (peak), 116.4° C. (peak) and 140.9° C. (peak). ¹H NMR spectrum (FIG. 147) showed the molar ratio of THF to freeform was about 0.3:1.0 (˜2.6%), and the molar ratio of maleic acid to freeform was 1.1:1.0.

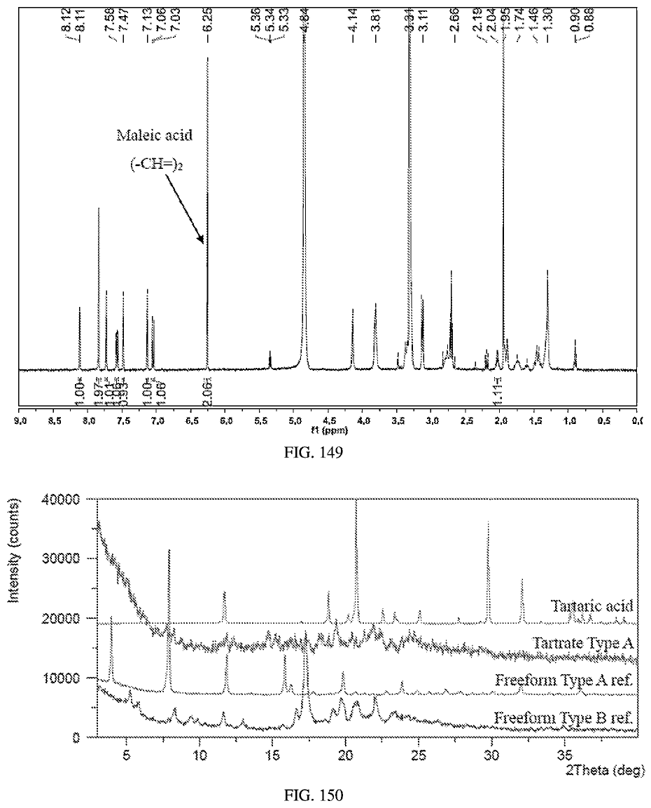

TGA/DSC curves of maleate Type B in FIG. 148 showed a weight loss of 10.3% up to 140° C. and two endotherms at 56.0° C. (onset) and 193.6° C. (peak). ¹H NMR spectrum (FIG. 149) showed no signals of ACN and the molar ratio of maleic acid to freeform was 1.0:1.0.

### Tartrate

One tartrate form named as tartrate Type A was obtained during salt formation experiments. XRPD pattern of the form is displayed in FIG. 150. Tartrate Type A sample was obtained by reaction of freeform with 1 equiv. tartaric acid in ACN/H₂O (19:1, v/v). TGA/DSC/¹H NMR of tartrate Type A was performed.

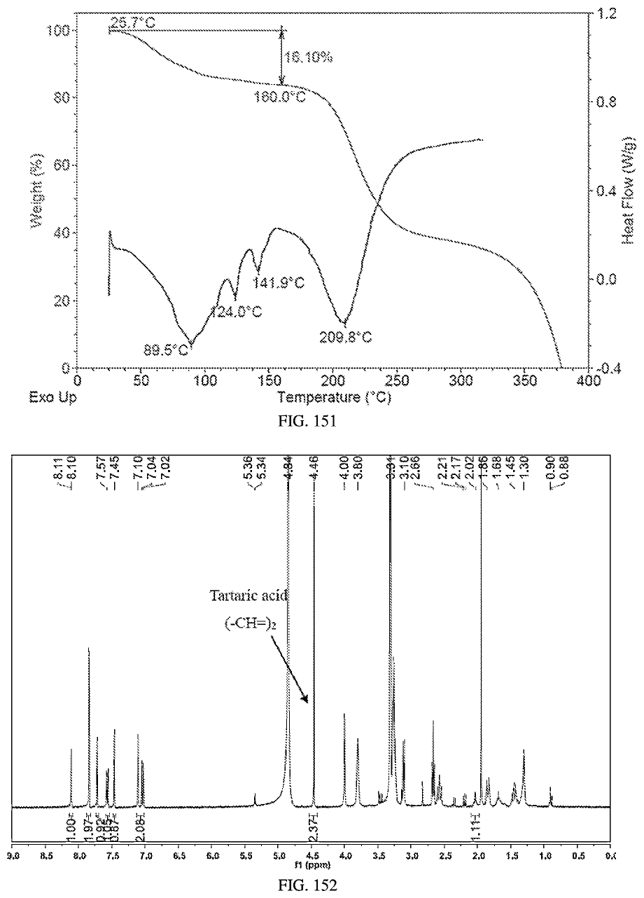

TGA/DSC curves of tartrate Type A in FIG. 151 showed a weight loss of 16.1% up to 160° C. and four endotherms at 89.5° C. (peak), 124.0° C. (peak), 141.9° C. (peak) and 209.8° C. (peak). ¹H NMR spectrum (FIG. 152) showed no signals of ACN, and the molar ratio of tartaric acid to freeform was 1.2:1.0.

### Fumarate

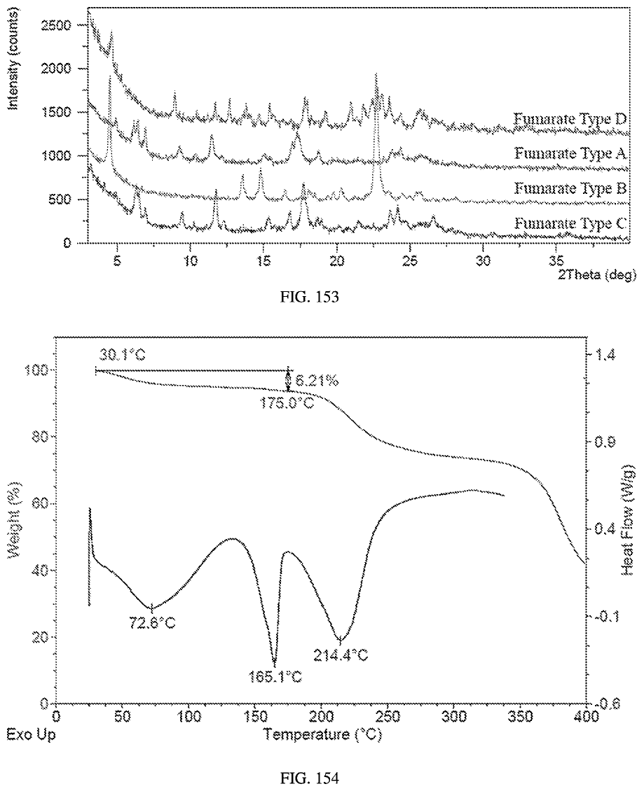

Four fumarate forms named as fumarate Type A, Type B, Type C and Type D were obtained during salt formation and re-preparation. XRPD overlay of the forms is displayed in FIG. 153. Fumarate Type A, Type B and Type C samples were obtained by reaction of freeform with 1 equiv. fumaric acid in MeOH, THF or EtOAc or acetone and ACN/H₂O (19:1, v/v), respectively. Fumarate salt Type D was observed during re-preparation of fumarate Type B in acetone, and the sample transferred to a mixture of fumarate Type A and Type B after vacuum drying overnight. TGA/DSC/¹H NMR of fumarate Type A, Type B and Type C was performed.

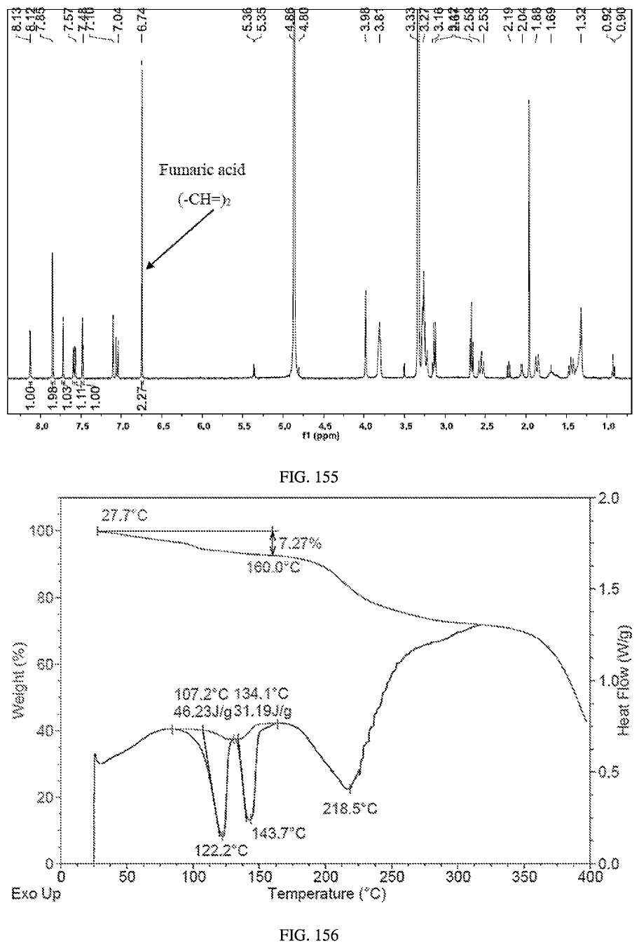

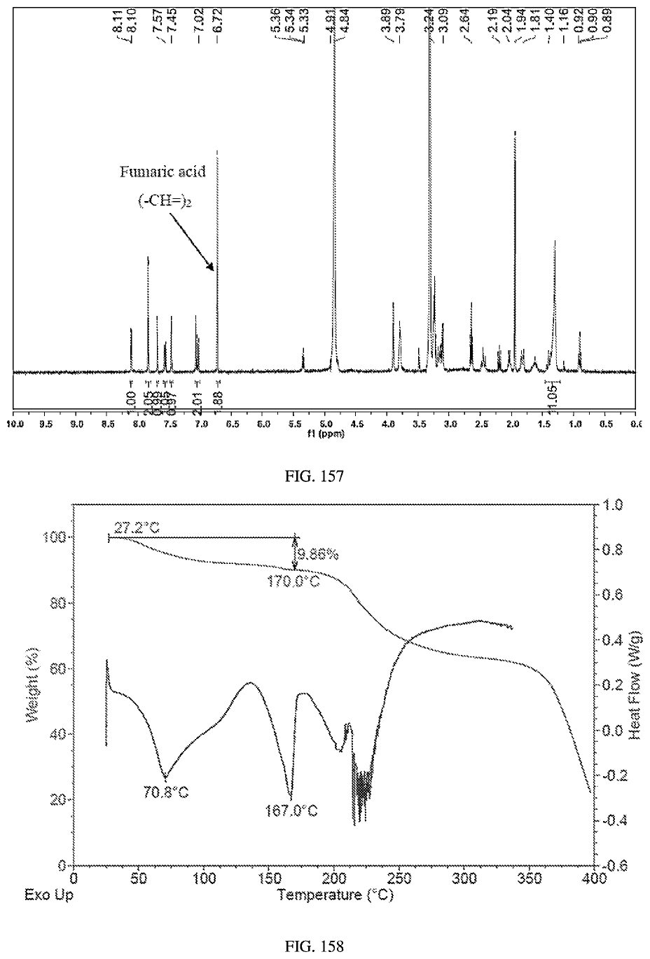

TGA/DSC curves of fumarate Type A in FIG. 154 showed a weight loss of 6.2% up to 175° C. and three endotherms at 72.6° C. (peak), 165.1° C. (peak) and 214.4° C. (peak). ¹H NMR spectrum (FIG. 155) showed the molar ratio of fumaric acid to freeform was 1.1:1.0. TGA/DSC curves of fumarate Type B in FIG. 156 showed a weight loss of 7.3% up to 160° C. and three endotherms at 107.2° C. (onset), 134.1° C. (onset) and 218.5° C. (peak)¹H NMR spectrum (FIG. 157) showed no signals of THF, and the molar ratio of fumaric acid to freeform was 0.9:1.0.

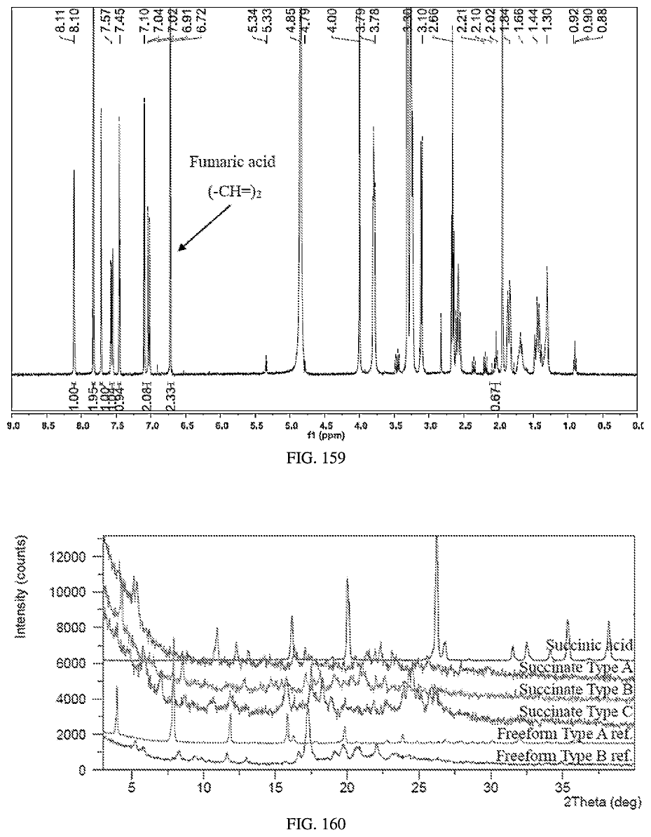

TGA/DSC curves of fumarate Type C in FIG. 158 showed a weight loss of 9.9% up to 170° C. and two endotherms at 70.8° C. (peak) and 167.0° C. (peak)¹H NMR spectrum (FIG. 159) showed no signals of ACN, and the molar ratio of fumaric acid to freeform was 1.2:1.0.

### Succinate

Three succinate forms named as succinate Type A, Type B and Type C were obtained during salt formation experiments. XRPD overlay of the forms is displayed in FIG. 160. Succinate Type A, Type B and Type C samples were obtained by reaction of freeform with 1 equiv. succinic acid in THF, IPA and ACN/H₂O (19:1, v/v), respectively. TGA/DSC/1H NMR of succinate Type A, Type B and Type C was performed.

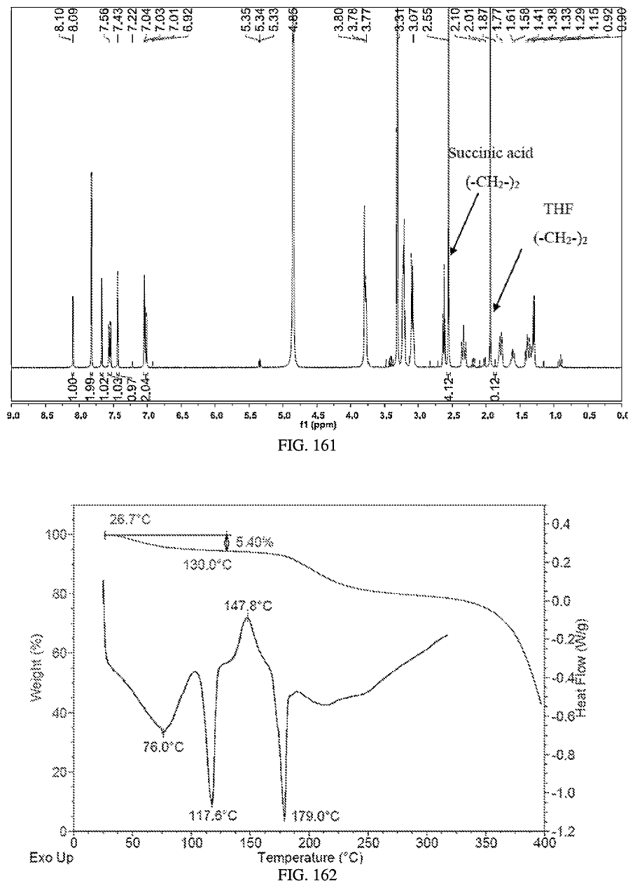

¹H NMR spectrum (FIG. 161) of succinate Type A showed the molar ratio of THF to freeform was about 0.03:1.0, and the molar ratio of succinic acid to freeform was 1.0:1.0.

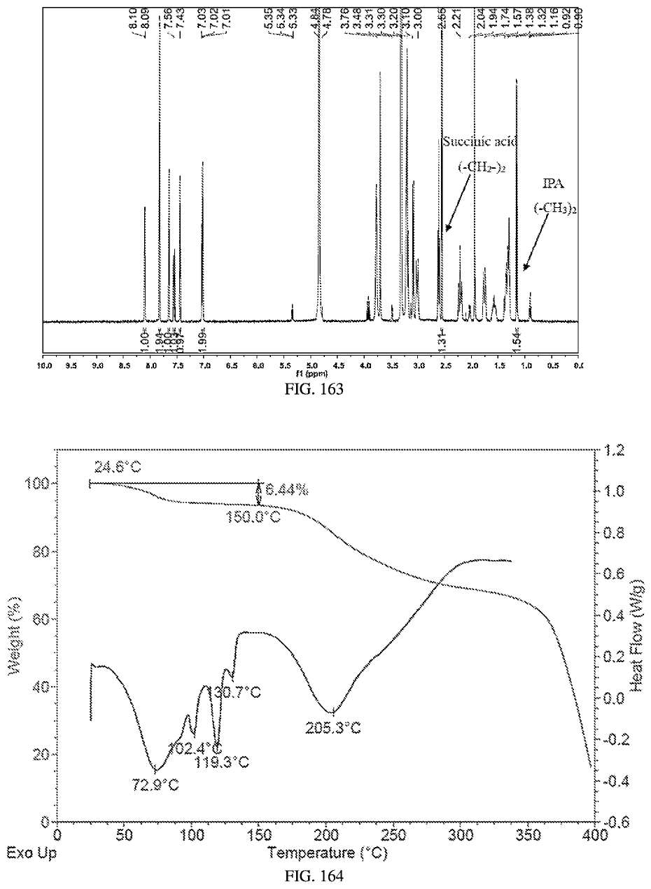

TGA/DSC curves of succinate Type B in FIG. 162 showed a weight loss of 5.4% up to 130° C. and three endotherms at 76.0° C. (peak), 117.6° C. (peak) and 179.0° C. (peak), and one exothermic peak at 147.8° C. (peak). ¹H NMR spectrum (FIG. 163) showed the molar ratio of IPA to freeform in was about 0.3:1.0 (˜2.6%), and the molar ratio of succinic acid to freeform was 0.3:1.0.

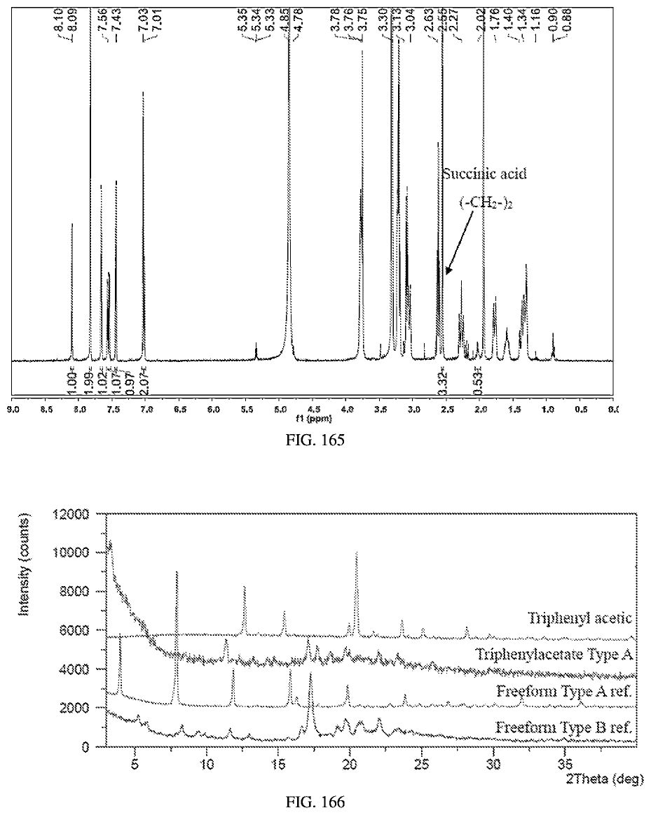

TGA/DSC curves of succinate Type C in FIG. 164 showed a weight loss of 6.4% up to 150° C. and five endotherms at 72.9° C. (peak), 102.4° C. (peak), 119.3° C. (peak), 130.7° C. (peak) and 205.3° C. (peak). ¹H NMR spectrum (FIG. 165) showed no signals of ACN, and the molar ratio of succinic acid to freeform was 0.8:1.0.

### Triphenylacetate

One triphenylacetate form named as triphenylacetate Type A was obtained during salt formation experiments. XRPD pattern of the form is displayed in FIG. 166. Triphenylacetate Type A sample was obtained by reaction of freeform with 1 equiv. triphenyl acetic in THF. TGA/DSC/¹H NMR of triphenylacetate Type A was obtained.

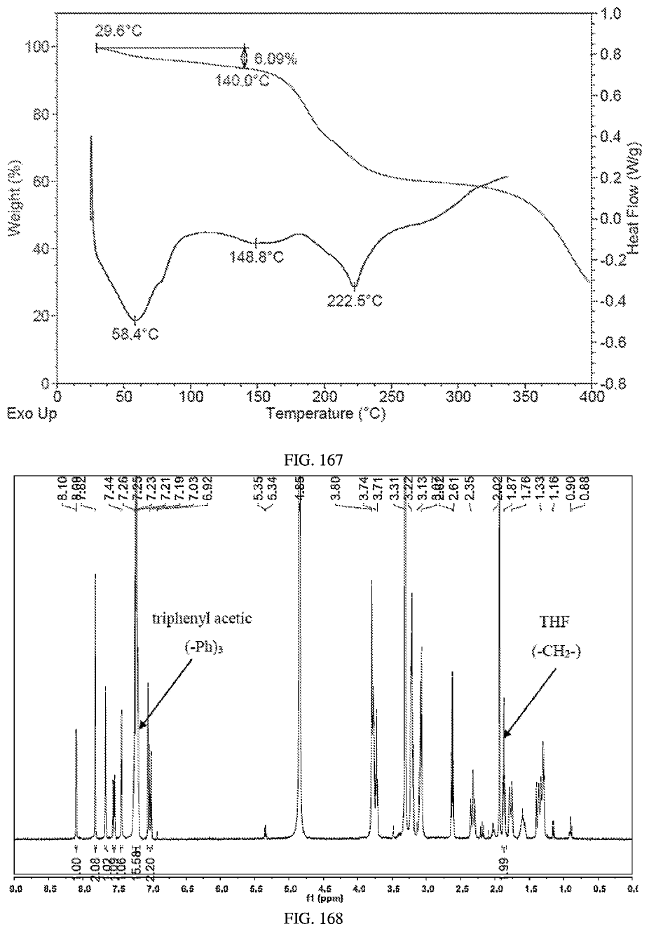

TGA/DSC curves of triphenylacetate Type A in FIG. 167 showed a weight loss of 6.1% up to 140° C. and three endotherms at 58.4° C. (peak), 148.8° C. (peak) and 222.5° C. (peak). ¹H NMR spectrum (FIG. 168) showed the molar ratio of THF to freeform was about 0.5:1.0 (˜3.7%), and the molar ratio of triphenyl acetic to freeform was 1.0:1.0.

### Xinafoic Salt

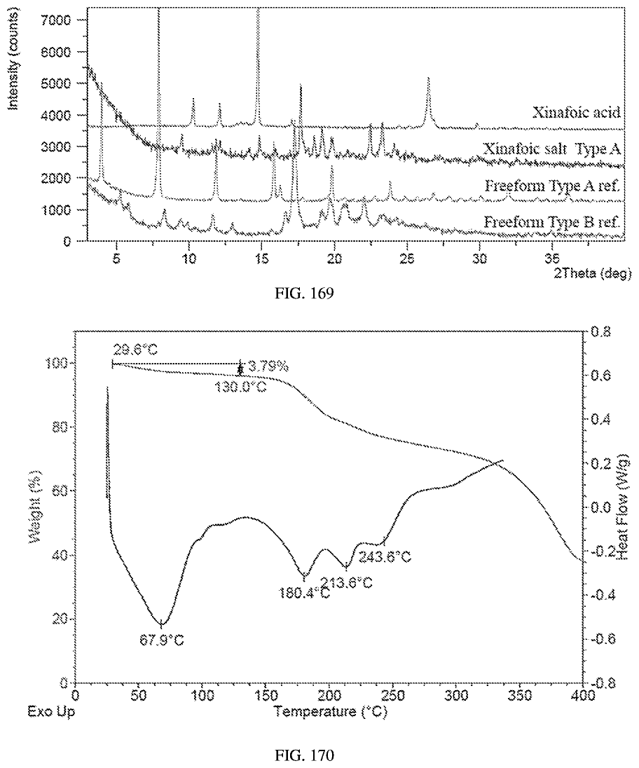

One xinafoic salt form named as xinafoic salt Type A was obtained during salt formation experiments. XRPD pattern of the form is displayed in FIG. 169. Xinafoic salt Type A sample was obtained by reaction of freeform with 1 equiv. xinafoic acid in THF. TGA/DSC/¹H NMR of xinafoic salt Type A was obtained.

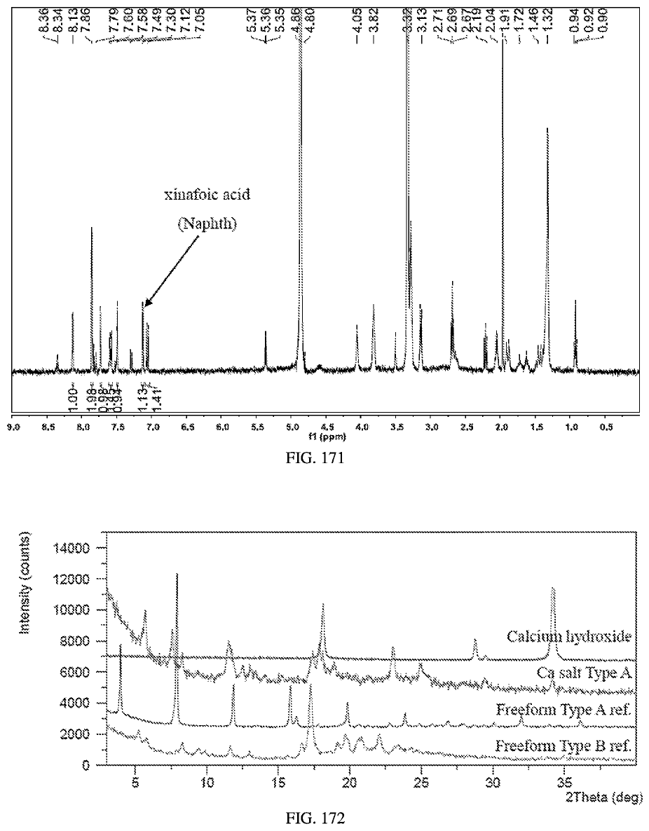

TGA/DSC curves of xinafoic salt Type A in FIG. 170 showed a weight loss of 3.8% up to 130° C. and four endotherms at 67.9° C. (peak), 180.4° C. (peak), 213.6° C. (peak) and 243.6° C. (peak). ¹H NMR spectrum (FIG. 171) showed no signals of THF, and the molar ratio of xinafoic acid to freeform was 1.1:1.0.

### Ca²⁺ Salt

One Ca²⁺ salt form named as Ca²⁺ salt Type A was obtained during salt formation experiments. XRPD pattern of the form is displayed in FIG. 172. Ca²⁺ salt Type A sample was obtained by reaction of freeform with 1 equiv. calcium hydroxide in ACN/H₂O (19:1, v/v). TGA/DSC/¹H NMR/HPLC/IC of Ca²⁺ salt Type A was obtained.

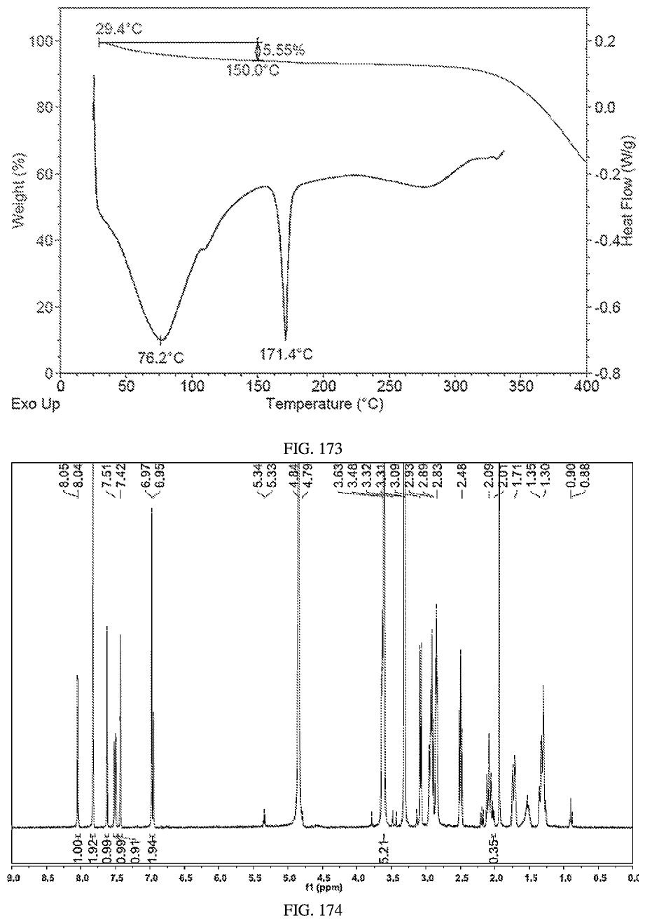

TGA/DSC curves of Ca²⁺ salt Type A in FIG. 173 showed a weight loss of 5.6% up to 150° C. and two endotherms at 76.2° C. (peak) and 171.4° C. (peak). ¹H NMR spectrum (FIG. 174) showed no signals of ACN. HPLC/IC results showed the molar ratio of Ca⁺ to freeform was 0.4:1.0.

### Tromethamine Salt

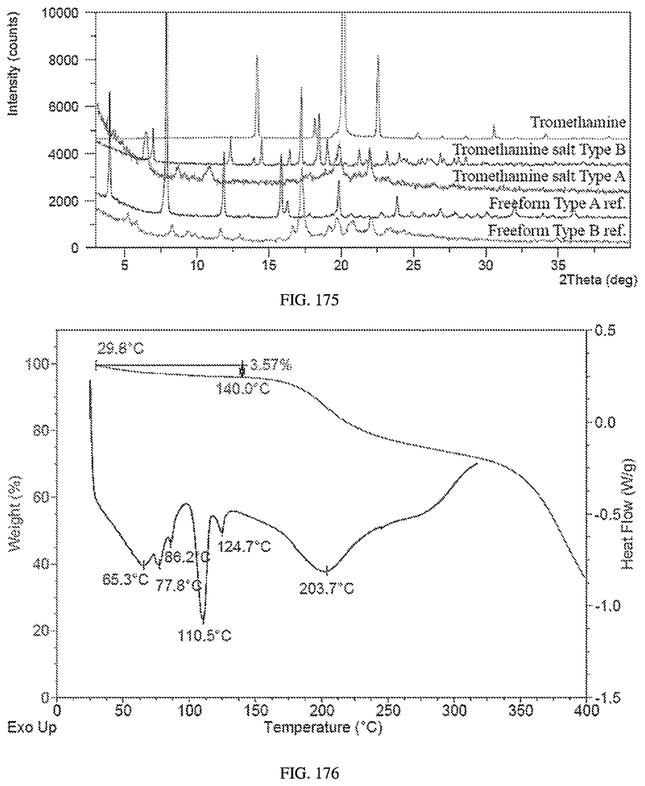

Two tromethamine salt forms named as tromethamine salt Type A and Type B were obtained during salt formation experiments. XRPD pattern of the form is displayed in FIG. 175. Tromethamine salt Type A and Type B samples were obtained by reaction of freeform with 1 equiv. tromethamine in IPA and ACN/H₂O (19:1, v/v), respectively. TGA/DSC/¹H NMR of tromethamine salt Type A and Type B was obtained.

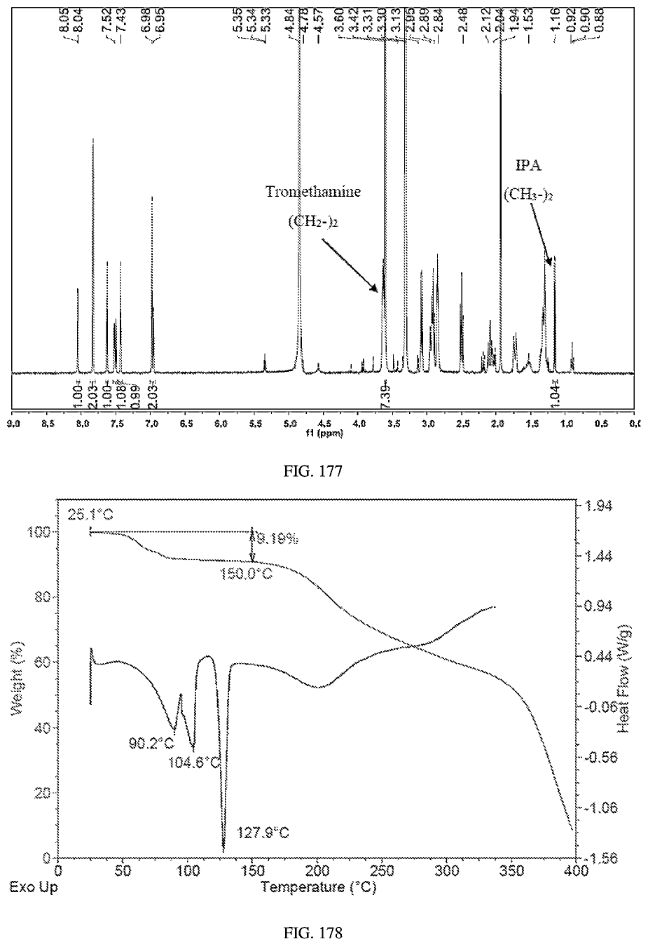

TGA/DSC curves of tromethamine salt Type A in FIG. 176 showed a weight loss of 3.6% up to 140° C. and six endotherms at 65.3° C. (peak), 77.8° C. (peak), 86.2° C. (peak), 110.5° C. (peak), 124.7° C. (peak) and 203.7° C. (peak). ¹H NMR spectrum (FIG. 177) showed the molar ratio of IPA to freeform was about 0.2:1.0 (1.4%), and the molar ratio of tromethamine to freeform was 1.8:1.0.

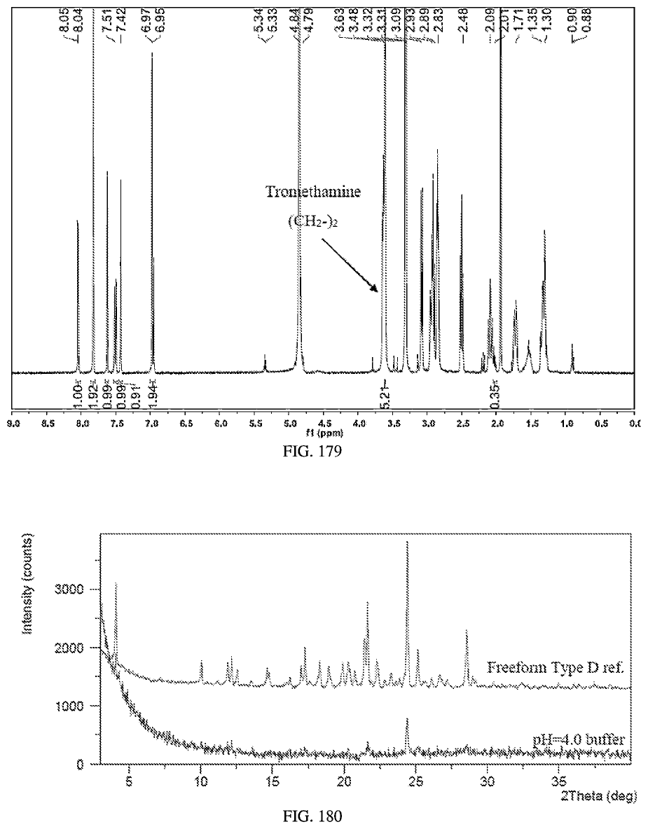

TGA/DSC curves of tromethamine salt Type B in FIG. 178 showed a weight loss of 9.2% up to 150° C. and three endotherms at 90.2° C. (peak), 104.6° C. (peak) and 127.9° C. (peak). ¹H NMR spectrum (FIG. 179) showed no signals of ACN, and the molar ratio of tromethamine to freeform was 1.3:1.0.

Solubility of Freeform Type D with Sugars

To determine the impact of different sugars on solubility of freeform Type D, equilibrium solubility of freeform Type D in the presence of sugars (lactose or sucrose) at pH=5.0 was tested at RT. ˜35 mg of freeform Type D solids and 9.6 mg citric acid were suspended into 1 mL water, and two samples were prepared. The samples were sonicated for about 2 minutes, and the samples were almost clear (little solid was observed) and filtered for clear solution, pH of the solutions were 3.5. About 68.5 mg lactose and sucrose were added into the two solutions, respectively. The two samples were clear after adding sugar, and pH of the two samples remained 3.5. pH of the two solutions was adjusted to 5.1 by adding 1 M NaOH, and no solid precipitated out after pH adjustment. Another sample without sugar was also prepared, ˜21 mg of freeform Type D solids and 5.8 mg citric acid were suspended into 1 mL water.

The three solutions were stored under 5° C. for three days, and no solid precipitated out after cooling. The concentration and pH were tested for each sample. The results are summarized in FIG. 136.

TABLE 9-16
Summary of equilibrium solubility results
in citrate/phosphate buffers at RT

| | | Concen- | | |
|---|---|---|---|---|
| | Initial | tration | End pH | Purity |
| Samples | pH | (mg/mL) | (24 hours) | (Area % ) |
| 30 mmol freeform Type | 3.6 | 18.11 | 3.6 | 99.8 |
| D + 30 mmol citric acid | | | | |
| 50 mmol freeform Type | 5.1 | 26.45 | 5.1 | 99.8 |
| D + 50 mmol citric | | | | |
| acid + ~200 mmol lactose | | | | |
| 50 mmol freeform Type | 5.1 | 26.50 | 5.3 | 99.8 |
| D + 50 mmol citric | | | | |
| acid + ~200 mmol sucrose | | | | |

* The weak intensity might result from limited amount of solid for XRPD test.

Solubility of Freeform Type D in pH=4.0 and 5.0 Citrate/Phosphate Buffers

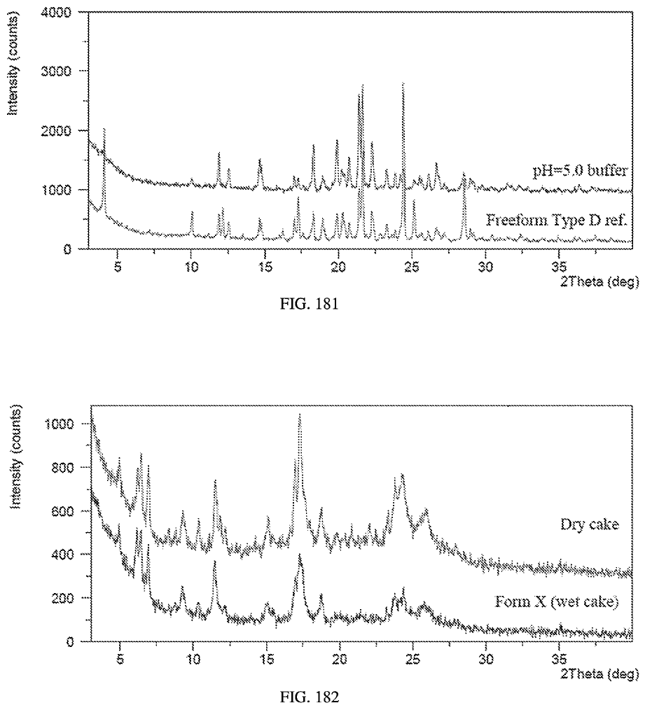

Equilibrium solubility of freeform Type D in pH=4.0 and 5.0 citrate/phosphate was evaluated at RT. ˜35 mg of solid was suspended into 1 mL of each medium with dosing conc. at ˜35 mg/mL. The suspension was equilibrated via stirring (1000 rpm) for 24 hours. The suspension was centrifuged to obtain precipitate and supernatants. Solubility and pH were tested for the supernatants after filtration, and isolated precipitate was tested by XRPD. The equilibrium solubility results are summarized in Table 9-17, and the XRPD results are shown in FIG. 180 and FIG. 181. Low crystallinity sample was obtained after equilibrium in pH=4.0 citrate/phosphate for 24 hours, and no form change of freeform Type D was observed after equilibrium in pH=5.0 citrate/phosphate for 24 hours.

TABLE 9-17
Summary of equilibrium solubility results
in citrate/phosphate buffers at RT

| | Initial | Solubility | End pH | |
|---|---|---|---|---|
| Buffer | pH | (mg/mL) | (24 hours) | Form |
| citrate/phosphate | 4.0 | 30.61 | 5.2 | Low crystallinity* |
| buffer | 5.0 | 1.29 | 5.2 | Freeform Type D |

*The weak intensity might result from limited amount of solid for XRPD test.

Formulation Development with NaCl

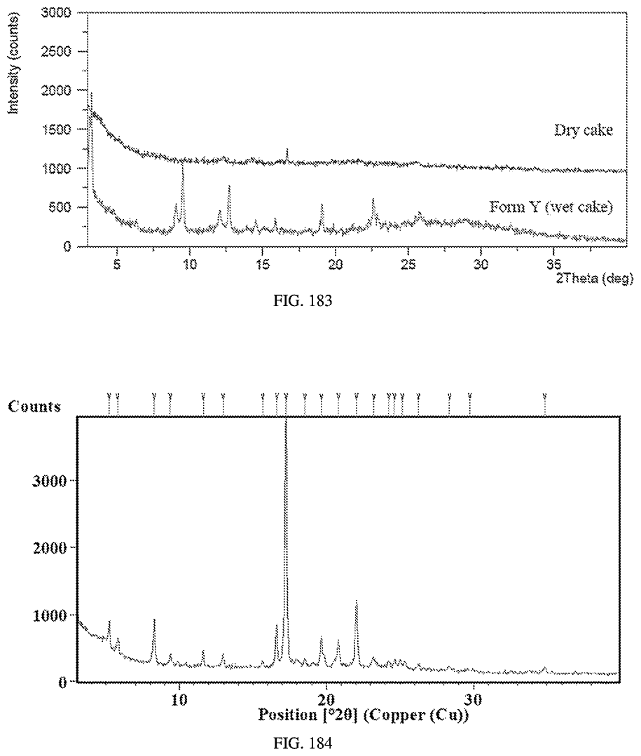

NaCl was selected for osmolarity adjustment of the formulation before the sugars were selected. ˜35 mg freeform Type D was combined with ˜9.6 mg citric acid+˜6.1 mg NaCl in water for 24 hours with stirring, and a suspension was obtained. The concentration of freeform in the suspension was 0.60 mg/mL (pH 3.8). The solid obtained was a new form (named as Form X, FIG. 182), HPLC/IC results showed the molar ratio between Cl⁻ to freeform was 1.0:1.0. The data suggest that the sample is an HCl salt. The experiment was redone, and a clear solution (a little cloudy) was obtained immediately after adding 1 mL water into ˜32 mg/mL API+9.6 mg/mL critic acid, solid precipitated out after addition of NaCl. The solid obtained was another new form (named as form Y, FIG. 183), and the form Y transformed to low crystallinity sample after vacuum drying. HPLC/IC results showed the molar ratio of Cl⁻ to freeform was 1.2:1.0.

### pH Buffer Preparation

1. pH=3.0 buffer (50 mM HCl+KCl):
   Weigh 365.39 mg KCl into a 100 mL volumetric flask (VF), and add 0.1 mL 1M HCl into the VF, and dilute to volume with water.
2. pH=4.0 buffer (50 mM citrate):
   Weigh 56.74 mg citric acid and 60.18 mg trisodium citrate into a 10 mL volumetric flask, and dilute to volume with water.
3. pH=5.0 buffer (50 mM citrate)
   Weigh 11.12 mg citric acid and 95.38 mg trisodium citrate into a 10 mL volumetric flask, and dilute to volume with water.
4. pH=6.0 buffer (50 mM citrate)
   Weigh 11.04 mg citric acid and 130.2 mg trisodium citrate into a 10 mL volumetric flask, and dilute to volume with water.
5. pH=6.0 buffer (50 mM phosphate)
   Weigh 8.7 mg Na₂HPO₄ and 52.6 mg NaH₂PO₄ into a 10 mL volumetric flask, and dilute to volume with water.
6. pH=7.0 buffer (50 mM phosphate)
   Weigh 23.47 mg Na₂HPO₄ and 43.34 mg NaH₂PO₄into a 10 mL volumetric flask, and dilute to volume with water.
7. pH=8.0 buffer (50 mM phosphate)
   Weigh 31.64 mg Na₂HPO₄ and 67.18 mg NaH2PO₄into a 10 mL volumetric flask, and dilute to volume with water.

### Instruments and Methods

### XRPD

For XRPD analysis, PANalytical X′Pert3 X-ray powder diffractometer was used. The XRPD parameters used are listed in Table 9-18.

TABLE 9-18
Parameters for XRPD test

| | | |
|---|---|---|
| | Empyrean | X′ Pert³ |
| Parameters | (reflection mode) | (reflection mode) |

| | |
|---|---|
| X-Ray | Cu, Kα, Kα1 (Å): 1.540598; |
| | Kα2 (Å): 1.544426 |
| | Kα2/Kα1 intensity ratio: 0.50 |

| | | |
|---|---|---|
| X-Ray tube setting | 45 kV, 40 mA | 45 kV, 40 mA |
| Divergence slit | Automatic | ⅛° |
| Scan mode | Continuous | Continuous |
| Scan range (2Theta) | 3°~40° | 3°~40° |
| Scan step time (s) | 0.0167° | 0.0263° |
| Step size (2Theta) | 17.78 | 46.67 |
| Test time (min) | ~5.5 min | ~5 min |

### TGA and DSC

TGA data were collected using a TA Discovery5500/Q5000 TGA from TA Instruments. DSC was performed using a TA Discovery2500/Q2000 DSC from TA Instruments. Detailed parameters used are listed in Table 9-19.

TABLE 9-19
Parameters for TGA and DSC test

| Parameters | TGA | DSC |
|---|---|---|
| Method | Ramp | Ramp |
| Sample pan | Aluminum, open | Aluminum, crimped/open |
| Temperature | RT - desired temperature | 25° C. - desired temperature |
| Heating rate | 10° C./min | 10° C./min |
| Purge gas | N₂ | N₂ |

### DVS

DVS was measured via a SMS (Surface Measurement Systems) DVS Intrinsic. Parameters for DVS test are listed in Table 9-20.

TABLE 9-20
Parameters for DVS test

| Parameters | Value | |
|---|---|---|
| Temperature | 25° | C. |
| Sample size | 10-20 | mg |
| Gas and flow rate | N₂, 200 mL/min | |
| dm/dt | 0.002%/min | |
| Min. dm/dt stability | 10 | min |
| duration | | |
| Max. equilibrium time | 180 | min |

| | |
|---|---|
| RH range | 0% RH~95% RH~0% RH |
| RH step size | 10% (0% RH-90% RH-0% RH) |
| | 5% (90% RH-95% RH and |
| | 95% RH-90% RH) |

### IC

ThermoFisher ICS-1100 was used for Ionic Chromatography (IC) analysis of stoichiometry. Detailed method is shown in Table 9-21.

TABLE 9-21
Parameters for IC test

| Parameter | IC (Thermo ICS1100) | |
|---|---|---|
| Column | IonPac AS18 Analytical | |
| | Column (4 × 250 mm) | |
| Mobile phase | 25 mM NaOH | |
| Injection volume | 25 | μL |
| Flow rate | 1.0 | mL/min |
| Cell temperature | 35° | C. |
| Column temperature | 35° | C. |
| Current | 80 | mA |
| Run time | 18 | mins |

### UPLC

Waters H-class UPLC (ultra-performance liquid chromatography) was used for purity, solubility, assay and stability test. Detailed method is shown in Table 9-22, Table 9-23 and Table 9-24.

TABLE 9-22
Chromatographic conditions and parameters
for stability measurement (Example 6)

| UPLC | Waters H-class |
|---|---|
| Column | Waters Ascentis Express |
| | C18, 3.0 × 150 mm, 2.7 μm |
| Mobile phase | A: 0.1% TFA in Water |
| | B: 0.1% TFA in ACN |

| Gradient table | Time (min) | % B |
|---|---|---|
| | 0.0 | 10 |
| | 12.0 | 95 |
| | 14.0 | 95 |
| | 14.1 | 95 |
| | 16.0 | 10 |

| | |
|---|---|
| Run time | 16.0 min |
| Post time | 0.0 min |
| Flow rate | 0.6 mL/min |
| Injection volume | 5 μL |
| Detector wavelength | 254 nm |
| Column temperature | 35° C. |
| Sampler temperature | RT |
| Diluent | Acetonitrile/water (1:1) |

TABLE 9-23
Chromatographic conditions and parameters
for stability measurement (Example 6)

| UPLC | Waters H-class |
|---|---|
| Column | Acquity UPLC BEH Shield |
| | RP18 100 mm × 2.1 mm, 1.7 μm |
| Mobile phase | A: 10 mM NH₄HCO₃ in H₂O |
| | B: ACN |

| Gradient table | Time (min) | % B |
|---|---|---|
| | 0.0 | 10 |
| | 12.0 | 45 |
| | 16.0 | 95 |
| | 20.0 | 95 |
| | 20.1 | 10 |
| | 25.0 | 10 |

| | |
|---|---|
| Run time | 25.0 min |
| Post time | 0.00 min |
| Flow rate | 0.5 mL/min |
| Injection volume | 5 μL |
| Detector wavelength | 254 nm |
| Column temperature | 35° C. |
| Sampler temperature | RT |
| Diluent | Acetonitrile/water (1:1) |

TABLE 9-24
Chromatographic conditions and parameters for solubility test

| UPLC | Waters H-class |
|---|---|
| Column | Acquity UPLC BEH C18, |
| | 50 mm × 2.1 mm, 1.7 μm |
| Mobile phase | A: 0.1% NH₃H₂O in H₂O |
| | B: ACN |

| Gradient table | Time (min) | % B |
|---|---|---|
| | 0.0 | 5 |
| | 3.0 | 95 |
| | 3.5 | 95 |
| | 4.0 | 5 |
| | 5.0 | 5 |

| | |
|---|---|
| Run time | 5.0 min |
| Post time | 0.00 min |
| Flow rate | 0.8 mL/min |
| Injection volume | 5 μL |
| Detector wavelength | 254 nm |
| Column temperature | 40° C. |
| Sampler temperature | RT |
| Diluent | Acetonitrile/water (1:1) |

### KF

Metrohm 870 KF Titrinoplus was used for KF test, and the instrument calibrated using purified water and the titration reagent was Hydranal® R-Composite 5 provided by Sigma-Aldrich. HPLC grade methanol was used to dissolve samples.

### ¹H NMR

¹H NMR was collected on Bruker 400M NMR Spectrometer using MeOH-d6 as the solvent

### Microscope

The image of the single crystal sample was captured using Shanghai Cewei PXS9-T stereo microscope. PLM images were captured using Axio Scope A1 microscope from Carl Zeiss German.

### Example 8. PK Testing of Compound (I)

### Materials and Methods

### Animals

Fifty (50) inbred, 6-8 week old, Sigmodon hispidus female and male cotton rats (source: Sigmovir Biosystems, Inc., Rockville MD) were maintained and handled under veterinary supervision in accordance with the National Institutes of Health guidelines and Sigmovir Institutional Animal Care and Use Committee's approved animal study protocol (IACUC Protocol #15). Fifteen animals were used in Phase I and 35 animals in Phase II studies. Each group of 5 animals included 3 females (the first three animals in each group) and 2 males (the last 2 animals in each group). Groups of even number of animals included equal number of males and females. Cotton rats were housed in clear polycarbonate cages and provided with standard rodent chow (Harlan #7004) and tap water ad lib.

### Preparation of Compound (I) Excipient Solution

To prepare excipient solution, 125 mg of citric acid anhydrous and 972 mg of lactose anhydrous were dissolved in water by adding 6 ml to the powder. The solution was vortexed. Final volume was adjusted to 10 mL with water for final concentration of 65 mM citric acid anhydrous and 284 mM lactose anhydrous. The solution was stored at 4±2° C.

### Experimental Study Design

### Phase I. PK Study.

### Day 0

Step 1. Divided 15 young S. hispidus (6-8 week old) between 3 groups (3 females and 2 males per group). Eartaged, weighed, and eyebleed all animals under isoflurane anesthesia for serum and plasma. Treated all animals intranasally with the solution indicated in Table 8-1 below, 5 0 μl/100 g animal (administered in both nostrils).

TABLE 8-1
Intranasal treatment regimen

| | # of | Treatment | Dose of | Time of |
|---|---|---|---|---|
| Group | animals | Route | Compound (I) | Sacrifice |
| 1 | 5 | IN | 0.1 mg/kg | D 4-5 |
| 2 | 5 | IN | 0.3 mg/kg | D 4-5 |
| 3 | 5 | IN | 1.0 mg/kg | D 4-5 |

### Day 1

Step 2. Measured weight, collected clinical observations (e.g., changes in appearance, movement, posture), and repeated treatments on all animals as in Step 1.

### Day 2

Step 3. Measured weight, collected clinical observations (e.g., changes in appearance, movement, posture), and repeated treatments on all animals as in Step 1.

### Day 3

Step 4. Measured weight, collected clinical observations (e.g., changes in appearance, movement, posture), and repeated treatments on all animals as in Step 1.

### Day 4

Step 5. Measured weight, collected clinical observations, and repeated treatment on all animals as described in Step 1. After 1 h Step 6, terminally bled one animal from each group, followed by the necropsy with gross pathology examination and collection of BAL (right lung) and lung samples (left lung) for the PK assessment. After 3 h Step 7, terminally bled the second animal from each group, followed by the necropsy with gross pathology examination and collection of BAL and lung samples for the PK assessment.

Step 8. Terminally bled the third animal from each group, followed by the necropsy with gross pathology examination and collection of BAL and lung samples for the PK assessment.

Step 9. Terminally bled the fourth animal from each group, followed by the necropsy with gross pathology examination and collection of BAL and lung samples for the PK assessment.

### Day 5

Step 10. Measured weight and collected clinical observations on the remaining animals. Terminally bled animals, followed by the necropsy with gross pathology examination and collection of BAL and lung samples for the PK assessment.

TABLE 8-2
Summary of sample collection times and procedures

| Sample | | |
|---|---|---|
| Collections | Day(s) of Collection | # of Sets |
| Plasma | 0, 4 (1, 3, 6, 12 post | At terminal time points, |
| | dosing at day 4), 5 (24 | anesthetize rats with 2% |
| | hours post dose at day 4) | isoflurane inhaled with pure |
| | | oxygen and collect blood |
| | | samples by retro orbital |
| | | bleed. Transfer blood into |
| | | K2EDTA tubes. Centrifuge |
| | | at 5,000 rpm for 10 min at |
| | | 4° C. to separate plasma |
| | | and aliquot, snap-freeze in |
| | | liquid nitrogen and store |
| | | at −80° C. before |
| | | shipment for Compound (I) |
| | | concentration analysis |
| | | (AIT Bioscience). |
| Lungs | Day 4-5 (1 animal at each | Following blood collection, |
| | time point per dose group: | euthanatize rats by CO₂ |
| | 1 hour post dosing, 3 hours | asphyxiation. Remove lungs |
| | post dosing, 6 hours post | from the thorax, clean to |
| | dosing, 12 hours post | remove excess tissue, weigh, |
| | dosing), day 5 (24 hours | tie off the left lung, cut |
| | post dosing at day 4) | off the upper lobe, bisect, |
| | | snapfreeze (in the same tube) |
| | | and store at −80° C. until |
| | | shipment for analysis of |
| | | drug concentration. |
| BAL | Day 4-5 (1 animal at each | After cutting off the left |
| | time point per dose group: | lung, collect BAL from the |
| | 1 hour post dosing, 3 hours | right lung by sequential |
| | post dosing, 6 hours post | infusions/aspirations of |
| | dosing, 12 hours post | PBS, snap-freeze in 2 |
| | dosing), day 5 (24 hours | different tubes, and store |
| | post dosing at day 4) | at −80° C. until |
| | | shipment for analysis of |
| | | drug concentration. |

TABLE 8-3
Determination of administration volume (for intranasal delivery).

| Body weight (g) | Test material volume (μL) |
|---|---|
| 100-91 | 25 |
| 90-81 | 22.5 |
| 80-71 | 20 |
| 70-61 | 17.5 |

### Phase I. PK Study.

In the Phase I study, three different doses of Compound (I): 0.1 mg/kg, 0.3 mg/kg, and 1 mg/kg per day were tested in young cotton rats S. hispidus, when administered once a day (QD) via the intranasal route. Treatments were given for 5 consecutive days. After the last treatment, lungs, BAL, and plasma were collected for PK analysis at 1 h, 3 h, 6 h, 12 h, and 24 h post-final-Compound (I) administration and shipped to AIT Bioscience, as requested. Overall, treatments with Compound (I) were well tolerated by cotton rats. The only noticeable effect was an apparent increase in blood clotting time in one of the animals treated with the highest dose of Compound (I) (1 mg/kg/day). Blood could not be collected effectively from that animal at the time of terminal bleed as it clotted too quickly.

Results of the PK analysis showed that Compound (I) was detectable in the plasma and lungs (but much less so in BALF) of Compound (I)-treated cotton rats. A dose-dependent effect on Compound (I) concentration in plasma and lungs was seen, with 1 mg/kg treatment resulting in the highest level of Compound (I) detected in plasma and lungs 1 hr post-treatment, followed by 0.3 and 0.1 mg/kg doses of Compound (I). The level of Compound (I) detectable in plasma and lungs of treated animals remained elevated for several hours (depending on the dose of treatment).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A pharmaceutical composition comprising Compound (I), of the formula: 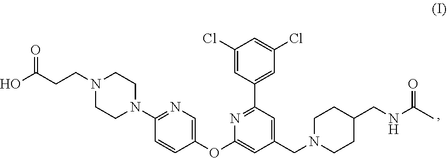 [SMILES: CC(=O)NCC1CCN(Cc2cc(Oc3ccc(N4CCN(CCC(=O)O)CC4)nc3)nc(-c3cc(Cl)cc(Cl)c3)c2)CC1 ; ","]
or a pharmaceutically acceptable salt, solvate, tautomer, stereoisomer, or isotopically labeled derivative thereof, or a polymorph thereof; and characterized in that it comprises an organic acid.

2. A pharmaceutical composition comprising Compound (I), of the formula: 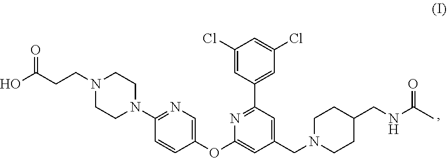 [SMILES: CC(=O)NCC1CCN(Cc2cc(Oc3ccc(N4CCN(CCC(=O)O)CC4)nc3)nc(-c3cc(Cl)cc(Cl)c3)c2)CC1 ; ","]
or a pharmaceutically acceptable salt, solvate, tautomer, stereoisomer, or isotopically labeled derivative thereof or a polymorph thereof; and characterized in that it comprises an organic acid; and a pharmaceutically acceptable excipient.

3. The pharmaceutical composition of claim 1 or 2, wherein the organic acid is selected from the group consisting of vitamin C, citric acid, fumaric acid, acetic acid, dehydroacetic acid, ascorbic acid, sorbic acid, phytic acid, and any combination thereof.

4. The pharmaceutical composition of any one of claims 1 to 3, wherein the organic acid is citric acid.

5. The pharmaceutical composition of claim 4, wherein the citric acid is present in a molar ratio to Compound (I) of between about 0.8 to about 1.2, preferably between about 0.9 to about 1.1.

6. The pharmaceutical composition of any one of claims 2 to 5, wherein the pharmaceutically acceptable excipient is a tonicity agent.

7. The pharmaceutical composition of any one of claims 2 to 6, wherein the pharmaceutically acceptable excipient is selected from the group consisting of dextrose, mannitol, sodium chloride, potassium chloride, lactose, trehalose, propylene glycol, glycerin, and any combination thereof.

8. The pharmaceutical composition of any one of claims 2 to 7, wherein the pharmaceutically acceptable excipient is lactose.

9. The pharmaceutical composition of claim 8, wherein the lactose is present in amount to achieve isotonicity with human tissue.

10. The pharmaceutical composition of any one of claims 1 to 9, wherein the composition is an aqueous solution.

11. The pharmaceutical composition of claim 10, wherein the pH of the aqueous solution is between about 2 and about 8.

12. The pharmaceutical composition of claim 10 or 11, wherein the pH of the aqueous solution is between about 3.5 and about 6.

13. The pharmaceutical composition of claim any one of claims 10 to 12, wherein the concentration of Compound (I) in the aqueous solution is between 10 and 50 mM, preferable between 35 and 45 mM.

14. The pharmaceutical composition of any one of claims 10 to 13, wherein the concentration of Compound (I) is 40 mM, the concentration of citric acid is 40 mM, and the concentration of lactose is 173 mM.

15. The pharmaceutical composition of any one of claims 10 to 14, wherein the solution is isotonic with human bodily fluid or human tissue.

16. The pharmaceutical composition of any one of claims 1 to 9, wherein at least a portion of Compound (I) or the pharmaceutically acceptable salt, solvate, tautomer, stereoisomer, or isotopically labeled derivative thereof, is in the form of a polymorph.

17. The pharmaceutical composition of any one of claims 1 to 9, or 16, wherein at least a portion of Compound (I) is in the form of a polymorph, wherein the polymorph is Freeform Type D.

18. The pharmaceutical composition of claim 16 or 17, wherein the polymorph is greater than or equal to 95% by weight Freeform Type D.

19. The pharmaceutical composition of any one of claims 16 to 18, wherein the polymorph is greater than or equal to 99% by weight Freeform Type D.

20. The pharmaceutical composition of claim 16 or 17, wherein the molar ratio of the amount of Freeform Type D to the sum of the amounts of other forms is equal to or greater than 90:10.

21. The pharmaceutical composition of any one of claims 16, 17, or 20, wherein the molar ratio of the amount of Freeform Type D to the sum of the amounts of other forms is equal to or greater than 95:5.

22. The pharmaceutical composition of any one of claims 16, 17, 20, or 21, wherein the molar ratio of the amount of Freeform Type D to the sum of the amounts of other forms is equal to or greater than 99:1.

23. The pharmaceutical composition of claim 16 or 17, wherein the polymorph comprises Freeform Type D in essentially pure form.

24. The pharmaceutical composition of any one of claims 1 to 9, or 16 comprising a polymorph of Compound (I), wherein the polymorph is Fumarate Type A.

25. The pharmaceutical composition of claim 16 or 24, wherein the polymorph is greater than or equal to 95% by weight Fumarate Type A.

26. The pharmaceutical composition of any one of claims 16, 24, or 25, wherein the polymorph is greater than or equal to 99% by weight Fumarate Type A.

27. The pharmaceutical composition of claim 16, or 24, wherein the molar ratio of the amount of Fumarate Type A to the sum of the amounts of other forms is equal to or greater than 90:10.

28. The pharmaceutical composition of claim 16, or 24, wherein the molar ratio of the amount of Fumarate Type A to the sum of the amounts of other forms is equal to or greater than 95:5.

29. The pharmaceutical composition of claim 16, or 24, wherein the molar ratio of the amount of Fumarate Type A to the sum of the amounts of other forms is equal to or greater than 99:1.

30. The pharmaceutical composition of claim 16, or 24, wherein the polymorph comprises Fumarate Type A in essentially pure form.

31. The pharmaceutical composition of any one of claims 1 to 9, comprising an amorphous form of Compound (I), or a pharmaceutically acceptable salt, solvate, tautomer, stereoisomer, or isotopically labeled derivative thereof.

32. The pharmaceutical composition of any one of claims 1 to 9, or 16 to 31, wherein the composition is a powder.

33. The pharmaceutical composition of any one of claims 1 to 9, or 16 to 32, wherein composition is obtained by lyophilization of the aqueous solution of any one of claims 10 to 15.

34. The pharmaceutical composition of any one of claims 1 to 33, wherein the composition is formulated for oral or nasal inhalation.

35. The pharmaceutical composition of any one of claims 1 to 34, wherein the composition is formulated for administration with a nebulizer or a dry powder inhaler.

36. The pharmaceutical composition of any one of claims 1 to 35, for use in therapy.

37. The pharmaceutical composition of any one of claims 1 to 36, for use in in the treatment of fibrotic disease.

38. The pharmaceutical composition of claim 37, wherein the fibrotic disease is pulmonary fibrosis.

39. The pharmaceutical composition of any one of claims 1 to 36, for use in in the treatment of cystic fibrosis.

40. Use of the pharmaceutical composition of any one of claims 1 to 36, in the manufacture of a medicament for the treatment of a fibrotic disease.

41. The use of claim 40, wherein the fibrotic disease is pulmonary fibrosis.

42. Use of the pharmaceutical composition of any one of claims 1 to 35, in the manufacture of a medicament for the treatment of a cystic fibrosis.

43. A method of treating a fibrotic disease or condition comprising administering to a subject in need thereof a therapeutically effective amount of the pharmaceutical composition of any one of claims 1 to 36.

44. The method of claim 43, wherein the fibrotic disease or condition is pulmonary fibrosis.

45. A method of treating a cystic fibrosis comprising administering to a subject in need thereof a therapeutically effective amount of the pharmaceutical composition of any one of claims 1 to 36.

46. A kit comprising:
a pharmaceutical composition of any one of claims 1 to 27; and
instructions for using the pharmaceutical composition.

47. The kit of claim 46, wherein the instructions are for using the kit to treat a fibrotic disease.

48. The kit of claim 46 or 47, wherein the instructions are for using the kit to treat pulmonary fibrosis.

49. The kit of claim 46, wherein the instructions are for using the kit to treat cystic fibrosis.

50. The kit of any one of claims 46 to 49, wherein the kit comprises a nebulizer, or a dry powder inhaler.
